(19) United States
(12) Patent Application Publication
(10) Pub. No.: US 2021/0043852 A1
(43) Pub. Date: Feb. 11, 2021

(54) HETEROCYCLIC COMPOUND AND ORGANIC LIGHT-EMITTING DEVICE INCLUDING THE SAME

(71) Applicant: Samsung Display Co., Ltd., Yongin-si (KR)

(72) Inventors: Minji KIM, Yongin-si (KR); Dongjun KIM, Yongin-si (KR); Eunjae JEONG, Yongin-si (KR); Sohee JO, Yongin-si (KR); Sanghyun HAN, Yongin-si (KR)

(21) Appl. No.: 16/885,001

(22) Filed: May 27, 2020

(30) Foreign Application Priority Data
Aug. 6, 2019 (KR) 10-2019-0095637

(51) Int. Cl. H01L 51/00; H01L 51/50; H01L 51/42

(52) U.S. Cl. CPC H01L 51/0074; H01L 51/0072; H01L 51/0073; H01L 51/5092; H01L 51/5056; H01L 51/4273; H01L 51/5072; H01L 51/5024

(57) ABSTRACT

A heterocyclic compound is represented by Formula 1. An organic light-emitting device includes: a first electrode; a second electrode facing the first electrode; and an organic layer between the first electrode and the second electrode and comprising an emission layer, wherein the organic layer comprises at least one of the heterocyclic compound represented by Formula 1.

### CROSS-REFERENCE TO RELATED APPLICATION

This application claims priority to and the benefit of Korean Patent Application No. 10-2019-0095637, filed on Aug. 6, 2019, in the Korean Intellectual Property Office, the entire content of which is incorporated herein in its entirety by reference.

### BACKGROUND

### 1. Field

One or more embodiments of the present disclosure relate to a heterocyclic compound and an organic light-emitting device including the same.

### 2. Description of the Related Art

Organic light-emitting devices are self-emission devices that produce full-color images, and also have wide viewing angles, high contrast ratios, short response times, as well as excellent characteristics in terms of brightness, driving voltage, and response speed.

The organic light-emitting device may include a first electrode on a substrate, and a hole transport region, an emission layer, an electron transport region, and a second electrode, which are sequentially on the first electrode. Holes provided from the first electrode may move toward the emission layer through the hole transport region, and electrons provided from the second electrode may move toward the emission layer through the electron transport region. Carriers, such as holes and electrons, recombine in the emission layer to produce excitons. These excitons transit from an excited state to a ground state, thereby generating light.

### SUMMARY

One or more embodiments include a novel heterocyclic compound and an organic light-emitting device including the same.

Additional aspects of embodiments will be set forth in part in the description which follows and, in part, will be apparent from the description, or may be learned by practice of the presented embodiments.

An aspect of an embodiment of the present disclosure provides a heterocyclic compound represented by Formula 1:

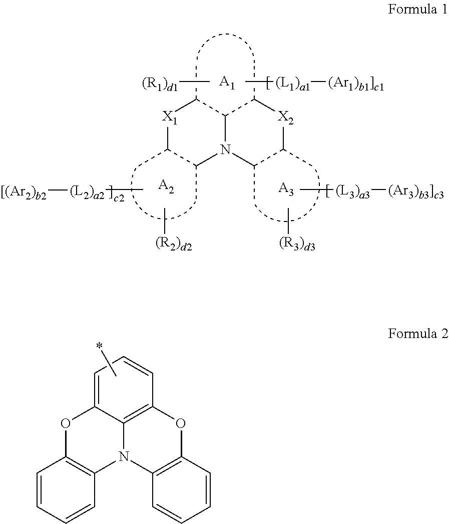

In Formula 1,

ring A₁ to ring A₃ may each independently be a C₅-C₆₀ carbocyclic group or a C₁-C₆₀ heterocyclic group,

X₁ and X₂ may each independently be O or S,

L₁ to L₃ may each independently be a substituted or unsubstituted C₅-C₆₀ carbocyclic group or a substituted or unsubstituted C₁-C₆₀ heterocyclic group,

a1 to a3 may each independently be an integer from 0 to 3,

Ar₁ to Ar₃ may each independently be a substituted or unsubstituted C₅-C₆₀ carbocyclic group or a substituted or unsubstituted C₁-C₆₀ heterocyclic group, wherein each of Ar₁ to Ar₃ is not a group represented by Formula 2,

b1 to b3 may each independently be an integer from 1 to 3,

c1 to c3 may each independently be an integer from 0 to 4,

c1+c2+c3≥1,

R₁ to R₃ may each independently be selected from deuterium, —F, —Cl, —Br, —I, a hydroxyl group, a cyano group, a nitro group, an amino group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₂-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₁)(Q₂), —B(Q₁)(Q₂), —C(═O)(Q₁), —S(═O)₂(Q₁), and —P(═O)(Q₁)(Q₂),

d1 to d3 may each independently be selected from 0 to 4,

at least one substituent of the substituted C₅-C₆₀ carbocyclic group, the substituted C₁-C₆₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₂-C₆₀ heteroaryl group, the substituted monovalent non-aromatic condensed polycyclic group, and the substituted monovalent non-aromatic condensed heteropolycyclic group may be selected from:

deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —Cl, —Br, —I, a hydroxyl group, a cyano group, a nitro group, an amidino group, a hydrazino group, a hydrazono group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), and —P(═O)(Q₁₁)(Q₁₂);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, a biphenyl group, and a terphenyl group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, a biphenyl group, and a terphenyl group, each substituted with at least one selected from deuterium, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, a biphenyl group, a terphenyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), and —P(═O)(Q₂₁)(Q₂₂); and

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂),

Q₁ to Q₃, Q₁₁ to Q₁₃, Q₂₁ to Q₂₃, and Q₃₁ to Q₃₃ may each independently be selected from hydrogen, deuterium, —C, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with a C₁-C₆₀ alkyl group, a C₁-C₆₀ heteroaryl group, a monovalent non-aromatic condensed polycyclic group, a monovalent non-aromatic condensed heteropolycyclic group, a biphenyl group, and a terphenyl group, and

* indicates a binding site to a neighboring atom.

Another aspect of an embodiment of the present disclosure provides an organic light-emitting device including: a first electrode; a second electrode facing the first electrode; and an organic layer between the first electrode and the second electrode and including an emission layer, wherein the organic layer includes at least one of the heterocyclic compound represented by Formula 1.

### BRIEF DESCRIPTION OF THE DRAWINGS

These and/or other aspects of embodiments will become apparent and more readily appreciated from the following description of the embodiments, taken in conjunction with the accompanying drawings in which:

FIGS. 1 to 4 are each a schematic view of an organic light-emitting device according to an embodiment.

### DETAILED DESCRIPTION

Reference will now be made in more detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described herein below, by referring to the figures, to explain aspects of embodiments of the present description. As used herein, the term “and/or” includes any and all combinations of one or more of the associated listed items. Expressions such as “at least one of,” when preceding a list of elements, modify the entire list of elements and do not modify the individual elements of the list.

An aspect of an embodiment of the present disclosure provides a heterocyclic compound represented by Formula 1 below:

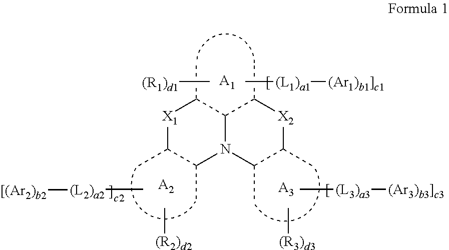

Ring A₁ to ring A₃ in Formula 1 may each independently be a C₅-C₆₀ cyclic group or a C₁-C₆₀ heterocyclic group.

In one embodiment, ring A₁ to ring A₃ in Formula 1 may each independently be selected from a benzene group, a pentalene group, an indene group, a naphthalene group, an anthracene group, a fluorene group, a phenanthrene group, a triphenylene group, a pyrene group, a chrysene group, a naphthacene group, a pyrrole group, an imidazole group, a pyrazole group, a pyridine group, a pyrimidine group, a pyrazine group, a pyridazine group, an indole group, an isoindole group, an indazole group, a quinoline group, an isoquinoline group, a benzoquinoline group, a naphthyridine group, a quinoxaline group, a quinazoline group, a cinnoline group, a phenanthridine group, an acridine group, a phenanthroline group, a phenazine group, a benzimidazole group, a furan group, a benzofuran group, a thiophene group, a benzothiophene group, a thiazole group, an isothiazole group, a benzothiazole group, an oxazole group, an isoxazole group, a benzoxazole group, a triazole group, an oxadiazole group, a triazine group, a carbazole group, a dibenzofuran group, a dibenzothiophene group, a benzocarbazole group, and a dibenzocarbazole group.

For example, ring A₁ to ring A₃ may each independently be selected from a benzene group, a naphthalene group, an anthracene group, a fluorene group, a pyridine group, a pyrimidine group, a pyrazine group, a pyridazine group, a quinoline group, an isoquinoline group, a benzoquinoline group, a naphthyridine group, a quinoxaline group, a quinazoline group, a triazine group, a carbazole group, a dibenzofuran group, a dibenzothiophene group, a benzocarbazole group, and a dibenzocarbazole group.

In one embodiment, ring A₁ to ring A₃ may be identical to each other.

In Formula 1, X₁ and X₂ may each independently be O or S.

For example, X₁ and X₂ may each be O. For example, X₁ may be O, and X₂ may be S. For example, X₁ may be S, and X₂ may be O. For example, each of X₁ and X₂ may be S.

In Formula 1, L₁ to L₃ may each independently be a substituted or unsubstituted C₅-C₆₀ carbocyclic group or a substituted or unsubstituted C₁-C₆₀ heterocyclic group.

In one embodiment, L₁ to L₃ in Formula 1 may each independently be selected from:

a benzene group, a pentalene group, an indene group, a naphthalene group, an azulene group, a heptalene group, an indacene group, an acenaphthalene group, a fluorene group, a spiro-bifluorene group, a benzofluorene group, a dibenzofluorene group, a phenalene group, a phenanthrene group, an anthracene group, a fluoranthene group, a pyrene group, a chrysene group, a naphthacene group, a picene group, a perylene group, a pyrrole group, a thiophene group, a furan group, a silole group, an imidazole group, a pyrazole group, a thiazole group, an isothiazole group, an oxazole group, an isoxazole group, a pyridine group, a pyrazine group, a pyrimidine group, a pyridazine group, a triazine group, a benzofuran group, a benzothiophene group, a dibenzofuran group, a dibenzothiophene group, a carbazole group, a benzosilole group, a dibenzosilole group, a quinoline group, an isoquinoline group, a benzimidazole group, an imidazopyridine group, and an imidazopyrimidine group; and

a benzene group, a pentalene group, an indene group, a naphthalene group, an azulene group, a heptalene group, an indacene group, an acenaphthalene group, a fluorene group, a spiro-bifluorene group, a benzofluorene group, a dibenzofluorene group, a phenalene group, a phenanthrene group, an anthracene group, a fluoranthene group, a pyrene group, a chrysene group, a naphthacene group, a picene group, a perylene group, a pyrrole group, a thiophene group, a furan group, a silole group, an imidazole group, a pyrazole group, a thiazole group, an isothiazole group, an oxazole group, an isoxazole group, a pyridine group, a pyrazine group, a pyrimidine group, a pyridazine group, a triazine group, a benzofuran group, a benzothiophene group, a dibenzofuran group, a dibenzothiophene group, a carbazole group, a benzosilole group, a dibenzosilole group, a quinoline group, an isoquinoline group, a benzimidazole group, an imidazopyridine group, and an imidazopyrimidine group, each substituted with at least one selected from deuterium, —F, —Cl, —Br, —I, a hydroxyl group, a cyano group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a pyrenyl group, a chrysenyl group, a naphthacenyl group, a picenyl group, a perylenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyrdinyl group, a pyrazinyl group, a pyrimidinyl group, a pyridazinyl group, a triazinyl group, a benzofuranyl group, a benzothiophenyl group, a dibenzofuranyl group, a dibenzothiophenyl group, a carbazolyl group, a benzosilolyl group, a dibenzosilolyl group, a quinolinyl group, an isoquinolinyl group, a benzimidazolyl group, an imidazopyridinyl group, an imidazopyrimidinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂), and

Q₃₁ to Q₃₃ may each independently be selected from a C₁-C₂₀ alkyl group, a C₁-C₂₀ alkoxy group, a phenyl group, a biphenyl group, a terphenyl group, a naphthyl group, and a pyridinyl group.

For example, L₁ to L₃ may each independently be a group represented by one of Formulae 3-1 to 3-39:

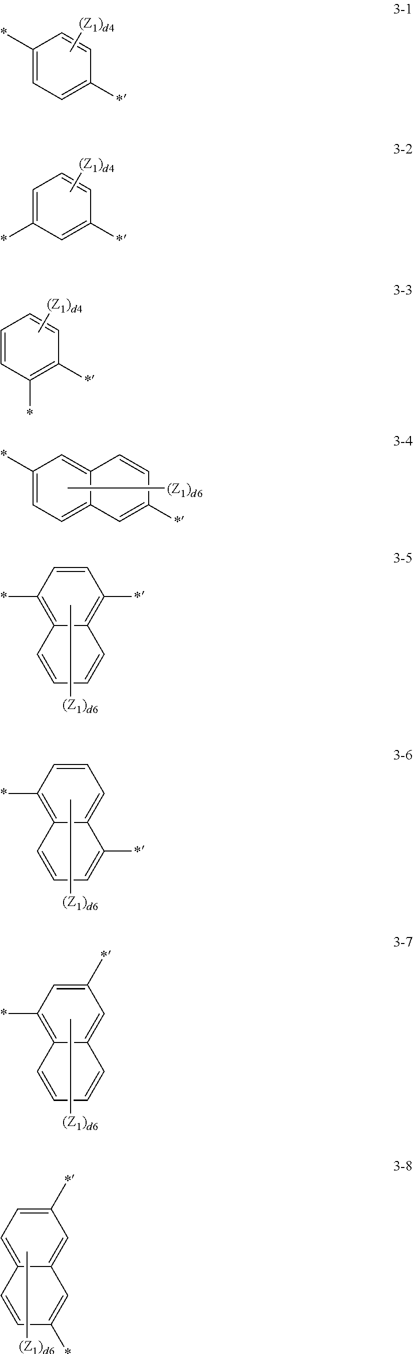

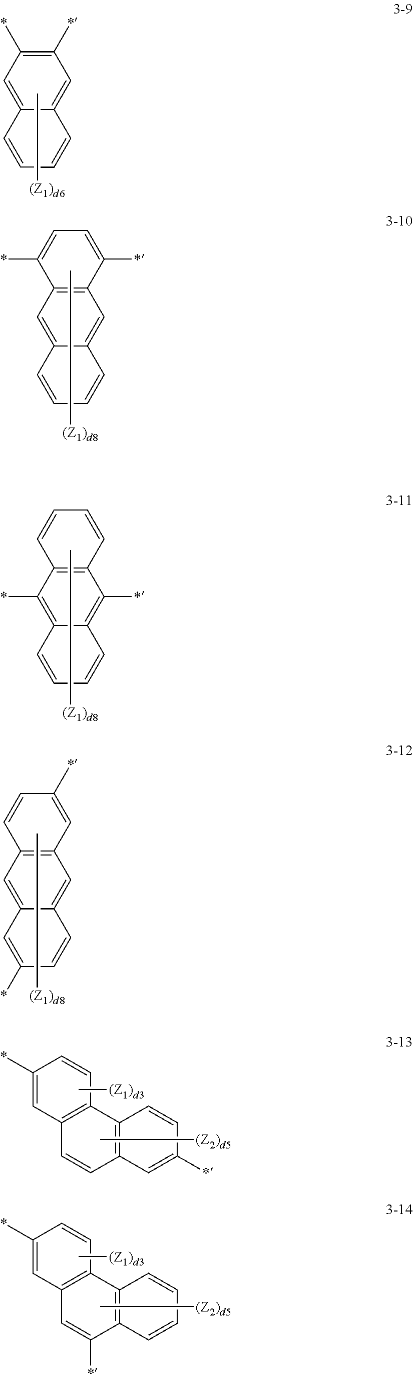

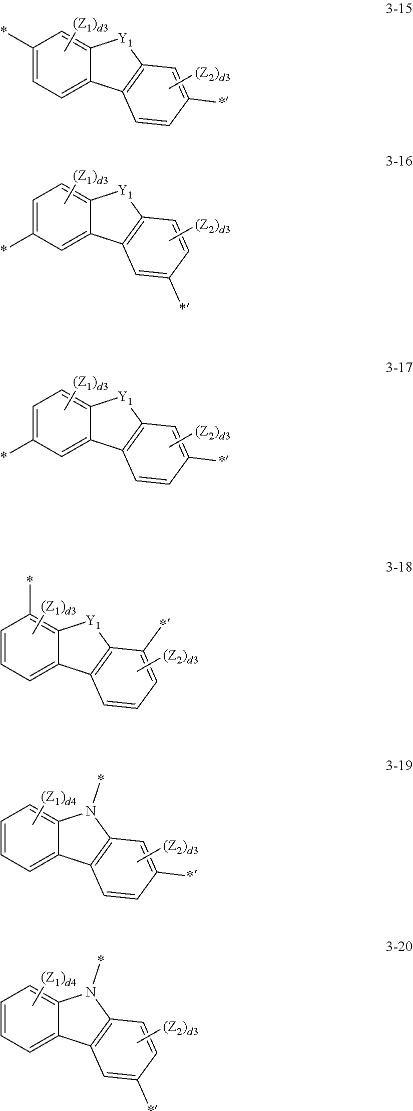

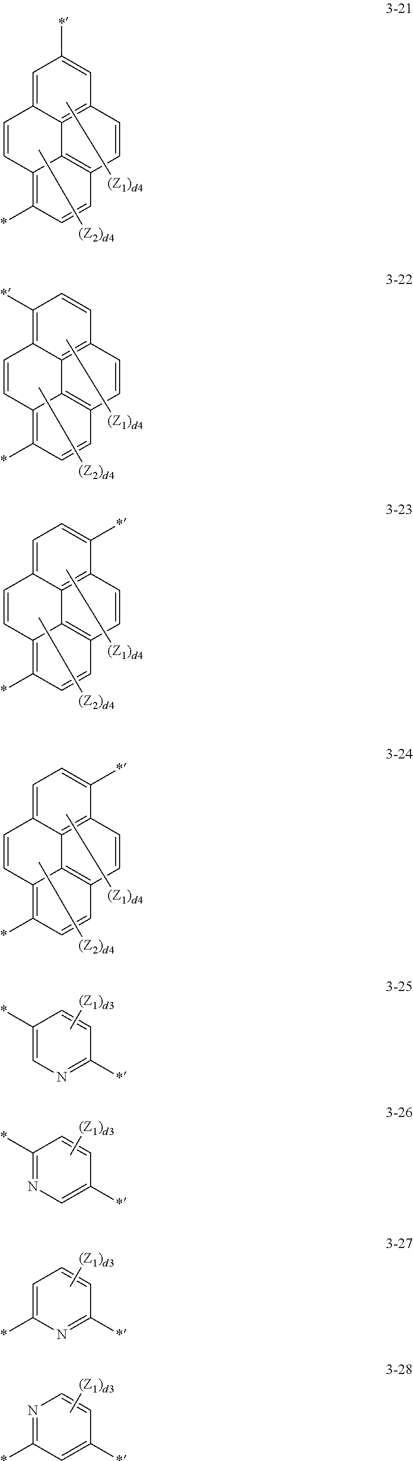

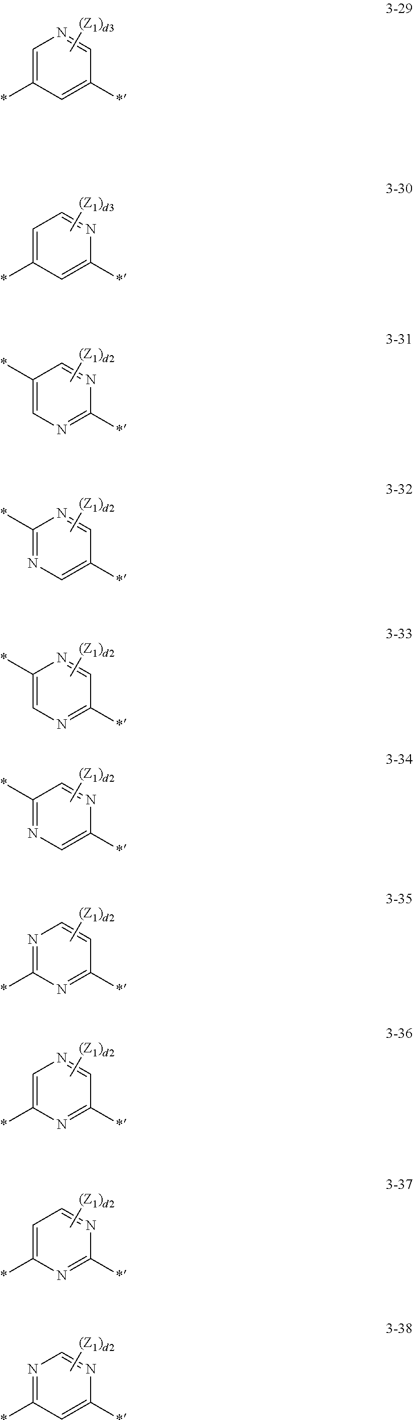

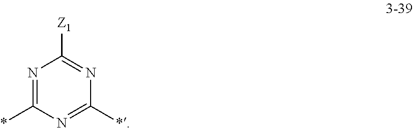

In Formulae 3-1 to 3-39,

Y₁ may be O, S, C(Z₃)(Z₄), N(Z₃), or Si(Z₃)(Z₄),

Z₁ to Z₄ may each independently be selected from hydrogen, deuterium, —F, —Cl, —Br, —I, a hydroxyl group, a cyano group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a naphthyl group, a fluorenyl group, a spiro-bifluorenyl group, a benzofluorenyl group, a dibenzofluorenyl group, a phenalenyl group, a phenanthrenyl group, a n anthracenyl group, a fluoranthenyl group, a pyrenyl group, a chrysenyl group, a naphthacenyl group, a pyrrolyl group, a thiophenyl group, a furanyl group, a silolyl group, a n imidazolyl group, a pyrazolyl group, a thiazolyl group, a n isothiazolyl group, a n oxazolyl group, a n isoxazolyl group, a pyridinyl group, a pyrazinyl group, a pyrimidinyl group, a pyrndazinyl group, a triazinyl group, a benzofuranyl group, a benzothiophenyl group, a dibenzofuranyl group, a dibenzothiophenyl group, a carbazolyl group, a benzosilolyl group, a dibenzosilolyl group, a quinolinyl group, a n isoquinolinyl group, a benzimidazolyl group, an imidazopyridinyl group, an imidazopyrimidinyl group, and —Si(Q₃₁)(Q₃₂)(Q₃₃), and

Q₁ to Q₃₃ may each independently be selected from a C₁-C₂₀ alkyl group, a C₁-C₂₀ alkoxy group, a phenyl group, a biphenyl group, a terphenyl group, a naphthyl group, and a pyridinyl group,

Z₃ and Z₄ may optionally be linked to each other to form a substituted or unsubstituted C₅-C₁₀ carbocyclic group or a substituted or unsubstituted C₁-C₁₀ heterocyclic group,

d2 may be an integer from 0 to 2,

d3 may be an integer from 0 to 3,

d4 may be an integer from 0 to 4,

d5 may be an integer from 0 to 5,

d6 may be an integer from 0 to 6,

d8 may be an integer from 0 to 8, and

* and *′ each indicate a binding site to a neighboring atom.

In Formula 1, a1 to a3 may each independently be an integer from 0 to 3. When a1 is 2 or more, two or more L₁(s) may be identical to or different from each other. When a2 is 2 or more, two or more L₂(s) may be identical to or different from each other. When a3 is 2 or more, two or more L₃(s) may be identical to or different from each other.

In Formula 1, Ar₁ to Ar₃ may each independently be a substituted or unsubstituted C₅-C₆₀ carbocyclic group or a substituted or unsubstituted C₁-C₆₀ heterocyclic group, wherein each of Ar₁ to Ar₃ is not a group represented by Formula 2.

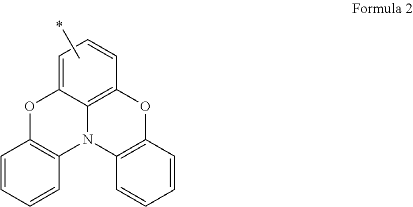

In Formula 2, * indicates a binding site to the heterocyclic compound represented by Formula 1.

In one embodiment, Ar₁ to Ar₃ in Formula 1 may each independently be selected from:

a benzene group, a pentalene group, an indene group, a naphthalene group, an azulene group, a heptalene group, an indacene group, an acenaphthalene group, a fluorene group, a spiro-bifluorene group, a benzofluorene group, a dibenzofluorene group, a phenalene group, a phenanthrene group, an anthracene group, a fluoranthene group, a pyrene group, a chrysene group, a naphthacene group, a picene group, a perylene group, a pyrrole group, a thiophene group, a furan group, a silole group, an imidazole group, a pyrazole group, a thiazole group, an isothiazole group, an oxazole group, an isoxazole group, a pyridine group, a pyrazine group, a pyrimidine group, a pyridazine group, a triazine group, a benzofuran group, a benzothiophene group, a dibenzofuran group, a dibenzothiophene group, a carbazole group, a benzosilole group, a dibenzosilole group, a quinoline group, an isoquinoline group, a benzimidazole group, an imidazopyridine group, and an imidazopyrimidine group;

a benzene group, a pentalene group, an indene group, a naphthalene group, an azulene group, a heptalene group, an indacene group, an acenaphthalene group, a fluorene group, a spiro-bifluorene group, a benzofluorene group, a dibenzofluorene group, a phenalene group, a phenanthrene group, an anthracene group, a fluoranthene group, a pyrene group, a chrysene group, a naphthacene group, a picene group, a perylene group, a pyrrole group, a thiophene group, a furan group, a silole group, an imidazole group, a pyrazole group, a thiazole group, an isothiazole group, an oxazole group, an isoxazole group, a pyridine group, a pyrazine group, a pyrimidine group, a pyridazine group, a triazine group, a benzofuran group, a benzothiophene group, a dibenzofuran group, a dibenzothiophene group, a carbazole group, a benzosilole group, a dibenzosilole group, a quinoline group, an isoquinoline group, a benzimidazole group, an imidazopyridine group, and an imidazopyrimidine group, each substituted with at least one selected from deuterium, —F, —Cl, —Br, —I, a hydroxyl group, a cyano group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a pyrenyl group, a chrysenyl group, a naphthacenyl group, a picenyl group, a perylenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyrdinyl group, a pyrazinyl group, a pyrimidinyl group, a pyridazinyl group, a triazinyl group, a benzofuranyl group, a benzothiophenyl group, a dibenzofuranyl group, a dibenzothiophenyl group, a carbazolyl group, a benzosilolyl group, a dibenzosilolyl group, a quinolinyl group, an isoquinolinyl group, a benzimidazolyl group, an imidazopyridinyl group, an imidazopyrimidinyl group, —Si(Q₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂), and

Q₃₁ to Q₃₃ may each independently be selected from a C₁-C₂₀ alkyl group, a C₁-C₂₀ alkoxy group, a phenyl group, a biphenyl group, a terphenyl group, a naphthyl group, and a pyridinyl group.

For example, Ar₁ to Ar₃ may each independently be a group represented by one of Formulae 4-1 to 4-24:

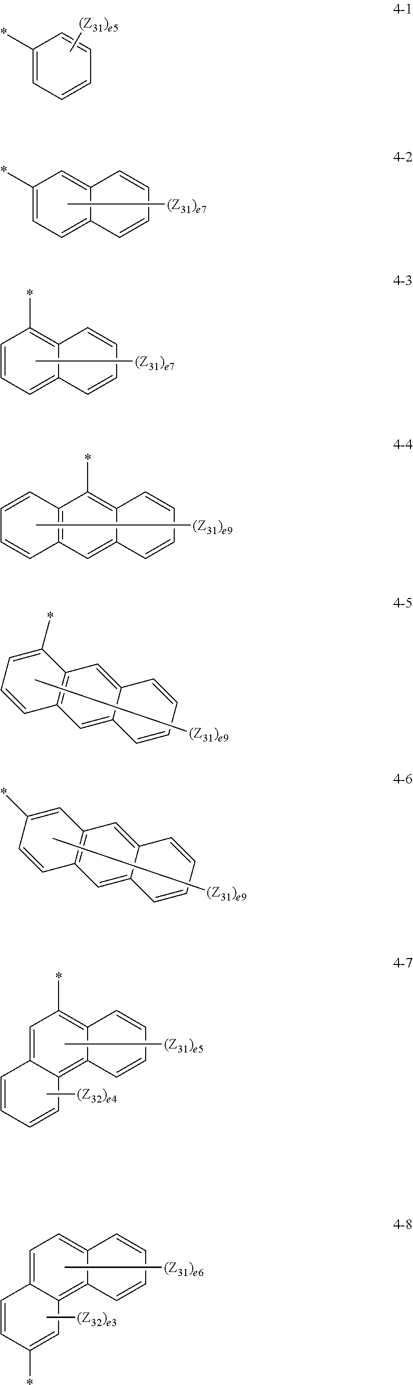

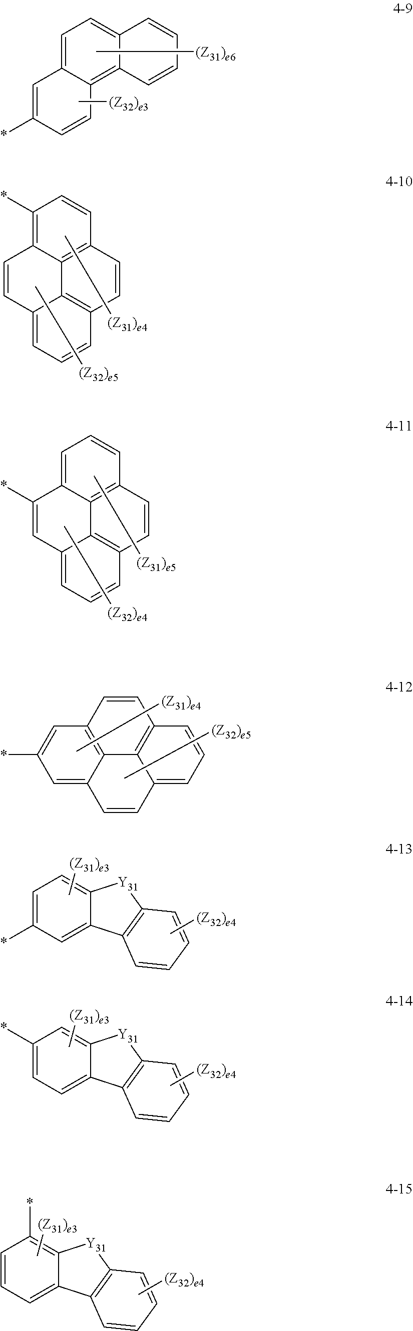

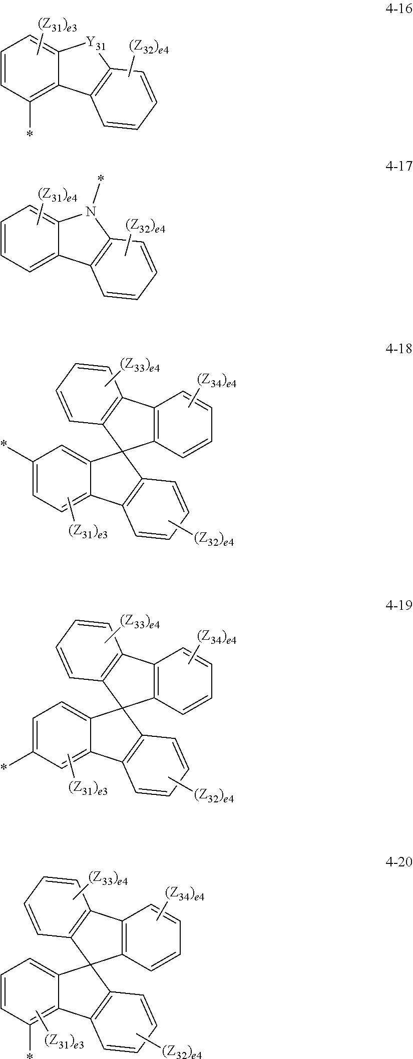

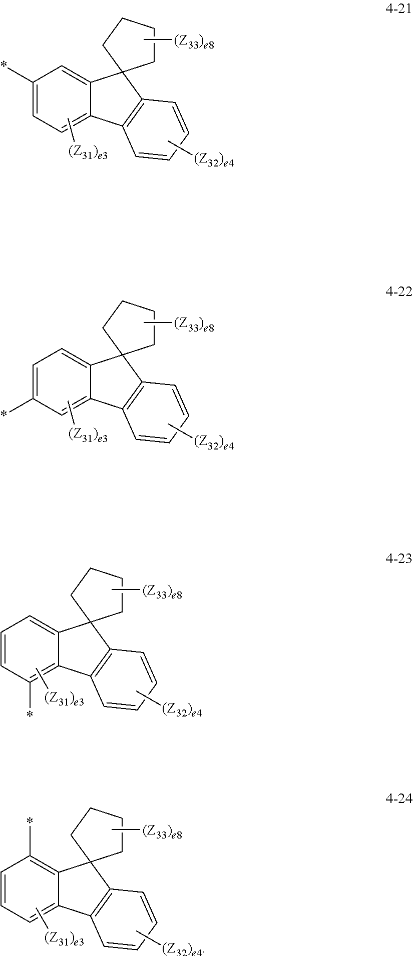

In Formulae 4-1 to 4-24,

Y₃₁ may be O, S, C(Z₃₅)(Z₃₆), N(Z₃₅), or Si(Z₃₅)(Z₃₆),

Z₃₁ to Z₃₆ may each independently be selected from hydrogen, deuterium, —F, —Cl, —Br, —I, a hydroxyl group, a cyano group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a naphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a pyrenyl group, a chrysenyl group, a naphthacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, a benzofuranyl group, a benzothiophenyl group, a dibenzofuranyl group, a dibenzothiophenyl group, a carbazolyl group, a benzosilolyl group, a dibenzosilolyl group, a quinolinyl group, an isoquinolinyl group, a benzimidazolyl group, an imidazopyridinyl group, an imidazopyrimidinyl group, and —Si(Q₃₁)(Q₃₂)(Q₃₃), and

Q₃₁ to Q₃₃ may each independently be selected from a C₁-C₂₀ alkyl group, a C₁-C₂₀ alkoxy group, a phenyl group, a biphenyl group, a terphenyl group, a naphthyl group, and a pyridinyl group,

e3 may be an integer from 0 to 3,

e4 may be an integer from 0 to 4,

e5 may be an integer from 0 to 5,

e6 may be an integer from 0 to 6,

e7 may be an integer from 0 to 7,

e8 may be an integer from 0 to 8,

e9 may be an integer from 0 to 9, and

* indicates a binding site to a neighboring atom.

For example, Ar₁ to Ar₃ may each independently be a group represented by one of Formulae 5-1 to 5-41:

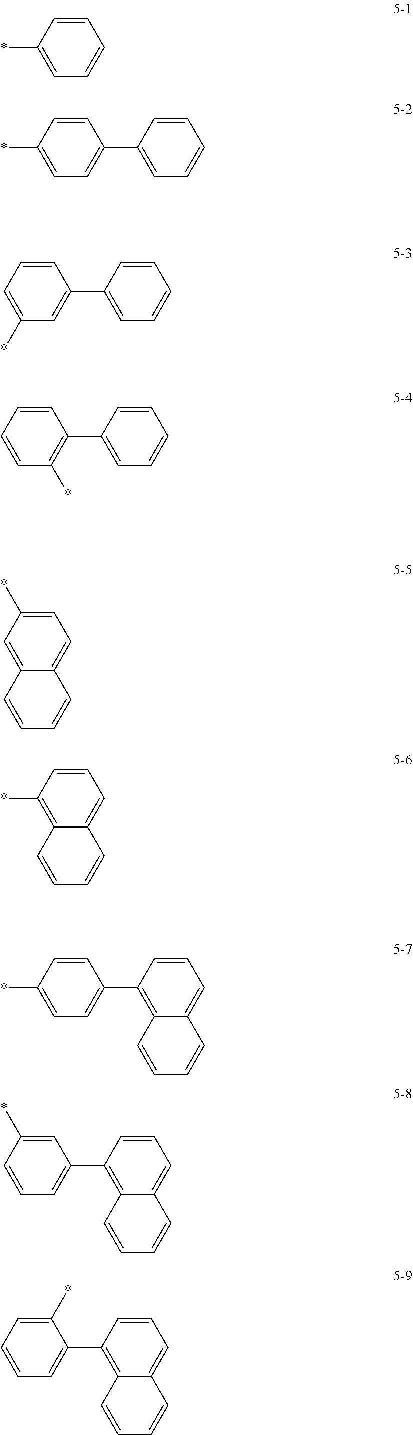

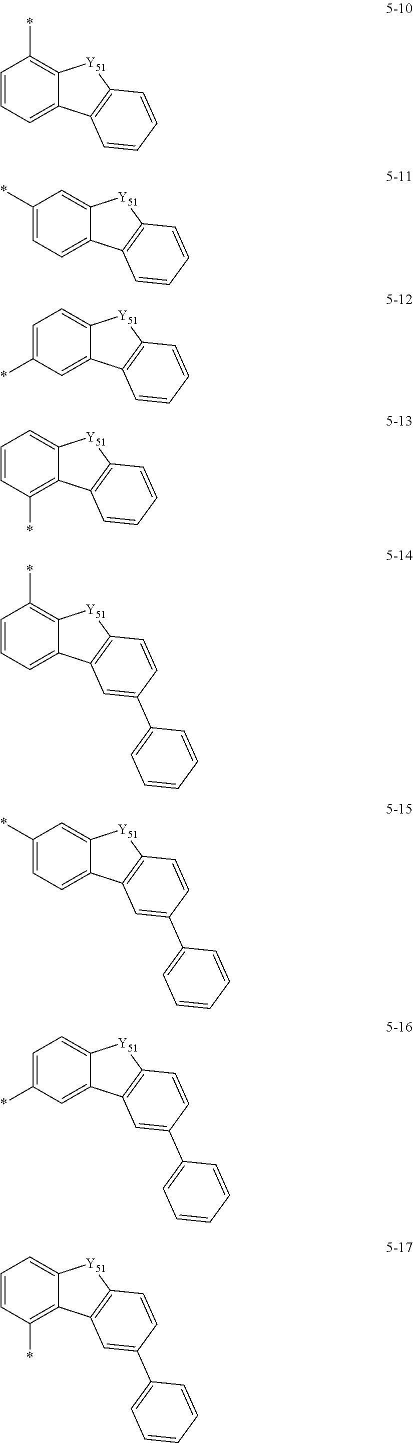

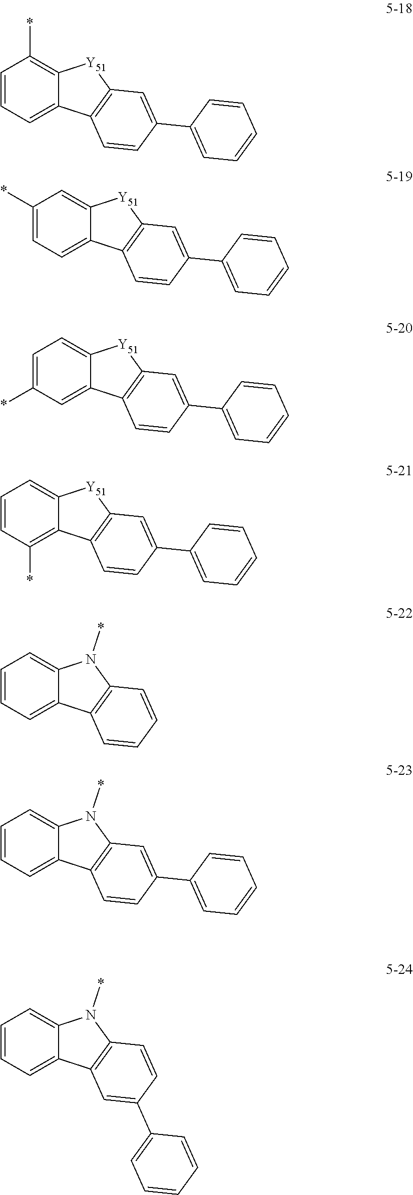

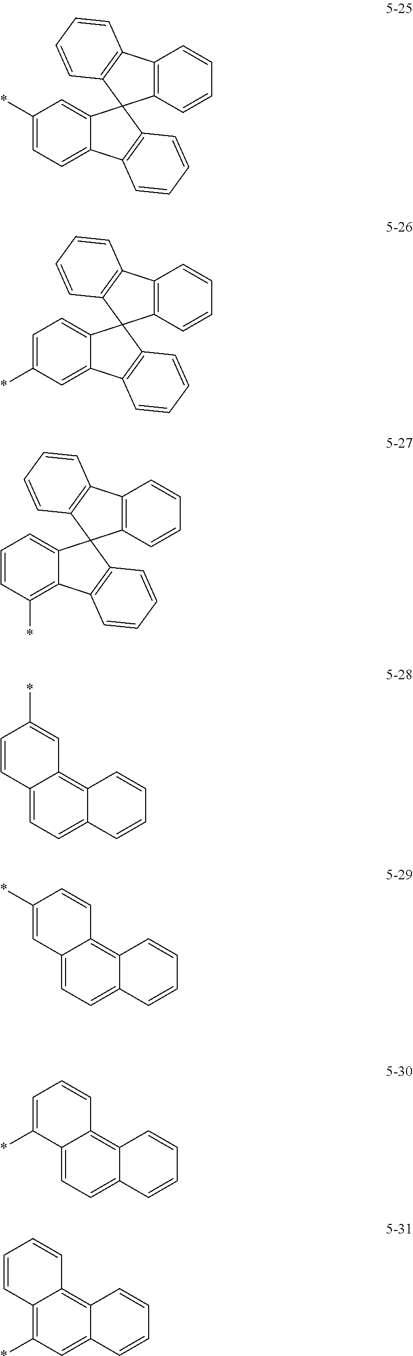

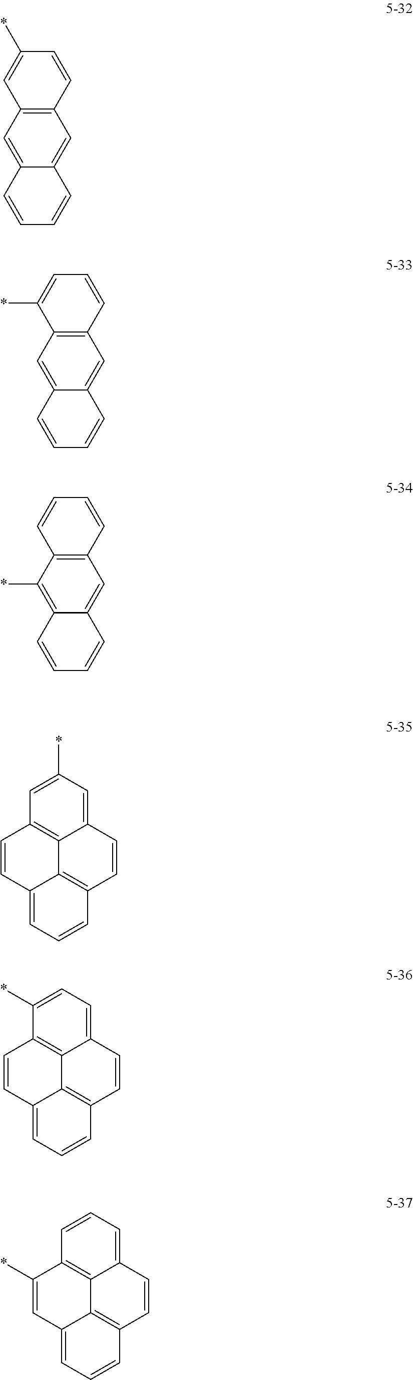 [SMILES: *c1ccc2cc3ccccc3cc2c1 ; *c1cccc2cc3ccccc3cc12 ; *c1c2ccccc2cc2ccccc12 ; *c1cc2ccc3cccc4ccc(c1)c2c34 ; *c1ccc2ccc3cccc4ccc1c2c34 ; *c1cc2cccc3ccc4cccc1c4c32]

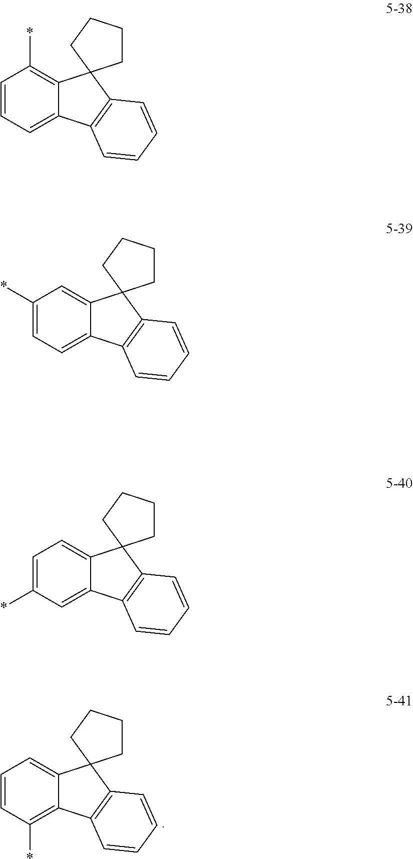 [SMILES: *c1cccc2c1C1(CCCC1)c1ccccc1-2 ; *c1ccc2c(c1)C1(CCCC1)c1ccccc1-2 ; *c1ccc2c(c1)-c1ccccc1C21CCCC1 ; *c1cccc2c1-c1ccccc1C21CCCC1]

In Formulae 5-10 to 5-41′

Y₅₁ may be O, S, C(Z₅₁)(Z₅₂), N(Z₅₁), or Si(Z₅₁)(Z₅₂),

Z₅₁ and Z₅₂ may each independently be selected from hydrogen, deuterium, —F, —Cl, —Br, —I, a hydroxyl group, a cyano group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a naphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a pyrenyl group, a chrysenyl group, a naphthacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, a benzofuranyl group, a benzothiophenyl group, a dibenzofuranyl group, a dibenzothiophenyl group, a carbazolyl group, a benzosilolyl group, a dibenzosilolyl group, a quinolinyl group, an isoquinolinyl group, a benzimidazolyl group, an imidazopyridinyl group, an imidazopyrimidinyl group, and —Si(Q₃₁)(Q₃₂)(Q₃₃), and

Q₃₁ to Q₃₃ may each independently be selected from a C₁-C₂₀ alkyl group, a C₁-C₂₀ alkoxy group, a phenyl group, a biphenyl group, a terphenyl group, a naphthyl group, and a pyridinyl group, and

* indicates a binding site to a neighboring atom.

In Formula 1, b1 to b3 may each independently be an integer from 1 to 3. When b1 is 2 or more, two or more Ar₁(s) may be identical to or different from each other, when b2 is 2 or more, two or more Ar₂(s) may be identical to or different from each other, and when b3 is 2 or more, two or more Ar₃(s) may be identical to or different from each other.

In Formula 1, c1 to c3 may each independently be an integer from 0 to 4, and c1+c2+c3≥1.

When c1 is 2 or more, two or more *-(L₁)ₐ₁-(Ar₁)_{b1}(s) may be identical to or different from each other, when c2 is 2 or more, two or more *-(L₂)ₐ₂-(Ar₂)_{b2}(s) may be identical to or different from each other, and when c3 is 2 or more, two or more *-(L₃)ₐ₃-(Ar₃)_{b3}(s) may be identical to or different from each other.

In one embodiment, when c1 is 1, c2 and c3 may each independently be an integer from 0 to 4. When c2 is 1, c1 and c3 may each independently be an integer from 0 to 4. When c3 is 1, c1 and c2 may each independently be an integer from 0 to 4.

In one or more embodiments, when c1 is 1, c2 may be 1 and c3 may be 0, or when c1 is 1, c2 may be 0 and c3 may be 1. In one or more embodiments, when c2 is 1, c1 may be 1 and c3 may be 0, or when c2 is 1, c1 may be 0 and c3 may be 1. In one or more embodiments, when c3 is 1, c1 may be 1 and c2 may be 0, or when c3 is 1, c1 may be 0 and c2 may be 1.

In one or more embodiments, each of c1, c2, and c3 may be 1.

In Formula 1, R₁ to R₃ may each independently be selected from deuterium, —F, —Cl, —Br, —I, a hydroxyl group, a cyano group, a nitro group, an amino group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁ cycloalkyl group, a substituted or unsubstituted C₂-C₁₀ heterocycloalkyl group, a substituted or unsubstituted C₃-C₁ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₂-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₁)(Q₂), —B(Q₁)(Q₂), —C(═O)(Q₁), —S(═O)₂(Q₁), and —P(═O)(Q₁)(Q₂).

In one embodiment, R₁ to R₃ may each independently be selected from:

deuterium, a methyl group, an ethyl group, an n-propyl group, an isopropyl group, an n-butyl group, an isobutyl group, a sec-butyl group, a tert-butyl group, an n-pentyl group, a tert-pentyl group, a neo-pentyl group, an isopentyl group, a sec-pentyl group, 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a secoctyl group, a tert-octyl group, an n-nonyl group, an isononyl group, a sec-nonyl group, a tert-nonyl group, an n-decyl group, an isodecyl group, a sec-decyl group, and a tert-decyl group; and

a methyl group, an ethyl group, an n-propyl group, an isopropyl group, an n-butyl group, an isobutyl group, a sec-butyl group, a tert-butyl group, an n-pentyl group, a tert-pentyl group, a neo-pentyl group, an isopentyl group, a sec-pentyl group, 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group, each substituted with at least one selected from deuterium, —F, —Cl, —Br, and —I.

In Formula 1, d1 to d3 may each independently be selected from 0 to 4. When d1 is 2 or more, two or more R₁(s) may be identical to or different from each other, when d2 is 2 or more, two or more R₂(s) may be identical to or different from each other, and when d3 is 2 or more, two or more R₃(s) may be identical to or different from each other.

In one embodiment, d1 to d3 may each be 0. A case in which d1 is 0 may mean that hydrogen present in ring A₁ is not substituted with R₁. A case in which d2 is 0 may mean that hydrogen present in ring A₂ is not substituted with R₂. A case in which d3 is 0 may mean that hydrogen present in ring A₃ is not substituted with R₃.

The heterocyclic compound represented by Formula 1 may be represented by one of Formulae 1-1 to 1-7:

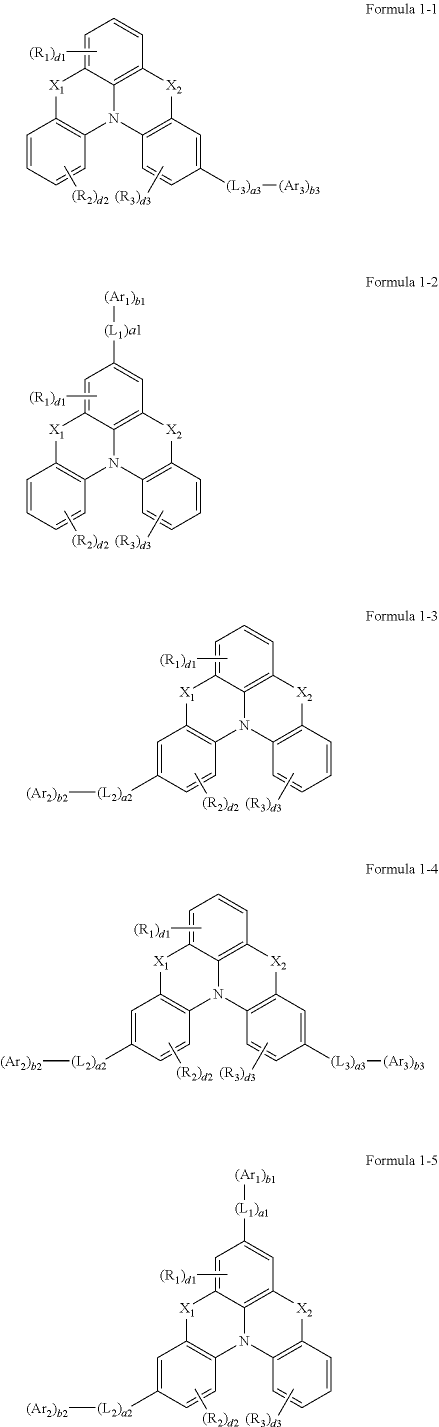

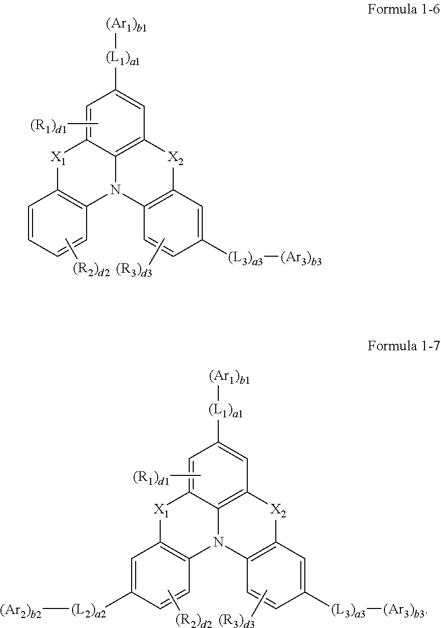

In Formulae 1-1 to 1-7,

X₁, X₂, L₁ to L₃, a1 to a3, Ar₁ to Ar₃, b1 to b3, and R₁ to R₃ may each independently be the same as described herein above, d1 may be to 0 to 3 or 0 to 2, and d2 and d3 may each independently be 0 to 4 or 0 to 3.

In one embodiment, in Formulae 1-1 to 1-7, d1 to d3 may each be 0 and X₁ and X₂ may each be O.

In one embodiment, the heterocyclic compound may be selected from Compounds 1 to 144:

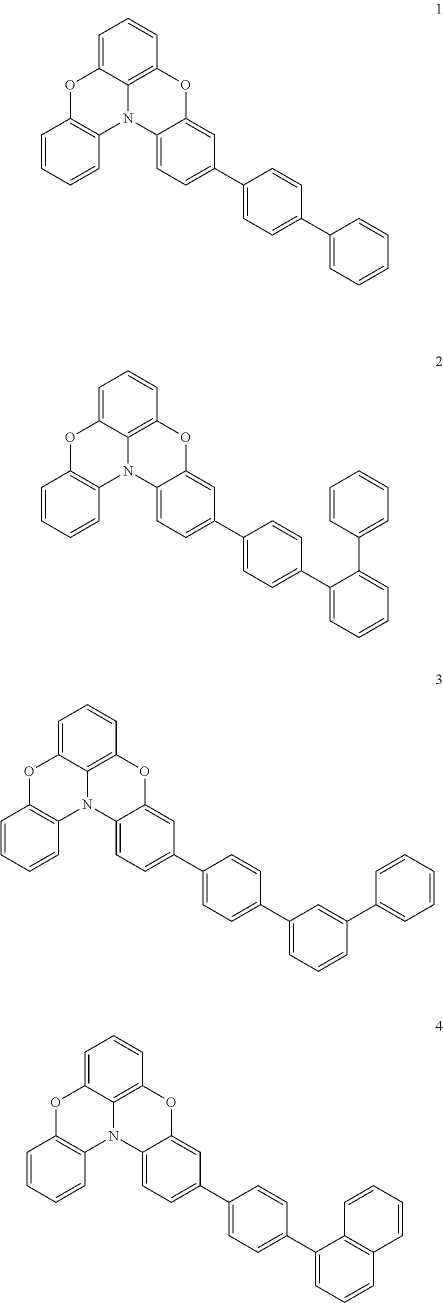 [SMILES: c1ccc(-c2ccc(-c3ccc4c(c3)Oc3cccc5c3N4c3ccccc3O5)cc2)cc1 ; c1ccc(-c2ccccc2-c2ccc(-c3ccc4c(c3)Oc3cccc5c3N4c3ccccc3O5)cc2)cc1 ; c1ccc(-c2cccc(-c3ccc(-c4ccc5c(c4)Oc4cccc6c4N5c4ccccc4O6)cc3)c2)cc1 ; c1ccc2c(c1)Oc1cccc3c1N2c1ccc(-c2ccc(-c4cccc5ccccc45)cc2)cc1O3]

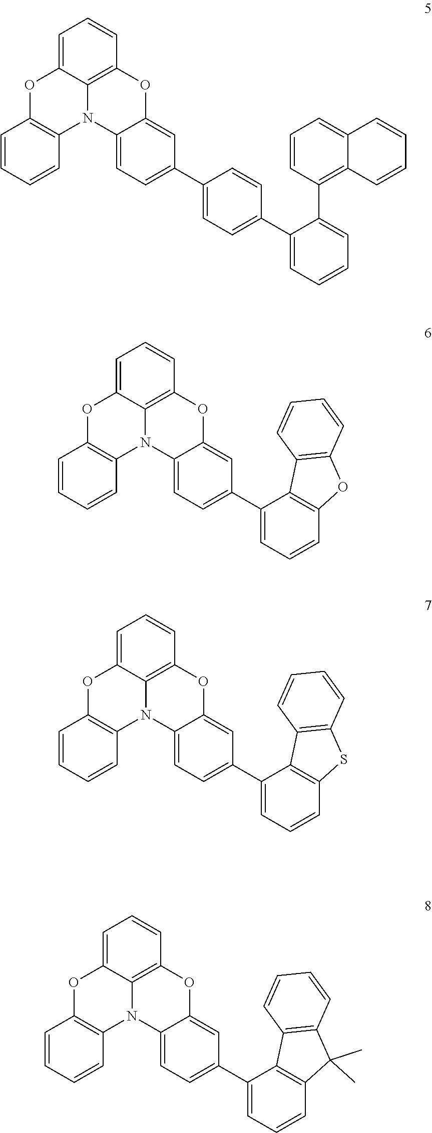 [SMILES: c1ccc2c(c1)Oc1cccc3c1N2c1ccc(-c2ccc(-c4ccccc4-c4cccc5ccccc45)cc2)cc1O3 ; c1ccc2c(c1)Oc1cccc3c1N2c1ccc(-c2cccc4oc5ccccc5c24)cc1O3 ; c1ccc2c(c1)Oc1cccc3c1N2c1ccc(-c2cccc4sc5ccccc5c24)cc1O3 ; CC1(C)c2ccccc2-c2c(-c3ccc4c(c3)Oc3cccc5c3N4c3ccccc3O5)cccc21]

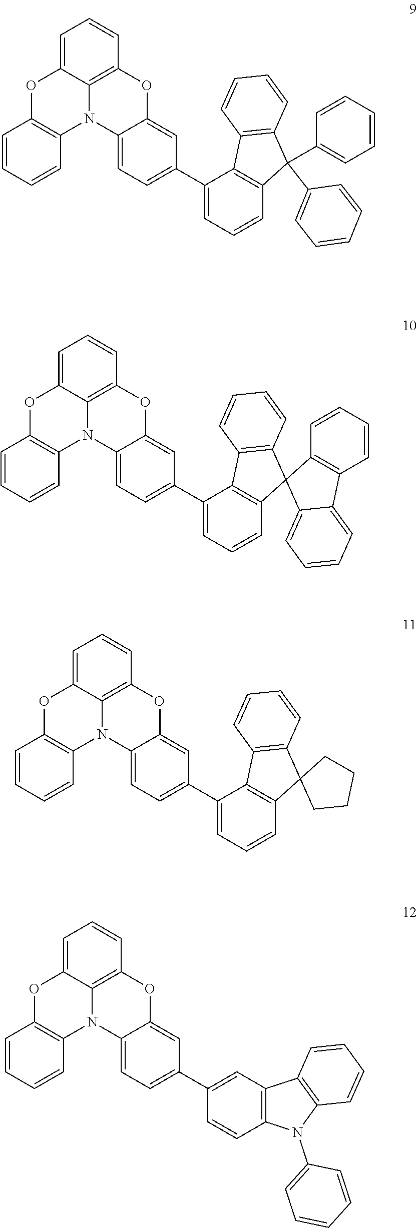 [SMILES: c1ccc(C2(c3ccccc3)c3ccccc3-c3c(-c4ccc5c(c4)Oc4cccc6c4N5c4ccccc4O6)cccc32)cc1 ; c1ccc2c(c1)Oc1cccc3c1N2c1ccc(-c2cccc4c2-c2ccccc2C42c4ccccc4-c4ccccc42)cc1O3 ; c1ccc2c(c1)Oc1cccc3c1N2c1ccc(-c2cccc4c2-c2ccccc2C42CCCC2)cc1O3 ; c1ccc(-n2c3ccccc3c3cc(-c4ccc5c(c4)Oc4cccc6c4N5c4ccccc4O6)ccc32)cc1]

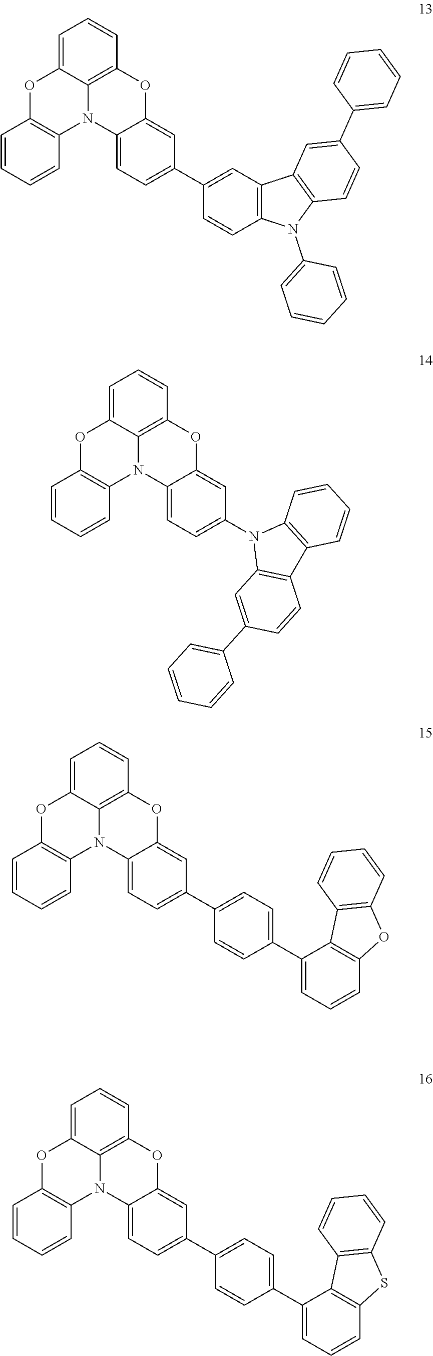 [SMILES: c1ccc(-c2ccc3c(c2)c2cc(-c4ccc5c(c4)Oc4cccc6c4N5c4ccccc4O6)ccc2n3-c2ccccc2)cc1 ; c1ccc(-c2ccc3c4ccccc4n(-c4ccc5c(c4)Oc4cccc6c4N5c4ccccc4O6)c3c2)cc1 ; c1ccc2c(c1)Oc1cccc3c1N2c1ccc(-c2ccc(-c4cccc5oc6ccccc6c45)cc2)cc1O3 ; c1ccc2c(c1)Oc1cccc3c1N2c1ccc(-c2ccc(-c4cccc5sc6ccccc6c45)cc2)cc1O3]

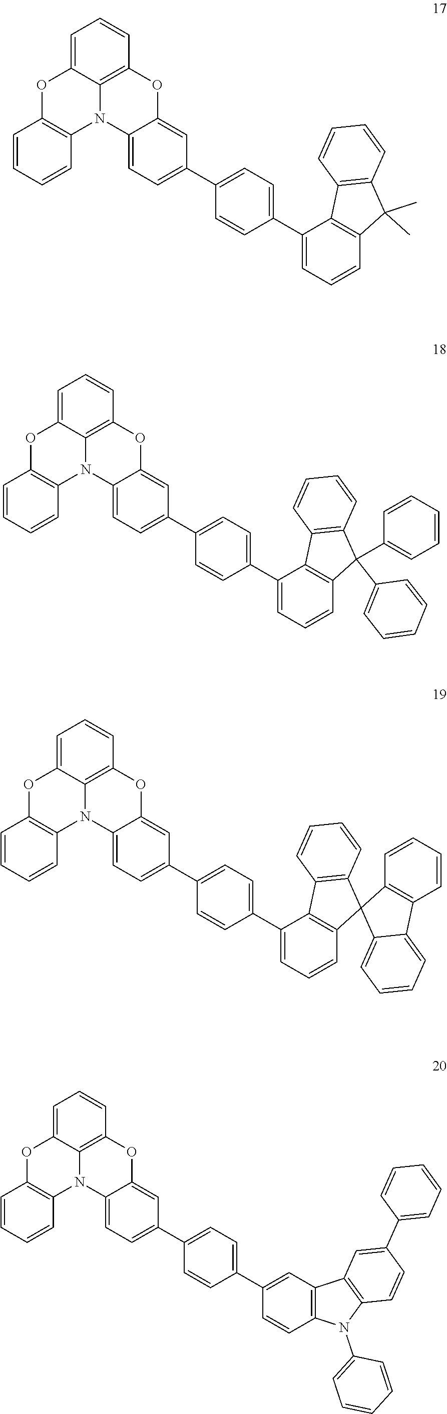 [SMILES: CC1(C)c2ccccc2-c2c(-c3ccc(-c4ccc5c(c4)Oc4cccc6c4N5c4ccccc4O6)cc3)cccc21 ; c1ccc(C2(c3ccccc3)c3ccccc3-c3c(-c4ccc(-c5ccc6c(c5)Oc5cccc7c5N6c5ccccc5O7)cc4)cccc32)cc1 ; c1ccc2c(c1)Oc1cccc3c1N2c1ccc(-c2ccc(-c4cccc5c4-c4ccccc4C54c5ccccc5-c5ccccc54)cc2)cc1O3 ; c1ccc(-c2ccc3c(c2)c2cc(-c4ccc(-c5ccc6c(c5)Oc5cccc7c5N6c5ccccc5O7)cc4)ccc2n3-c2ccccc2)cc1]

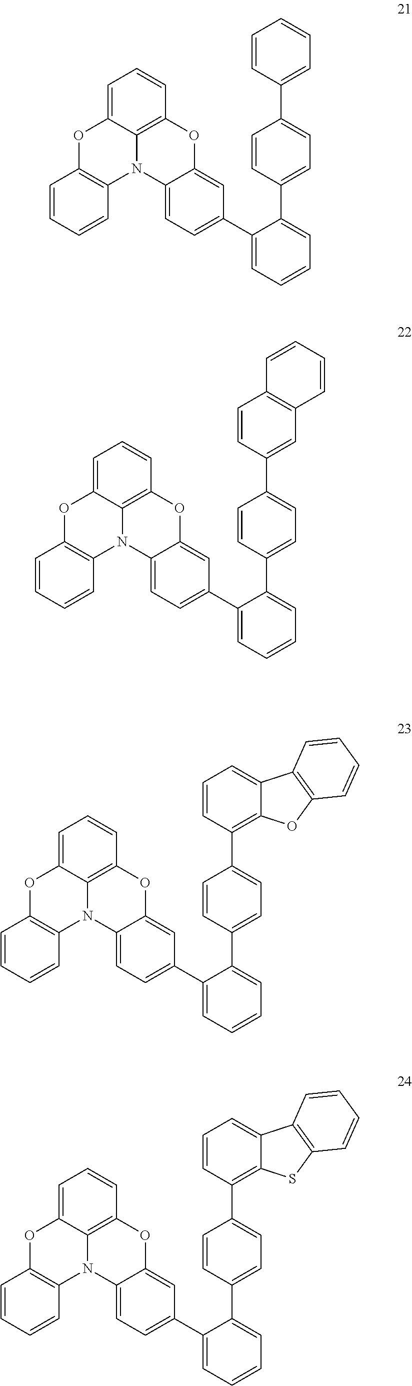 [SMILES: c1ccc(-c2ccc(-c3ccccc3-c3ccc4c(c3)Oc3cccc5c3N4c3ccccc3O5)cc2)cc1 ; c1ccc2c(c1)Oc1cccc3c1N2c1ccc(-c2ccccc2-c2ccc(-c4ccc5ccccc5c4)cc2)cc1O3 ; c1ccc2c(c1)Oc1cccc3c1N2c1ccc(-c2ccccc2-c2ccc(-c4cccc5c4oc4ccccc45)cc2)cc1O3 ; c1ccc2c(c1)Oc1cccc3c1N2c1ccc(-c2ccccc2-c2ccc(-c4cccc5c4sc4ccccc45)cc2)cc1O3]

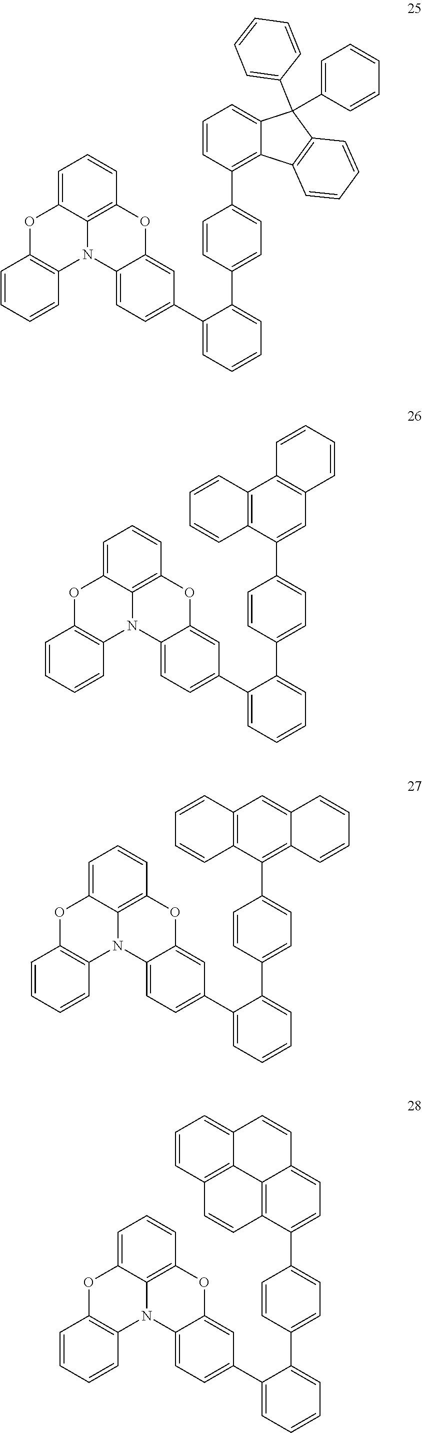 [SMILES: c1ccc(C2(c3ccccc3)c3ccccc3-c3c(-c4ccc(-c5ccccc5-c5ccc6c(c5)Oc5cccc7c5N6c5ccccc5O7)cc4)cccc32)cc1 ; c1ccc2c(c1)Oc1cccc3c1N2c1ccc(-c2ccccc2-c2ccc(-c4cc5ccccc5c5ccccc45)cc2)cc1O3 ; c1ccc2c(c1)Oc1cccc3c1N2c1ccc(-c2ccccc2-c2ccc(-c4c5ccccc5cc5ccccc45)cc2)cc1O3 ; c1ccc2c(c1)Oc1cccc3c1N2c1ccc(-c2ccccc2-c2ccc(-c4ccc5ccc6cccc7ccc4c5c67)cc2)cc1O3]

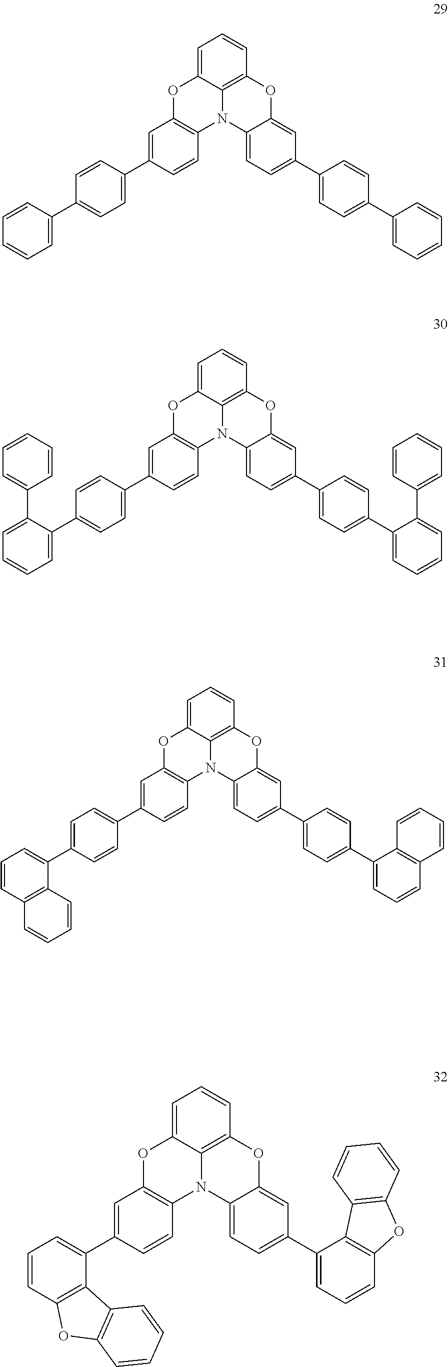 [SMILES: c1ccc(-c2ccc(-c3ccc4c(c3)Oc3cccc5c3N4c3ccc(-c4ccc(-c6ccccc6)cc4)cc3O5)cc2)cc1 ; c1ccc(-c2ccccc2-c2ccc(-c3ccc4c(c3)Oc3cccc5c3N4c3ccc(-c4ccc(-c6ccccc6-c6ccccc6)cc4)cc3O5)cc2)cc1 ; c1cc2c3c(c1)Oc1cc(-c4ccc(-c5cccc6ccccc56)cc4)ccc1N3c1ccc(-c3ccc(-c4cccc5ccccc45)cc3)cc1O2 ; c1cc2c3c(c1)Oc1cc(-c4cccc5oc6ccccc6c45)ccc1N3c1ccc(-c3cccc4oc5ccccc5c34)cc1O2]

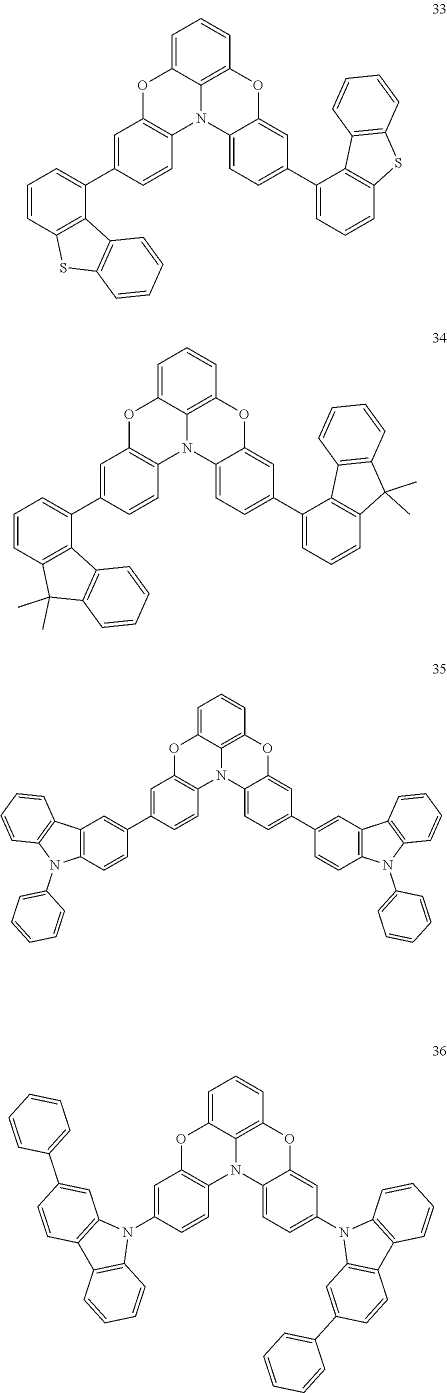 [SMILES: c1cc2c3c(c1)Oc1cc(-c4cccc5sc6ccccc6c45)ccc1N3c1ccc(-c3cccc4sc5ccccc5c34)cc1O2 ; CC1(C)c2ccccc2-c2c(-c3ccc4c(c3)Oc3cccc5c3N4c3ccc(-c4cccc6c4-c4ccccc4C6(C)C)cc3O5)cccc21 ; c1ccc(-n2c3ccccc3c3cc(-c4ccc5c(c4)Oc4cccc6c4N5c4ccc(-c5ccc7c(c5)c5ccccc5n7-c5ccccc5)cc4O6)ccc32)cc1 ; c1ccc(-c2ccc3c4ccccc4n(-c4ccc5c(c4)Oc4cccc6c4N5c4ccc(-n5c7ccccc7c7ccc(-c8ccccc8)cc75)cc4O6)c3c2)cc1]

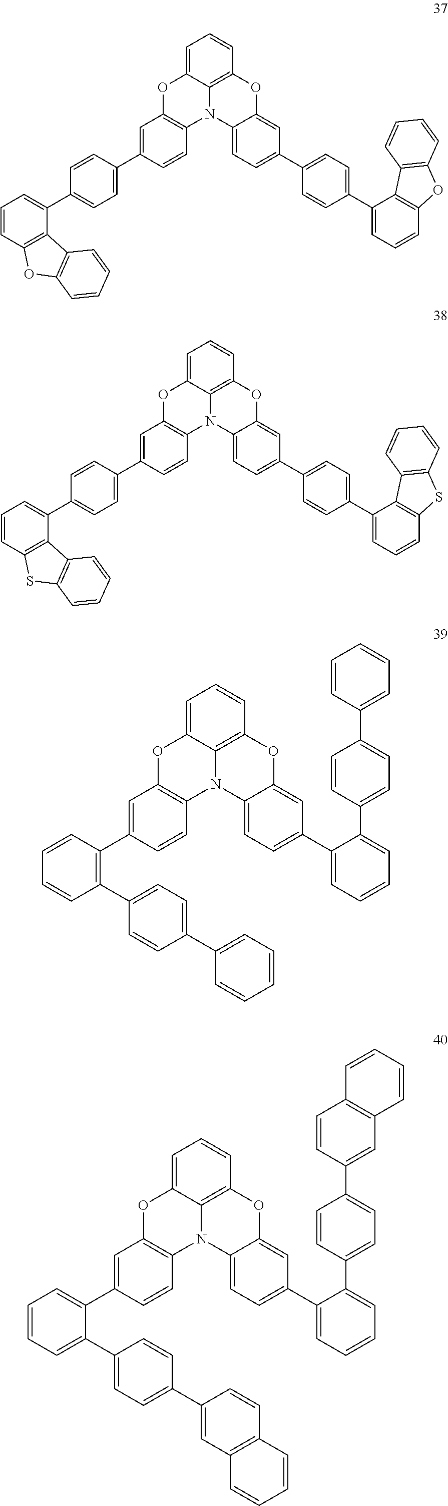 [SMILES: c1cc2c3c(c1)Oc1cc(-c4ccc(-c5cccc6oc7ccccc7c56)cc4)ccc1N3c1ccc(-c3ccc(-c4cccc5oc6ccccc6c45)cc3)cc1O2 ; c1cc2c3c(c1)Oc1cc(-c4ccc(-c5cccc6sc7ccccc7c56)cc4)ccc1N3c1ccc(-c3ccc(-c4cccc5sc6ccccc6c45)cc3)cc1O2 ; c1ccc(-c2ccc(-c3ccccc3-c3ccc4c(c3)Oc3cccc5c3N4c3ccc(-c4ccccc4-c4ccc(-c6ccccc6)cc4)cc3O5)cc2)cc1 ; c1cc2c3c(c1)Oc1cc(-c4ccccc4-c4ccc(-c5ccc6ccccc6c5)cc4)ccc1N3c1ccc(-c3ccccc3-c3ccc(-c4ccc5ccccc5c4)cc3)cc1O2]

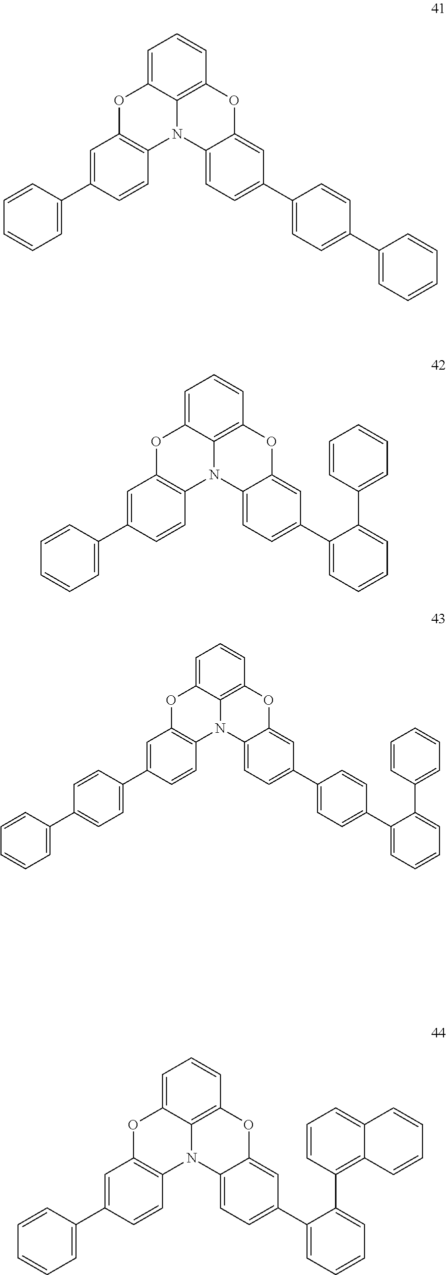 [SMILES: c1ccc(-c2ccc(-c3ccc4c(c3)Oc3cccc5c3N4c3ccc(-c4ccccc4)cc3O5)cc2)cc1 ; c1ccc(-c2ccc3c(c2)Oc2cccc4c2N3c2ccc(-c3ccccc3-c3ccccc3)cc2O4)cc1 ; c1ccc(-c2ccc(-c3ccc4c(c3)Oc3cccc5c3N4c3ccc(-c4ccc(-c6ccccc6-c6ccccc6)cc4)cc3O5)cc2)cc1 ; c1ccc(-c2ccc3c(c2)Oc2cccc4c2N3c2ccc(-c3ccccc3-c3cccc5ccccc35)cc2O4)cc1]

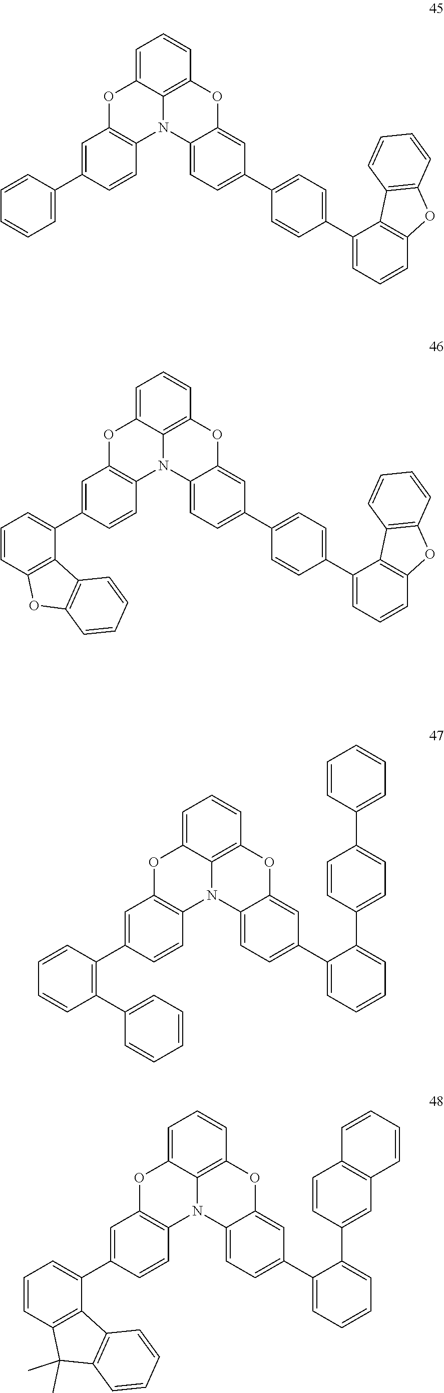 [SMILES: c1ccc(-c2ccc3c(c2)Oc2cccc4c2N3c2ccc(-c3ccc(-c5cccc6oc7ccccc7c56)cc3)cc2O4)cc1 ; c1cc2c3c(c1)Oc1cc(-c4cccc5oc6ccccc6c45)ccc1N3c1ccc(-c3ccc(-c4cccc5oc6ccccc6c45)cc3)cc1O2 ; c1ccc(-c2ccc(-c3ccccc3-c3ccc4c(c3)Oc3cccc5c3N4c3ccc(-c4ccccc4-c4ccccc4)cc3O5)cc2)cc1 ; CC1(C)c2ccccc2-c2c(-c3ccc4c(c3)Oc3cccc5c3N4c3ccc(-c4ccccc4-c4ccc6ccccc6c4)cc3O5)cccc21]

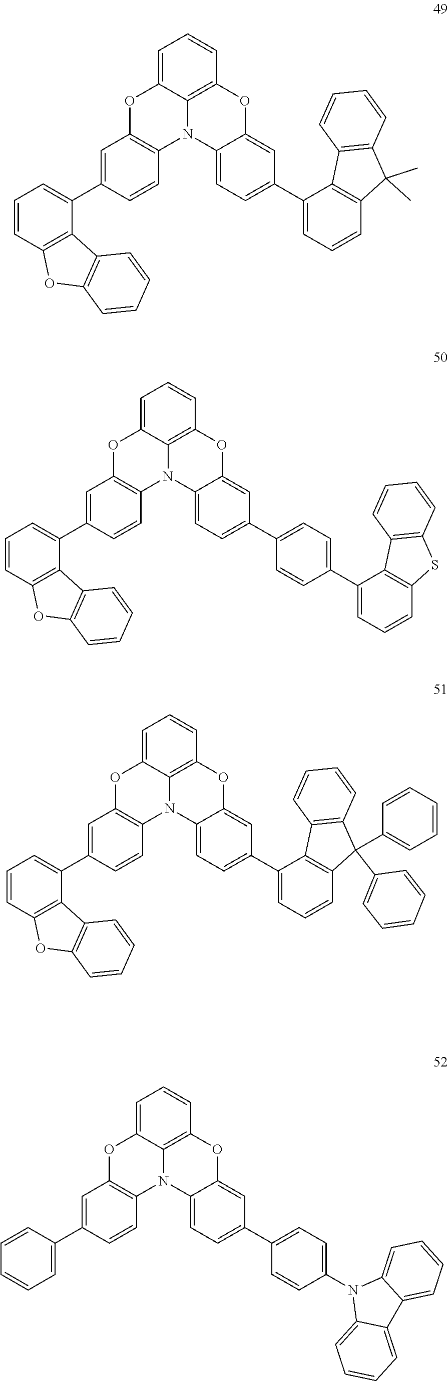 [SMILES: CC1(C)c2ccccc2-c2c(-c3ccc4c(c3)Oc3cccc5c3N4c3ccc(-c4cccc6oc7ccccc7c46)cc3O5)cccc21 ; c1cc2c3c(c1)Oc1cc(-c4cccc5oc6ccccc6c45)ccc1N3c1ccc(-c3ccc(-c4cccc5sc6ccccc6c45)cc3)cc1O2 ; c1ccc(C2(c3ccccc3)c3ccccc3-c3c(-c4ccc5c(c4)Oc4cccc6c4N5c4ccc(-c5cccc7oc8ccccc8c57)cc4O6)cccc32)cc1 ; c1ccc(-c2ccc3c(c2)Oc2cccc4c2N3c2ccc(-c3ccc(-n5c6ccccc6c6ccccc65)cc3)cc2O4)cc1]

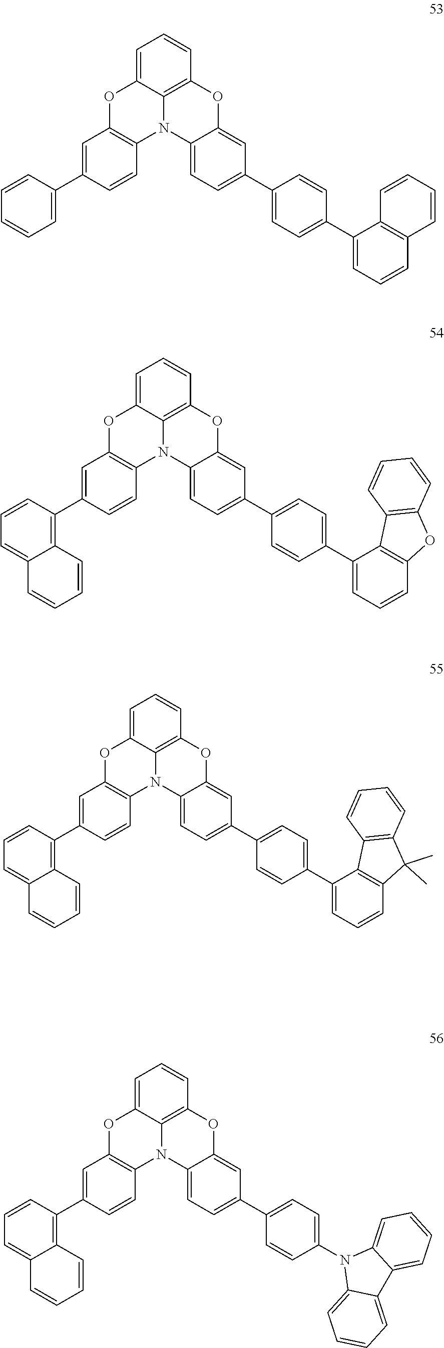 [SMILES: c1ccc(-c2ccc3c(c2)Oc2cccc4c2N3c2ccc(-c3ccc(-c5cccc6ccccc56)cc3)cc2O4)cc1 ; c1cc2c3c(c1)Oc1cc(-c4cccc5ccccc45)ccc1N3c1ccc(-c3ccc(-c4cccc5oc6ccccc6c45)cc3)cc1O2 ; CC1(C)c2ccccc2-c2c(-c3ccc(-c4ccc5c(c4)Oc4cccc6c4N5c4ccc(-c5cccc7ccccc57)cc4O6)cc3)cccc21 ; c1cc2c3c(c1)Oc1cc(-c4cccc5ccccc45)ccc1N3c1ccc(-c3ccc(-n4c5ccccc5c5ccccc54)cc3)cc1O2]

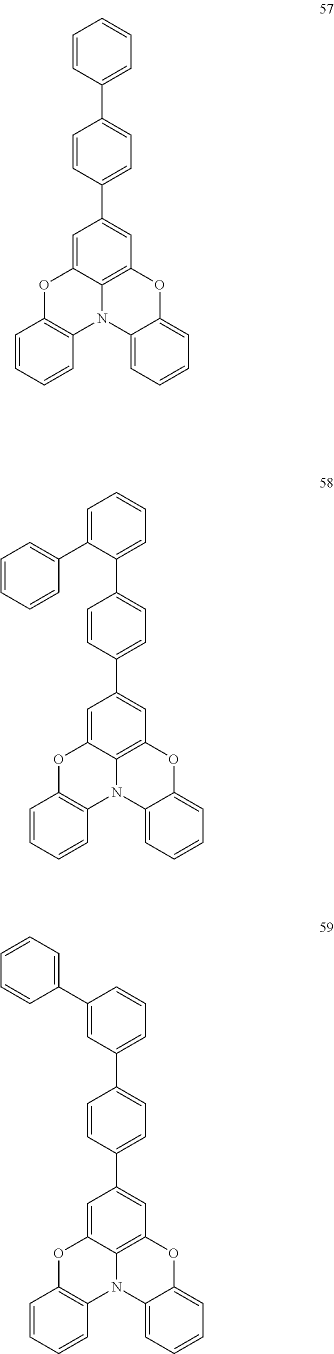 [SMILES: c1ccc(-c2ccc(-c3cc4c5c(c3)Oc3ccccc3N5c3ccccc3O4)cc2)cc1 ; c1ccc(-c2ccccc2-c2ccc(-c3cc4c5c(c3)Oc3ccccc3N5c3ccccc3O4)cc2)cc1 ; c1ccc(-c2cccc(-c3ccc(-c4cc5c6c(c4)Oc4ccccc4N6c4ccccc4O5)cc3)c2)cc1]

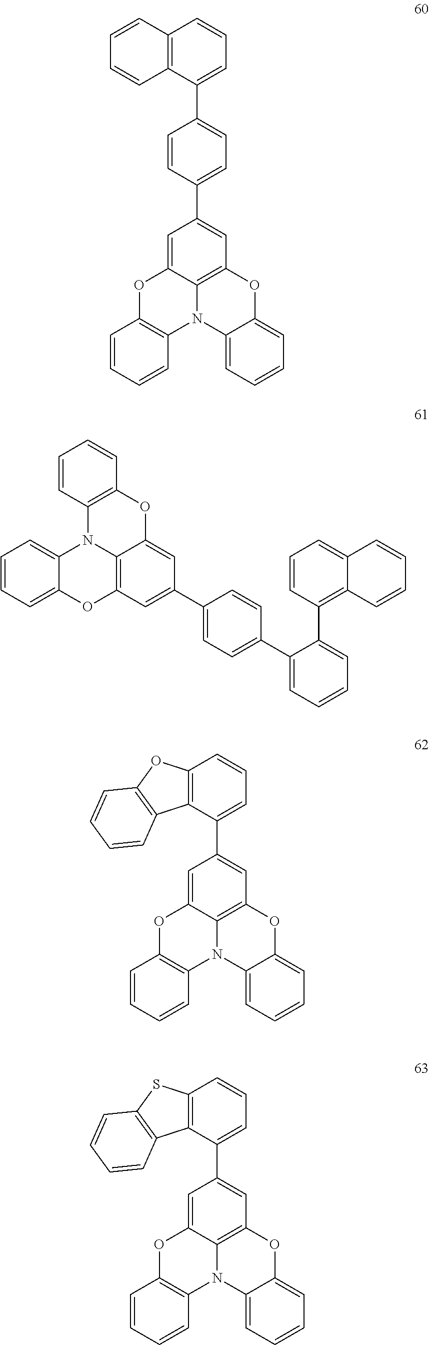 [SMILES: c1ccc2c(c1)Oc1cc(-c3ccc(-c4cccc5ccccc45)cc3)cc3c1N2c1ccccc1O3 ; c1ccc2c(c1)Oc1cc(-c3ccc(-c4ccccc4-c4cccc5ccccc45)cc3)cc3c1N2c1ccccc1O3 ; c1ccc2c(c1)Oc1cc(-c3cccc4oc5ccccc5c34)cc3c1N2c1ccccc1O3 ; c1ccc2c(c1)Oc1cc(-c3cccc4sc5ccccc5c34)cc3c1N2c1ccccc1O3]

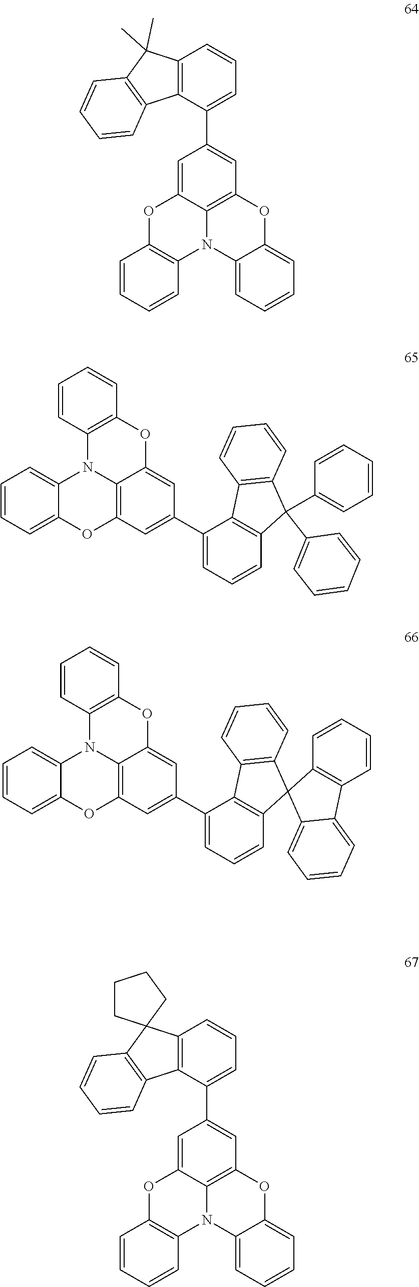 [SMILES: CC1(C)c2ccccc2-c2c(-c3cc4c5c(c3)Oc3ccccc3N5c3ccccc3O4)cccc21 ; c1ccc(C2(c3ccccc3)c3ccccc3-c3c(-c4cc5c6c(c4)Oc4ccccc4N6c4ccccc4O5)cccc32)cc1 ; c1ccc2c(c1)Oc1cc(-c3cccc4c3-c3ccccc3C43c4ccccc4-c4ccccc43)cc3c1N2c1ccccc1O3 ; c1ccc2c(c1)Oc1cc(-c3cccc4c3-c3ccccc3C43CCCC3)cc3c1N2c1ccccc1O3]

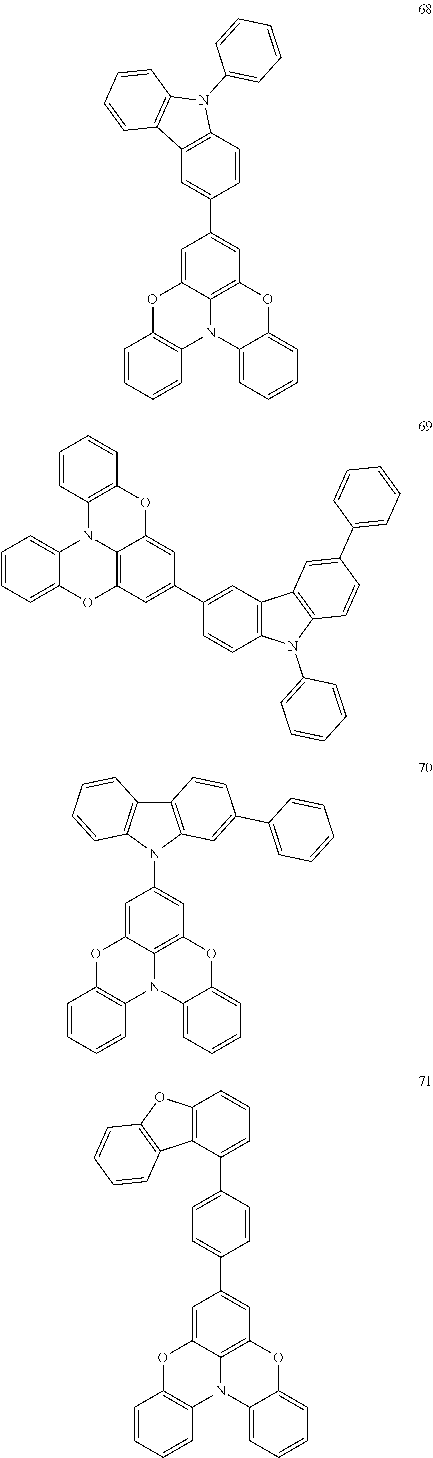

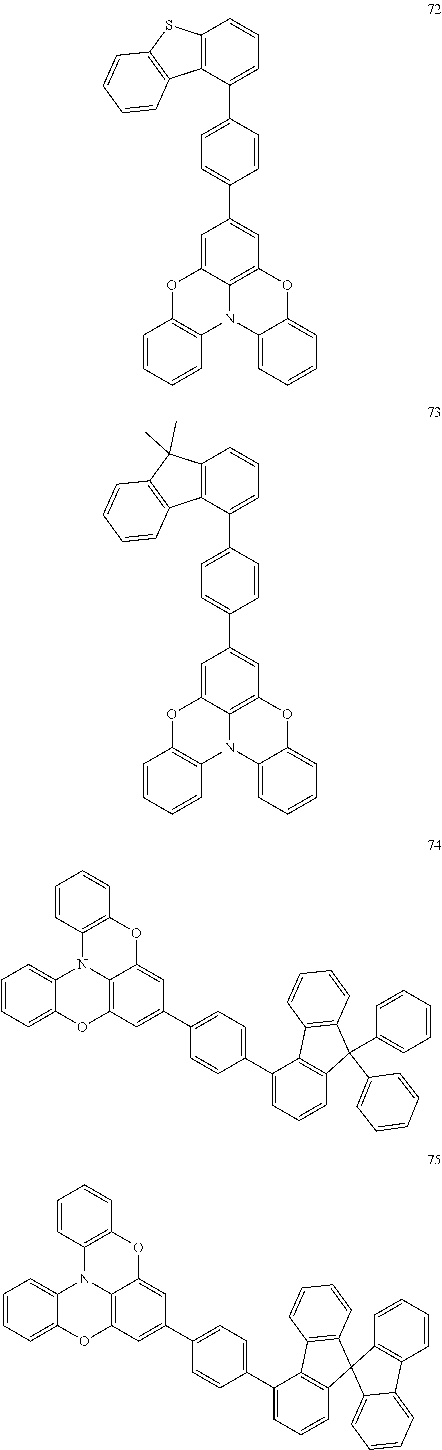

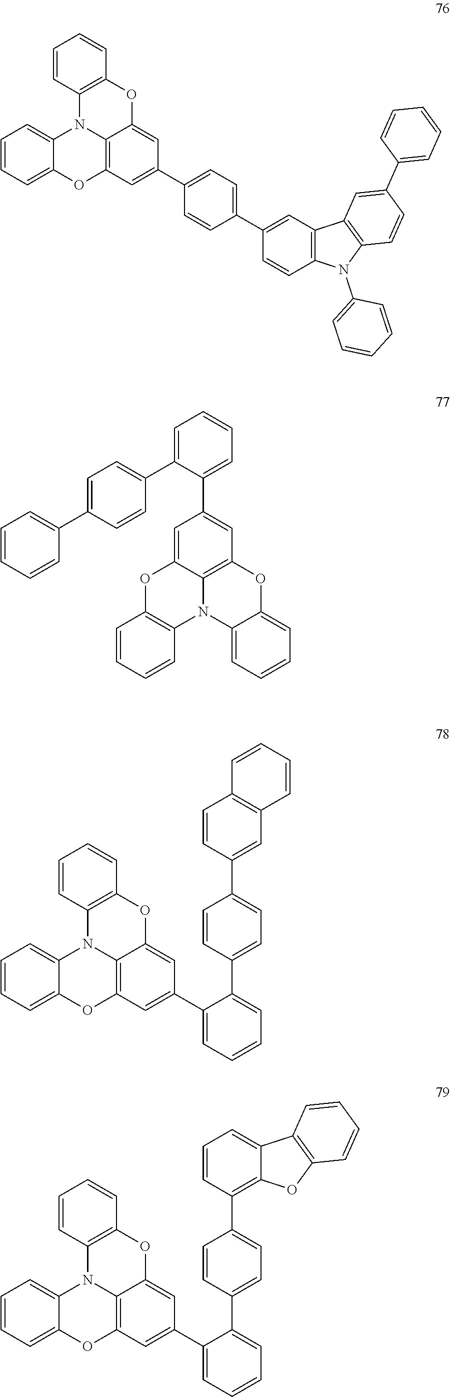 [SMILES: c1ccc(-c2ccc3c(c2)c2cc(-c4ccc(-c5cc6c7c(c5)Oc5ccccc5N7c5ccccc5O6)cc4)ccc2n3-c2ccccc2)cc1 ; c1ccc(-c2ccc(-c3ccccc3-c3cc4c5c(c3)Oc3ccccc3N5c3ccccc3O4)cc2)cc1 ; c1ccc2c(c1)Oc1cc(-c3ccccc3-c3ccc(-c4ccc5ccccc5c4)cc3)cc3c1N2c1ccccc1O3 ; c1ccc2c(c1)Oc1cc(-c3ccccc3-c3ccc(-c4cccc5c4oc4ccccc45)cc3)cc3c1N2c1ccccc1O3]

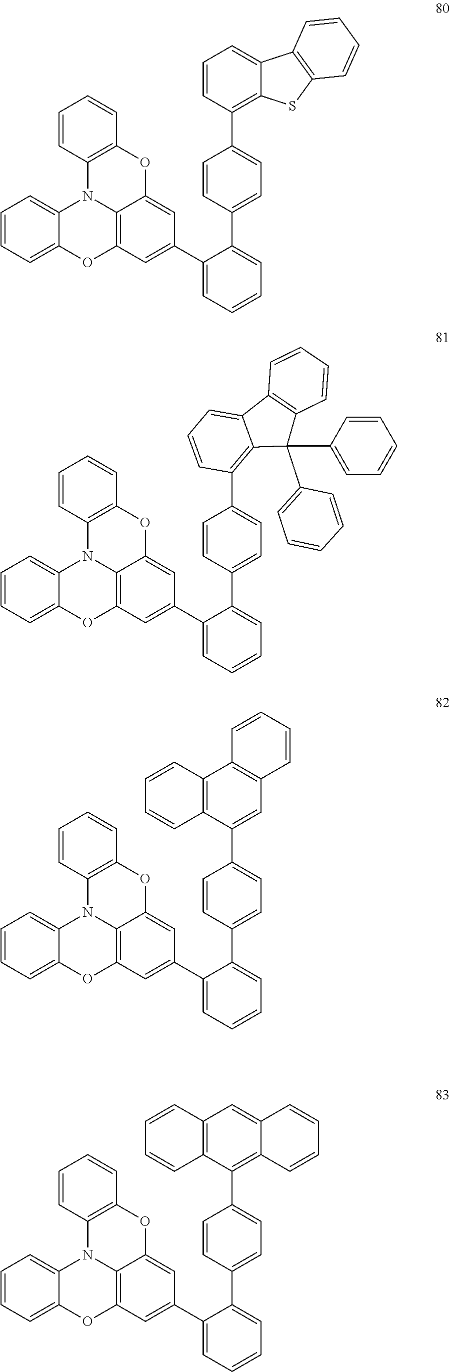 [SMILES: c1ccc2c(c1)Oc1cc(-c3ccccc3-c3ccc(-c4cccc5c4sc4ccccc45)cc3)cc3c1N2c1ccccc1O3 ; c1ccc(C2(c3ccccc3)c3ccccc3-c3cccc(-c4ccc(-c5ccccc5-c5cc6c7c(c5)Oc5ccccc5N7c5ccccc5O6)cc4)c32)cc1 ; c1ccc2c(c1)Oc1cc(-c3ccccc3-c3ccc(-c4cc5ccccc5c5ccccc45)cc3)cc3c1N2c1ccccc1O3 ; c1ccc2c(c1)Oc1cc(-c3ccccc3-c3ccc(-c4c5ccccc5cc5ccccc45)cc3)cc3c1N2c1ccccc1O3]

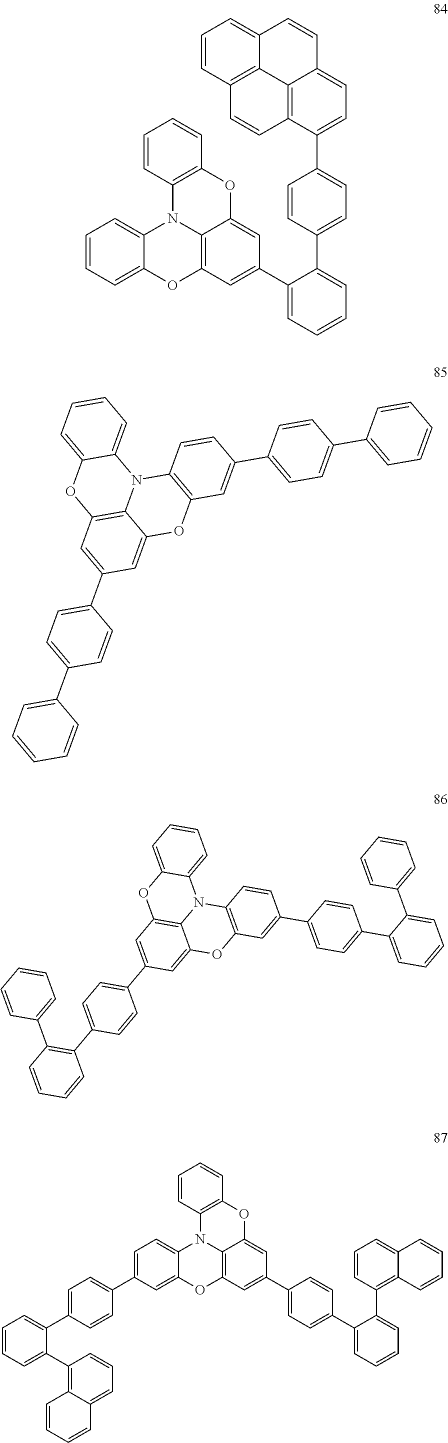 [SMILES: c1ccc2c(c1)Oc1cc(-c3ccccc3-c3ccc(-c4ccc5ccc6cccc7ccc4c5c67)cc3)cc3c1N2c1ccccc1O3 ; c1ccc(-c2ccc(-c3ccc4c(c3)Oc3cc(-c5ccc(-c6ccccc6)cc5)cc5c3N4c3ccccc3O5)cc2)cc1 ; c1ccc(-c2ccccc2-c2ccc(-c3ccc4c(c3)Oc3cc(-c5ccc(-c6ccccc6-c6ccccc6)cc5)cc5c3N4c3ccccc3O5)cc2)cc1 ; c1ccc2c(c1)Oc1cc(-c3ccc(-c4ccccc4-c4cccc5ccccc45)cc3)cc3c1N2c1ccc(-c2ccc(-c4ccccc4-c4cccc5ccccc45)cc2)cc1O3]

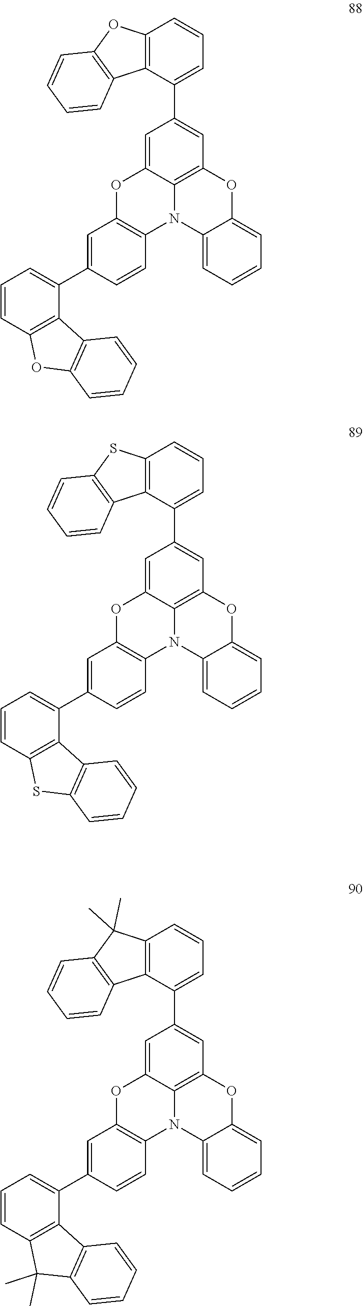 [SMILES: c1ccc2c(c1)Oc1cc(-c3cccc4oc5ccccc5c34)cc3c1N2c1ccc(-c2cccc4oc5ccccc5c24)cc1O3 ; c1ccc2c(c1)Oc1cc(-c3cccc4sc5ccccc5c34)cc3c1N2c1ccc(-c2cccc4sc5ccccc5c24)cc1O3 ; CC1(C)c2ccccc2-c2c(-c3ccc4c(c3)Oc3cc(-c5cccc6c5-c5ccccc5C6(C)C)cc5c3N4c3ccccc3O5)cccc21]

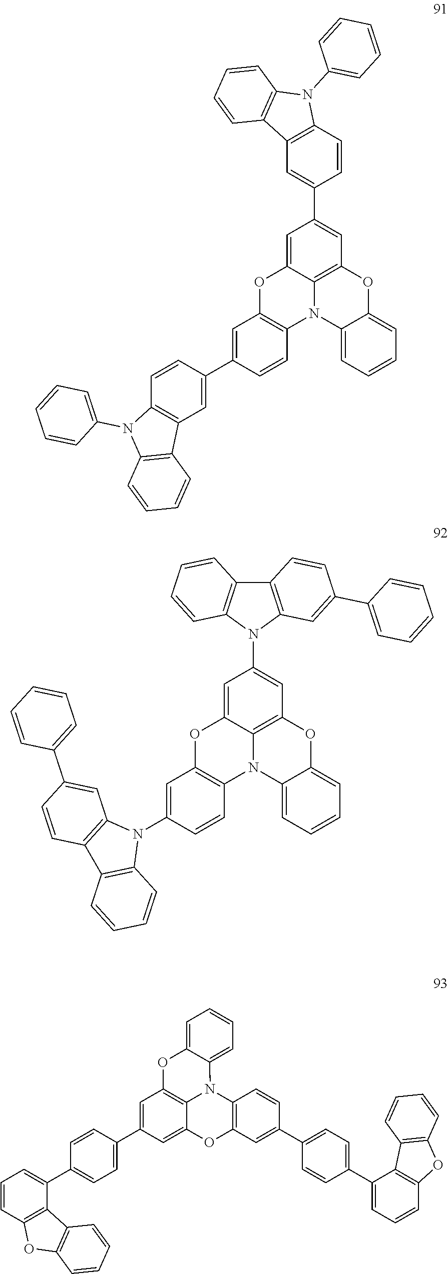 [SMILES: c1ccc(-n2c3ccccc3c3cc(-c4ccc5c(c4)Oc4cc(-c6ccc7c(c6)c6ccccc6n7-c6ccccc6)cc6c4N5c4ccccc4O6)ccc32)cc1 ; c1ccc(-c2ccc3c4ccccc4n(-c4ccc5c(c4)Oc4cc(-n6c7ccccc7c7ccc(-c8ccccc8)cc76)cc6c4N5c4ccccc4O6)c3c2)cc1 ; c1ccc2c(c1)Oc1cc(-c3ccc(-c4cccc5oc6ccccc6c45)cc3)cc3c1N2c1ccc(-c2ccc(-c4cccc5oc6ccccc6c45)cc2)cc1O3]

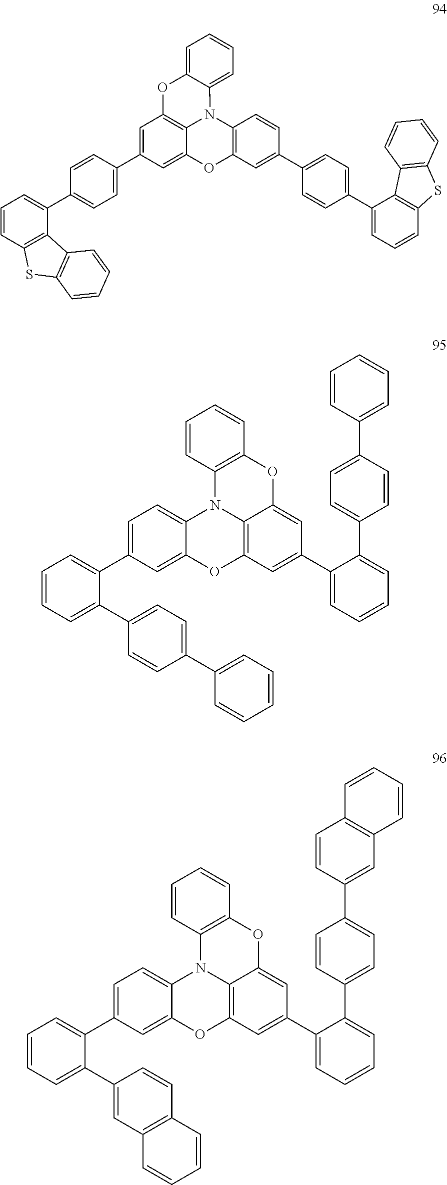 [SMILES: c1ccc2c(c1)Oc1cc(-c3ccc(-c4cccc5sc6ccccc6c45)cc3)cc3c1N2c1ccc(-c2ccc(-c4cccc5sc6ccccc6c45)cc2)cc1O3 ; c1ccc(-c2ccc(-c3ccccc3-c3ccc4c(c3)Oc3cc(-c5ccccc5-c5ccc(-c6ccccc6)cc5)cc5c3N4c3ccccc3O5)cc2)cc1 ; c1ccc2c(c1)Oc1cc(-c3ccccc3-c3ccc(-c4ccc5ccccc5c4)cc3)cc3c1N2c1ccc(-c2ccccc2-c2ccc4ccccc4c2)cc1O3]

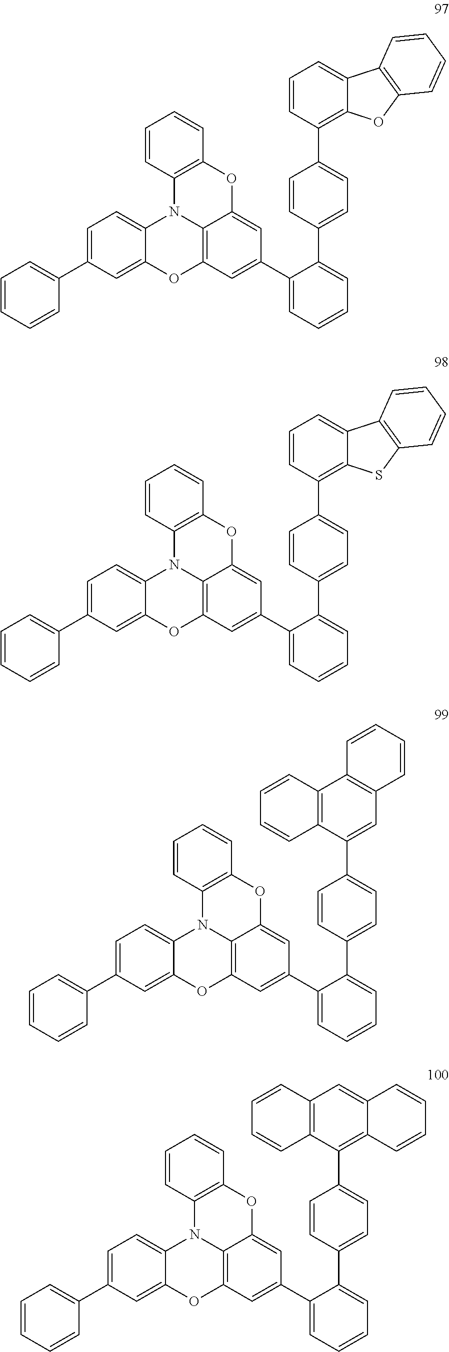

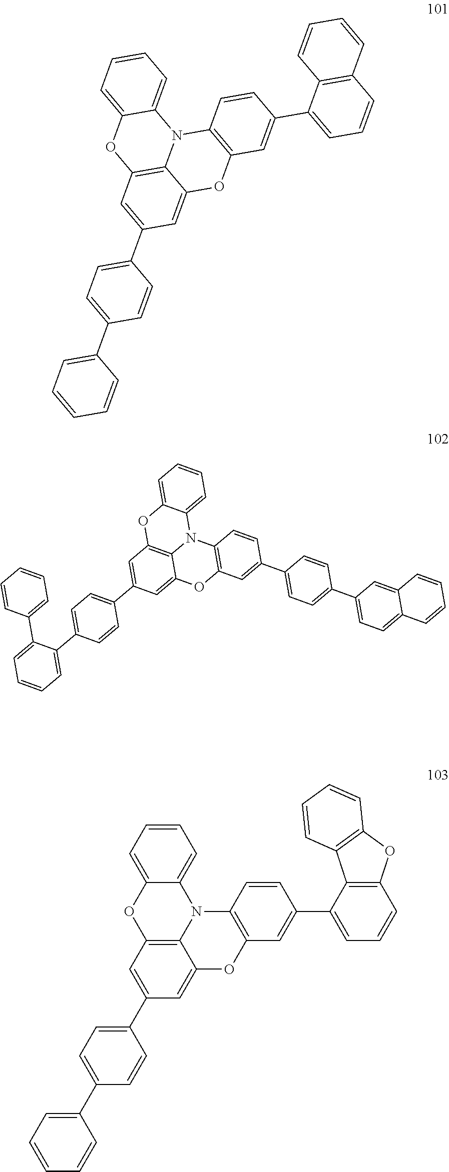 [SMILES: c1ccc(-c2ccc(-c3cc4c5c(c3)Oc3cc(-c6cccc7ccccc67)ccc3N5c3ccccc3O4)cc2)cc1 ; c1ccc(-c2ccccc2-c2ccc(-c3cc4c5c(c3)Oc3cc(-c6ccc(-c7ccc8ccccc8c7)cc6)ccc3N5c3ccccc3O4)cc2)cc1 ; c1ccc(-c2ccc(-c3cc4c5c(c3)Oc3cc(-c6cccc7oc8ccccc8c67)ccc3N5c3ccccc3O4)cc2)cc1]

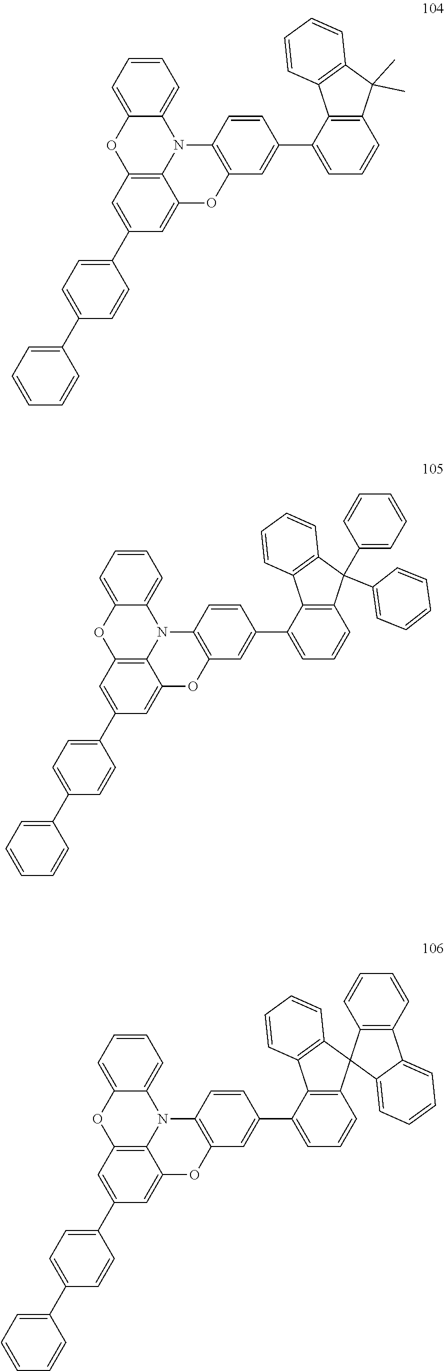 [SMILES: CC1(C)c2ccccc2-c2c(-c3ccc4c(c3)Oc3cc(-c5ccc(-c6ccccc6)cc5)cc5c3N4c3ccccc3O5)cccc21 ; c1ccc(-c2ccc(-c3cc4c5c(c3)Oc3cc(-c6cccc7c6-c6ccccc6C7(c6ccccc6)c6ccccc6)ccc3N5c3ccccc3O4)cc2)cc1 ; c1ccc(-c2ccc(-c3cc4c5c(c3)Oc3cc(-c6cccc7c6-c6ccccc6C76c7ccccc7-c7ccccc76)ccc3N5c3ccccc3O4)cc2)cc1]

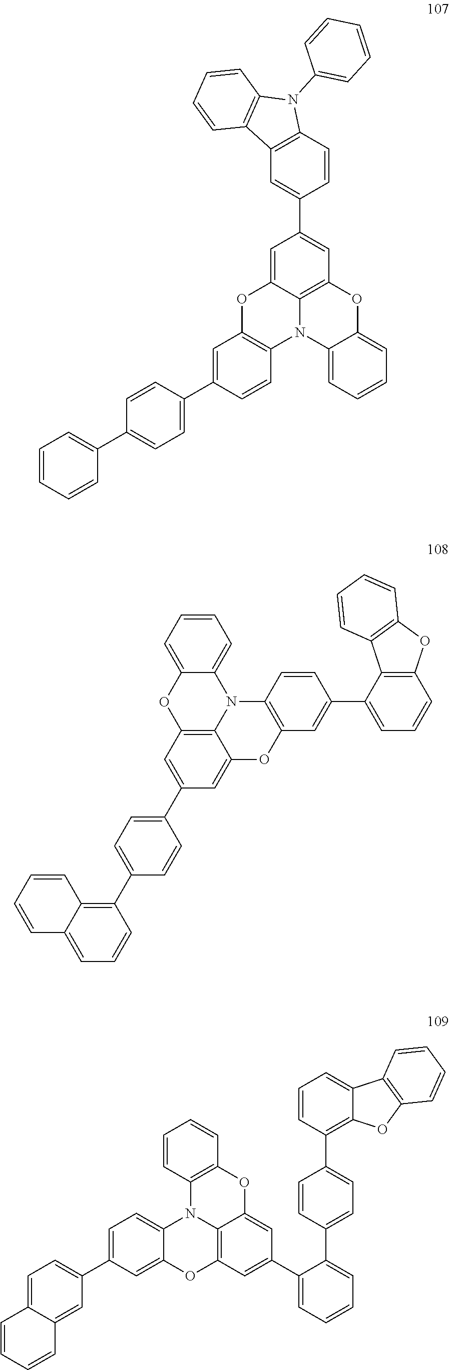 [SMILES: c1ccc(-c2ccc(-c3ccc4c(c3)Oc3cc(-c5ccc6c(c5)c5ccccc5n6-c5ccccc5)cc5c3N4c3ccccc3O5)cc2)cc1 ; c1ccc2c(c1)Oc1cc(-c3ccc(-c4cccc5ccccc45)cc3)cc3c1N2c1ccc(-c2cccc4oc5ccccc5c24)cc1O3 ; c1ccc2c(c1)Oc1cc(-c3ccccc3-c3ccc(-c4cccc5c4oc4ccccc45)cc3)cc3c1N2c1ccc(-c2ccc4ccccc4c2)cc1O3]

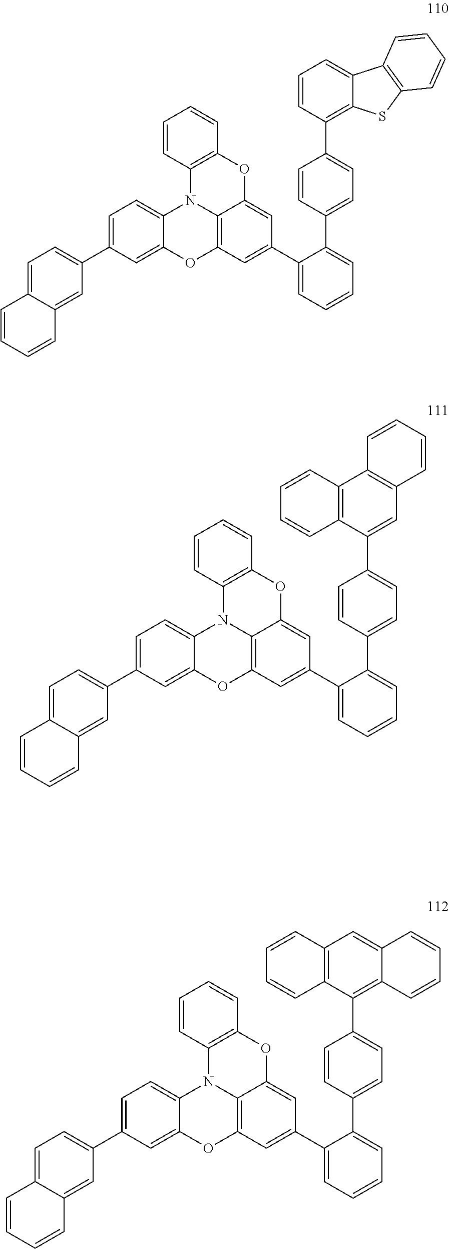 [SMILES: c1ccc2c(c1)Oc1cc(-c3ccccc3-c3ccc(-c4cccc5c4sc4ccccc45)cc3)cc3c1N2c1ccc(-c2ccc4ccccc4c2)cc1O3 ; c1ccc2c(c1)Oc1cc(-c3ccccc3-c3ccc(-c4cc5ccccc5c5ccccc45)cc3)cc3c1N2c1ccc(-c2ccc4ccccc4c2)cc1O3 ; c1ccc2c(c1)Oc1cc(-c3ccccc3-c3ccc(-c4c5ccccc5cc5ccccc45)cc3)cc3c1N2c1ccc(-c2ccc4ccccc4c2)cc1O3]

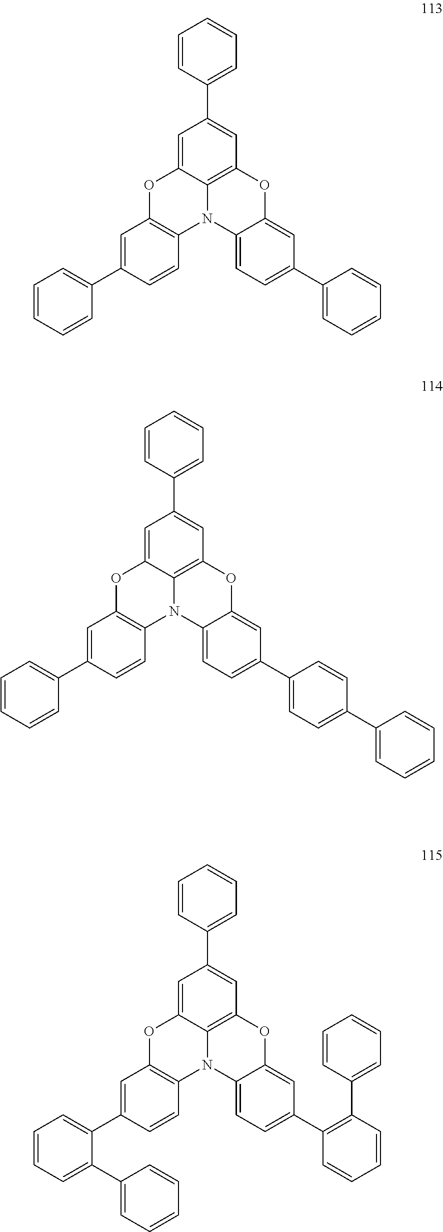 [SMILES: c1ccc(-c2ccc3c(c2)Oc2cc(-c4ccccc4)cc4c2N3c2ccc(-c3ccccc3)cc2O4)cc1 ; c1ccc(-c2ccc(-c3ccc4c(c3)Oc3cc(-c5ccccc5)cc5c3N4c3ccc(-c4ccccc4)cc3O5)cc2)cc1 ; c1ccc(-c2cc3c4c(c2)Oc2cc(-c5ccccc5-c5ccccc5)ccc2N4c2ccc(-c4ccccc4-c4ccccc4)cc2O3)cc1]

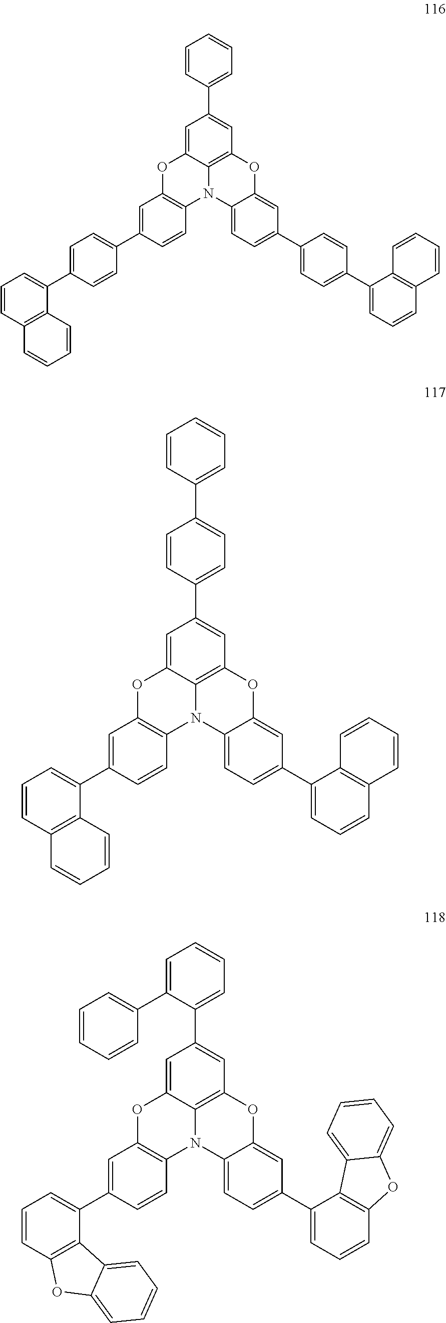 [SMILES: c1ccc(-c2cc3c4c(c2)Oc2cc(-c5ccc(-c6cccc7ccccc67)cc5)ccc2N4c2ccc(-c4ccc(-c5cccc6ccccc56)cc4)cc2O3)cc1 ; c1ccc(-c2ccc(-c3cc4c5c(c3)Oc3cc(-c6cccc7ccccc67)ccc3N5c3ccc(-c5cccc6ccccc56)cc3O4)cc2)cc1 ; c1ccc(-c2ccccc2-c2cc3c4c(c2)Oc2cc(-c5cccc6oc7ccccc7c56)ccc2N4c2ccc(-c4cccc5oc6ccccc6c45)cc2O3)cc1]

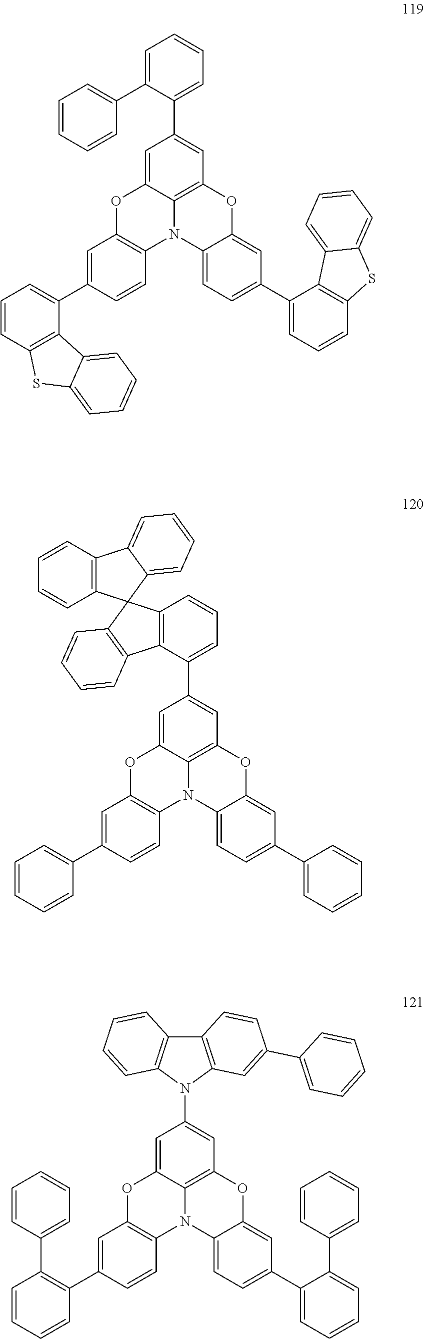 [SMILES: c1ccc(-c2ccccc2-c2cc3c4c(c2)Oc2cc(-c5cccc6sc7ccccc7c56)ccc2N4c2ccc(-c4cccc5sc6ccccc6c45)cc2O3)cc1 ; c1ccc(-c2ccc3c(c2)Oc2cc(-c4cccc5c4-c4ccccc4C54c5ccccc5-c5ccccc54)cc4c2N3c2ccc(-c3ccccc3)cc2O4)cc1 ; c1ccc(-c2ccc3c4ccccc4n(-c4cc5c6c(c4)Oc4cc(-c7ccccc7-c7ccccc7)ccc4N6c4ccc(-c6ccccc6-c6ccccc6)cc4O5)c3c2)cc1]

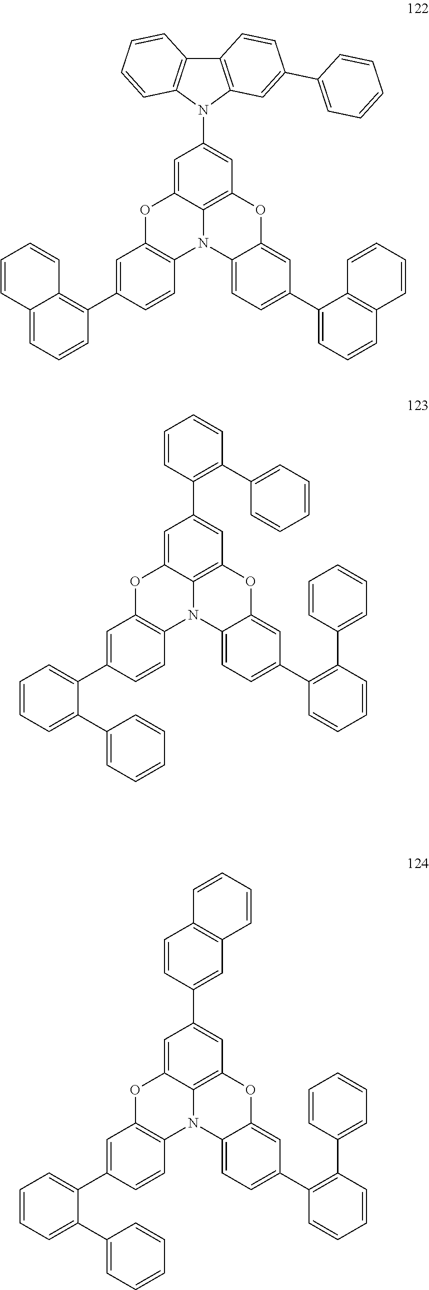 [SMILES: c1ccc(-c2ccc3c4ccccc4n(-c4cc5c6c(c4)Oc4cc(-c7cccc8ccccc78)ccc4N6c4ccc(-c6cccc7ccccc67)cc4O5)c3c2)cc1 ; c1ccc(-c2ccccc2-c2ccc3c(c2)Oc2cc(-c4ccccc4-c4ccccc4)cc4c2N3c2ccc(-c3ccccc3-c3ccccc3)cc2O4)cc1 ; c1ccc(-c2ccccc2-c2ccc3c(c2)Oc2cc(-c4ccc5ccccc5c4)cc4c2N3c2ccc(-c3ccccc3-c3ccccc3)cc2O4)cc1]

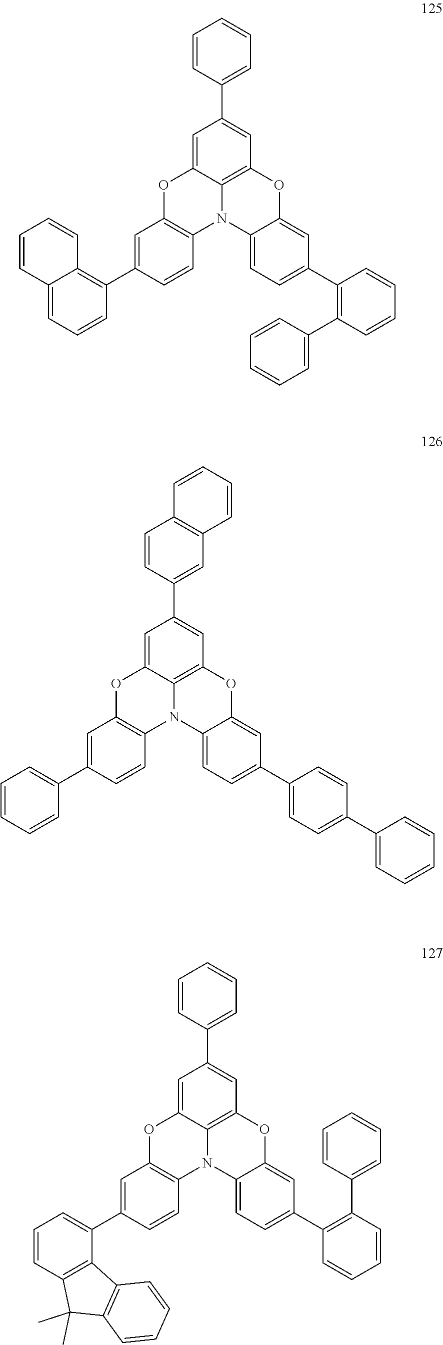 [SMILES: c1ccc(-c2cc3c4c(c2)Oc2cc(-c5cccc6ccccc56)ccc2N4c2ccc(-c4ccccc4-c4ccccc4)cc2O3)cc1 ; c1ccc(-c2ccc(-c3ccc4c(c3)Oc3cc(-c5ccc6ccccc6c5)cc5c3N4c3ccc(-c4ccccc4)cc3O5)cc2)cc1 ; CC1(C)c2ccccc2-c2c(-c3ccc4c(c3)Oc3cc(-c5ccccc5)cc5c3N4c3ccc(-c4ccccc4-c4ccccc4)cc3O5)cccc21]

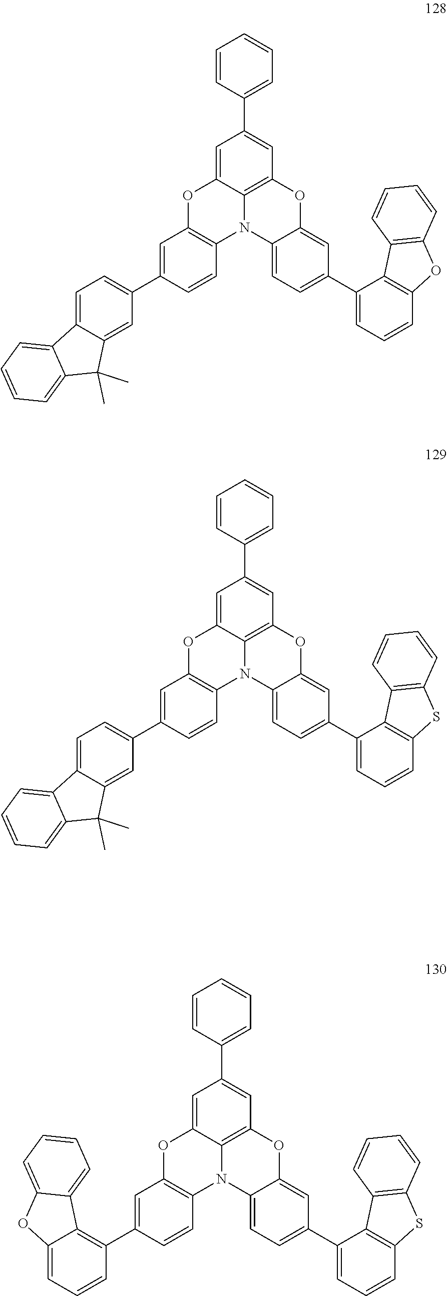 [SMILES: CC1(C)c2ccccc2-c2ccc(-c3ccc4c(c3)Oc3cc(-c5ccccc5)cc5c3N4c3ccc(-c4cccc6oc7ccccc7c46)cc3O5)cc21 ; CC1(C)c2ccccc2-c2ccc(-c3ccc4c(c3)Oc3cc(-c5ccccc5)cc5c3N4c3ccc(-c4cccc6sc7ccccc7c46)cc3O5)cc21 ; c1ccc(-c2cc3c4c(c2)Oc2cc(-c5cccc6sc7ccccc7c56)ccc2N4c2ccc(-c4cccc5oc6ccccc6c45)cc2O3)cc1]

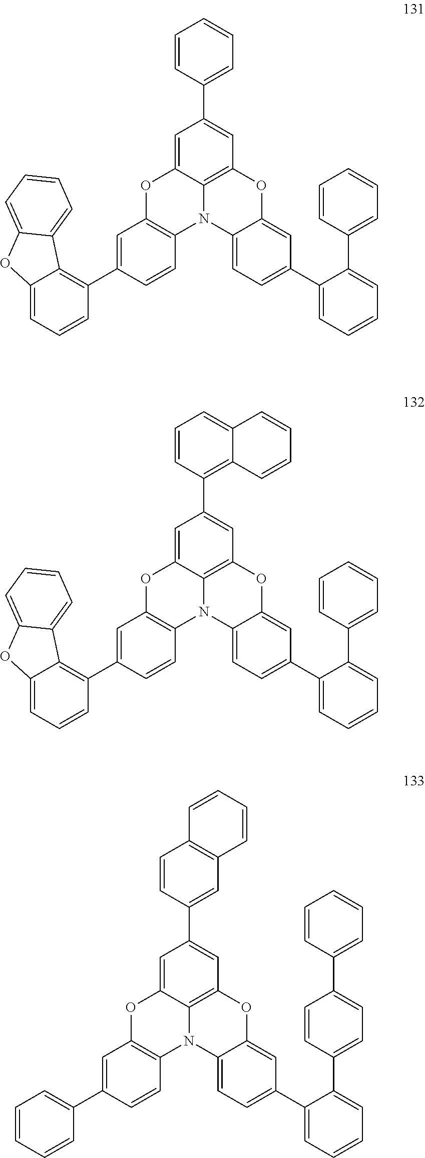 [SMILES: c1ccc(-c2cc3c4c(c2)Oc2cc(-c5cccc6oc7ccccc7c56)ccc2N4c2ccc(-c4ccccc4-c4ccccc4)cc2O3)cc1 ; c1ccc(-c2ccccc2-c2ccc3c(c2)Oc2cc(-c4cccc5ccccc45)cc4c2N3c2ccc(-c3cccc5oc6ccccc6c35)cc2O4)cc1 ; c1ccc(-c2ccc(-c3ccccc3-c3ccc4c(c3)Oc3cc(-c5ccc6ccccc6c5)cc5c3N4c3ccc(-c4ccccc4)cc3O5)cc2)cc1]

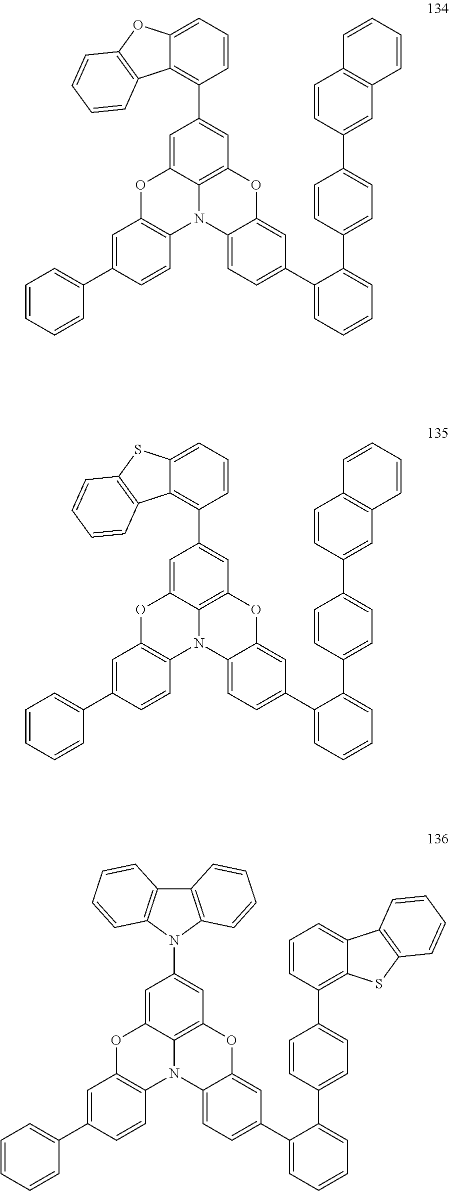 [SMILES: c1ccc(-c2ccc3c(c2)Oc2cc(-c4cccc5oc6ccccc6c45)cc4c2N3c2ccc(-c3ccccc3-c3ccc(-c5ccc6ccccc6c5)cc3)cc2O4)cc1 ; c1ccc(-c2ccc3c(c2)Oc2cc(-c4cccc5sc6ccccc6c45)cc4c2N3c2ccc(-c3ccccc3-c3ccc(-c5ccc6ccccc6c5)cc3)cc2O4)cc1 ; c1ccc(-c2ccc3c(c2)Oc2cc(-n4c5ccccc5c5ccccc54)cc4c2N3c2ccc(-c3ccccc3-c3ccc(-c5cccc6c5sc5ccccc56)cc3)cc2O4)cc1]

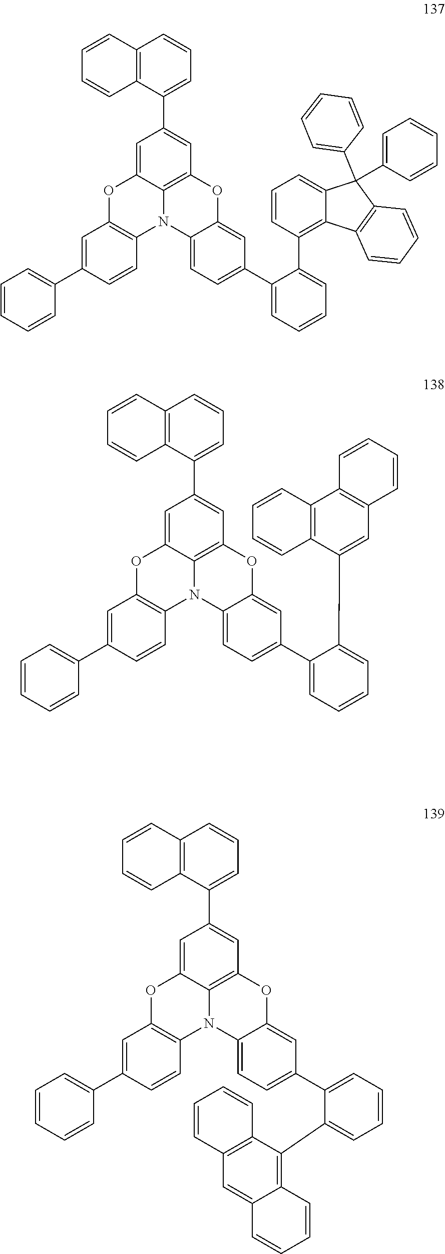 [SMILES: c1ccc(-c2ccc3c(c2)Oc2cc(-c4cccc5ccccc45)cc4c2N3c2ccc(-c3ccccc3-c3cccc5c3-c3ccccc3C5(c3ccccc3)c3ccccc3)cc2O4)cc1 ; c1ccc(-c2ccc3c(c2)Oc2cc(-c4cccc5ccccc45)cc4c2N3c2ccc(-c3ccccc3-c3cc5ccccc5c5ccccc35)cc2O4)cc1 ; c1ccc(-c2ccc3c(c2)Oc2cc(-c4cccc5ccccc45)cc4c2N3c2ccc(-c3ccccc3-c3c5ccccc5cc5ccccc35)cc2O4)cc1]

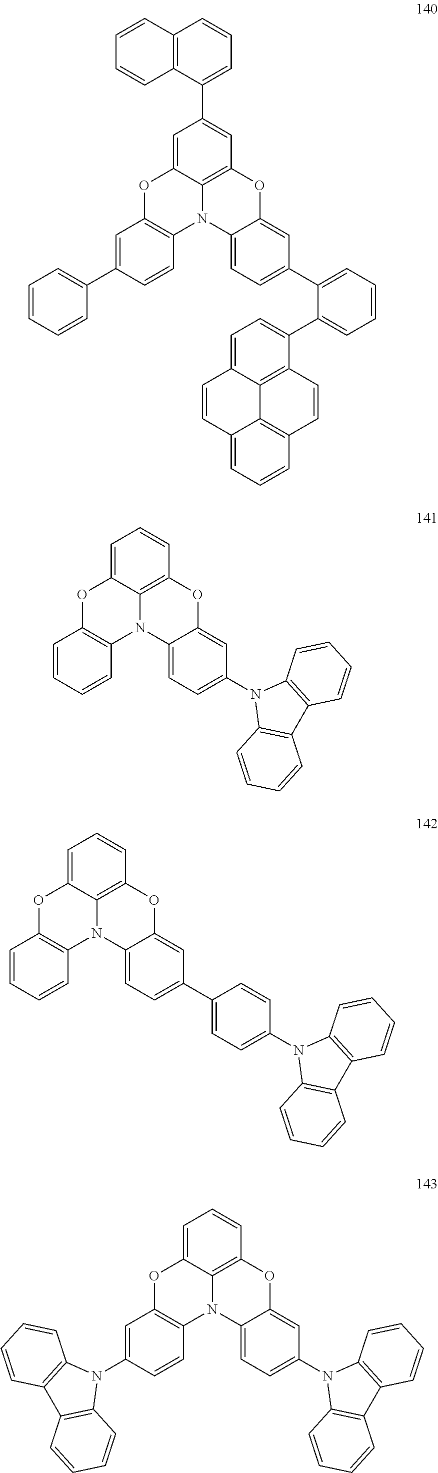 [SMILES: c1ccc(-c2ccc3c(c2)Oc2cc(-c4cccc5ccccc45)cc4c2N3c2ccc(-c3ccccc3-c3ccc5ccc6cccc7ccc3c5c67)cc2O4)cc1 ; c1ccc2c(c1)Oc1cccc3c1N2c1ccc(-n2c4ccccc4c4ccccc42)cc1O3 ; c1ccc2c(c1)Oc1cccc3c1N2c1ccc(-c2ccc(-n4c5ccccc5c5ccccc54)cc2)cc1O3 ; c1cc2c3c(c1)Oc1cc(-n4c5ccccc5c5ccccc54)ccc1N3c1ccc(-n3c4ccccc4c4ccccc43)cc1O2]

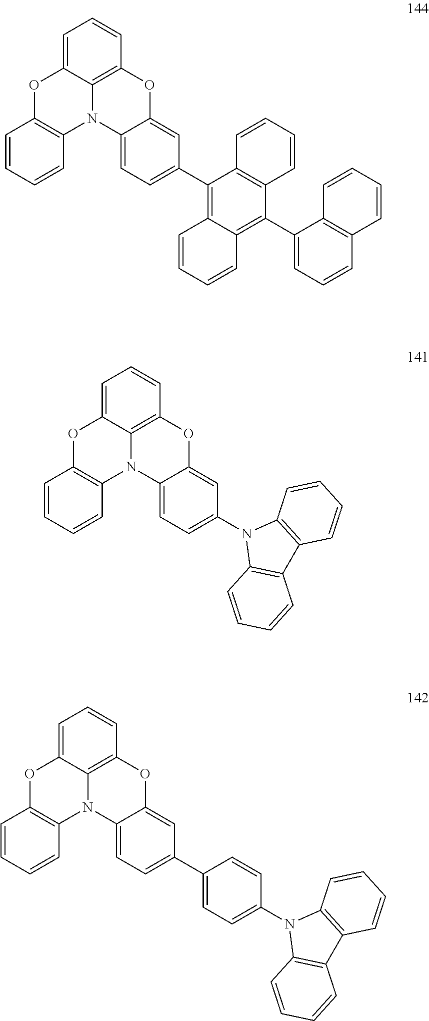 [SMILES: c1ccc2c(c1)Oc1cccc3c1N2c1ccc(-c2c4ccccc4c(-c4cccc5ccccc45)c4ccccc24)cc1O3 ; c1ccc2c(c1)Oc1cccc3c1N2c1ccc(-n2c4ccccc4c4ccccc42)cc1O3 ; c1ccc2c(c1)Oc1cccc3c1N2c1ccc(-c2ccc(-n4c5ccccc5c5ccccc54)cc2)cc1O3]

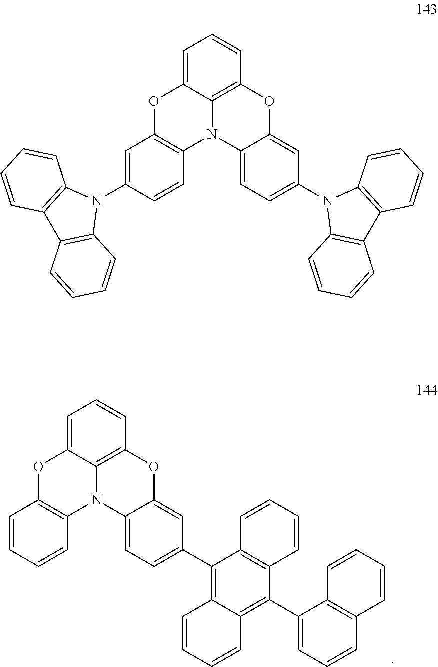 [SMILES: c1cc2c3c(c1)Oc1cc(-n4c5ccccc5c5ccccc54)ccc1N3c1ccc(-n3c4ccccc4c4ccccc43)cc1O2 ; c1ccc2c(c1)Oc1cccc3c1N2c1ccc(-c2c4ccccc4c(-c4cccc5ccccc45)c4ccccc24)cc1O3]

The heterocyclic compound represented by Formula 1 has a high glass transition temperature (Tg), compared to the compound which does not include the condensed ring moiety, due to the condensed ring structure. For example, in some embodiments ring A₁, ring A₂, ring A₃, N, X₁, and X₂ combine to form a condensed ring structure that results in the heterocyclic compound represented by Formula 1 having a high Tg. Therefore, heat resistance to Joule heat generated during the driving of the organic light-emitting device including the heterocyclic compound represented by Formula 1 may be improved, and thus, as a result the heterocyclic compound has long lifespan characteristics.

Also, because the heterocyclic compound represented by Formula 1 includes N at the center in a core structure and further includes a heteroatom of O or S as X₁ and X₂, the heterocyclic compound has a flat structure and a strong T-T bond strength, thereby increasing charge mobility characteristics of the heterocyclic compound represented by Formula 1. For example, in some embodiments ring A₁, ring A₂, ring A₃, N, X₁, and X₂ combine to form a condensed ring structure that is planar, thereby providing a delocalized T electron system (e.g., providing a high level of conjugation or aromaticity), and increasing the ability of the heterocyclic compound represented by Formula 1 to transport a charge.

Also, the heterocyclic compound represented by Formula 1 has a structure that includes (or essentially includes) one or more aryl (Ar) substituents and does not include a substituent having the same structure as that of the core. For example, in some embodiments ring A₁, ring A₂, ring A₃, N, X₁, and X₂ combine to form a condensed ring structure that is different from each of the substituents of the heterocyclic compound represented by Formula 1. Therefore, as a result, a difference occurs in electron cloud distribution of highest occupied molecular orbital (HOMO) and lowest unoccupied molecular orbital (LUMO), and the heterocyclic compound represented by Formula 1 has an energy level suitable or appropriate for use as a hole transport layer.

A synthesis method for the heterocyclic compound represented by Formula 1 would be apparent to those of ordinary skill in the art by referring to the following examples.

The heterocyclic compound of Formula 1 may be included in an organic layer between a pair of electrodes of an organic light-emitting device. For example, the heterocyclic compound may be included in at least one layer selected from a hole transport region, an electron transport region, and an emission layer. In one or more embodiments, the heterocyclic compound of Formula 1 may be used as a material for a capping layer located outside a pair of electrodes of an organic light-emitting device.

Accordingly, another aspect of an embodiment of the present disclosure provides an organic light-emitting device including: a first electrode; a second electrode facing the first electrode; an organic layer between the first electrode and the second electrode and including an emission layer; and at least one heterocyclic compound represented by Formula 1.

In one embodiment, the organic layer of the organic light-emitting device may include at least one heterocyclic compound represented by Formula 1.

The expression “(an organic layer) includes at least one heterocyclic compound,” as used herein, may include a case in which “(an organic layer) includes identical heterocyclic compounds represented by Formula 1” and a case in which “(an organic layer) includes two or more different heterocyclic compounds represented by Formula 1”.

For example, the organic layer may include, as the heterocyclic compound, only Compound 1. In one or more embodiments, the organic layer may include, as the heterocyclic compound, Compound 1 and Compound 2. Here, Compound 1 and Compound 2 may exist in an identical layer (for example, Compound 1 and Compound 2 may all exist in a hole transport layer), or different layers (for example, Compound 1 may exist in a first hole transport layer and Compound 2 may exist in a second hole transport layer).

In one embodiment, the first electrode is an anode, and the second electrode is a cathode, and the organic layer further includes a hole transport region between the first electrode and the emission layer and an electron transport region between the emission layer and the second electrode, and the hole transport region includes a hole injection layer, a hole transport layer, an emission auxiliary layer, an electron blocking layer, or any combination thereof, and the electron transport region includes a buffer layer, a hole blocking layer, an electron control layer, an electron transport layer, an electron injection layer, or any combination thereof.

In one or more embodiments, the hole transport region of the organic light-emitting device may include the heterocyclic compound represented by Formula 1.

In one or more embodiments, the hole transport region of the organic light-emitting device may include at least one of a hole injection layer and a hole transport layer, wherein the at least one of the hole injection layer and the hole transport layer may include the heterocyclic compound represented by Formula 1.

For example, the hole transport region of the organic light-emitting device may include the hole transport layer, wherein the hole transport layer includes the heterocyclic compound represented by Formula 1.

In one embodiment, the hole transport region of the organic light-emitting device may further include a p-dopant having a lowest unoccupied molecular orbital (LUMO) energy level of about −3.5 eV or less.

For example, the p-dopant may include at least one selected from a quinone derivative, a metal oxide, and a cyano group-containing compound.

For example, the hole transport region may include a hole transport layer, which includes the p-dopant, or may include a hole injection layer, which includes the p-dopant.

In one embodiment, the electron transport region of the organic light-emitting device may include an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or any combinations thereof.

In one embodiment, the emission layer of the organic light-emitting device may include at least one selected from an anthracene-based compound, a pyrene-based compound, a spiro-bifluorene-based compound, a carbazole-based compound, a benzimidazole-based compound, a phosphine oxide-based compound, a dibenzofuran-based compound, a silicon-based compound, and a triazine-based compound.

The term “organic layer,” as used herein, refers to a single layer and/or a plurality of layers between the first electrode and the second electrode of the organic light-emitting device. A material included in the “organic layer” is not limited to an organic material. For example, the organic layer may include an inorganic material

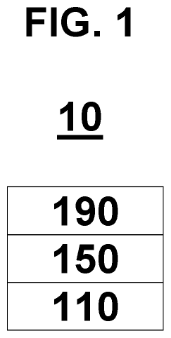

### Description of FIG. 1

FIG. 1 is a schematic cross-sectional view of an organic light-emitting device 10 according to an embodiment. The organic light-emitting device 10 includes a first electrode 110, an organic layer 150, and a second electrode 190.

Hereinafter, the structure of the organic light-emitting device 10 according to an embodiment and a method of manufacturing the organic light-emitting device 10 will be described in connection with FIG. 1.

### First Electrode 110

In FIG. 1, a substrate may be additionally under the first electrode 110 or above the second electrode 190. The substrate may be a glass substrate or a plastic substrate, each having excellent mechanical strength, thermal stability, transparency, surface smoothness, ease of handling, and water resistance.

The first electrode 110 may be formed by depositing or sputtering a material for forming the first electrode 110 on the substrate. When the first electrode 110 is an anode, the material for forming the first electrode 110 may be selected from materials with a high work function to facilitate hole injection.

The first electrode 110 may be a reflective electrode, a semi-reflective electrode, or a transmissive electrode. When the first electrode 110 is a transmissive electrode, a material for forming a first electrode may be selected from indium tin oxide (ITO), indium zinc oxide (IZO), tin oxide (SnO₂), zinc oxide (ZnO), and any combinations thereof, but embodiments of the present disclosure are not limited thereto. In one or more embodiments, when the first electrode 110 is a semi-transmissive electrode or a reflectable electrode, a material for forming a first electrode may be selected from magnesium (Mg), silver (Ag), aluminum (Al), aluminum-lithium (Al—Li), calcium (Ca), magnesium-indium (Mg—In), magnesium-silver (Mg—Ag), and any combinations thereof, but embodiments of the present disclosure are not limited thereto.

The first electrode 110 may have a single-layered structure, or a multi-layered structure including two or more layers. For example, the first electrode 110 may have a three-layered structure of ITO/Ag/ITO, but the structure of the first electrode 110 is not limited thereto.

### Organic Layer 150

The organic layer 150 is on the first electrode 110. The organic layer 150 may include an emission layer.

The organic layer 150 may further include a hole transport region between the first electrode 110 and the emission layer and an electron transport region between the emission layer and the second electrode 190.

### Hole Transport Region in Organic Layer 150

The hole transport region may have i) a single-layered structure including a single layer including a single material, ii) a single-layered structure including a single layer including a plurality of different materials, or iii) a multi-layered structure having a plurality of layers including a plurality of different materials.

The hole transport region may include at least one layer selected from a hole injection layer, a hole transport layer, an emission auxiliary layer, and an electron blocking layer.

For example, the hole transport region may have a single-layered structure including a single layer including a plurality of different materials, or a multi-layered structure having a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, wherein for each structure, constituting layers are sequentially stacked from the first electrode 110 in this stated order, but the structure of the hole transport region is not limited thereto.

The hole transport region may include the heterocyclic compound represented by Formula 1.

In one or more embodiments, the hole transport region may include at least one selected m-MTDATA, TDATA, 2-TNATA, NPB(NPD), β-NPB, TPD, spiro-TPD, spiro-NPB, methylated-NPB, TAPC, HMTPD, 4,4′,4″-trs(N-carbazolyl)trphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), a compound represented by Formula 201 below, and a compound represented by Formula 202 below:

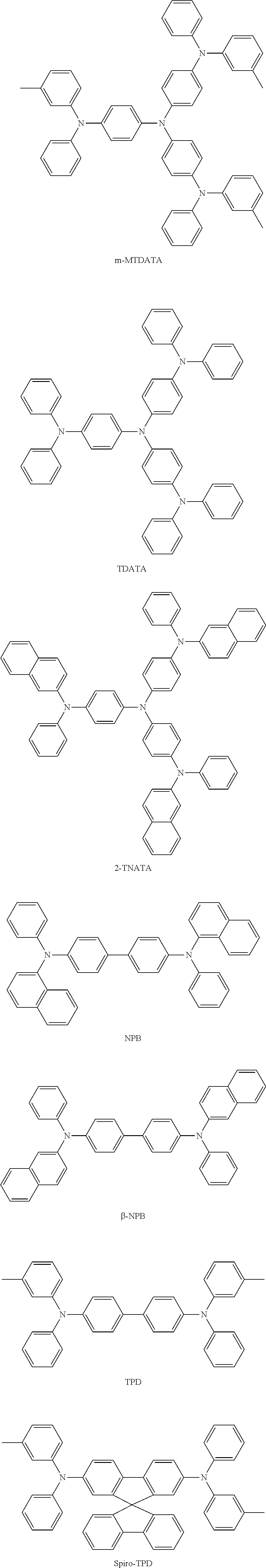

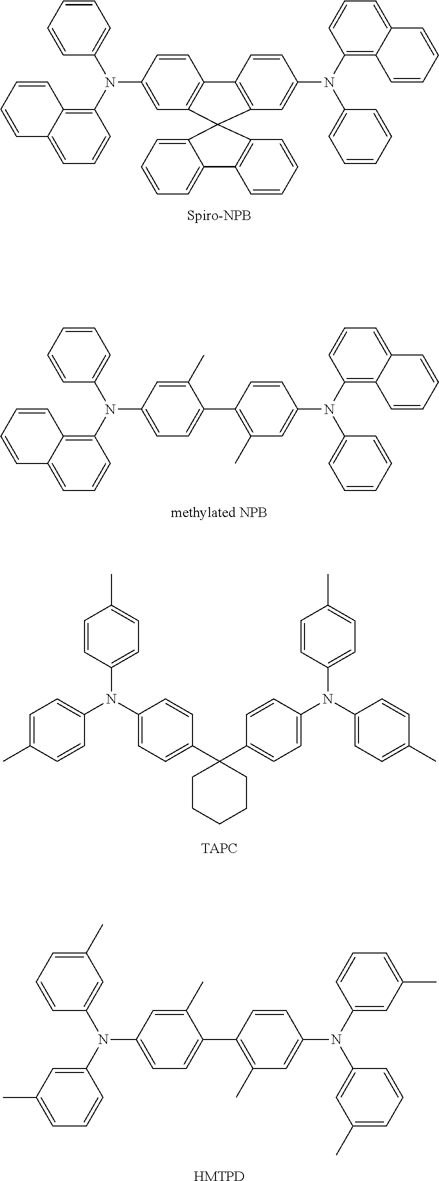

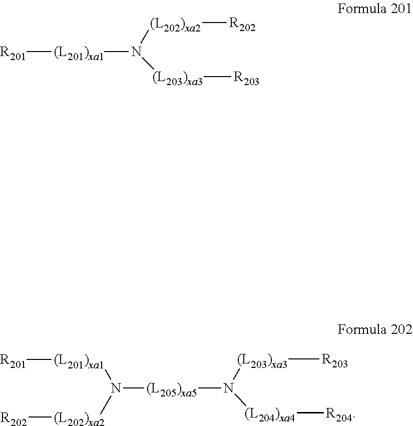

In Formulae 201 and 202,

L₂₀₁ to L₂₀₄ may each independently be selected from a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group,

L₂₀₅ may be selected from *—O—*, *—S—*′, *—N(Q₂₀₁)-*′, a substituted or unsubstituted C₁-C₂₀ alkylene group, a substituted or unsubstituted C₂-C₂₀ alkenylene group, a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group,

xa1 to xa4 may each independently bean integer from 0 to 3,

xa5 may be an integer 1 to 10, and

R₂₀₁ to R₂₀₄ and Q₂₀₁ may each independently be selected from a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

For example, in Formula 202, R₂₀₁ and R₂₀₂ may optionally be linked to each other via a single bond, a dimethyl-methylene group, or a diphenyl-methylene group, and R₂₀₃ and R₂₀₁ may optionally be linked to each other via a single bond, a dimethyl-methylene group, or a diphenyl-methylene group.

In one embodiment, in Formulae 201 and 202,

L₂₀₁ to L₂₀₅ may each independently be selected from:

a phenylene group, a pentalenylene group, an indenylene group, a naphthylene group, an azulenylene group, a heptalenylene group, an indacenylene group, an acenaphthylene group, a fluorenylene group, a spiro-bifluorenylene group, a benzofluorenylene group, a dibenzofluorenylene group, a phenalenylene group, a phenanthrenylene group, an anthracenylene group, a fluoranthenylene group, a triphenylenylene group, a pyrenylene group, a chrysenylene group, a naphthacenylene group, a picenylene group, a perylenylene group, a pentaphenylene group, a hexacenylene group, a pentacenylene group, a rubicenylene group, a coronenylene group, an ovalenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, and a pyridinylene group; and

a phenylene group, a pentalenylene group, an indenylene group, a naphthylene group, an azulenylene group, a heptalenylene group, an indacenylene group, an acenaphthylene group, a fluorenylene group, a spiro-bifluorenylene group, a benzofluorenylene group, a dibenzofluorenylene group, a phenalenylene group, a phenanthrenylene group, an anthracenylene group, a fluoranthenylene group, a triphenylenylene group, a pyrenylene group, a chrysenylene group, a naphthacenylene group, a picenylene group, a perylenylene group, a pentaphenylene group, a hexacenylene group, a pentacenylene group, a rubicenylene group, a coronenylene group, an ovalenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, and a pyridinylene group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a phenyl group substituted with a C₁-C₁₀ alkyl group, a phenyl group substituted with —F, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, —Si(Q₃₁)(Q₃₂)(Q₃₃), and —N(Q₃₁)(Q₃₂), and

Q₃₁ to Q₃₃ may each independently be selected from a C₁-C₁₀ alkyl group, a C₁-C₁₀ alkoxy group, a phenyl group, a biphenyl group, a terphenyl group, and a naphthyl group.

In one or more embodiments, xa1 to xa4 may each independently be 0, 1, or 2.

In one or more embodiments, xa5 may be 1, 2, 3, or 4.

In one or more embodiments, R₂₀₁ to R₂₀₄ and Q₂₀₁ may each independently be selected from:

a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, and a pyridinyl group; and

a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, and a pyridinyl group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a phenyl group substituted with a C₁-C₁₀ alkyl group, a phenyl group substituted with —F, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, —Si(Q₃₁)(Q₃₂)(Q₃₃), and —N(Q₃₁)(Q₃₂), and

Q₃₁ to Q₃₃ are the same as described herein above.

In one or more embodiments, at least one selected from R₂₀₁ to R₂₀₃ in Formula 201 may each independently be selected from:

a fluorenyl group, a spiro-bifluorenyl group, a carbazolyl group, a dibenzofuranyl group, and a dibenzothiophenyl group; and

a fluorenyl group, a spiro-bifluorenyl group, a carbazolyl group, a dibenzofuranyl group, and a dibenzothiophenyl group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a phenyl group substituted with a C₁-C₁₀ alkyl group, a phenyl group substituted with —F, a naphthyl group, a fluorenyl group, a spiro-bifluorenyl group, a carbazolyl group, a dibenzofuranyl group, and a dibenzothiophenyl group,

but embodiments of the present disclosure are not limited thereto.

In one or more embodiments, in Formula 202, i) R₂₀₁ and R₂₀₂ may be linked to each other via a single bond, and/or ii) R₂₀₃ and R₂₀₁ may be linked to each other via a single bond.

In one or more embodiments, at least one selected from R₂₀₁ to R₂₀₄ in Formula 501 may each independently be selected from:

a carbazolyl group; and

a carbazolyl group substituted with at least one selected from deuterium, —F, —C, —Br, —I, a hydroxyl group, a cyano group, a nitro group, an amidino group, a hydrazino group, a hydrazono group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a phenyl group substituted with a C₁-C₁₀ alkyl group, a phenyl group substituted with —F, a naphthyl group, a fluorenyl group, a spiro-bifluorenyl group, a carbazolyl group, a dibenzofuranyl group, and a dibenzothiophenyl group,

but embodiments of the present disclosure are not limited thereto.

The compound represented by Formula 201 may be represented by Formula 201A below:

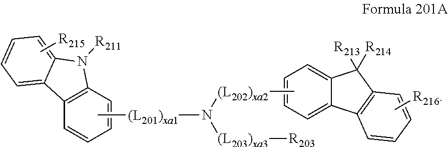

In one embodiment, the compound represented by Formula 201 may be represented by Formula 201A(1) below, but embodiments of the present disclosure are not limited thereto:

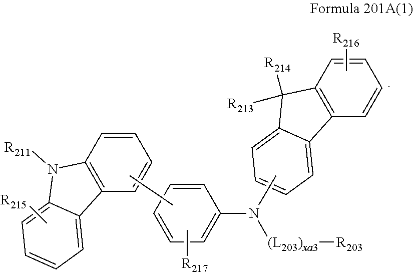

In one or more embodiments, the compound represented by Formula 201 may be represented by Formula 201A-1 below, but embodiments of the present disclosure are not limited thereto:

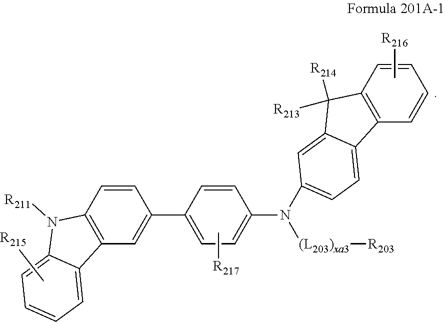

In one embodiment, the compound represented by Formula 202 may be represented by Formula 202A below:

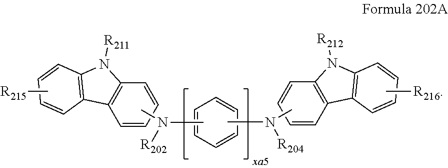

In one or more embodiments, the compound represented by Formula 202 may be represented by Formula 202A-1 below:

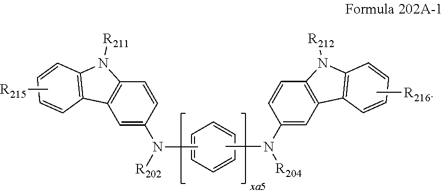

In Formulae 201A, 201A(1), 201A-1, 202A, and 202A-1,

L₂₀₁ to L₂₀₃, xa1 to xa3, xa5, and R₂₀₂ to R₂₀₄ are the same as described herein above,

R₂₁₁ and R₂₁₂ may be understood by referring to the description provided herein in connection with R₂₀₃, and

R₂₁₃ to R₂₁₇ may each independently be selected from hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a phenyl group substituted with a C₁-C₁₀ alkyl group, a phenyl group substituted with —F, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, and a pyridinyl group.

The hole transport region may include at least one compound selected from Compounds HT1 to HT39, but embodiments of the present disclosure are not limited thereto:

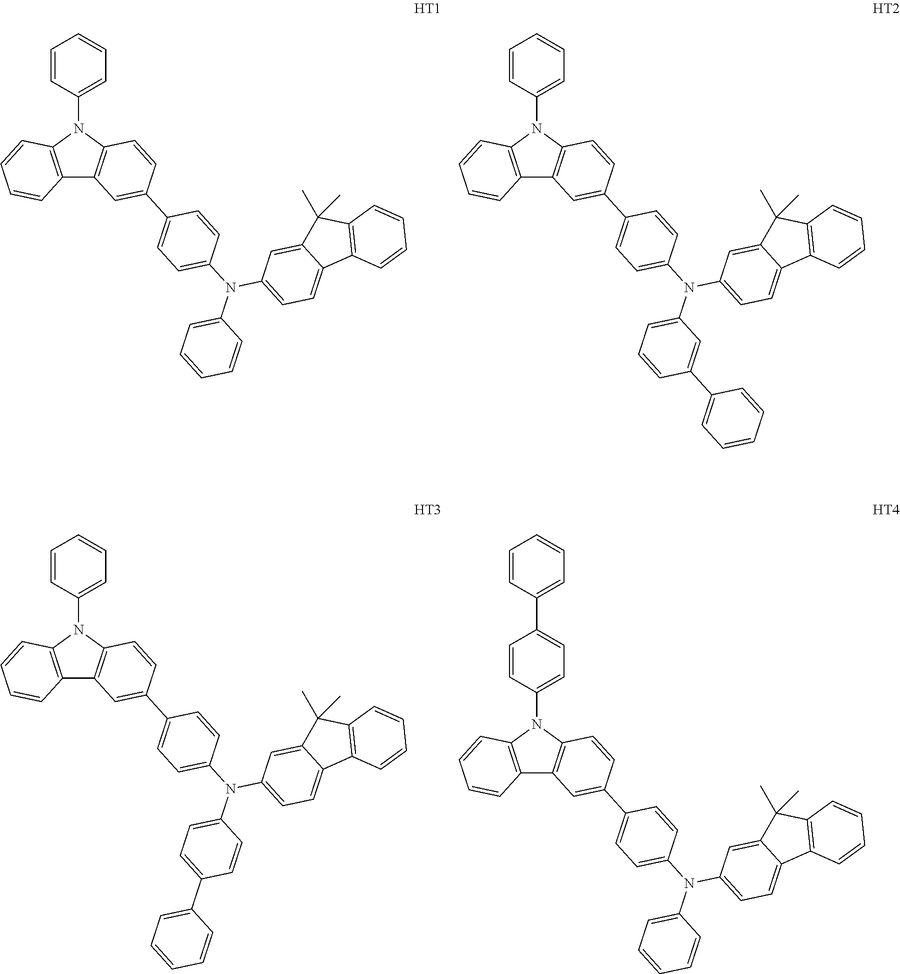 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

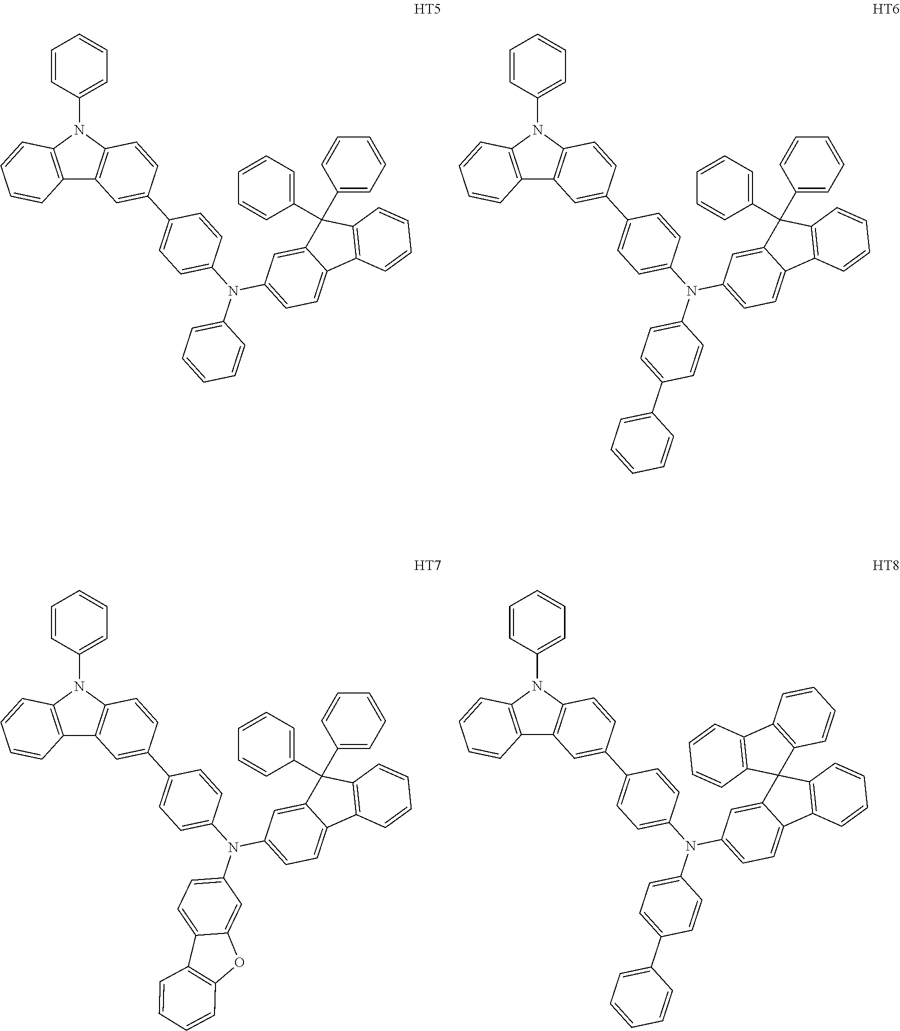

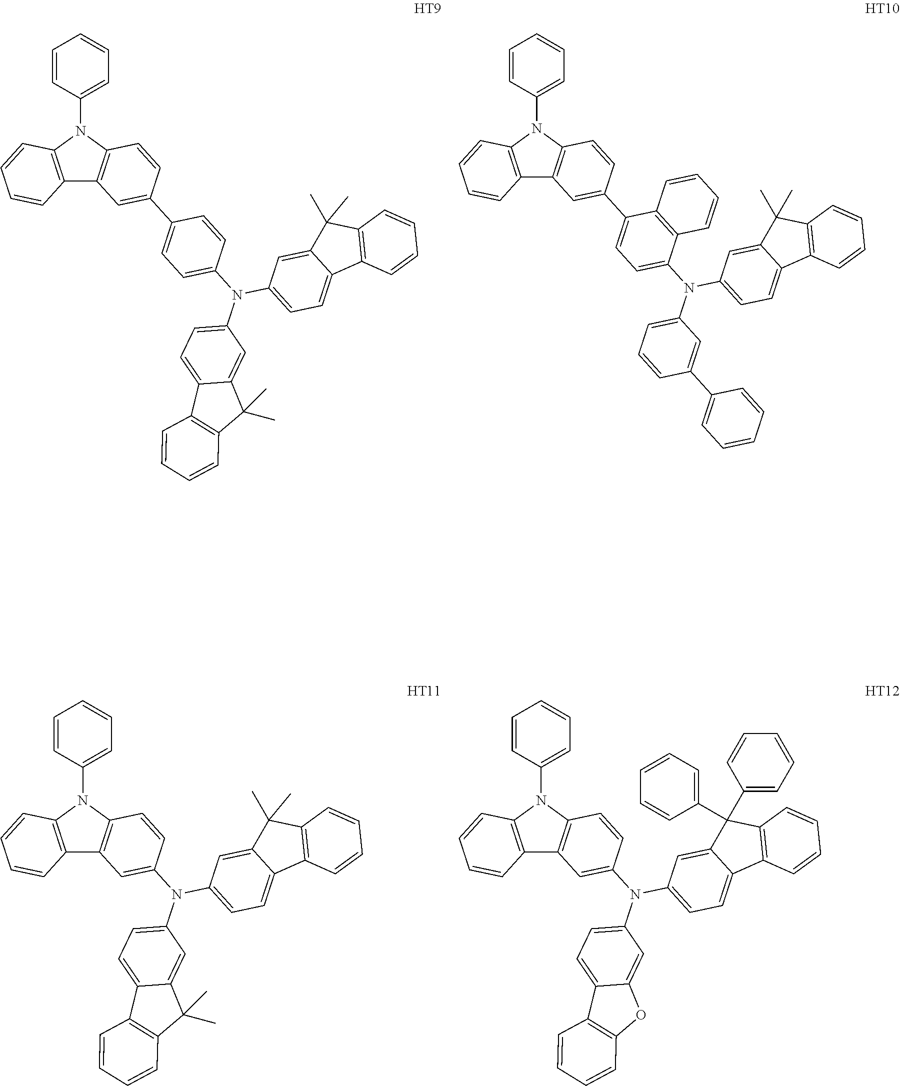 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

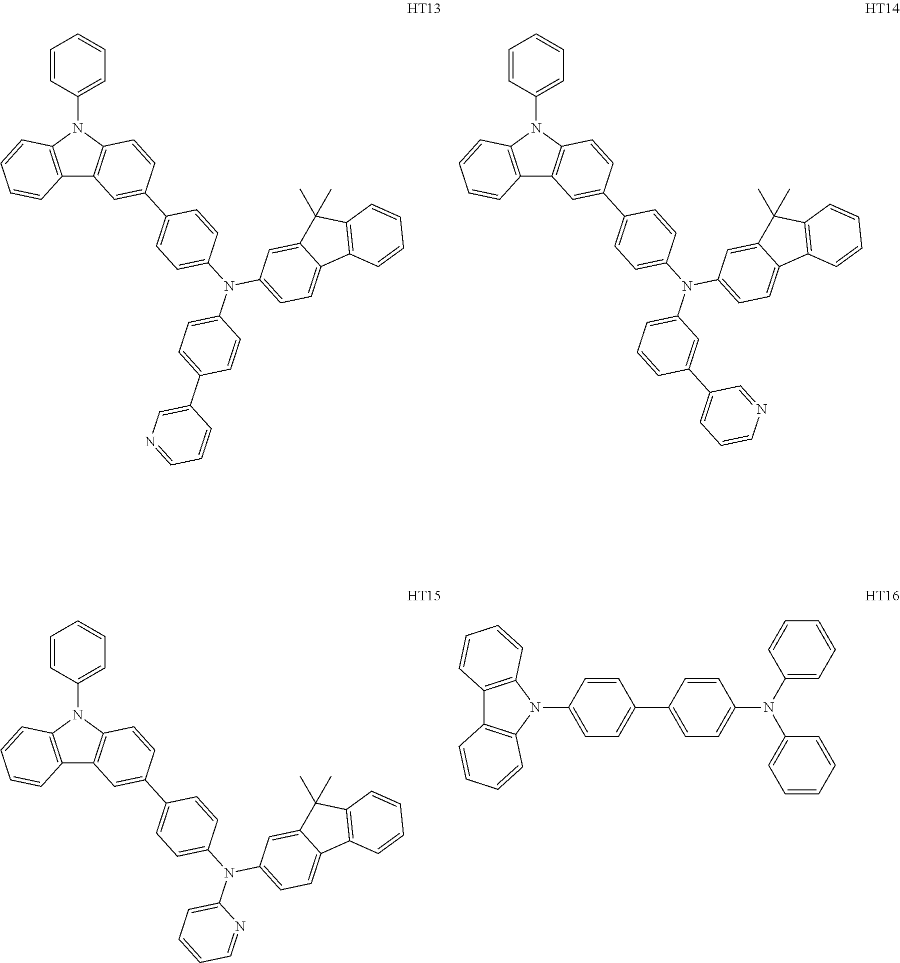 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

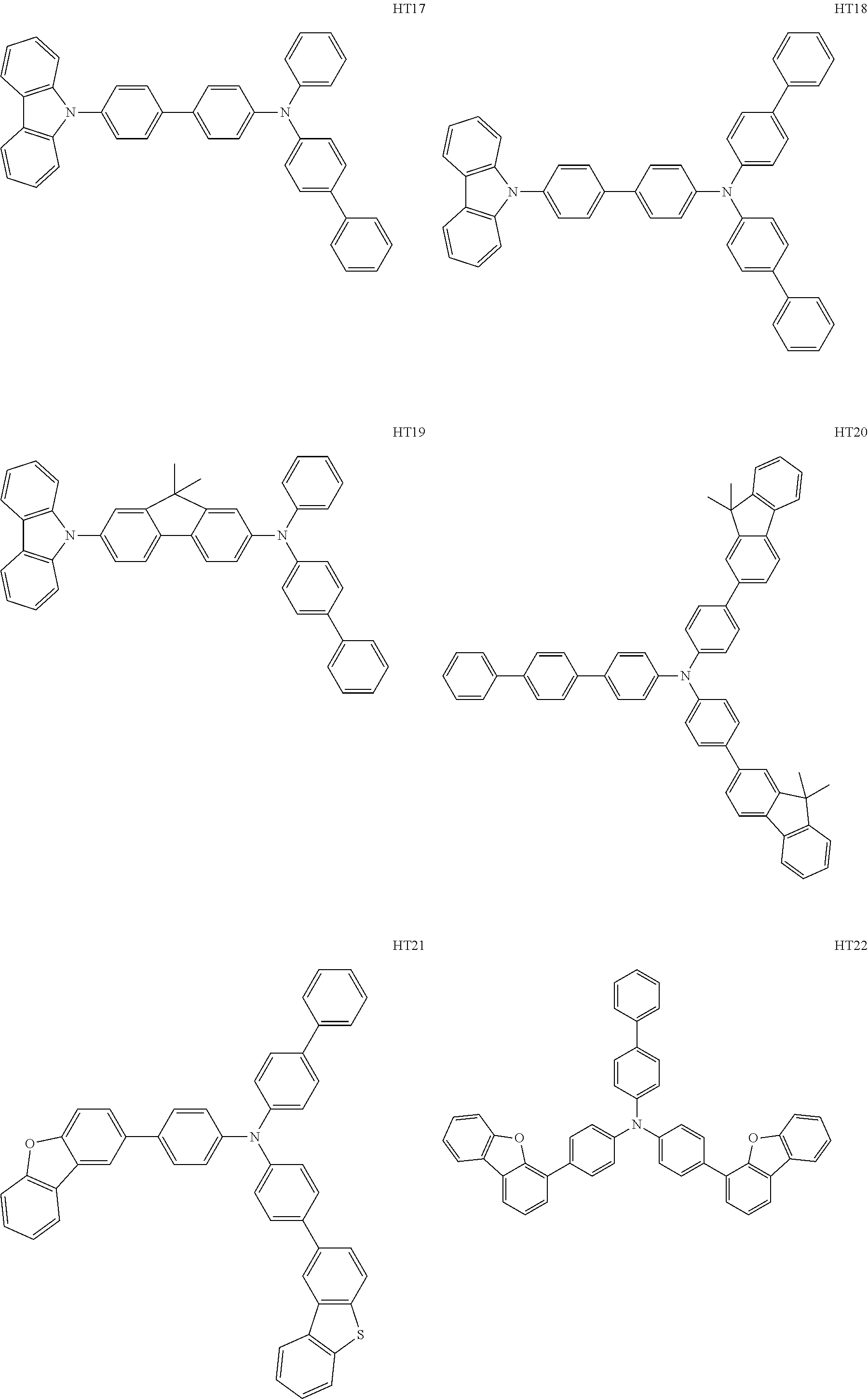

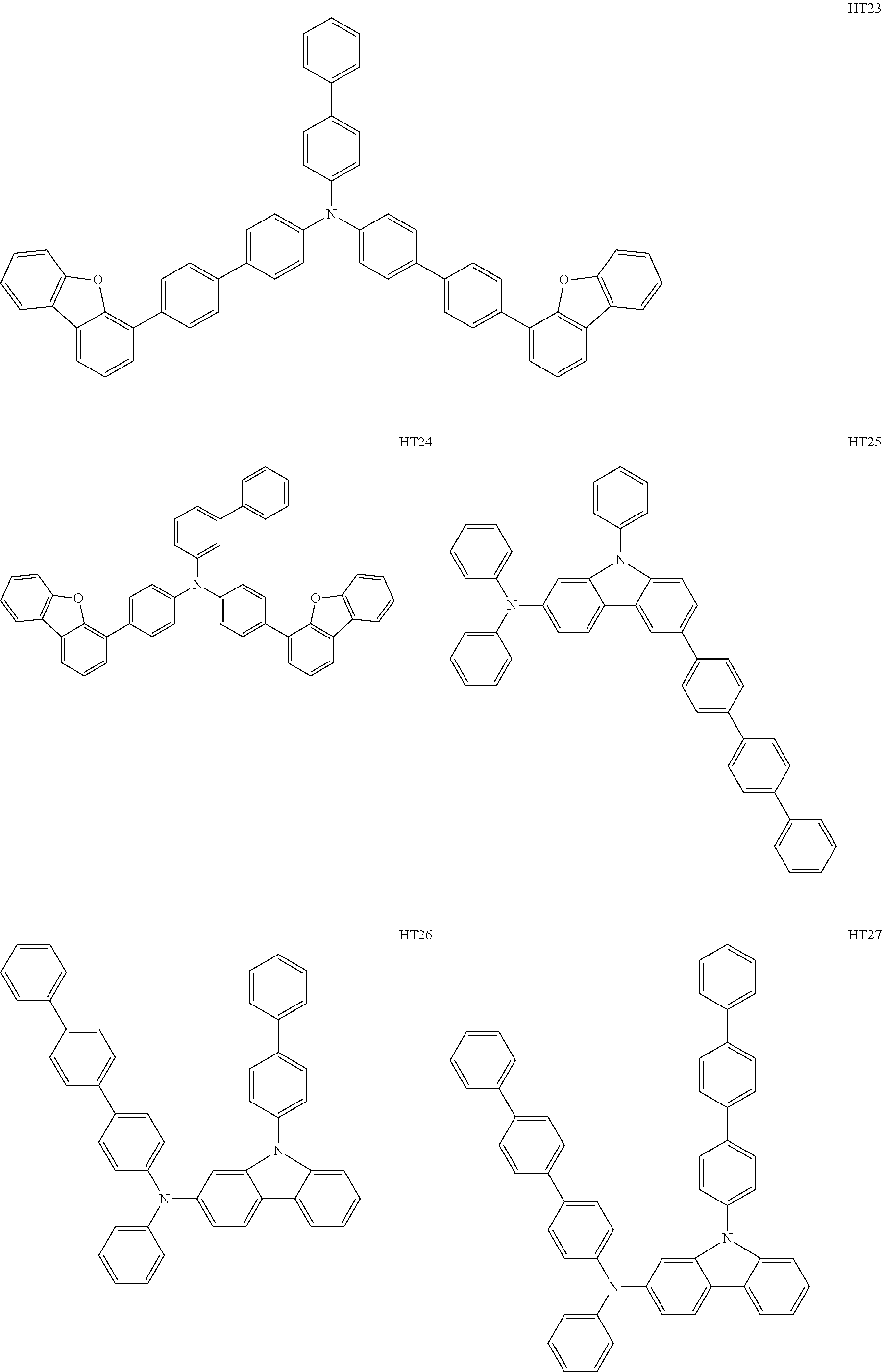 [SMILES: c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1 ; c1ccc(-c2cccc(N(c3ccc(-c4cccc5c4oc4ccccc45)cc3)c3ccc(-c4cccc5c4oc4ccccc45)cc3)c2)cc1 ; c1ccc(-c2ccc(-c3ccc(-c4ccc5c(c4)c4ccc(N(c6ccccc6)c6ccccc6)cc4n5-c4ccccc4)cc3)cc2)cc1 ; c1ccc(-c2ccc(-c3ccc(N(c4ccccc4)c4ccc5c6ccccc6n(-c6ccc(-c7ccccc7)cc6)c5c4)cc3)cc2)cc1 ; c1ccc(-c2ccc(-c3ccc(N(c4ccccc4)c4ccc5c6ccccc6n(-c6ccc(-c7ccc(-c8ccccc8)cc7)cc6)c5c4)cc3)cc2)cc1]

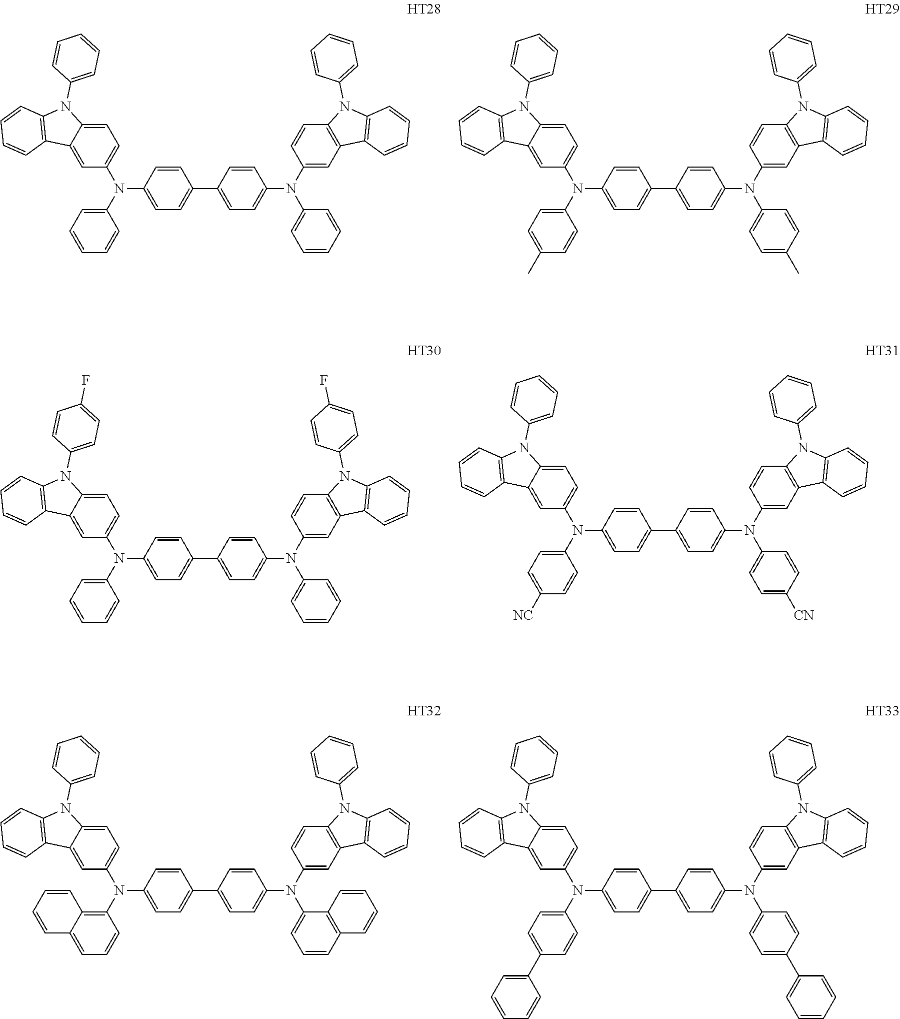

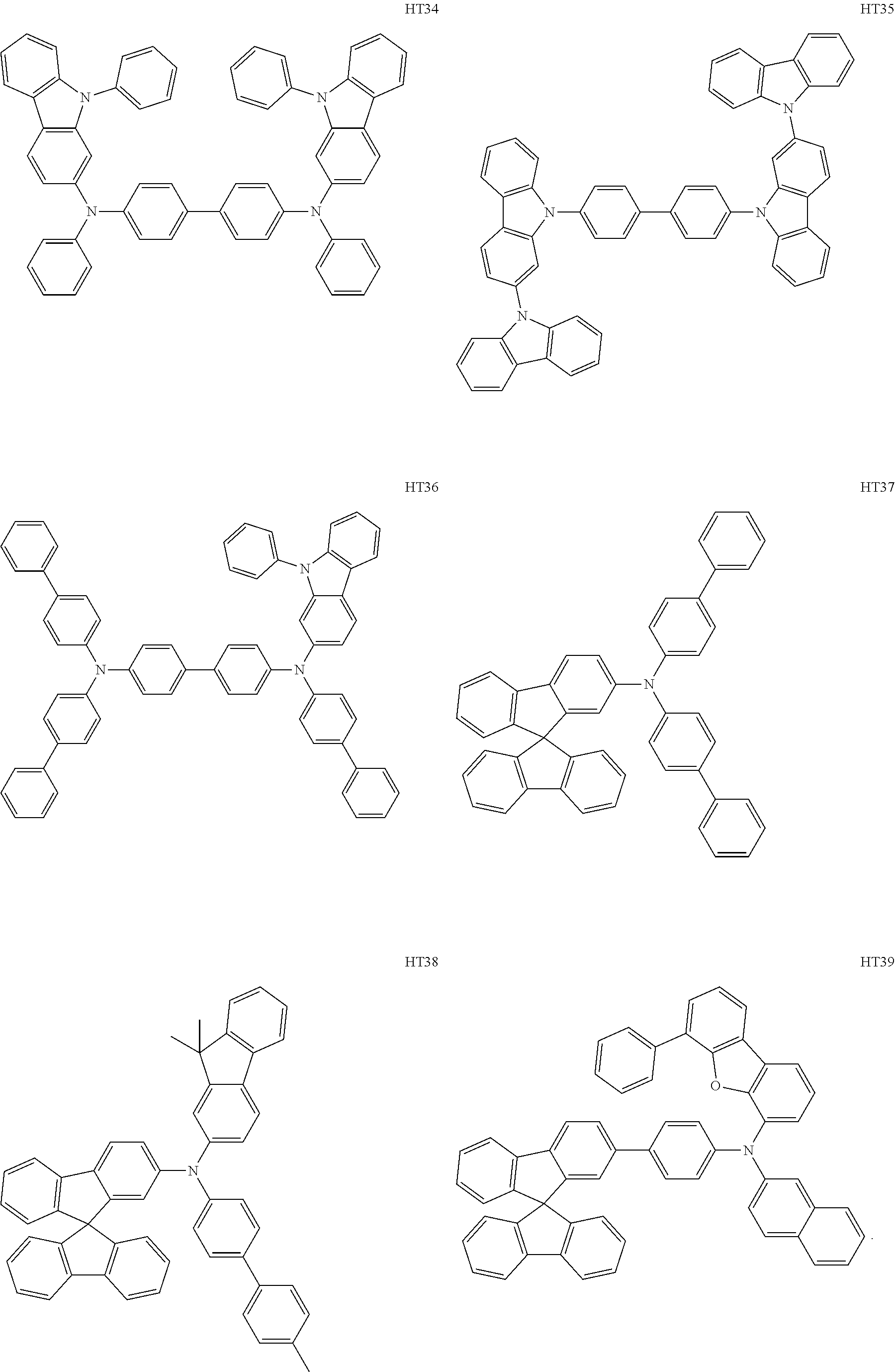

A thickness of the hole transport region may be from about 100 Å to about 10,000 Å, for example, about 100 Å to about 3,000 Å. When the hole transport region includes at least one selected from a hole injection layer and a hole transport layer, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example about 100 Å to about 1,500 Å. When the thicknesses of the hole transport region, the hole injection layer, and the hole transport layer are within these ranges, suitable or satisfactory hole transporting characteristics may be obtained without a substantial increase in driving voltage.

The emission auxiliary layer may increase light-emission efficiency by compensating for an optical resonance distance according to the wavelength of light emitted by an emission layer, and the electron blocking layer may block the flow of electrons from an electron transport region. The emission auxiliary layer and the electron blocking layer may include the materials as described herein above.

p-Dopant

The hole transport region may further include, in addition to these materials, a charge-generation material for the improvement of conductive properties. The charge-generation material may be homogeneously or non-homogeneously dispersed in the hole transport region.

The charge-generation material may be, for example, a p-dopant.

In one embodiment, the p-dopant may have a LUMO energy level of −3.5 eV or less.

The p-dopant may include at least one selected from a quinone derivative, a metal oxide, and a cyano group-containing compound, but embodiments of the present disclosure are not limited thereto.

In one embodiment, the p-dopant may include at least one selected from:

a quinone derivative, such as tetracyanoquinodimethane (TCNQ) or 2,3,5,6-tetrafluoro-7,7,8,8-tetracyanoquinodimethane (F4-TCNQ);

a metal oxide, such as tungsten oxide or molybdenum oxide;

1,4,5,8,9,12-hexaazatriphenylene-hexacarbonitrile (HAT-CN); and

a compound represented by Formula 221,

but embodiments of the present disclosure are not limited thereto:

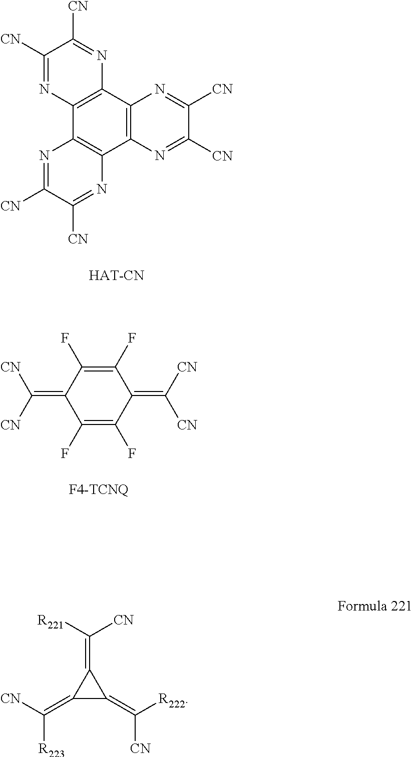

In Formula 221,

R₂₂₁ to R₂₂₃ may each independently be selected from a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group, wherein at least one selected from R₂₂₁ to R₂₂₃ may have at least one substituent selected from a cyano group, —F, —Cl, —Br, —I, a C₁-C₂₀ alkyl group substituted with —F, a C₁-C₂₀ alkyl group substituted with —Cl, a C₁-C₂₀ alkyl group substituted with —Br, and a C₁-C₂₀ alkyl group substituted with —I.

### Emission Layer in Organic Layer 150

When the organic light-emitting device 10 is a full-color organic light-emitting device, the emission layer may be patterned into a red emission layer, a green emission layer, or a blue emission layer, according to a sub-pixel. In one or more embodiments, the emission layer may have a stacked structure of two or more layers selected from a red emission layer, a green emission layer, and a blue emission layer, in which the two or more layers contact each other or are separated from each other. In one or more embodiments, the emission layer may include two or more materials selected from a red light-emitting material, a green light-emitting material, and a blue light-emitting material, in which the two or more materials are mixed with each other in a single layer to emit white light.

The emission layer may include a host and a dopant. The dopant may include at least one selected from a phosphorescent dopant and a fluorescent dopant.

In the emission layer, an amount of the dopant may be in a range of about 0.01 parts by weight to about 15 parts by weight based on 100 parts by weight of the host, but embodiments of the present disclosure are not limited thereto.

A thickness of the emission layer may be in a range of about 100 Å to about 1,000 Å, for example, about 200 Å to about 600 Å. When the thickness of the emission layer is within this range, excellent light-emission characteristics may be obtained without a substantial increase in driving voltage.

### Host in Emission Layer

In one or more embodiments, the host may include a compound represented by Formula 301 below:

[Ar₃₀₁]_{xb11}-[(L₃₀₁))_{xb1}-R₃₀₁]_{xb21}. Formula 301

In Formula 301,

Ar₃₀₁ may be a substituted or unsubstituted C₅-C₆₀ carbocyclic group or a substituted or unsubstituted C₁-C₆₀ heterocyclic group,

xb11 may be 1, 2, or 3,

L₃₀₁ may be selected from a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group,

xb1 may be an integer from 0 to 5,

R₃₀₁ may be selected from deuterium, —F, —Cl, —Br, —I, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), and —P(═O)(Q₃₀₁)(Q₃₀₂),

xb21 may be an integer from 1 to 5, and

Q₃₀₁ to Q₃₀₃ may each independently be selected from a C₁-C₁₀ alkyl group, a C₁-C₁₀ alkoxy group, a phenyl group, a biphenyl group, a terphenyl group, and a naphthyl group, but embodiments of the present disclosure are not limited thereto.

In one embodiment, Ar₃₀₁ in Formula 301 may be selected from:

a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, and a dibenzothiophene group; and

a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, and a dibenzothiophene group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂), and

Q₃₁ to Q₃₃ may each independently be selected from a C₁-C₁₀ alkyl group, a C₁-C₁₀ alkoxy group, a phenyl group, a biphenyl group, a terphenyl group, and a naphthyl group, but embodiments of the present disclosure are not limited thereto.

When xb11 in Formula 301 is 2 or more, two or more Ar₃₀₁(s) may be linked to each other via a single bond.

In one or more embodiments, the compound represented by Formula 301 may be represented by Formula 301-1 or 301-2 below:

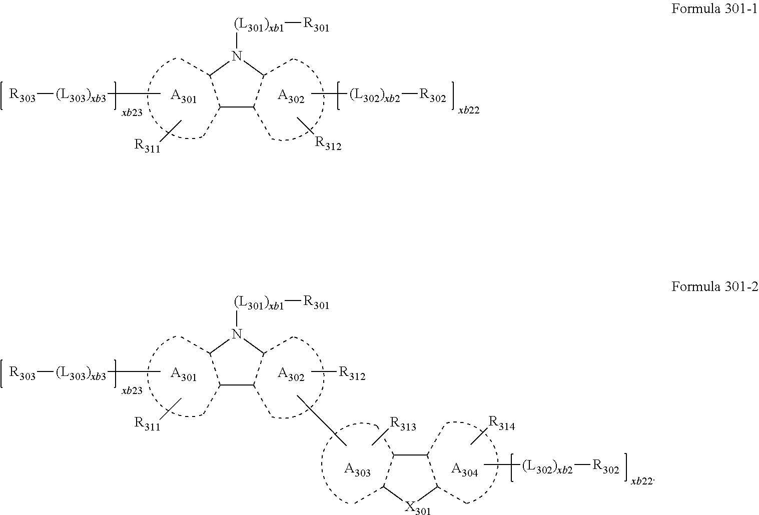

In Formulae 301-1 and 301-2,

ring A₃₀₁ to ring A₃₀₄ may each independently be a benzene ring, a naphthalene ring, a phenanthrene ring, a fluoranthene ring, a triphenylene ring, a pyrene ring, a chrysene ring, a pyridine ring, a pyrimidine ring, an indene ring, a fluorene ring, a spiro-bifluorene ring, a benzofluorene ring, a dibenzofluorene ring, an indole ring, a carbazole ring, a benzocarbazole ring, a dibenzocarbazole ring, a furan ring, a benzofuran ring, a dibenzofuran ring, a naphthofuran ring, a benzonaphthofuran ring, a dinaphthofuran ring, a thiophene ring, a benzothiophene ring, a dibenzothiophene ring, a naphthothiophene ring, a benzonaphthothiophene ring, or a dinaphthothiophene ring,

X₃₀₁ may be O, S, or N-[(L₃₀₄)_{xb4}-R₃₀₁],

R₃₁₁ to R₃₁₄ may each independently be selected from hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂),

xb22 and xb23 may each independently be 0, 1, or 2,

L₃₀₁, xb1, R₃₀₁, and Q₃₁ to Q₃₃ are the same as described herein above,

L₃₀₂ to L₃₀₄ may be understood by referring to the description presented in connection with L₃₀₁,

xb2 to xb4 may be understood by referring to the description presented in connection with xb1, and

R₃₀₂ to R₃₀₄ may be understood by referring to the description presented in connection with R₃₀₁.

For example, in Formulae 301, 301-1, and 301-2, L₃₀₁ to L₃₀₄ may each independently be selected from:

a phenylene group, a naphthylene group, a fluorenylene group, a spiro-bifluorenylene group, a benzofluorenylene group, a dibenzofluorenylene group, a phenanthrenylene group, an anthracenylene group, a fluoranthenylene group, a triphenylenylene group, a pyrenylene group, a chrysenylene group, a perylenylene group, a pentaphenylene group, a hexacenylene group, a pentacenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, a pyridinylene group, an imidazolylene group, a pyrazolylene group, a thiazolylene group, an isothiazolylene group, an oxazolylene group, an isoxazolylene group, a thiadiazolylene group, an oxadiazolylene group, a pyrazinylene group, a pyrimidinylene group, a pyridazinylene group, a triazinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a phenanthridinylene group, an acridinylene group, a phenanthrolinylene group, a phenazinylene group, a benzimidazolylene group, an isobenzothiazolylene group, a benzoxazolylene group, an isobenzoxazolylene group, a triazolylene group, a tetrazolylene group, an imidazopyridinylene group, an imidazopyrimidinylene group, and an azacarbazolylene group; and

a phenylene group, a naphthylene group, a fluorenylene group, a spiro-bifluorenylene group, a benzofluorenylene group, a dibenzofluorenylene group, a phenanthrenylene group, an anthracenylene group, a fluoranthenylene group, a triphenylenylene group, a pyrenylene group, a chrysenylene group, a perylenylene group, a pentaphenylene group, a hexacenylene group, a pentacenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, a pyridinylene group, an imidazolylene group, a pyrazolylene group, a thiazolylene group, an isothiazolylene group, an oxazolylene group, an isoxazolylene group, a thiadiazolylene group, an oxadiazolylene group, a pyrazinylene group, a pyrimidinylene group, a pyridazinylene group, a triazinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a phenanthridinylene group, an acridinylene group, a phenanthrolinylene group, a phenazinylene group, a benzimidazolylene group, an isobenzothiazolylene group, a benzoxazolylene group, an isobenzoxazolylene group, a triazolylene group, a tetrazolylene group, an imidazopyridinylene group, an imidazopyrimidinylene group, and an azacarbazolylene group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, an azacarbazolyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂), and

Q₃₁ to Q₃₃ are the same as described herein above.

In one embodiment, in Formulae 301, 301-1, and 301-2, R₃₀₁ to R₃₀₄ may each independently be selected from:

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, and an azacarbazolyl group; and

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, and an azacarbazolyl group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, an azacarbazolyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂), and

Q₃₁ to Q₃₃ are the same as described herein above.

In one or more embodiments, the host may include an alkaline earth metal complex. For example, the host may be selected from a Be complex (for example, Compound H55), a Mg complex, and a Zn complex.

The host may include at least one selected from 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di-9-carbazolylbenzene (mCP), 1,3,5-tri(carbazol-9-yl)benzene (TCP), and Compounds H1 to H55, but embodiments of the present disclosure are not limited thereto:

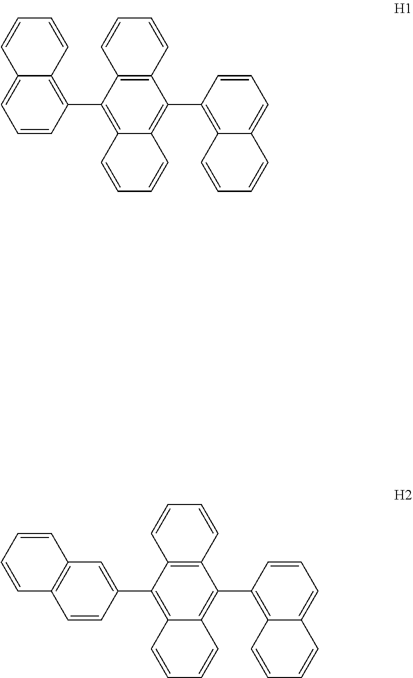

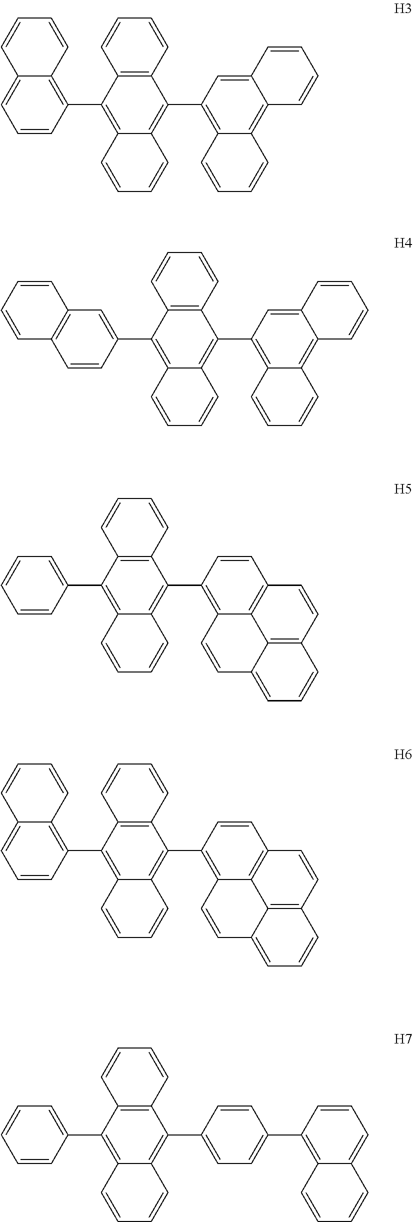

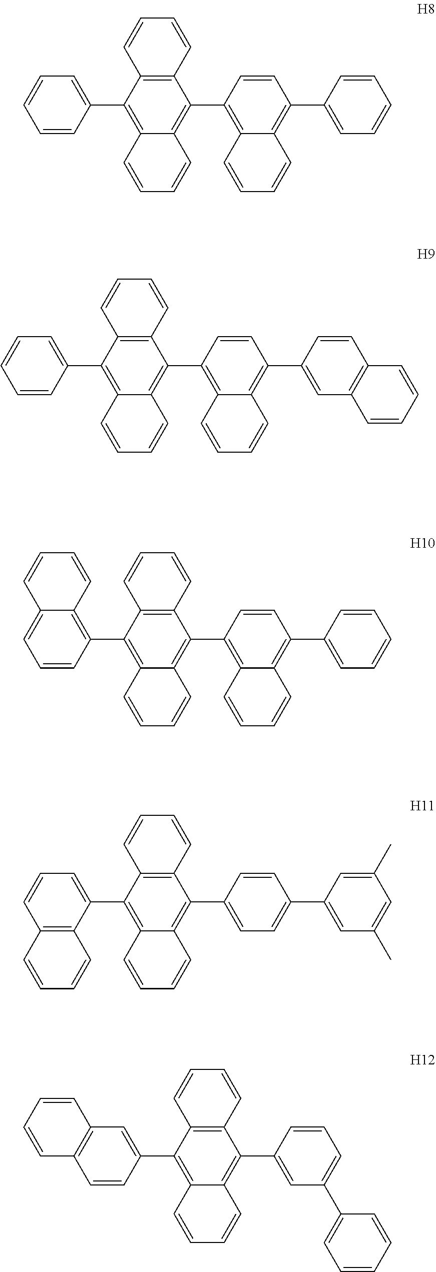

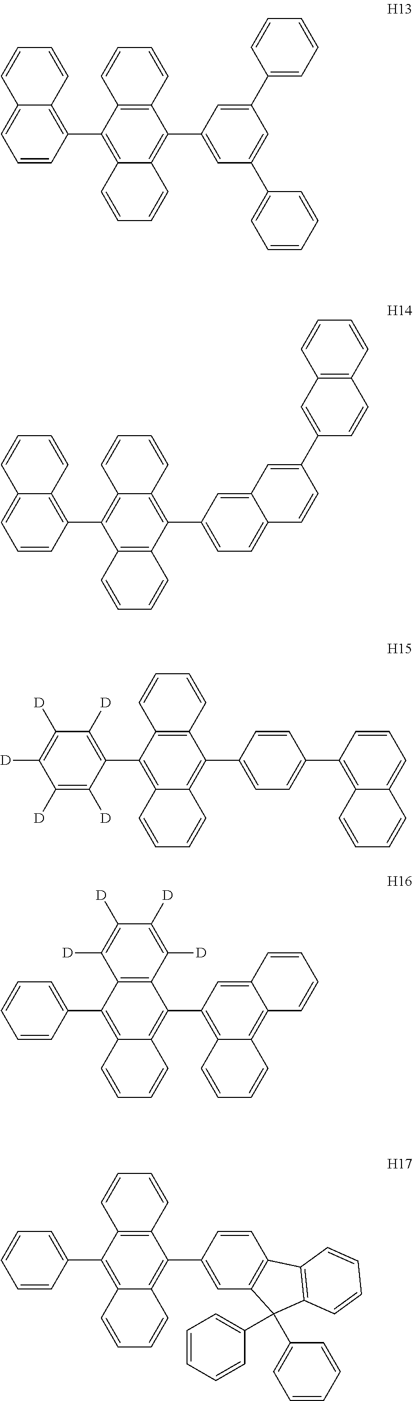

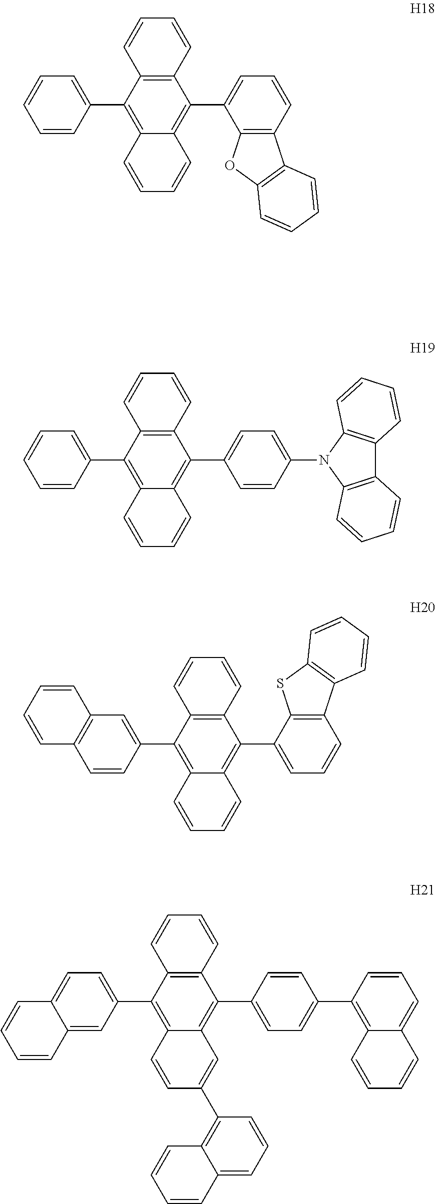

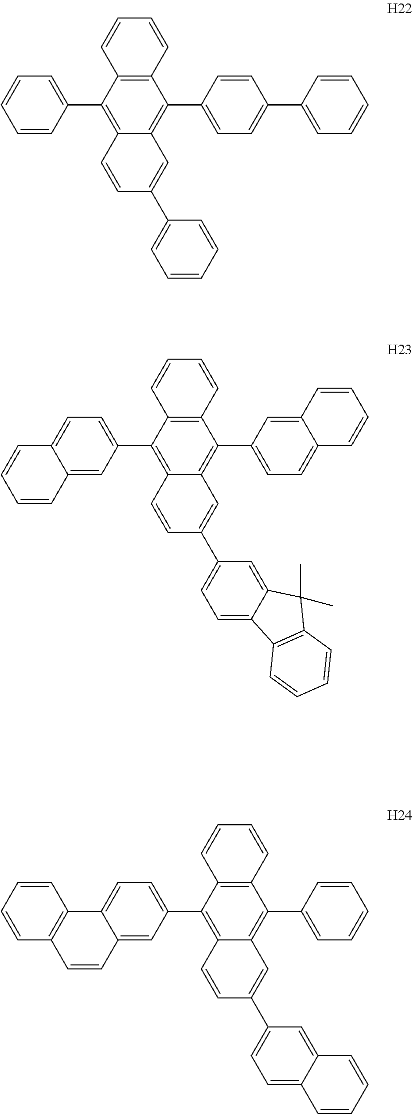

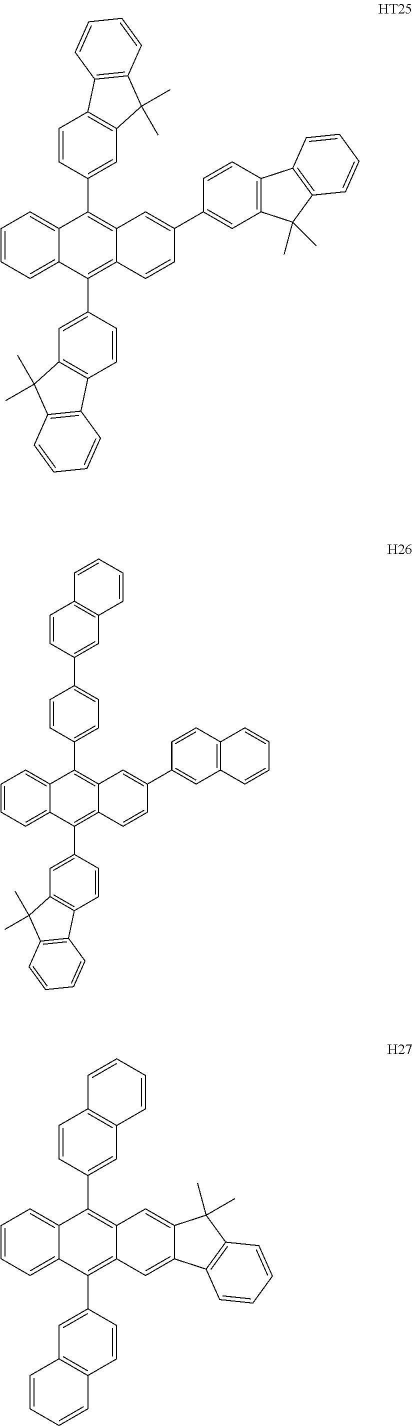

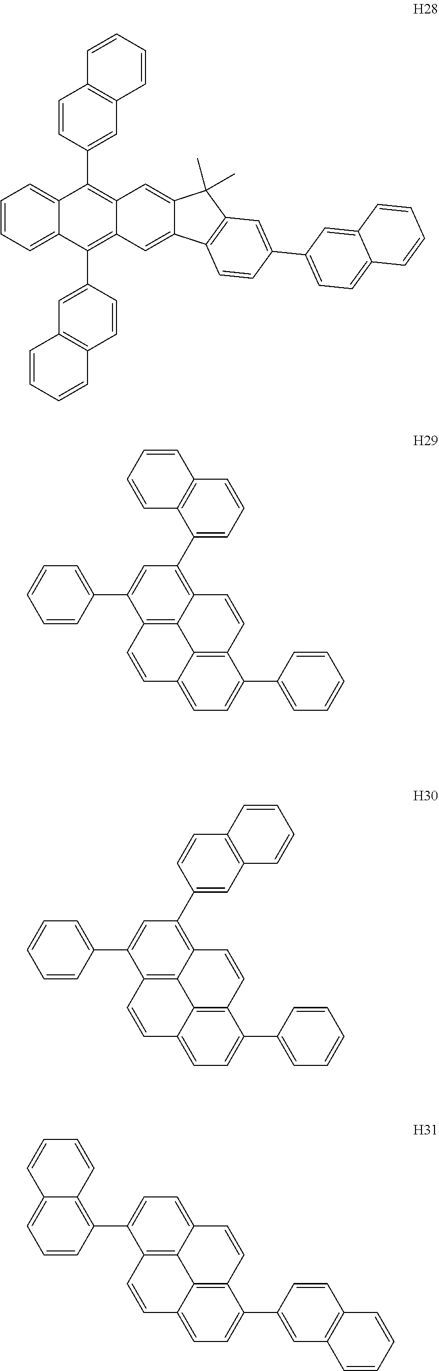

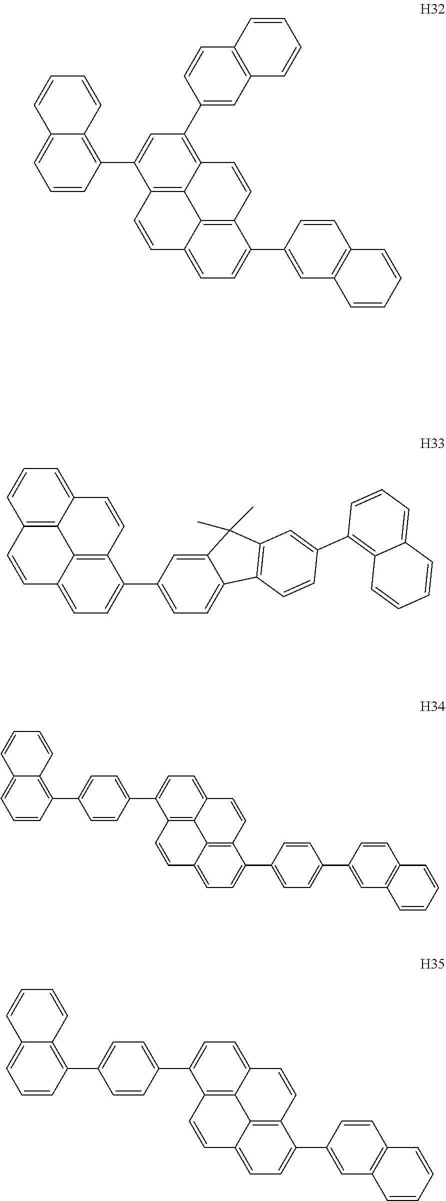

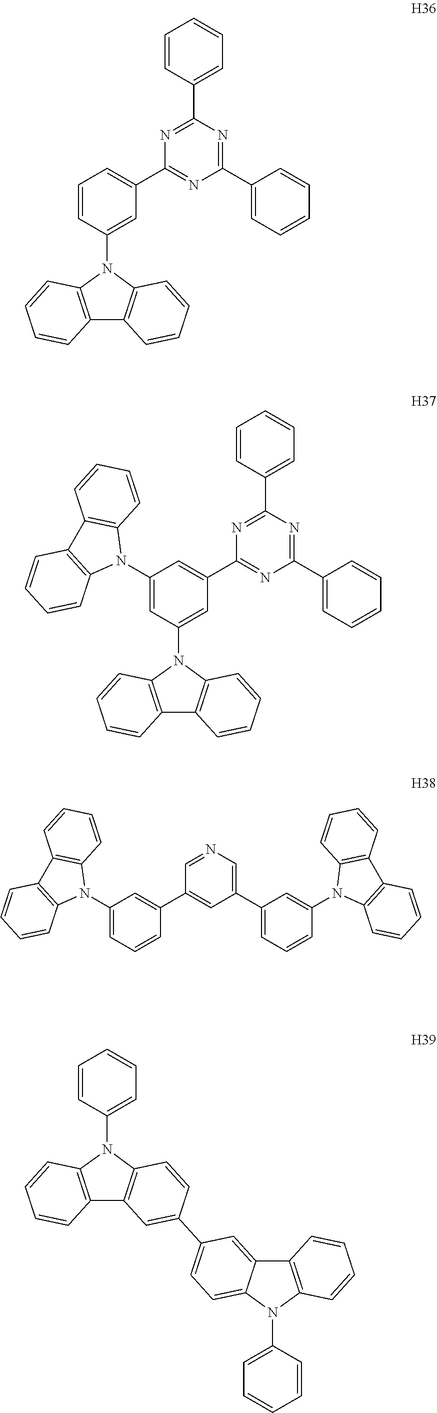 [SMILES: c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1]

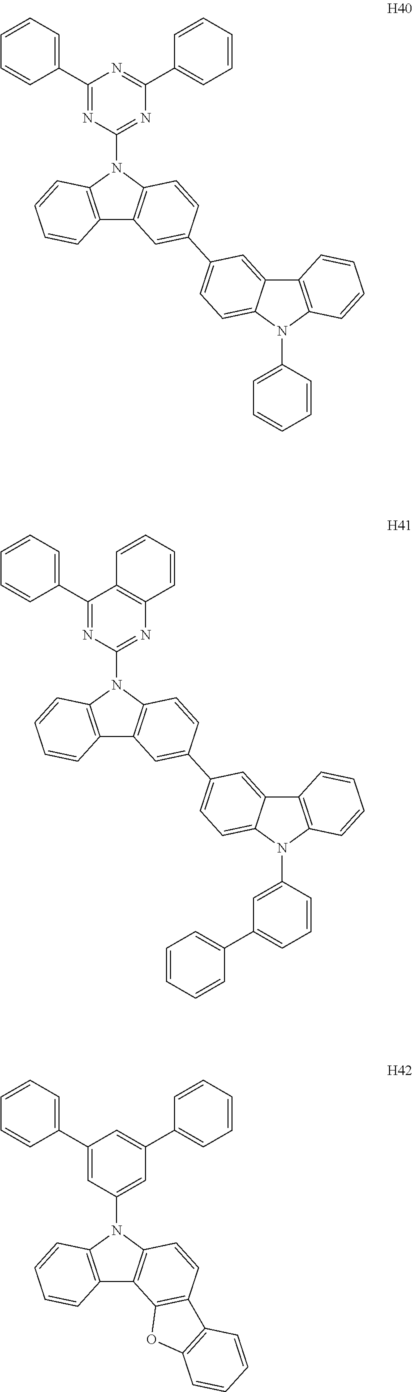 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

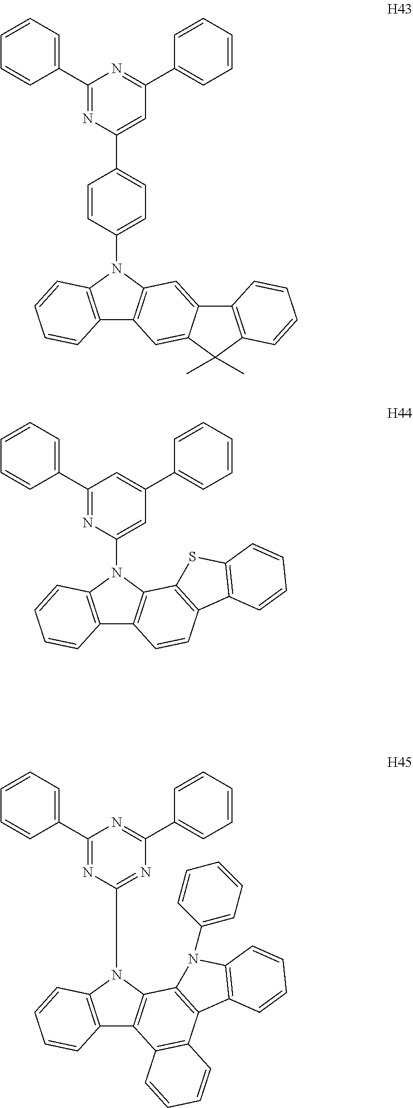 [SMILES: CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1]

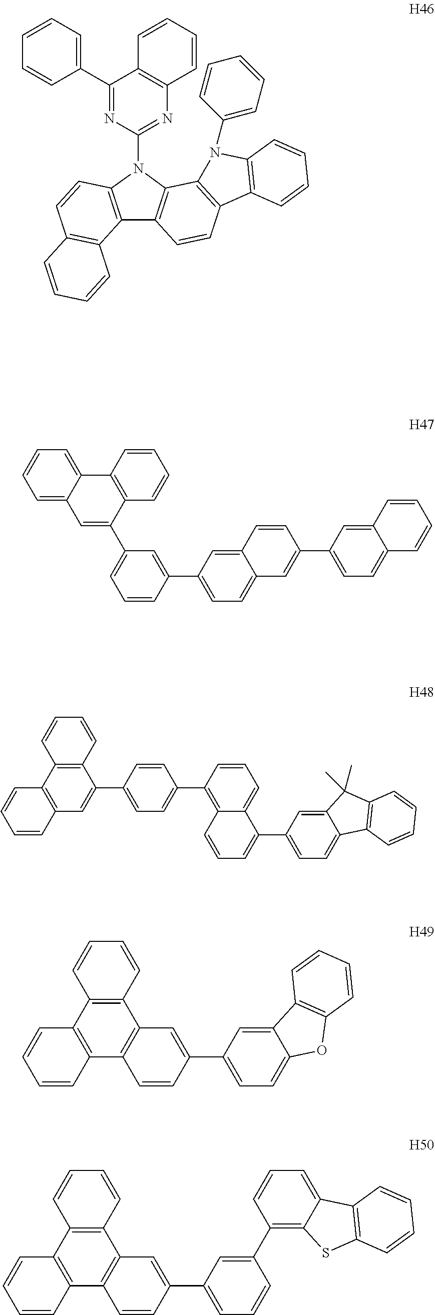

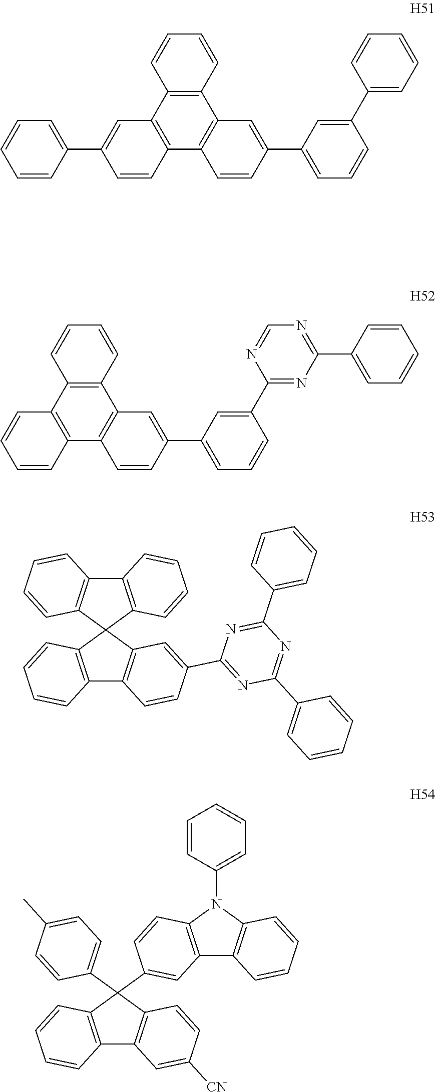

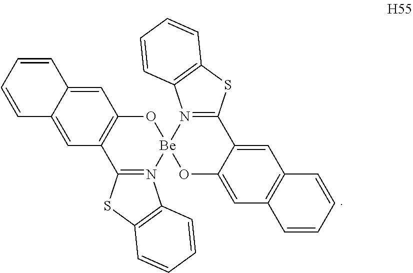

### Phosphorescent Dopant Included in Emission Layer of Organic Layer 150

The phosphorescent dopant may include an organometallic compound represented by Formula 401 below:

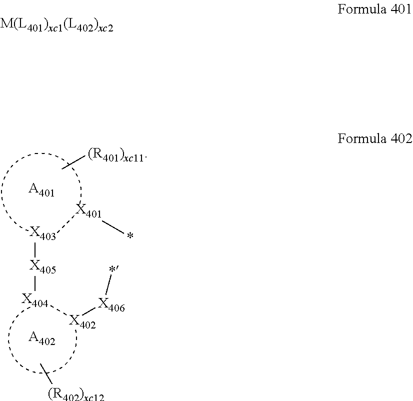

In Formulae 401 and 402,

M may be selected from iridium (Ir), platinum (Pt), palladium (Pd), osmium (Os), titanium (Ti), zirconium (Zr), hafnium (Hf), europium (Eu), terbium (Tb), rhodium (Rh), and thulium (Tm),

L₄₀₁ may be selected from ligands represented by Formula 402, and xc1 may be 1, 2, or 3, wherein, when xc1 is two or more, two or more L₄₀₁ (s) may be identical to or different from each other,

L₄₀₂ may be an organic ligand, and xc2 may be an integer from 0 to 4, wherein, when xc2 is two or more, two or more L₄₀₂ (s) may be identical to or different from each other,

X₄₀₁ to X₄₀₄ may each independently be nitrogen or carbon,

X₄₀₁ and X₄₀₃ may be linked to each other via a single bond or a double bond, and X₄₀₂ and X₄₀₄ may be linked to each other via a single bond or a double bond,

ring A₄₀₁ and ring A₄₀₂ may each independently be a C₅-C₆₀ carbocyclic group or a C₁-C₆₀ heterocyclic group,

X₄₀₅ may be a single bond, *—O—*′, *—C(═O)—*′, *—N(Q₄₁₁)-*′, *—C(Q₄₁₁)(Q₄₁₂)-*′, *—C(Q₄₁₁)═C(Q₄₁₂)-*′, *—C(Q₄₁₁)=*′, or *═C=*′, wherein Q₄₁₁ and Q₄₁₂ may each independently be hydrogen, deuterium, a C₁-C₂₀ alkyl group, a C₁-C₂₀ alkoxy group, a phenyl group, a biphenyl group, a terphenyl group, or a naphthyl group,

X₄₀₆ may be a single bond, O, or S,

R₄₀₁ and R₄₀₂ may each independently be selected from hydrogen, deuterium, —F, —Cl, —Br, —I, a hydroxyl group, a cyano group, a nitro group, an amidino group, a hydrazino group, a hydrazono group, a substituted or unsubstituted C₁-C₂₀ alkyl group, a substituted or unsubstituted C₁-C₂₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), and —P(═O)(Q₄₀₁)(Q₄₀₂), wherein Q₄₀₁ to Q₄₀₃ may each independently be selected from a C₁-C₁₀ alkyl group, a C₁-C₁₀ alkoxy group, a C₆-C₂₀ aryl group, and a C₁-C₂₀ heteroaryl group,

xc11 and xc12 may each independently bean integer from 0 to 10, and

in Formula 402, * and *′ each indicate a binding site to Min Formula 401.

In one embodiment, A₄₀₁ and A₄₀₂ in Formula 402 may each independently be selected from a benzene group, a naphthalene group, a fluorene group, a spiro-bifluorene group, an indene group, a pyrrole group, a thiophene group, a furan group, an imidazole group, a pyrazole group, a thiazole group, an isothiazole group, an oxazole group, an isoxazole group, a pyridine group, a pyrazine group, a pyrimidine group, a pyridazine group, a quinoline group, an isoquinoline group, a benzoquinoline group, a quinoxaline group, a quinazoline group, a carbazole group, a benzimidazole group, a benzofuran group, a benzothiophene group, an isobenzothiophene group, a benzoxazole group, an isobenzoxazole group, a triazole group, a tetrazole group, an oxadiazole group, a triazine group, a dibenzofuran group, and a dibenzothiophene group.

In one or more embodiments, in Formula 402, i) X₄₀₁ may be nitrogen, and X₄₀₂ may be carbon, or ii) X₄₀₁ and X₄₀₂ may each be nitrogen at the same time.

In one or more embodiments, R₄₀₁ and R₄₀₂ in Formula 402 may each independently be selected from:

hydrogen, deuterium, —F, —Cl, —Br, —I, a hydroxyl group, a cyano group, a nitro group, an amidino group, a hydrazino group, a hydrazono group, a C₁-C₂₀ alkyl group, and a C₁-C₂₀ alkoxy group;

a C₁-C₂₀ alkyl group and a C₁-C₂₀ alkoxy group, each substituted with at least one selected from deuterium, —F, —Cl, —Br, —I, a hydroxyl group, a cyano group, a nitro group, an amidino group, a hydrazino group, a hydrazono group, a phenyl group, a naphthyl group, a cyclopentyl group, a cyclohexyl group, an adamantly group, a norbomanyl group, and a norbomenyl group;

a cyclopentyl group, a cyclohexyl group, an adamantly group, a norbomanyl group, a norbomenyl group, a phenyl group, a biphenyl group, a terphenyl group, a naphthyl group, a fluorenyl group, a pyridinyl group, a pyrazinyl group, a pyrimidinyl group, a pyridazinyl group, a triazinyl group, a quinolinyl group, an isoquinolinyl group, a quinoxalinyl group, a quinazolinyl group, a carbazolyl group, a dibenzofuranyl group, and a dibenzothiophenyl group;

a cyclopentyl group, a cyclohexyl group, an adamantly group, a norbomanyl group, a norbomenyl group a phenyl group, a biphenyl group, a terphenyl group, a naphthyl group, a fluorenyl group, a pyridinyl group, a pyrazinyl group, a pyrimidinyl group, a pyridazinyl group, a triazinyl group, a quinolinyl group, an isoquinolinyl group, a quinoxalinyl group, a quinazolinyl group, a carbazolyl group, a dibenzofuranyl group, and a dibenzothiophenyl group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a cyclopentyl group, a cyclohexyl group, an adamantly group, a norbomanyl group, a norbomenyl group, a phenyl group, a biphenyl group, a terphenyl group, a naphthyl group, a fluorenyl group, a pyridinyl group, a pyrazinyl group, a pyrimidinyl group, a pyridazinyl group, a triazinyl group, a quinolinyl group, an isoquinolinyl group, a quinoxalinyl group, a quinazolinyl group, a carbazolyl group, a dibenzofuranyl group, and a dibenzothiophenyl group; and

—Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), and —P(═O)(Q₄₀₁)(Q₄₀₂), and

Q₄₀₁ to Q₄₀₃ may each independently be selected from a C₁-C₁₀ alkyl group, a C₁-C₁₀ alkoxy group, a phenyl group, a biphenyl group, and a naphthyl group, but embodiments of the present disclosure are not limited thereto.

In one or more embodiments, when xc1 in Formula 401 is 2 or more, two A₄₀₁(s) in two or more L₄₀₁ (s) may optionally be linked to each other via X₄₀₇, which is a linking group, or two A₄₀₂(s) in two or more L₄₀₁ (s) may optionally be linked to each other via X₄₀₈, which is a linking group (see Compounds PD1 to PD4 and PD7). X₄₀₇ and X₄₀₈ may each independently be a single bond, *—O—*′, *—S—*′, *—C(═O)—*′, *—N(Q₄₁₃)-*′, *—C(Q₄₁₃)(Q₄₁₄)-*′, or *—C(Q₄₁₃)═C(Q₄₁₄)-*′ (wherein Q₄₁₃ and Q₄₁₄ may each independently be hydrogen, deuterium, a C₁-C₂₀ alkyl group, a C₁-C₂₀ alkoxy group, a phenyl group, a biphenyl group, a terphenyl group, or a naphthyl group), but embodiments of the present disclosure are not limited thereto.

In Formula 401, L₄₀₂ may be a monovalent, divalent, or trivalent organic ligand. For example, L₄₀₂ may be selected from halogen, diketone (for example, acetylacetonate), carboxylic acid (for example, picolinate), —C(═O), isonitrile, —CN, and phosphorus (for example, phosphine, or phosphite), but embodiments of the present disclosure are not limited thereto.

In one or more embodiments, the phosphorescent dopant may be selected from, for example, Compounds PD1 to PD25, but embodiments of the present disclosure are not limited thereto:

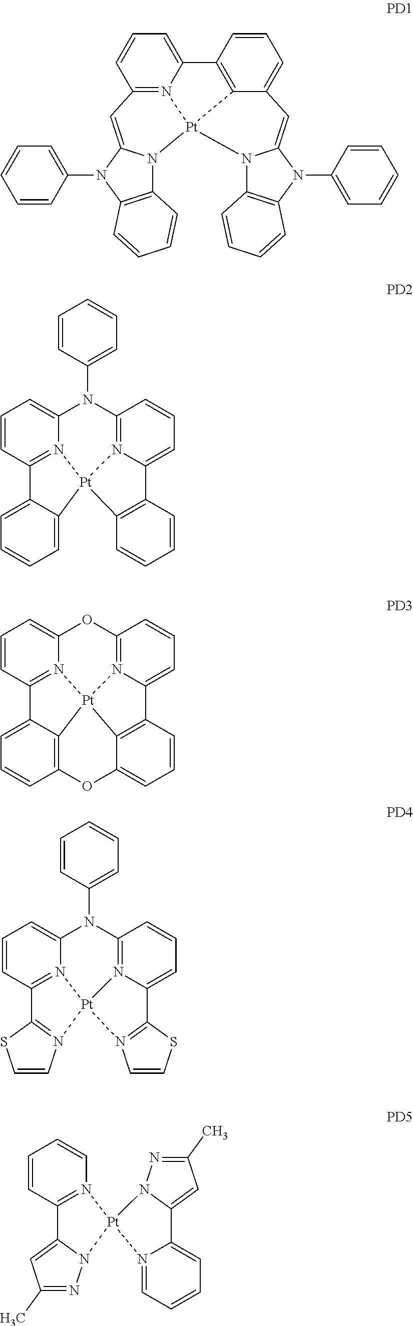

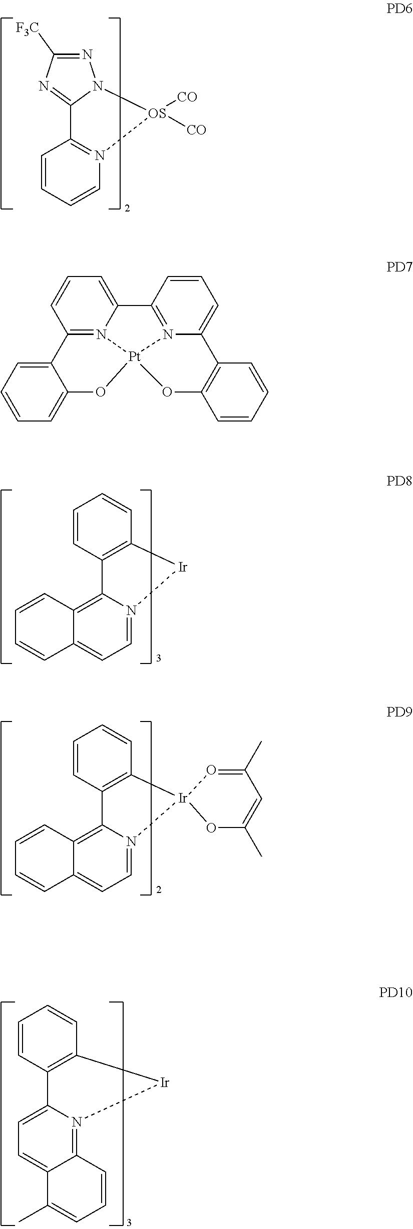

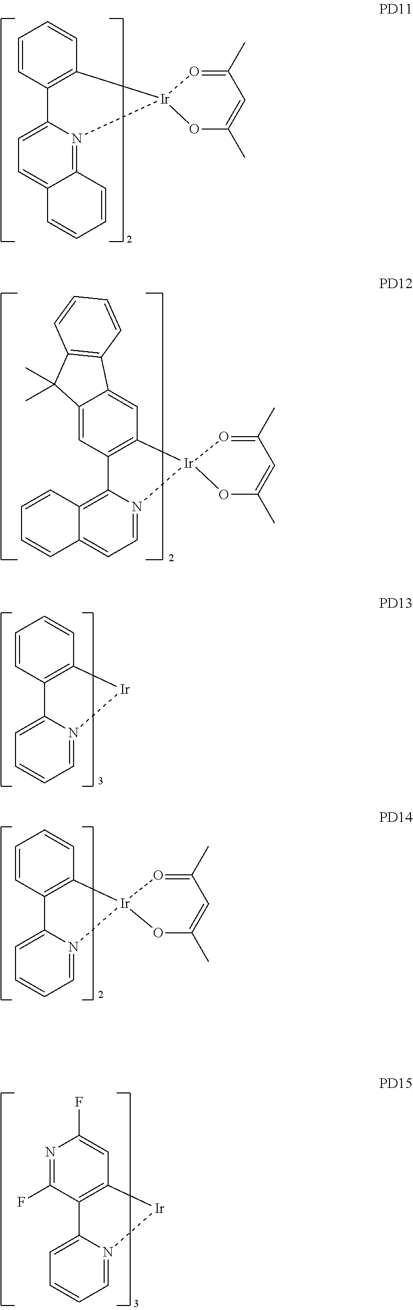

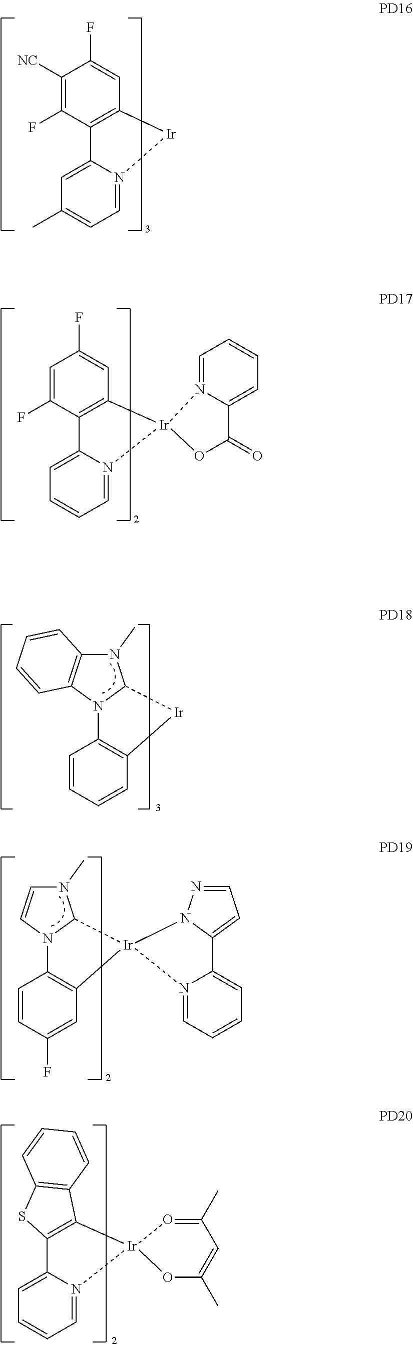

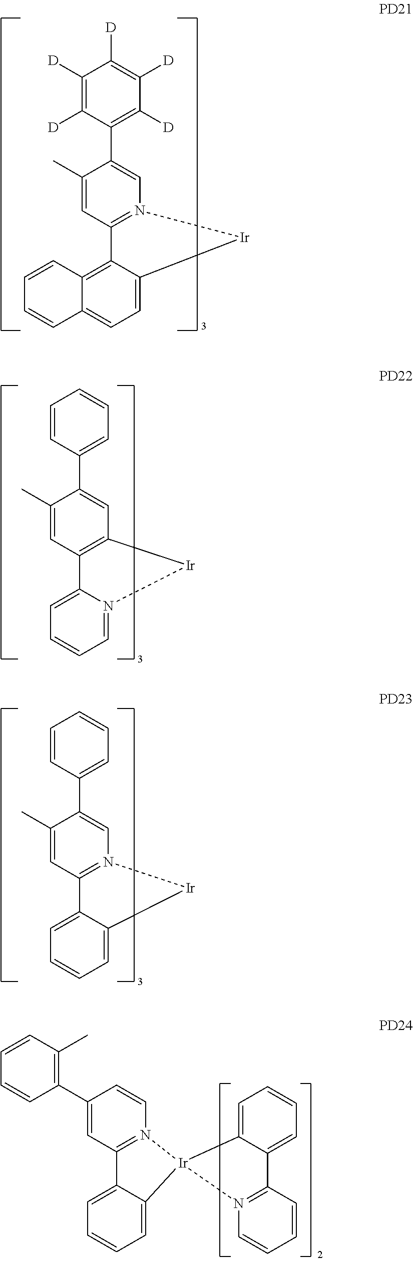

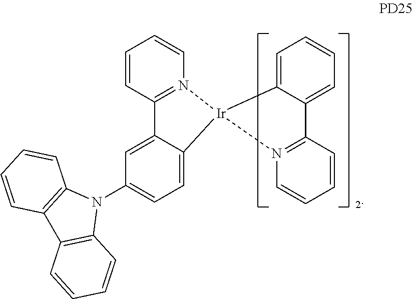

### Fluorescent Dopant in Emission Layer

The fluorescent dopant may include an arylamine compound or a styrylamine compound.

The fluorescent dopant may include a compound represented by Formula 501 below:

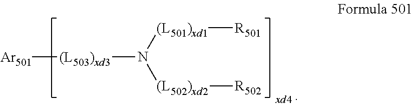

In Formula 501,

Ar₅₀₁ may be a substituted or unsubstituted C₅-C₆₀ carbocyclic group or a substituted or unsubstituted C₁-C₆₀ heterocyclic group,

L₅₀₁ to L₅₀₃ may each independently be selected from a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group,

xd1 to xd3 may each independently be an integer from 0 to 3,

R₅₀₁ and R₅₀₂ may each independently be selected from a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₃₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group, and

xd4 may be an integer from 1 to 6.

In one embodiment, Ar₅₀₁ in Formula 501 may be selected from:

a naphthalene group, a heptalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, and an indenophenanthrene group; and

a naphthalene group, a heptalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, and an indenophenanthrene group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, and a naphthyl group.

In one or more embodiments, L₅₀₁ to L₅₀₃ in Formula 501 may each independently be selected from:

a phenylene group, a naphthylene group, a fluorenylene group, a spiro-bifluorenylene group, a benzofluorenylene group, a dibenzofluorenylene group, a phenanthrenylene group, an anthracenylene group, a fluoranthenylene group, a triphenylenylene group, a pyrenylene group, a chrysenylene group, a perylenylene group, a pentaphenylene group, a hexacenylene group, a pentacenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, and a pyridinylene group; and

a phenylene group, a naphthylene group, a fluorenylene group, a spiro-bifluorenylene group, a benzofluorenylene group, a dibenzofluorenylene group, a phenanthrenylene group, an anthracenylene group, a fluoranthenylene group, a triphenylenylene group, a pyrenylene group, a chrysenylene group, a perylenylene group, a pentaphenylene group, a hexacenylene group, a pentacenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, and a pyridinylene group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, and a pyridinyl group.

In one or more embodiments, R₅₀₁ and R₅₀₂ in Formula 501 may each independently be selected from:

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, and a pyridinyl group; and

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, and a pyridinyl group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, and —Si(Q₃₁)(Q₃₂)(Q₃₃), and

Q₃₁ to Q₃₃ may each independently be selected from a C₁-C₁₀ alkyl group, a C₁-C₁₀ alkoxy group, a phenyl group, a biphenyl group, a terphenyl group, and a naphthyl group.

In one or more embodiments, xd4 in Formula 501 may be 2, but embodiments of the present disclosure are not limited thereto.

For example, the fluorescent dopant may be selected from Compounds FD1 to FD22:

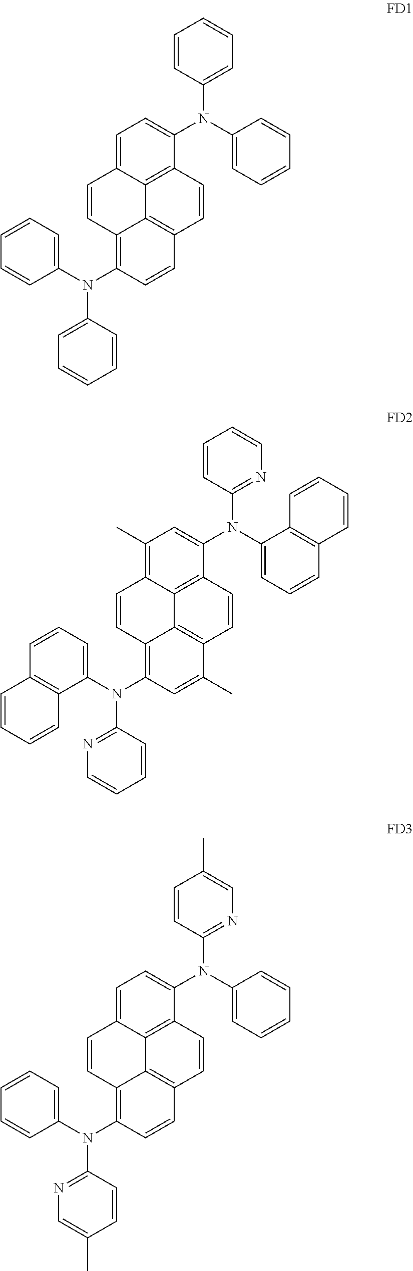 [SMILES: c1ccc(N(c2ccccc2)c2ccc3ccc4c(N(c5ccccc5)c5ccccc5)ccc5ccc2c3c54)cc1 ; Cc1cc(N(c2ccccn2)c2cccc3ccccc23)c2ccc3c(C)cc(N(c4ccccn4)c4cccc5ccccc45)c4ccc1c2c34 ; Cc1ccc(N(c2ccccc2)c2ccc3ccc4c(N(c5ccccc5)c5ccc(C)cn5)ccc5ccc2c3c54)nc1]

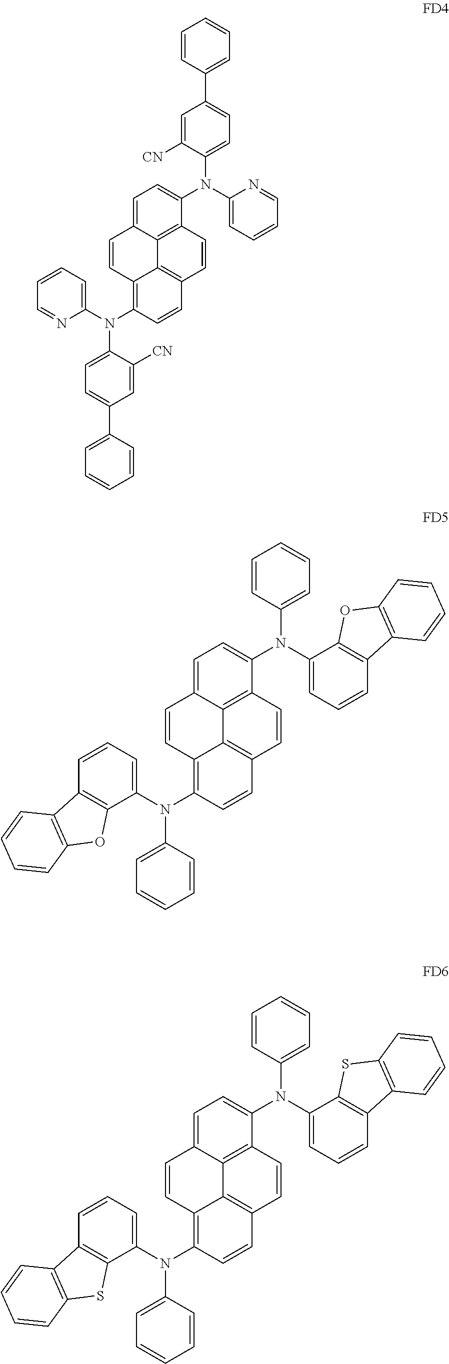

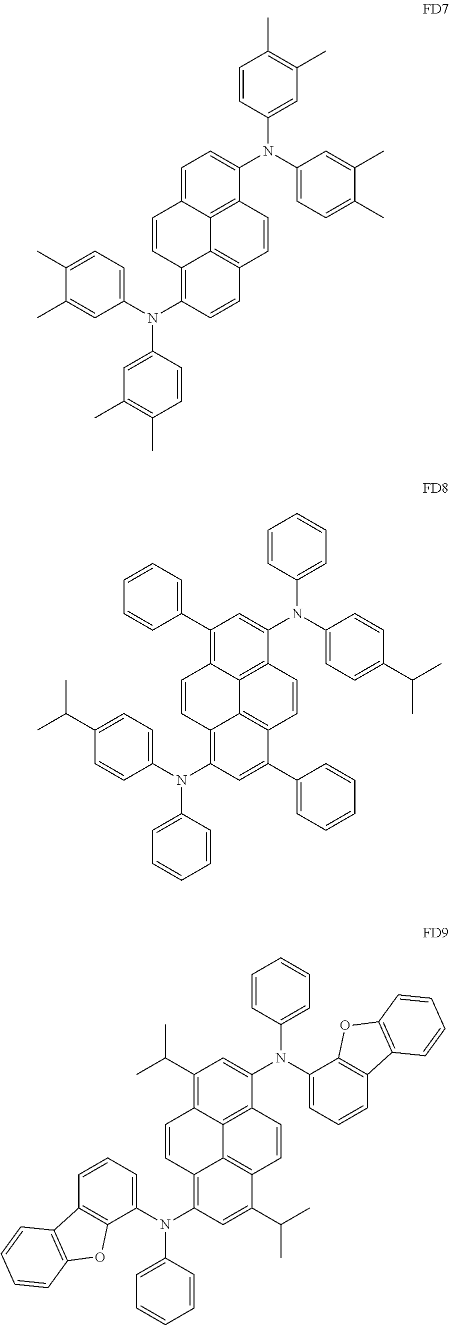

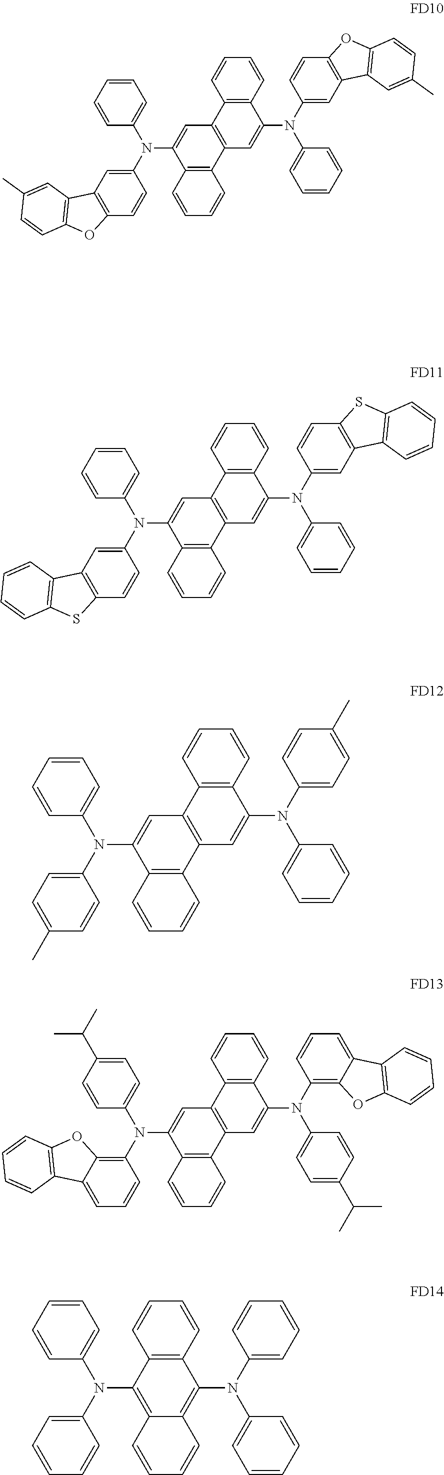

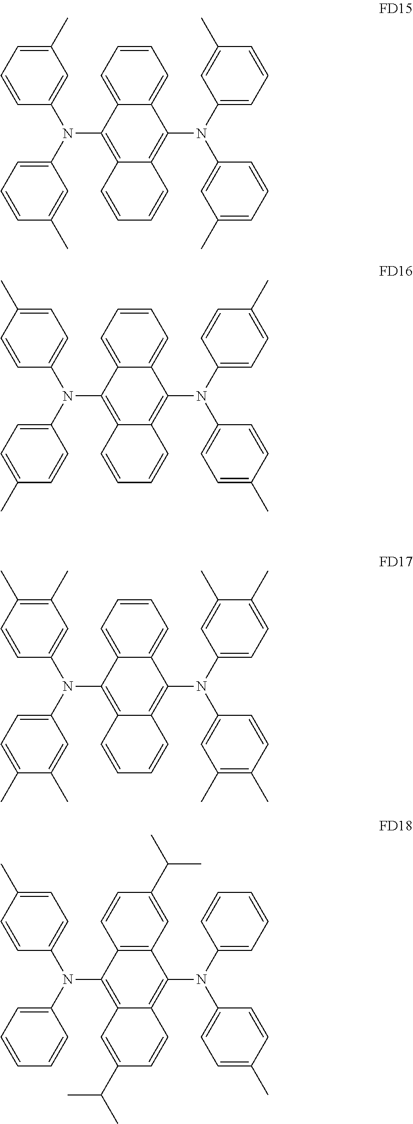 [SMILES: Cc1cccc(N(c2cccc(C)c2)c2c3ccccc3c(N(c3cccc(C)c3)c3cccc(C)c3)c3ccccc23)c1 ; Cc1ccc(N(c2ccc(C)cc2)c2c3ccccc3c(N(c3ccc(C)cc3)c3ccc(C)cc3)c3ccccc23)cc1 ; Cc1ccc(N(c2ccc(C)c(C)c2)c2c3ccccc3c(N(c3ccc(C)c(C)c3)c3ccc(C)c(C)c3)c3ccccc23)cc1C ; Cc1ccc(N(c2ccccc2)c2c3ccc(C(C)C)cc3c(N(c3ccccc3)c3ccc(C)cc3)c3ccc(C(C)C)cc23)cc1]

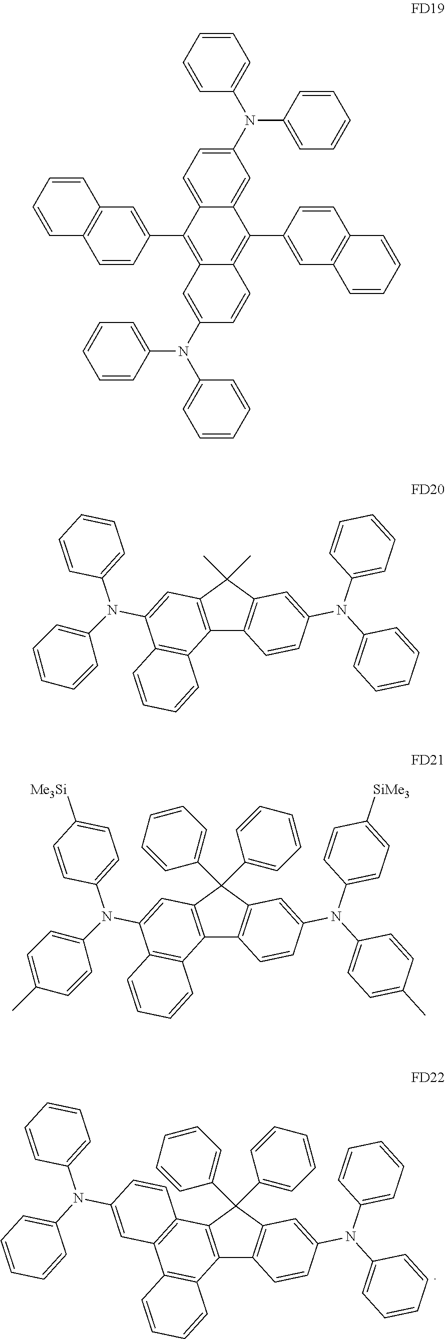

In one or more embodiments, the fluorescent dopant may be selected from the following compounds, but embodiments of the present disclosure are not limited thereto:

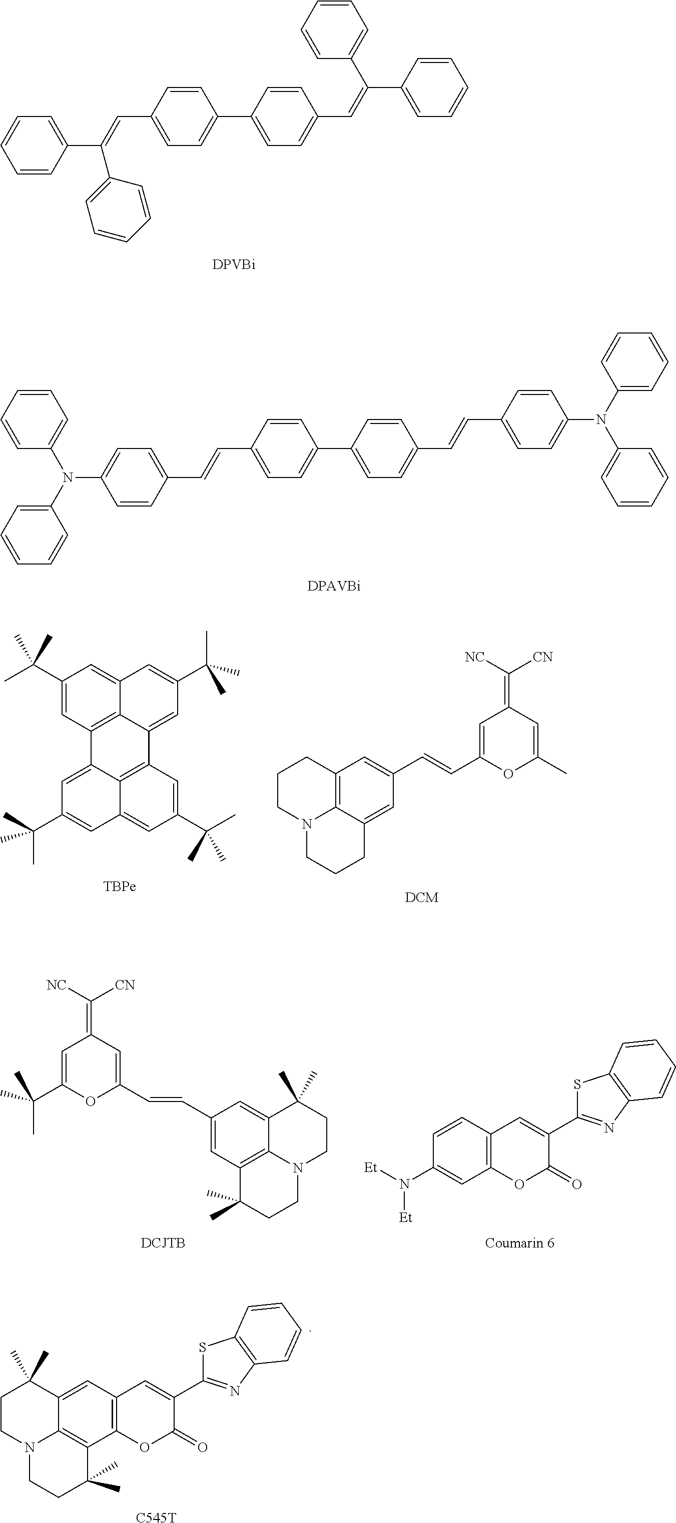

### Electron Transport Region in Organic Layer 150

The electron transport region may have i) a single-layered structure including a single layer including a single material, ii) a single-layered structure including a single layer including a plurality of different materials, or iii) a multi-layered structure having a plurality of layers including a plurality of different materials.

The electron transport region may include at least one selected from a buffer layer, a hole blocking layer, an electron control layer, an electron transport layer, and an electron injection layer, but embodiments of the present disclosure are not limited thereto.

For example, the electron transport region may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, wherein for each structure, constituting layers are sequentially stacked from an emission layer. However, embodiments of the structure of the electron transport region are not limited thereto.

The electron transport region (for example, a buffer layer, a hole blocking layer, an electron control layer, or an electron transport layer in the electron transport region) may include a metal-free compound containing at least one Tr electron-depleted nitrogen-containing ring.

The term “π electron-depleted nitrogen-containing ring,” as used herein, indicates a C₁-C₆₀ heterocyclic group having at least one *—N=*′ moiety as a ring-forming moiety.

For example, the “π electron-depleted nitrogen-containing ring” may be i) a 5-membered to 7-membered heteromonocyclic group having at least one *—N=*′ moiety, ii) a heteropolycyclic group in which two or more 5-membered to 7-membered heteromonocyclic groups each having at least one *—N=*′ moiety are condensed with each other (e.g., combined together), or iii) a heteropolycyclic group in which at least one of 5-membered to 7-membered heteromonocyclic groups, each having at least one *—N=*′ moiety, is condensed with (e.g., condensed with) at least one C₅-C₆₀ carbocyclic group.

Examples of the T electron-depleted nitrogen-containing ring include an imidazole ring, a pyrazole ring, a thiazole ring, an isothiazole ring, an oxazole ring, an isoxazole ring, a pyridine ring, a pyrazine ring, a pyrimidine ring, a pyridazine ring, an indazole ring, a purine ring, a quinoline ring, an isoquinoline ring, a benzoquinoline ring, a phthalazine ring, a naphthyridine ring, a quinoxaline ring, a quinazoline ring, a cinnoline ring, a phenanthridine ring, an acridine ring, a phenanthroline ring, a phenazine ring, a benzimidazole ring, an isobenzothiazole ring, a benzoxazole ring, an isobenzoxazole ring, a triazole ring, a tetrazole ring, an oxadiazole ring, a triazine ring, a thiadiazole ring, an imidazopyridine ring, an imidazopyrimidine ring, and an azacarbazole ring, but are not limited thereto.

For example, the electron transport region may include a compound represented by Formula 601 below:

[Ar₆₀₁]ₓₑ₁₁-[(L₆₀₁)ₓₑ₁-R₆₀₁]ₓₑ₂₁. Formula 601

In Formula 601,

Ar₆₀₁ may be a substituted or unsubstituted C₅-C₆₀ carbocyclic group or a substituted or unsubstituted C₁-C₆₀ heterocyclic group, xe11 may be 1, 2, or 3,

L₆₀₁ may be selected from a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group,

xe1 may be an integer from 0 to 5,

R₆₀₁ may be selected from a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₆₀₁)(Q₆₀₂)(Q₆₀₃), —C(═O)(Q₆₀₁), —S(═O)₂(Q₆₀₁), and —P(═O)(Q₆₀₁)(Q₆₀₂),

Q₆₀₁ to Q₆₀₃ may each independently be a C₁-C₁₀ alkyl group, a C₁-C₁₀ alkoxy group, a phenyl group, a biphenyl group, a terphenyl group, or a naphthyl group, and

xe21 may be an integer from 1 to 5.

In one embodiment, at least one of Ar₆₀₁(s) in the number of xe11 and R₆₀₁(s) in the number of xe21 may include the Tr electron-depleted nitrogen-containing ring.

In one embodiment, Ar₆₀₁ in Formula 601 may be selected from:

a benzene group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, a dibenzothiophene group, a carbazole group, an imidazole group, a pyrazole group, a thiazole group, an isothiazole group, an oxazole group, an isoxazole group, a pyridine group, a pyrazine group, a pyrimidine group, a pyridazine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, and an azacarbazole group; and

a benzene group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, a dibenzothiophene group, a carbazole group, an imidazole group, a pyrazole group, a thiazole group, an isothiazole group, an oxazole group, an isoxazole group, a pyridine group, a pyrazine group, a pyrimidine group, a pyridazine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, and an azacarbazole group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, —Si(Q₃₁)(Q₃₂)(Q₃₃), —S(═O)₂(Q₃₁), and —P(═O)(Q₃₁)(Q₃₂), and

Q₃₁ to Q₃₃ may each independently be selected from a C₁-C₁₀ alkyl group, a C₁-C₁₀ alkoxy group, a phenyl group, a biphenyl group, a terphenyl group, and a naphthyl group.

When xe11 in Formula 601 is 2 or more, two or more Ar₆₀ₗ(s) may be linked via a single bond.

In one or more embodiments, Ar₆₀₁ in Formula 601 may be an anthracene group.

In one or more embodiments, a compound represented by Formula 601 may be represented by Formula 601-1 below:

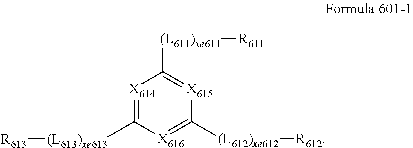

In Formula 601-1,

X₆₁₄ may be N or C(R₆₁₄), X₆₁₅ may be N or C(R₆₁₅), X₆₁₆ may be N or C(R₆₁₆), and at least one selected from X₆₁₄ to X₆₁₆ may be N,

L₆₁₁ to L₆₁₃ may each independently be the same as described in connection with L₆₀₁,

xe611 to xe613 may each independently be defined the same as xe1,

R₆₁₁ to R₆₁₃ may each independently be understood by referring to the description presented in connection with R₆₀₁, and

R₆₁₄ to R₆₁₆ may each independently be selected from hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, and a naphthyl group.

In one embodiment, L₆₀₁ and L₆₁₁ to L₆₁₃ in Formulae 601 and 601-1 may each independently be selected from:

a phenylene group, a naphthylene group, a fluorenylene group, a spiro-bifluorenylene group, a benzofluorenylene group, a dibenzofluorenylene group, a phenanthrenylene group, an anthracenylene group, a fluoranthenylene group, a triphenylenylene group, a pyrenylene group, a chrysenylene group, a perylenylene group, a pentaphenylene group, a hexacenylene group, a pentacenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, a pyridinylene group, an imidazolylene group, a pyrazolylene group, a thiazolylene group, an isothiazolylene group, an oxazolylene group, an isoxazolylene group, a thiadiazolylene group, an oxadiazolylene group, a pyrazinylene group, a pyrimidinylene group, a pyridazinylene group, a triazinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a phenanthridinylene group, an acridinylene group, a phenanthrolinylene group, a phenazinylene group, a benzimidazolylene group, an isobenzothiazolylene group, a benzoxazolylene group, an isobenzoxazolylene group, a triazolylene group, a tetrazolylene group, an imidazopyridinylene group, an imidazopyrimidinylene group, and an azacarbazolylene group; and

a phenylene group, a naphthylene group, a fluorenylene group, a spiro-bifluorenylene group, a benzofluorenylene group, a dibenzofluorenylene group, a phenanthrenylene group, an anthracenylene group, a fluoranthenylene group, a triphenylenylene group, a pyrenylene group, a chrysenylene group, a perylenylene group, a pentaphenylene group, a hexacenylene group, a pentacenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, a pyridinylene group, an imidazolylene group, a pyrazolylene group, a thiazolylene group, an isothiazolylene group, an oxazolylene group, an isoxazolylene group, a thiadiazolylene group, an oxadiazolylene group, a pyrazinylene group, a pyrimidinylene group, a pyridazinylene group, a triazinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a phenanthridinylene group, an acridinylene group, a phenanthrolinylene group, a phenazinylene group, a benzimidazolylene group, an isobenzothiazolylene group, a benzoxazolylene group, an isobenzoxazolylene group, a triazolylene group, a tetrazolylene group, an imidazopyridinylene group, an imidazopyrimidinylene group, and an azacarbazolylene group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, and an azacarbazolyl group,

but embodiments of the present disclosure are not limited thereto.

In one or more embodiments, xe1 and xe611 to xe613 in Formulae 601 and 601-1 may each independently be 0, 1, or 2.

In one or more embodiments, R₆₀₁ and R₆₁₁ to R₆₁₃ in Formulae 601 and 601-1 may each independently be selected from:

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, and an azacarbazolyl group;

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, and an azacarbazolyl group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, and an azacarbazolyl group; and

—S(═O)₂(Q₆₀₁) and —P(═O)(Q₆₀₁)(Q₆₀₂), and

Q₆₀₁ and Q₆₀₂ are the same as described herein above.

The electron transport region may include at least one compound selected from Compounds ET1 to ET36, but embodiments of the present disclosure are not limited thereto:

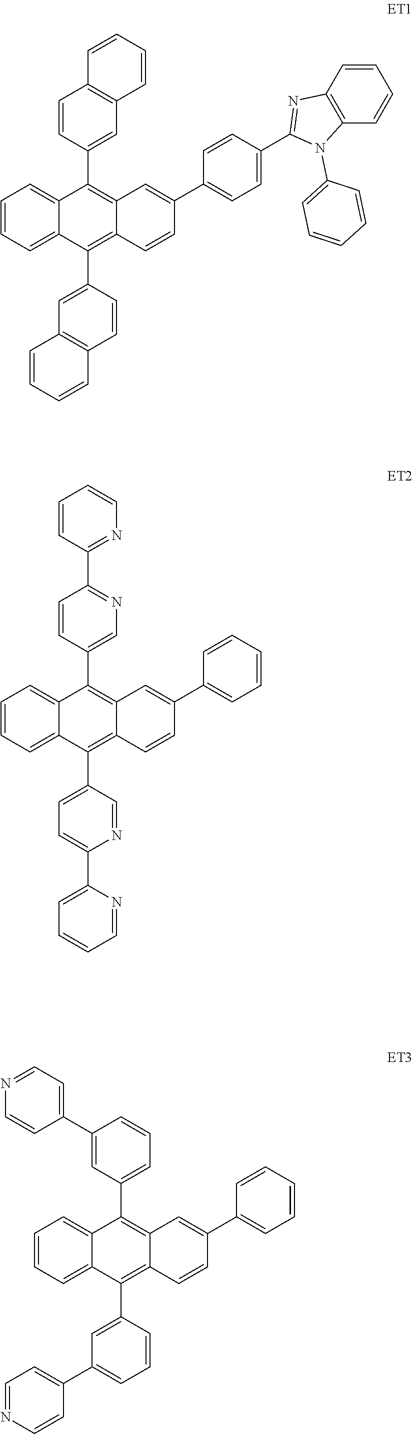 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

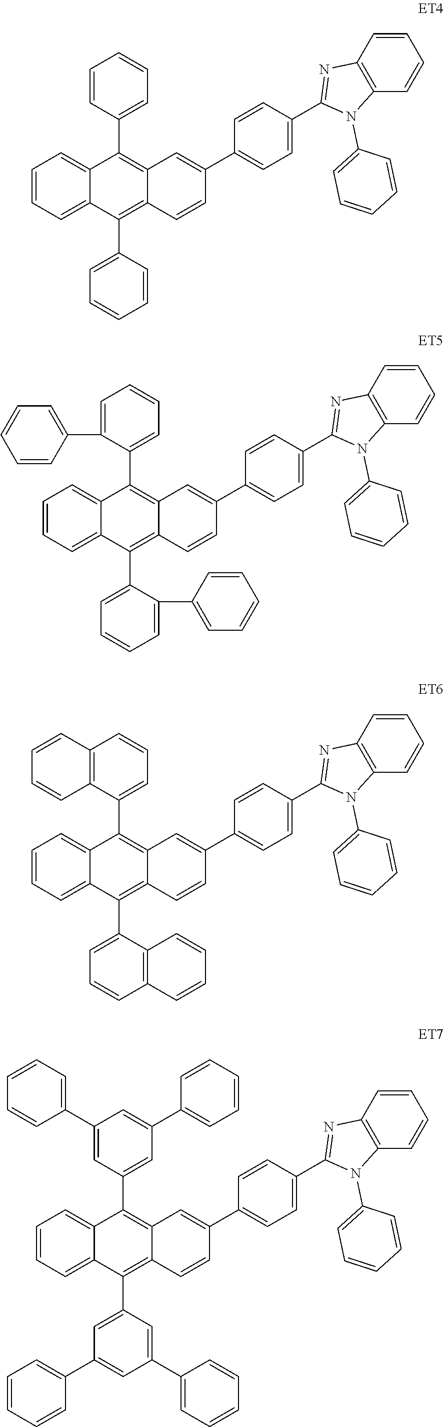 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

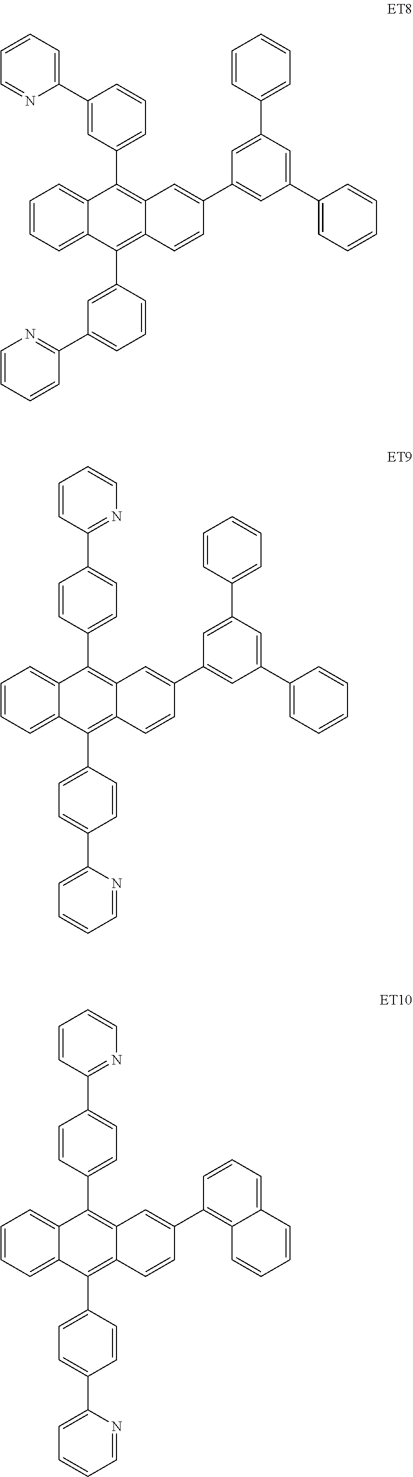 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

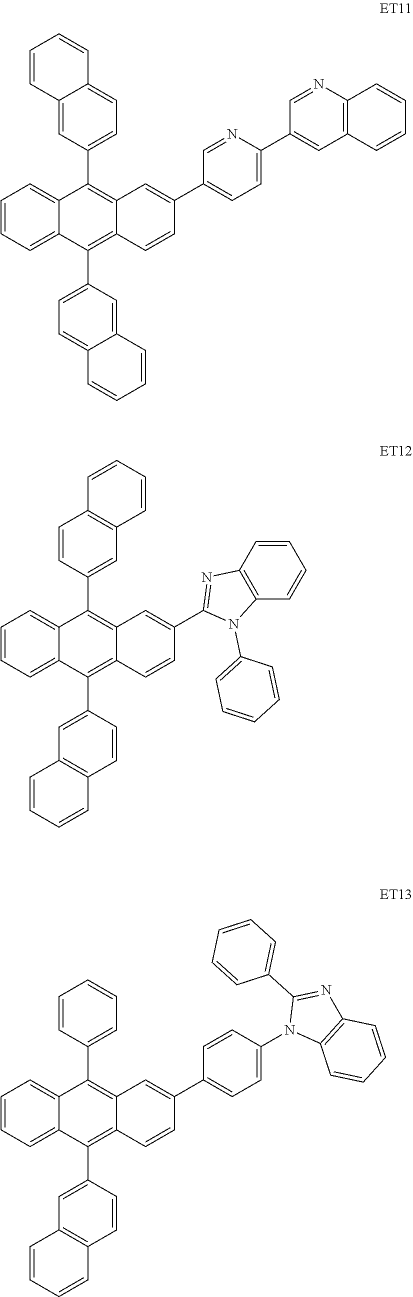 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

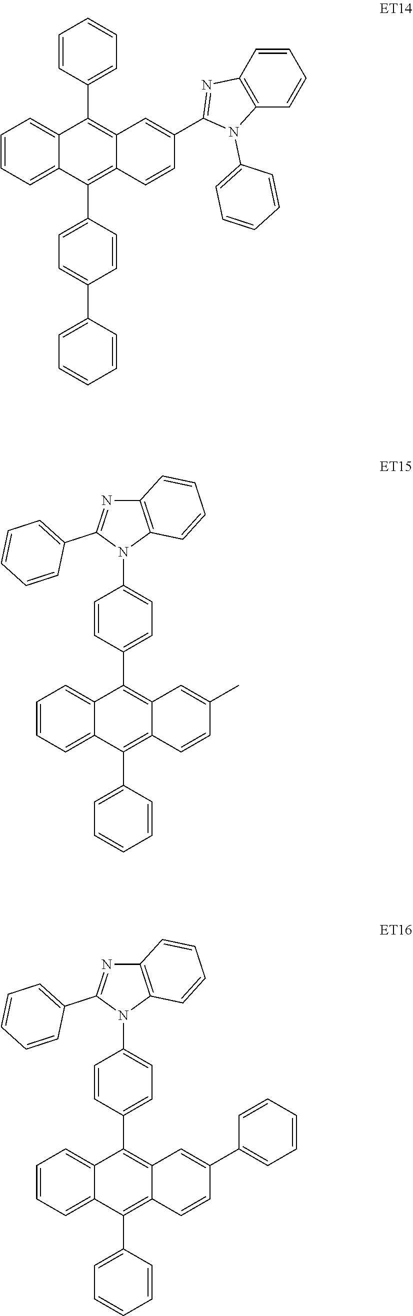 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

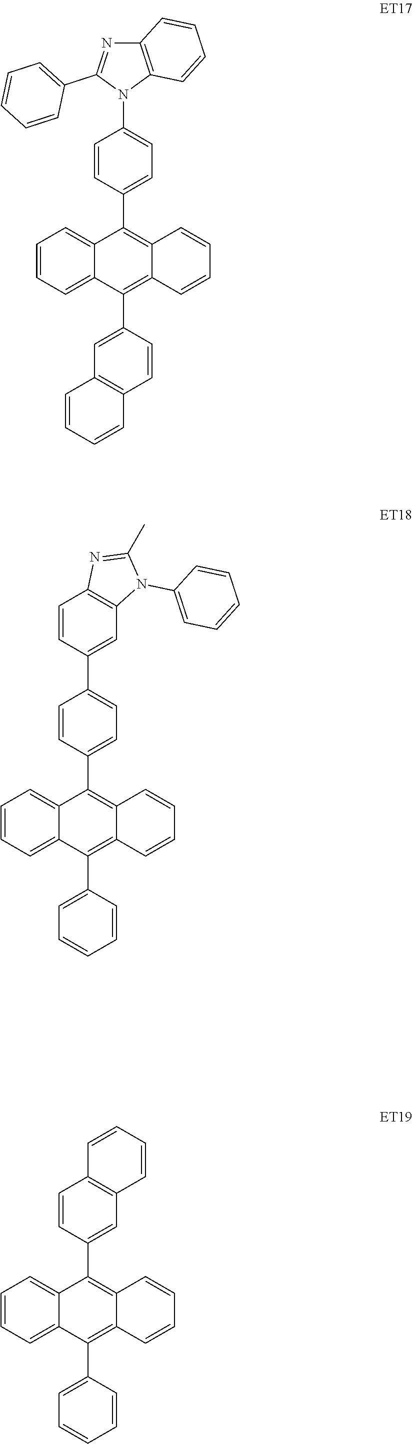

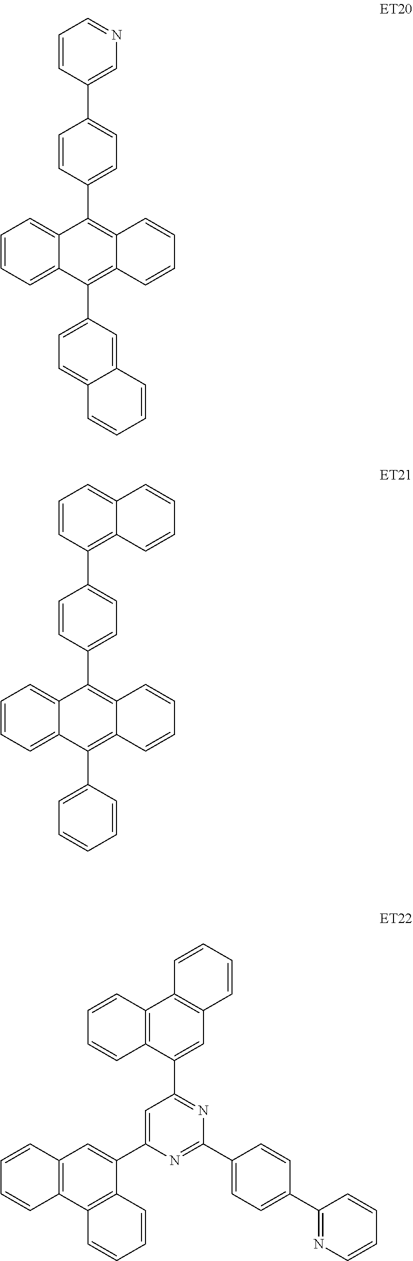

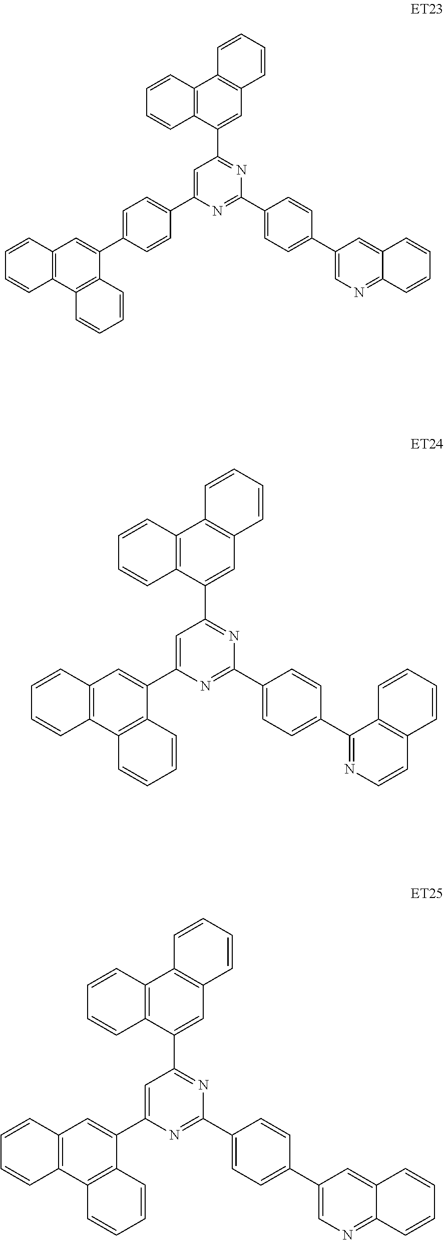 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)cc(-c5cc6ccccc6c6ccccc56)n4)cc3)cc2c1]

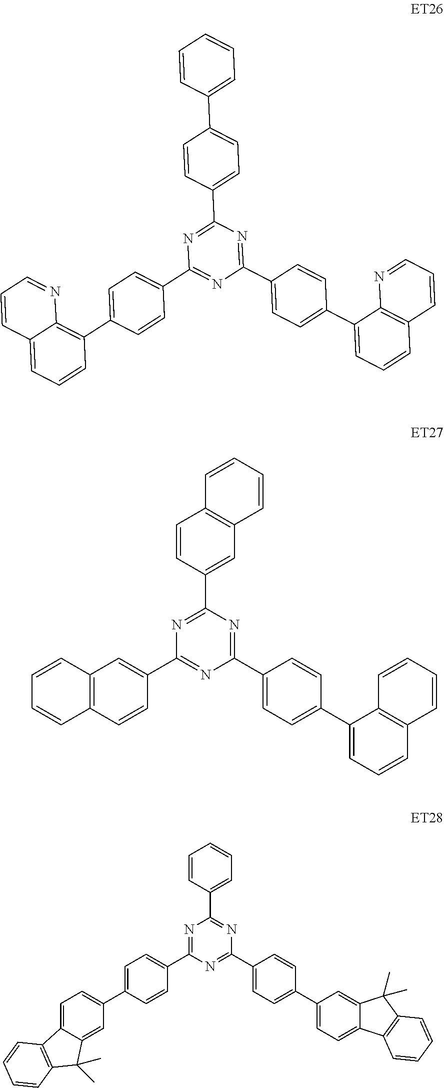 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6ccccc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

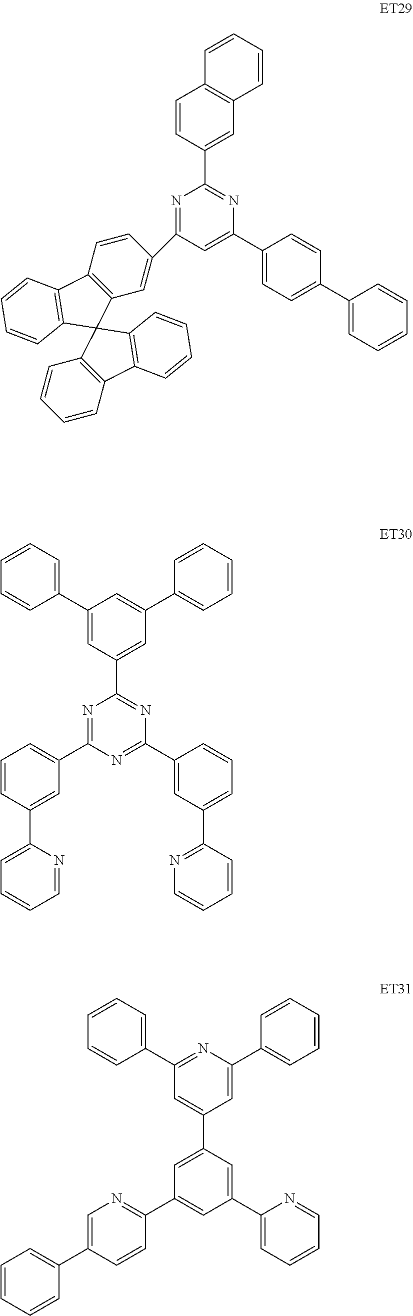 [SMILES: c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1]

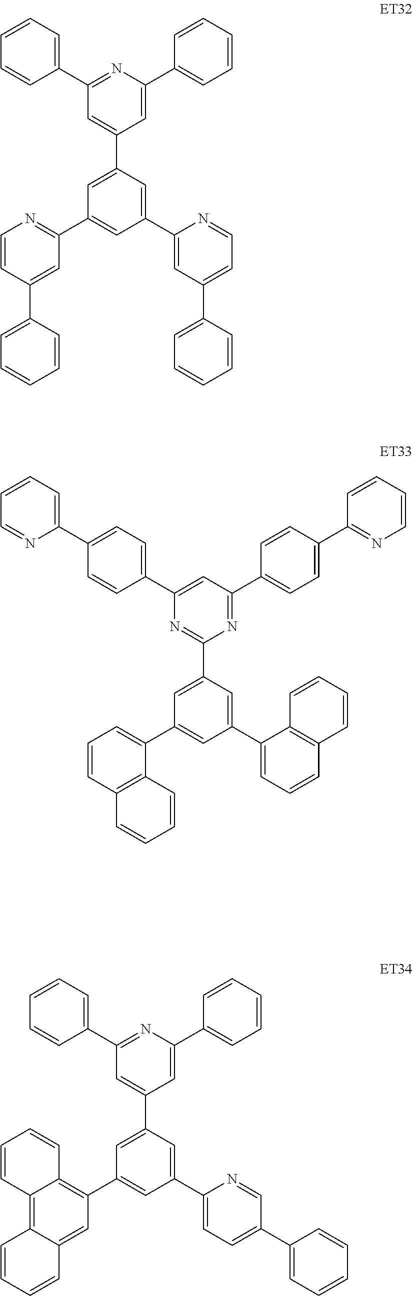 [SMILES: c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

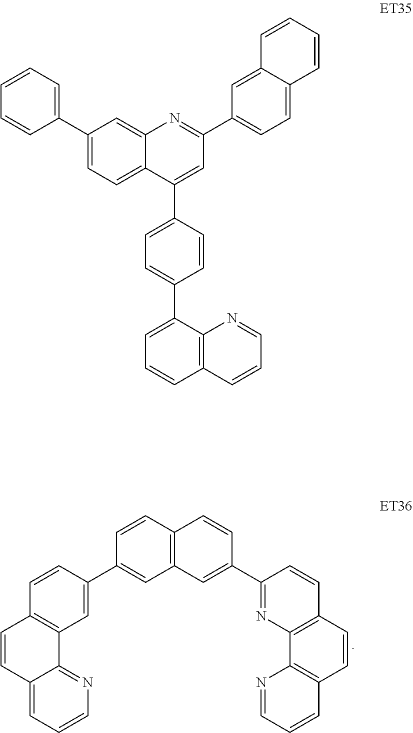 [SMILES: c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)cc12]

In one or more embodiments, the electron transport region may include at least one compound selected from 2,9-dimethyl-4,7-diphenyl-1,10-phenanthroline (BCP), 4,7-diphenyl-1,10-phenanthroline (Bphen), Alq3, BAlq, 3-(biphenyl-4-yl)-5-(4-tert-butylphenyl)-4-phenyl-4H-1,2,4-triazole (TAZ), and NTAZ:

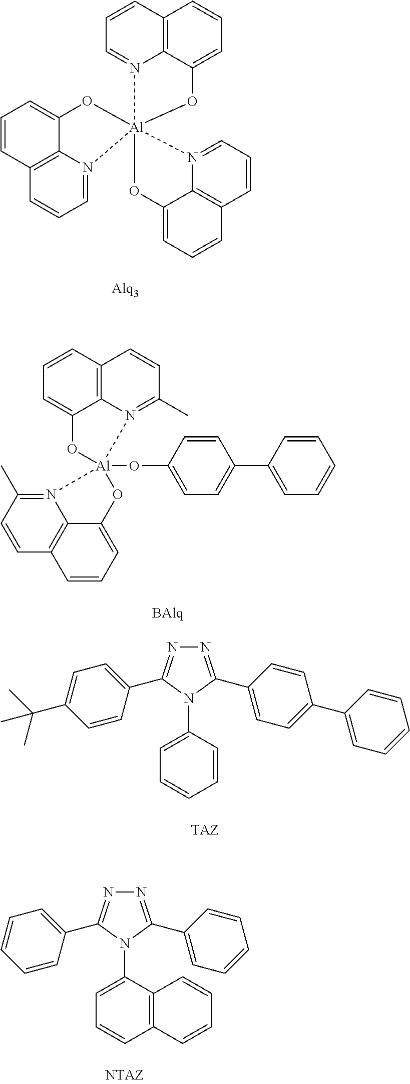

A thickness of the buffer layer, the hole blocking layer, or the electron control layer may be in a range of about 20 Å to about 1,000 Å, for example, about 30 Å to about 300 Å. When the thicknesses of the buffer layer, the hole blocking layer, and the electron control layer are within these ranges, the electron blocking layer may have excellent electron blocking characteristics or electron control characteristics without a substantial increase in driving voltage.

A thickness of the electron transport layer may be from about 100 Å to about 1,000 Å, for example, about 150 Å to about 500 Å. When the thickness of the electron transport layer is within the range described herein above, the electron transport layer may have suitable or satisfactory electron transport characteristics without a substantial increase in driving voltage.

The electron transport region (for example, the electron transport layer in the electron transport region) may further include, in addition to the materials described herein above, a metal-containing material.

The metal-containing material may include at least one selected from alkali metal complex and alkaline earth-metal complex. The alkali metal complex may include a metal ion selected from a Li ion, a Na ion, a K ion, a Rb ion, and a Cs ion, and the alkaline earth-metal complex may include a metal ion selected from a Be ion, a Mg ion, a Ca ion, a Sr ion, and a Ba ion. A ligand coordinated with the metal ion of the alkali metal complex or the alkaline earth-metal complex may be selected from a hydroxy quinoline, a hydroxy isoquinoline, a hydroxy benzoquinoline, a hydroxy acridine, a hydroxy phenanthridine, a hydroxy phenyloxazole, a hydroxy phenylthiazole, a hydroxy diphenyloxadiazole, a hydroxy diphenylthiadiazole, a hydroxy phenylpyridine, a hydroxy phenylbenzimidazole, a hydroxy phenylbenzothiazole, a bipyridine, a phenanthroline, and a cyclopentadiene, but embodiments of the present disclosure are not limited thereto.

For example, the metal-containing material may include a Li complex. The Li complex may include, for example, Compound ET-D1 (lithium quinolate, LiQ) or ET-D2:

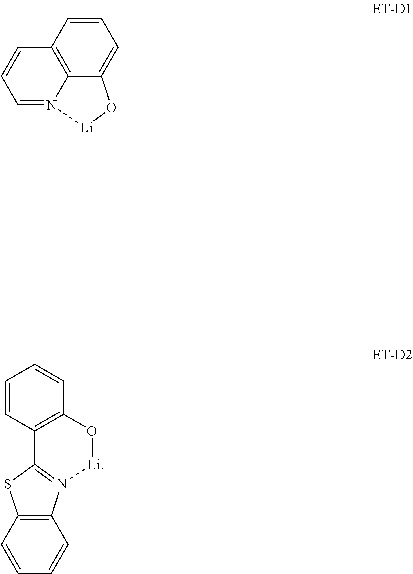

The electron transport region may include an electron injection layer that facilitates electron injection from the second electrode 190. The electron injection layer may directly contact the second electrode 190.

The electron injection layer may have i) a single-layered structure including a single layer including a single material, ii) a single-layered structure including a single layer including a plurality of different materials, or iii) a multi-layered structure having a plurality of layers including a plurality of different materials.

The electron injection layer may include an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or any combinations thereof.

The alkali metal may be selected from Li, Na, K, Rb, and Cs. In one embodiment, the alkali metal may be Li, Na, or Cs. In one or more embodiments, the alkali metal may be Li or Cs, but embodiments of the present disclosure are not limited thereto.

The alkaline earth metal may be selected from Mg, Ca, Sr, and Ba.

The rare earth metal may be selected from Sc, Y, Ce, Tb, Yb, and Gd.

The alkali metal compound, the alkaline earth-metal compound, and the rare earth metal compound may be selected from oxides and halides (for example, fluorides, chlorides, bromides, or iodides) of the alkali metal, the alkaline earth-metal, and the rare earth metal.

The alkali metal compound may be selected from alkali metal oxides, such as Li₂O, Cs₂O, or K₂O, and alkali metal halides, such as LiF, NaF, CsF, KF, LiI, NaI, CsI, or KI. In one embodiment, the alkali metal compound may be selected from LiF, Li₂O, NaF, LiI, NaI, CsI, and KI, but embodiments of the present disclosure are not limited thereto.

The alkaline earth-metal compound may be selected from alkaline earth-metal oxides, such as BaO, SrO, CaO, BaₓSr₁₋ₓO (0<x<1), or BaₓCa₁₋ₓO (0<x<1). In one embodiment, the alkaline earth-metal compound may be selected from BaO, SrO, and CaO, but embodiments of the present disclosure are not limited thereto.

The rare earth metal compound may be selected from YbF₃, ScF₃, ScO₃, Y₂O, Ce₂O₃, GdF₃, and TbF₃. In one embodiment, the rare earth metal compound may be selected from YbF₃, ScF₃, TbF₃, YbI₃, ScI₃, and TbI₃, but embodiments of the present disclosure are not limited thereto.

The alkali metal complex, the alkaline earth-metal complex, and the rare earth metal complex may include an ion of alkali metal, alkaline earth-metal, and rare earth metal as described herein above, and a ligand coordinated with a metal ion of the alkali metal complex, the alkaline earth-metal complex, or the rare earth metal complex may be selected from hydroxy quinoline, hydroxy isoquinoline, hydroxy benzoquinoline, hydroxy acridine, hydroxy phenanthridine, hydroxy phenyloxazole, hydroxy phenylthiazole, hydroxy diphenyloxadiazole, hydroxy diphenylthiadiazole, hydroxy phenylpyridine, hydroxy phenylbenzimidazole, hydroxy phenylbenzothiazole, bipyridine, phenanthroline, and cyclopentadiene, but embodiments of the present disclosure are not limited thereto.

The electron injection layer may consist of an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or any combinations thereof, as described herein above. In one or more embodiments, the electron injection layer may further include an organic material. When the electron injection layer further includes an organic material, an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or any combinations thereof may be homogeneously or non-homogeneously dispersed in a matrix including the organic material.

A thickness of the electron injection layer may be in a range of about 1 Å to about 100 Å, for example, about 3 Å to about 90 Å. When a thickness of the electron injection layer is within these ranges, suitable or satisfactory electron injection characteristics may be obtained without substantial increase in driving voltage.

### Second Electrode 190

The second electrode 190 may be on the organic layer 150 having such a structure. The second electrode 190 may be a cathode which is an electron injection electrode, and in this regard, a material for forming the second electrode 190 may be selected from metal, an alloy, an electrically conductive compound, and a combination thereof, which have a relatively low work function.

The second electrode 190 may include at least one selected from lithium (Li), silver (Ag), magnesium (Mg), aluminum (Al), aluminum-lithium (Al—Li), calcium (Ca), magnesium-indium (Mg—In), magnesium-silver (Mg—Ag), ITO, and IZO, but embodiments of the present disclosure are not limited thereto. The second electrode 190 may be a transmissive electrode, a semi-transmissive electrode, or a reflective electrode.

The second electrode 190 may have a single-layered structure, or a multi-layered structure including two or more layers.

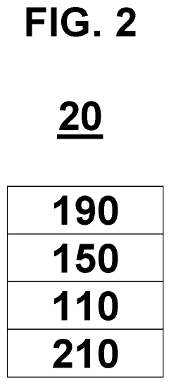

### Description of FIGS. 2 to 4

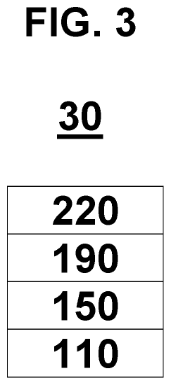

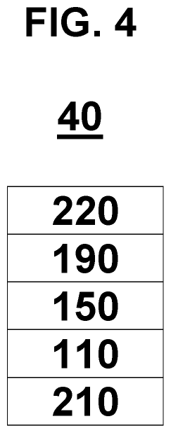

FIG. 2 is a schematic view of an organic light-emitting device 20 according to an embodiment. The organic light-emitting device 20 includes a first capping layer 210, the first electrode 110, the organic layer 150, and the second electrode 190, which are sequentially stacked in this stated order. FIG. 3 is a schematic view of an organic light-emitting device 30 according to an embodiment. The organic light-emitting device 30 includes the first electrode 110, the organic layer 150, the second electrode 190, and a second capping layer 220, which are sequentially stacked in this stated order. FIG. 4 is a schematic view of an organic light-emitting device 40 according to an embodiment. The organic light-emitting device 40 includes the first capping layer 210, the first electrode 110, the organic layer 150, the second electrode 190, and the second capping layer 220, which are sequentially stacked in this stated order.

Regarding FIGS. 2 to 4, the first electrode 110, the organic layer 150, and the second electrode 190 may be understood by referring to the description presented in connection with FIG. 1.

In the organic layer 150 of each of the organic light-emitting devices 20 and 40, light generated in an emission layer may pass through the first electrode 110 and the first capping layer 210 toward the outside, wherein the first electrode 110 may be a semi-transmissive electrode or a transmissive electrode. In the organic layer 150 of each of the organic light-emitting devices 30 and 40, light generated in an emission layer may pass through the second electrode 190 and the second capping layer 220 toward the outside, wherein the second electrode 190 may be a semi-transmissive electrode or a transmissive electrode.

The first capping layer 210 and the second capping layer 220 may increase external luminescence efficiency according to the principle of constructive interference.

The first capping layer 210 and the second capping layer 220 may each independently be an organic capping layer including an organic material, an inorganic capping layer including an inorganic material, or a composite capping layer including an organic material and an inorganic material.

At least one selected from the first capping layer 210 and the second capping layer 220 may each independently include at least one material selected from carbocyclic compounds, heterocyclic compounds, amine-based compounds, porphyrine derivatives, phthalocyanine derivatives, a naphthalocyanine derivatives, alkali metal complexes, and alkaline earth-based complexes. The carbocyclic compound, the heterocyclic compound, and the amine-based compound may be optionally substituted with a substituent containing at least one element selected from O, N, S, Se, Si, F, C, Br, and I. In one embodiment, at least one selected from the first capping layer 210 and the second capping layer 220 may each independently include an amine-based compound.

In one embodiment, at least one selected from the first capping layer 210 and the second capping layer 220 may each independently include the compound represented by Formula 201 or the compound represented by Formula 202.

In one or more embodiments, at least one selected from the first capping layer 210 and the second capping layer 220 may each independently include a compound selected from Compounds HT28 to HT33 and Compounds CP1 to CP5, but embodiments of the present disclosure are not limited thereto:

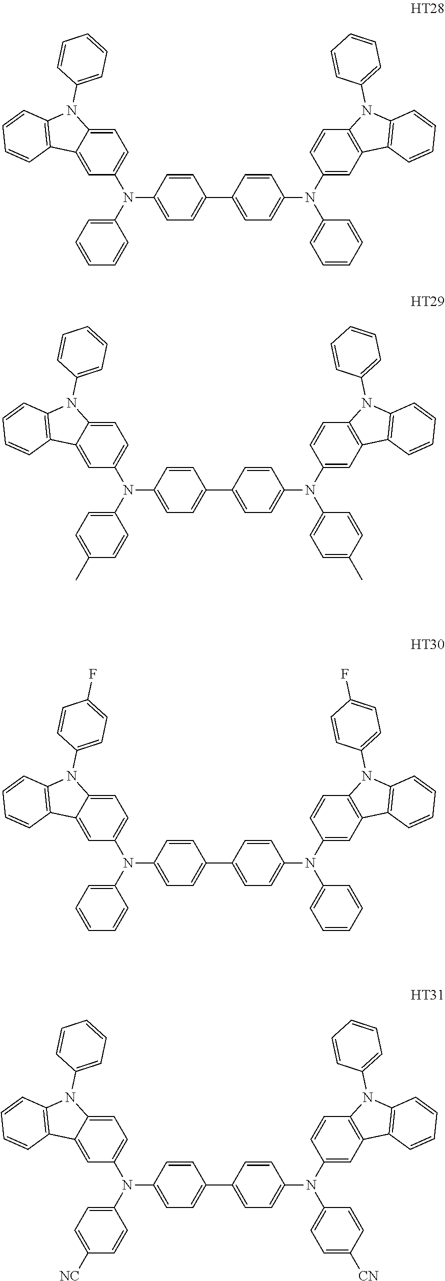 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1 ; N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1]

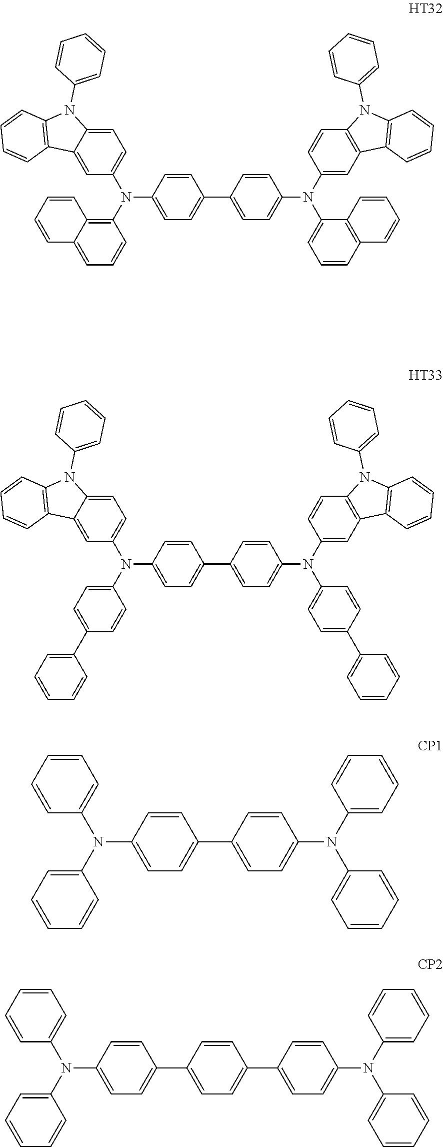 [SMILES: c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1]

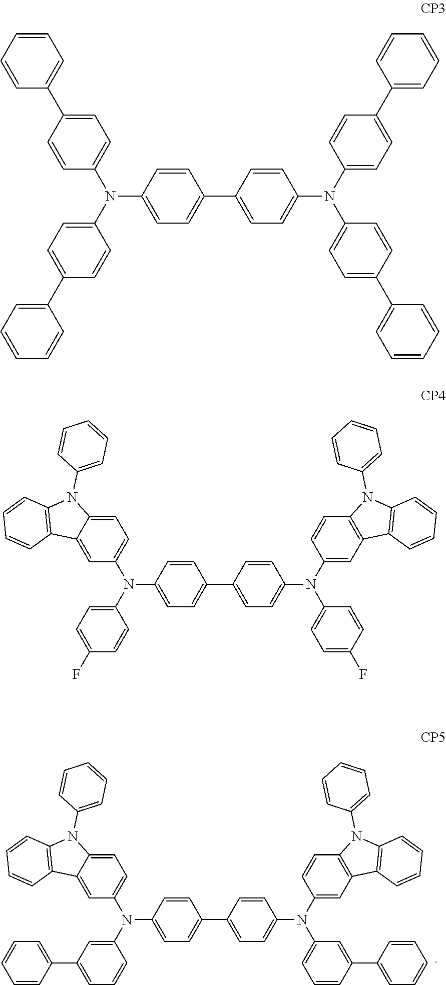 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

Hereinbefore, the organic light-emitting device according to an embodiment has been described in connection with FIGS. 1 to 4, but embodiments of the present disclosure are not limited thereto.

Layers constituting the hole transport region, an emission layer, and layers constituting the electron transport region may be formed in a certain region by using one or more suitable methods selected from vacuum deposition, spin coating, casting, Langmuir-Blodgett (LB) deposition, ink-jet printing, laser-printing, and laser-induced thermal imaging.

When layers constituting the hole transport region, an emission layer, and layers constituting the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec by taking into account a material to be included in a layer to be formed, and the structure of a layer to be formed.

When layers constituting the hole transport region, an emission layer, and layers constituting the electron transport region are formed by spin coating, the spin coating may be performed at a coating speed of about 2,000 rpm to about 5,000 rpm and at a heat treatment temperature of about 80° C. to 200° C. by taking into account a material to be included in a layer to be formed, and the structure of a layer to be formed.

### Apparatus

The organic light-emitting device may be included in various suitable apparatuses. For example, a light-emitting apparatus, an authentication apparatus, or an electronic apparatus, which includes the organic light-emitting device, may be provided.

The light-emitting apparatus may further include, in addition to the organic light-emitting device, a thin film transistor including a source electrode and a drain electrode. One of the source electrode and the drain electrode of the thin film transistor may be in electrical contact with one of the first electrode and the second electrode of the organic light-emitting device. The light-emitting apparatus may be used as various suitable displays, light sources, and the like. For example, light-emitting apparatus may be used as a display for a television, mobile phone, or the like, but the present disclosure is not limited thereto.

The authentication apparatus may be, for example, a biometric authentication apparatus for authenticating an individual by using biometric information of a biometric body (for example, a finger tip, a pupil, or the like).

The authentication apparatus may further include, in addition to the organic light-emitting device, a biometric information collector.

The electronic apparatus may be applied to personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram (ECG) displays, ultrasonic diagnostic devices, or endoscope displays), fish finders, various suitable measuring instruments, meters (for example, meters for a vehicle, an aircraft, and a vessel), projectors, and the like, but embodiments of the present disclosure are not limited thereto.

### General Definition of at Least Some of the Substituents

The term “C₁-C₆₀ alkyl group,” as used herein, refers to a linear or branched aliphatic saturated hydrocarbon monovalent group having 1 to 60 carbon atoms, and examples thereof include a methyl group, an ethyl group, a propyl group, an isobutyl group, a sec-butyl group, a tert-butyl group, a pentyl group, an isoamyl group, and a hexyl group. The term “C₁-C₆₀ alkylene group,” as used herein, refers to a divalent group having substantially the same structure as the C₁-C₆₀ alkyl group.

The term “C₂-C₆₀ alkenyl group,” as used herein, refers to a hydrocarbon group having at least one carbon-carbon double bond at a main chain (e.g., in the middle) or at a terminal end (e.g., the terminus) of the C₂-C₆₀ alkyl group, and examples thereof include an ethenyl group, a propenyl group, and a butenyl group. The term “C₂-C₆₀ alkenylene group,” as used herein, refers to a divalent group having substantially the same structure as the C₂-C₆₀ alkenyl group.

The term “C₂-C₆₀ alkynyl group,” as used herein, refers to a hydrocarbon group having at least one carbon-carbon triple bond at a main chain (e.g., in the middle) or at a terminal end (e.g., the terminus) of the C₂-C₆₀ alkyl group, and examples thereof include an ethynyl group, and a propynyl group. The term “C₂-C₆₀ alkynylene group,” as used herein, refers to a divalent group having substantially the same structure as the C₂-C₆₀ alkynyl group.

The term “C₁-C₆₀ alkoxy group,” as used herein, refers to a monovalent group represented by —OA₁₀₁ (wherein A₁₀₁ is the C₁-C₆₀ alkyl group), and examples thereof include a methoxy group, an ethoxy group, and an isopropyloxy group.

The term “C₃-C₁₀ cycloalkyl group,” as used herein, refers to a monovalent saturated hydrocarbon monocyclic group having 3 to 10 carbon atoms, and examples thereof include a cyclopropyl group, a cyclobutyl group, a cyclopentyl group, a cyclohexyl group, and a cycloheptyl group. The term “C₃-C₁₀ cycloalkylene group,” as used herein, refers to a divalent group having substantially the same structure as the C₃-C₁₀ cycloalkyl group.

The term “C₁-C₁₀ heterocycloalkyl group,” as used herein, refers to a monovalent monocyclic group having at least one heteroatom selected from N, O, Si, P, and S as a ring-forming atom and 1 to 10 carbon atoms, and examples thereof include a 1,2,3,4-oxatriazolidinyl group, a tetrahydrofuranyl group, and a tetrahydrothiophenyl group. The term “C₁-C₁₀ heterocycloalkylene group,” as used herein, refers to a divalent group having substantially the same structure as the C₁-C₁₀ heterocycloalkyl group.

The term “C₃-C₁₀ cycloalkenyl group,” as used herein, refers to a monovalent monocyclic group that has 3 to 10 carbon atoms and at least one carbon-carbon double bond in the ring thereof and no aromaticity (e.g., is not aromatic), and examples thereof include a cyclopentenyl group, a cyclohexenyl group, and a cycloheptenyl group. The term “C₃-C₁₀ cycloalkenylene group,” as used herein, refers to a divalent group having substantially the same structure as the C₃-C₁₀ cycloalkenyl group.

The term “C₁-C₁₀ heterocycloalkenyl group,” as used herein, refers to a monovalent monocyclic group that has at least one heteroatom selected from N, O, Si, P, and S as a ring-forming atom, 1 to 10 carbon atoms, and at least one carbon-carbon double bond in its ring. Non-limiting examples of the C₁-C₁₀ heterocycloalkenyl group include a 4,5-dihydro-1,2,3,4-oxatrazolyl group, a 2,3-dihydrofuranyl group, and a 2,3-dihydrothiophenyl group. The term “C₁-C₁₀ heterocycloalkenylene group,” as used herein, refers to a divalent group having substantially the same structure as the C₁-C₁₀ heterocycloalkenyl group.

The term “C₆-C₆₀ aryl group,” as used herein, refers to a monovalent group having a carbocyclic aromatic system having 6 to 60 carbon atoms, and the term “C₆-C₆₀ arylene group,” as used herein, refers to a divalent group having a carbocyclic aromatic system having 6 to 60 carbon atoms. Non-limiting examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other (e.g., combined together).

The term “C₁-C₆₀ heteroaryl group,” as used herein, refers to a monovalent group having a carbocyclic aromatic system that has at least one heteroatom selected from N, O, Si, P, and S as a ring-forming atom, in addition to 1 to 60 carbon atoms. The term “C₁-C₆₀ heteroarylene group,” as used herein, refers to a divalent group having a carbocyclic aromatic system that has at least one heteroatom selected from N, O, Si, P, and S as a ring-forming atom, in addition to 1 to 60 carbon atoms. Non-limiting examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and the C₁-C₆₀ heteroarylene group each include two or more rings, the rings may be condensed with each other (e.g., combined together).

The term “C₆-C₆₀ aryloxy group,” as used herein, refers to —OA₁₀₂ (wherein A₁₀₂ is the C₆-C₆₀ aryl group), and the term “C₆-C₆₀ arylthio group,” as used herein, indicates —SA₁₀₃ (wherein A₁₀₃ is the C₆-C₆₀ aryl group).

The term “monovalent non-aromatic condensed polycyclic group,” as used herein, refers to a monovalent group (for example, having 8 to 60 carbon atoms) having two or more rings condensed with each other (e.g., combined together), only carbon atoms as ring-forming atoms, and no aromaticity in its entire molecular structure (e.g., is not aromatic). A detailed example of the monovalent non-aromatic condensed polycyclic group is a fluorenyl group. The term “divalent non-aromatic condensed polycyclic group,” as used herein, refers to a divalent group having substantially the same structure as the monovalent non-aromatic condensed polycyclic group.

The term “monovalent non-aromatic condensed heteropolycyclic group,” as used herein, refers to a monovalent group (for example, having 1 to 60 carbon atoms) having two or more rings condensed to each other (e.g., combined together), at least one heteroatom selected from N, O, Si, P, and S, other than carbon atoms, as a ring-forming atom, and no aromaticity in its entire molecular structure (e.g., is not aromatic). An example of the monovalent non-aromatic condensed heteropolycyclic group is a carbazolyl group. The term “divalent non-aromatic condensed heteropolycyclic group,” as used herein, refers to a divalent group having substantially the same structure as the monovalent non-aromatic condensed heteropolycyclic group.

The term “C₅-C₆₀ carbocyclic group,” as used herein, refers to a monocyclic or polycyclic group having 5 to 60 carbon atoms in which a ring-forming atom is a carbon atom only. The term “C₅-C₆₀ carbocyclic group,” as used herein, refers to an aromatic carbocyclic group or a non-aromatic carbocyclic group. The C₅-C₆₀ carbocyclic group may be a ring, such as benzene, a monovalent group, such as a phenyl group, or a divalent group, such as a phenylene group. In one or more embodiments, depending on the number of substituents connected to the C₅-C₆₀ carbocyclic group, the C₅-C₆₀ carbocyclic group may be a trivalent group or a quadrivalent group.

The term “C₁-C₆₀ heterocyclic group,” as used herein, refers to a group having substantially the same structure as the C₅-C₆₀ carbocyclic group, except that as a ring-forming atom, at least one heteroatom selected from N, O, Si, P, and S is used in addition to carbon (the number of carbon atoms may be in a range of 1 to 60).

In the present specification, at least one substituent of the substituted C₅-C₆₀ carbocyclic group, the substituted C₁-C₆₀ heterocyclic group, the substituted C₃-C₁₀ cycloalkylene group, the substituted C₁-C₁₀ heterocycloalkylene group, the substituted C₃-C₁₀ cycloalkenylene group, the substituted C₁-C₁₀ heterocycloalkenylene group, the substituted C₆-C₆₀ arylene group, the substituted C₁-C₆₀ heteroarylene group, the substituted divalent non-aromatic condensed polycyclic group, the substituted divalent non-aromatic condensed heteropolycyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be selected from:

deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, a hydroxyl group, a cyano group, a nitro group, an amidino group, a hydrazino group, a hydrazono group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), and —P(═O)(Q₁₁)(Q₁₂);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —C, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), and —P(═O)(Q₂₁)(Q₂₂); and

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂), and

Q₁₁ to Q₁₃, Q₂₁ to Q₂₃, and Q₃₁ to Q₃₃ may each independently be selected from hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, a monovalent non-aromatic condensed heteropolycyclic group, a biphenyl group, and a terphenyl group.

The term “Ph,” as used herein, refers to a phenyl group, the term “Me,” as used herein, refers to a methyl group, the term “Et,” as used herein, refers to an ethyl group, the term “ter-Bu” or “Bu^{t},” as used herein, refers to a tert-butyl group, and the term “OMe,” as used herein, refers to a methoxy group.

The term “biphenyl group,” as used herein, refers to “a phenyl group substituted with a phenyl group.” In other words, the “biphenyl group” is a substituted phenyl group having a C₆-C₆₀ aryl group as a substituent.

The term “terphenyl group,” as used herein, refers to “a phenyl group substituted with a biphenyl group.” In other words, the “terphenyl group” is a phenyl group having, as a substituent, a C₆-C₆₀ aryl group substituted with a C₆-C₆₀ aryl group.

* and *′, as used herein, unless defined otherwise, each refer to a binding site to a neighboring atom in a corresponding formula.

Hereinafter, a compound according to embodiments and an organic light-emitting device according to embodiments will be described in more detail with reference to Synthesis Examples and Examples. The wording “B was used instead of A” used in describing Synthesis Examples refers to that an identical molar equivalent of B was used in place of A.

### EXAMPLES

### Synthesis of Intermediate C(1)

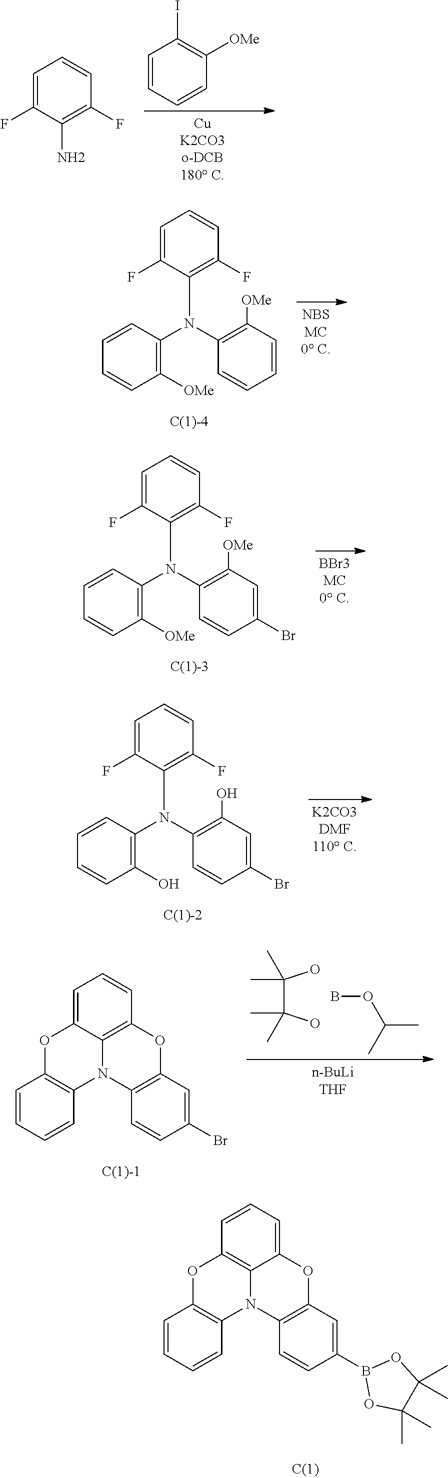

### Synthesis of Intermediate C(1)-4

2,6-difluoroaniline (4.55 g, 1 eq), 1-iodo-2-methoxybenzene (17 g, 2.1 eq), Cu (4.6 g, 2 eq), K₂CO₃ (12.5 g, 2.6 eq), and 60 ml of o-DCB were added to a one-neck round bottom flask and stirred at a temperature of 180° C. for 3 days. A residue obtained after the reaction was believed to be completed was separated and purified by column chromatography using a mixture of methylene chloride (MC) and hexane (1:2) as a developing solvent to obtain about 6.7 g (yield: 56%) of Intermediate C(1)-4.

### Synthesis of Intermediate C(1)-3

Intermediate C(1)-4 (9.5 g, 1 eq), NBS (4.9 g, 1 eq), and 150 ml of MC were added to a one-neck round bottom flask and stirred at room temperature for 12 hours. A residue obtained after the reaction was believed to be completed was separated and purified by column chromatography using a mixture of MC and hexane (1:5) as a developing solvent to obtain about 7 g (yield: 85%) of Intermediate C(1)-3.

### Synthesis of Intermediate C(1)-2

Intermediate C(1)-3 (7 g, 1 eq) and 250 ml of MC were added to a three-neck round bottom flask, and BBr₃ (3.89 ml, 2.5 eq) dissolved in 60 ml of MC was added dropwise thereto while stirring at a temperature of −78° C. After the dropwise addition was completed, the reaction mixture was stirred at a temperature of 0° C. for 4 hours. After the reaction was believed to be completed, an organic layer was extracted therefrom by using MC. The extracted organic layer was dried by using MgSO₄, and a solvent was evaporated therefrom to obtain about 6 g (yield: 92%) of Intermediate C(1)-2.

### Synthesis of Intermediate C(1)-1

Intermediate C(1)-2 (6 g, 1 eq), K₂CO₃ (6.34 g, 3 eq), and 200 ml of dimethylformamide (DMF) were added to a one-neck round bottom flask and stirred at a temperature of 110° C. for 8 hours. After the reaction was believed to be completed, DMF was removed therefrom. Then, a solid obtained by distilling a solvent off under reduced pressure through a silica gel short path column was recrystallized with ether to obtain about 4.8 g (yield: 89%) of Intermediate C(1)-1.

### Synthesis of Intermediate C(1)

Intermediate C(1)-1 (2 g, 1 eq) and 20 ml of tetrahydrofuran (THF) were added to a three-neck round bottom flask, and n-BuLi (2.7 ml, 1.2 eq) was added thereto at a temperature of −78° C. and stirred for 30 minutes. Then, 2-isopropoxy-4,4,5,5-tetramethyl-1,3,2-dioxaborolane (1.7 ml, 1.5 eq) was added dropwise thereto. After the dropwise addition was completed, the reaction mixture was stirred at room temperature for 3 hours. After the reaction was believed to be completed, the reaction product was separated and purified by column chromatography using a mixture of ethyl acetate (EA) and hexane (1:2) as a developing solvent to obtain about 0.4 g (yield: 20%) of Intermediate C(1).

### Synthesis of Intermediate C(2)

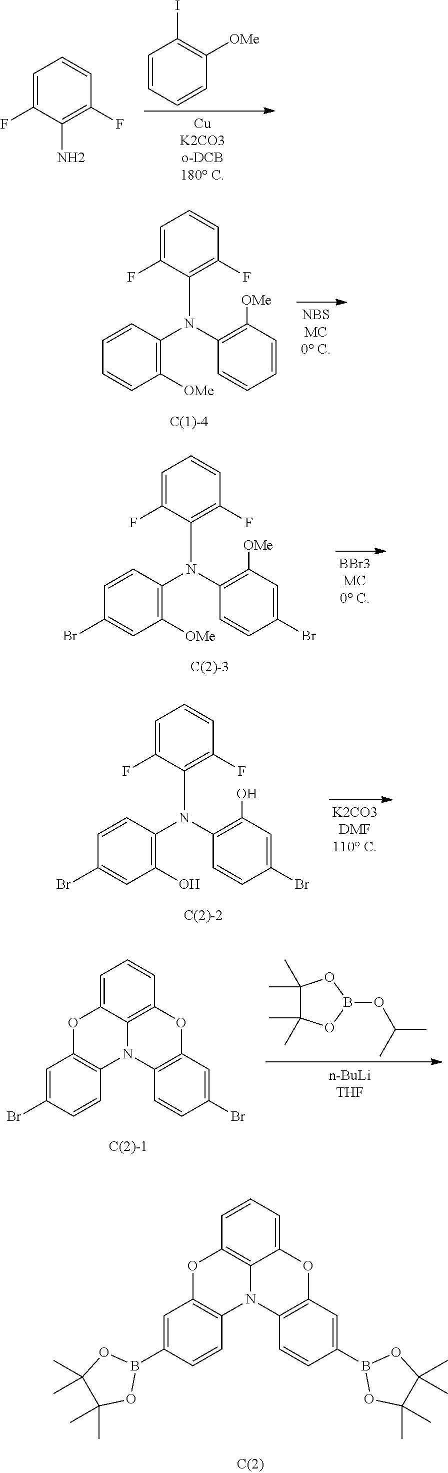

Intermediate C(2) was synthesized in substantially the same manner as in Intermediate C(1), except that NBS (2 eq) was used, instead of NBS (1 eq), in a process of synthesizing Intermediate C(2)-3.

### Synthesis Example 1: Synthesis of Compound 1

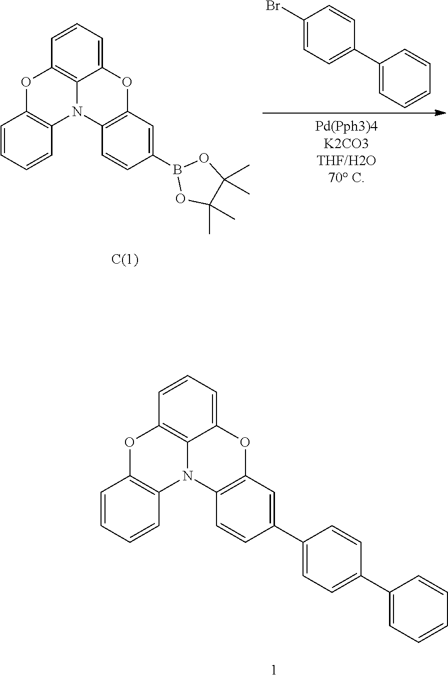 [SMILES: CC1(C)OB(c2ccc3c(c2)Oc2cccc4c2N3c2ccccc2O4)OC1(C)C ; Brc1ccc(-c2ccccc2)cc1 ; c1ccc(-c2ccc(-c3ccc4c(c3)Oc3cccc5c3N4c3ccccc3O5)cc2)cc1]

Intermediate C(1) (1.8 g, 1 eq), 4-bromo-1,1′-biphenyl (1.3 g, 1.2 eq), Pd(PPh₃)₄ (0.5 g, 0.05 eq), K₂CO₃ (2.4 g, 3 eq), and 40 ml/10 ml of THF/H20 were added to a one-neck round bottom flask and stirred at a temperature of 70° C. for 12 hours. After the reaction was believed to be completed, the obtained mixture was separated and purified by column chromatography using a mixture of MC and hexane (1:5) as a developing solvent to obtain about 1.1 g (yield: 58%; purity: 99%) of Compound 1. Then, sublimation purification was performed thereon to obtain about 1 g (purity: 99.99% or more) of Compound 1.

### Synthesis Example 2: Synthesis of Compound 4

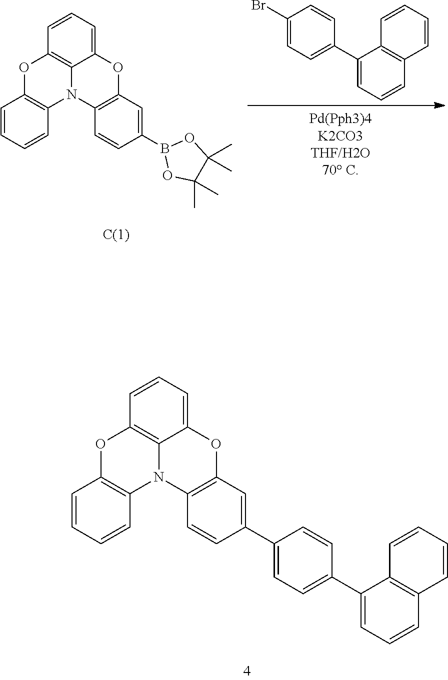 [SMILES: CC1(C)OB(c2ccc3c(c2)Oc2cccc4c2N3c2ccccc2O4)OC1(C)C ; Brc1ccc(-c2cccc3ccccc23)cc1 ; c1ccc2c(c1)Oc1cccc3c1N2c1ccc(-c2ccc(-c4cccc5ccccc45)cc2)cc1O3]

Intermediate C(1) (1.8 g, 1 eq), 1-(4-bromophenyl)naphthalene (1 g, 1.2 eq), Pd(PPh₃)₄ (0.5 g, 0.05 eq), K₂CO₃ (2.4 g, 3 eq), and 40 ml/10 ml of THF/H₂O were added to a one-neck round bottom flask and stirred at a temperature of 70° C. for 12 hours. After the reaction was believed to be completed, the obtained mixture was separated and purified by column chromatography using a mixture of MC and hexane (1:5) as a developing solvent to obtain about 1.5 g (yield: 65%; purity: 99.2%) of Compound 4. Then, sublimation purification was performed thereon to obtain about 1.2 g (purity: 99.99% or more) of Compound 4.

### Synthesis Example 3: Synthesis of Compound 6

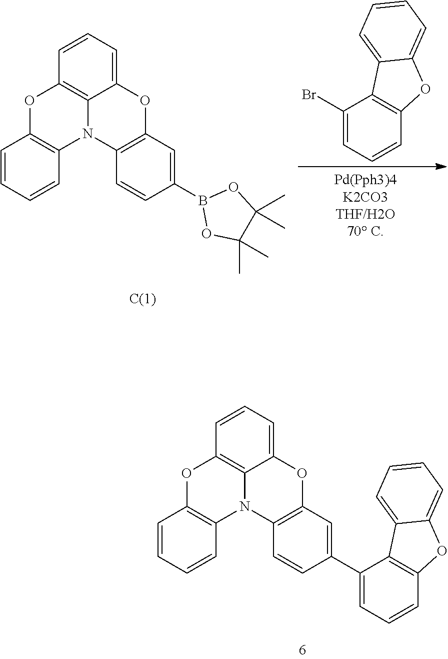 [SMILES: CC1(C)OB(c2ccc3c(c2)Oc2cccc4c2N3c2ccccc2O4)OC1(C)C ; Brc1cccc2oc3ccccc3c12 ; c1ccc2c(c1)Oc1cccc3c1N2c1ccc(-c2cccc4oc5ccccc5c24)cc1O3]

Intermediate C(1) (1.8 g, 1 eq), 1-bromodibenzo[b,d]furan (1.3 g, 1.2 eq), Pd(PPh₃)₄ (0.5 g, 0.08 eq), K₂CO₃ (3.3 g, 5 eq), and 40 ml/10 ml of THF/H₂O were added to a one-neck round bottom flask and stirred at a temperature of 70° C. for 12 hours. After the reaction was believed to be completed, the obtained mixture was separated and purified by column chromatography using a mixture of MC and hexane (1:5) as a developing solvent to obtain about 1.2 g (yield: 60%; purity: 99.5%) of Compound 6. Then, sublimation purification was performed thereon to obtain about 1 g (purity: 99.99% or more) of Compound 6.

### Synthesis Example 4: Synthesis of Compound 17

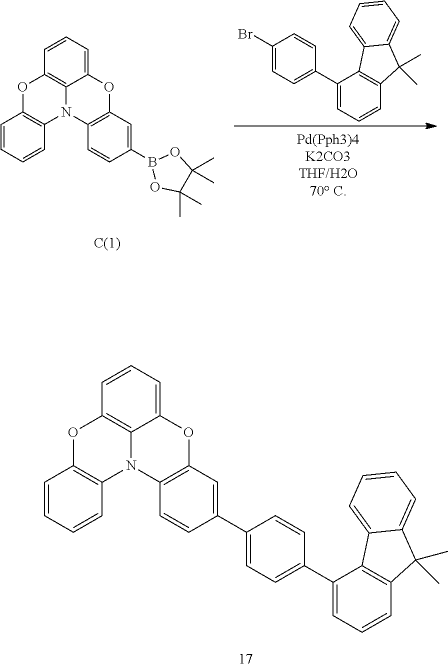 [SMILES: CC1(C)OB(c2ccc3c(c2)Oc2cccc4c2N3c2ccccc2O4)OC1(C)C ; CC1(C)c2ccccc2-c2c(-c3ccc(Br)cc3)cccc21 ; CC1(C)c2ccccc2-c2c(-c3ccc(-c4ccc5c(c4)Oc4cccc6c4N5c4ccccc4O6)cc3)cccc21]

Intermediate C(1) (1.8 g, 1 eq), 4-(4-bromophenyl)-9,9-dimethyl-9H-fluorene (1 g, 1.2 eq), Pd(PPh₃)₄ (0.5 g, 0.08 eq), K₂CO₃ (3.3 g, 5 eq), and 40 ml/10 ml of THF/H₂O were added to a one-neck round bottom flask and stirred at a temperature of 70° C. for 12 hours. After the reaction was believed to be completed, the obtained mixture was separated and purified by column chromatography using a mixture of MC and hexane (1:5) as a developing solvent to obtain about 1.2 g (yield: 63%; purity: 99.8%) of Compound 17. Then, sublimation purification was performed thereon to obtain about 1.1 g (purity: 99.99% or more) of Compound 17.

### Synthesis Example 5: Synthesis of Compound 31

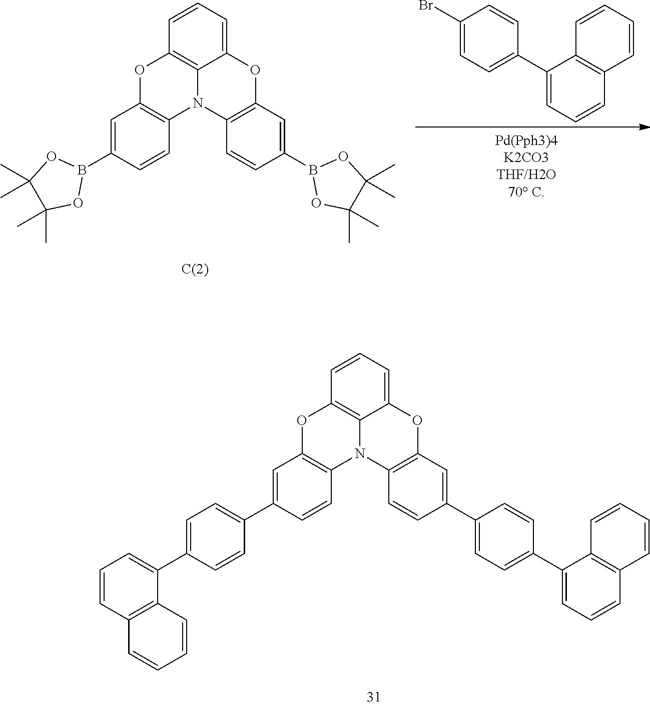

Intermediate C(2) (2 g, 1 eq), 1-(4-bromophenyl)naphthalene (2.7 g, 2.5 eq), Pd(PPh₃)₄ (0.22 g, 0.05 eq), K₂CO₃ (1.6 g, 3 eq), and 80 ml/20 ml of THF/H₂O were added to a one-neck round bottom flask and stirred at a temperature of 70° C. for 12 hours. After the reaction was believed to be completed, the obtained mixture was separated and purified by column chromatography using a mixture of MC and hexane (1:5) as a developing solvent to obtain about 2 g (yield: 78%; purity: 99.6%) of Compound 31. Then, sublimation purification was performed thereon to obtain about 1.7 g (purity: 99.99% or more) of Compound 31.

### Synthesis Example 6: Synthesis of Compound 37

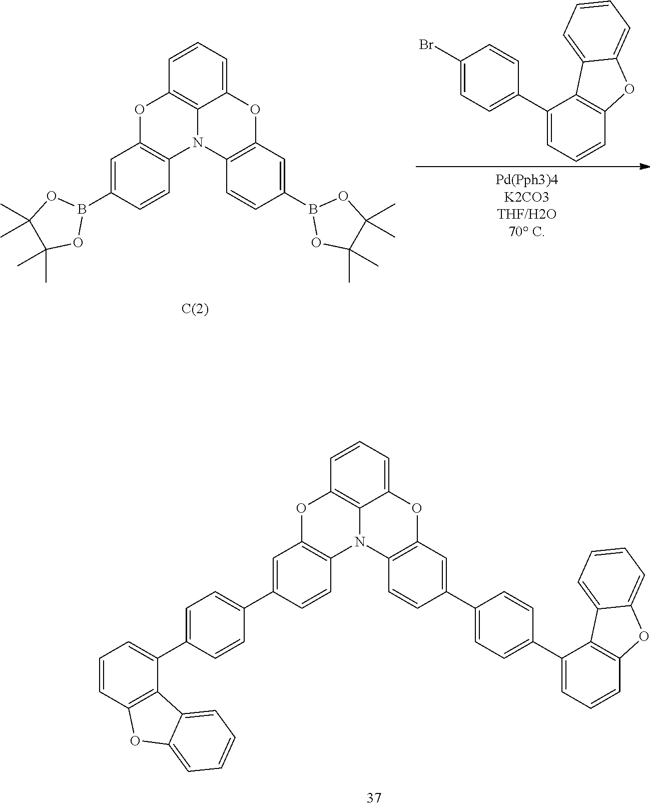

Intermediate C(2) (2 g, 1 eq), 1-(4-bromophenyl)dibenzo[b,d]furan (2.4 g, 2.5 eq), Pd(PPh₃)₄ (0.22 g, 0.05 eq), K₂CO₃ (1.6 g, 3 eq), and 80 ml/20 ml of THF/H₂O were added to a one-neck round bottom flask and stirred at a temperature of 70° C. for 12 hours. After the reaction was believed to be completed, the obtained mixture was separated and purified by column chromatography using a mixture of MC and hexane (1:5) as a developing solvent to obtain about 1.7 g (yield: 70%; purity: 99.8%) of Compound 37. Then, sublimation purification was performed thereon to obtain about 1.5 g (purity: 99.99% or more) of Compound 37.

### Synthesis Example 7: Synthesis of Compound 56

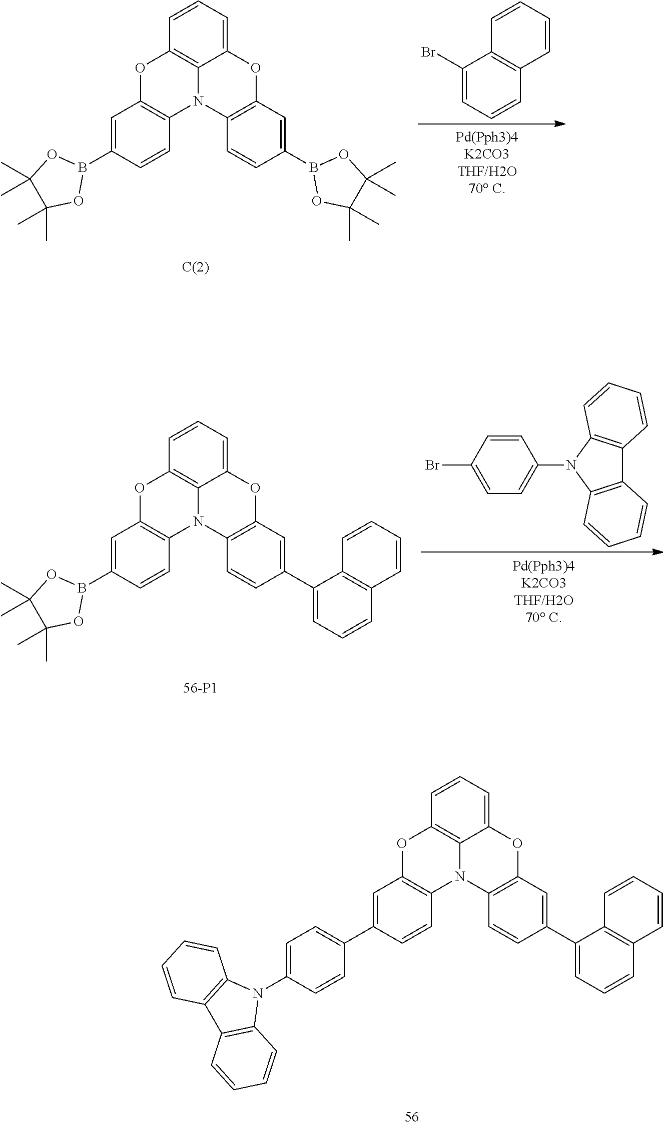

Intermediate C(2) (1.8 g, 1 eq), 1-bromonaphthalene (1.1 g, 1 eq), Pd(PPh₃)₄ (0.1 g, 0.03 eq), K₂CO₃ (1 g, 2 eq), and 40 ml/10 ml of THF/H₂O were added to a one-neck round bottom flask and stirred at a temperature of 70° C. for 7 hours. After the reaction was believed to be completed, the obtained mixture was separated and purified by column chromatography using a mixture of MC and hexane (1:5) as a developing solvent to obtain about 1.4 g (yield: 63%; purity: 99.3%) of Intermediate 56-P1.

Intermediate 56-P1 (1.4 g, 1 eq), 9-(4-bromophenyl)-9H-carbazole (1.1 g, 1 eq), Pd(PPh₃)₄ (0.2 g, 0.05 eq), K₂CO₃ (3 g, 5 eq), and 40 ml/10 ml of THF/H₂O were added to a one-neck round bottom flask and stirred at a temperature of 70° C. for 4 hours. After the reaction was believed to be completed, the obtained mixture was separated and purified by column chromatography using a mixture of MC and hexane (1:5) as a developing solvent to obtain about 1.4 g (yield: 60%; purity: 99.9%) of Compound 56. Then, sublimation purification was performed thereon to obtain about g (purity: 99.99% or more) of Compound 56.

¹H NMR and MS/FAB of Compounds synthesized according to Synthesis Examples 1 to 7 are shown in Table 1.

Synthesis methods of compounds other than Compounds shown in Table 1 may also be easily recognized by those of ordinary skill in the art upon referring to the synthesis mechanisms and source materials described herein above.

TABLE 1

| Com- | | MS/FAB | |
|---|---|---|---|
| pound | ¹H NMR (CDCl₃, 400 MHz) | found | calc. |
| 1 | δδ7.75 (dd, 2H), 7.49-7.25 (m, 10H), | 425.49 | 425.14 |
| | 7.14 (d, 1H), 7.01-6.96 (m, 4H), 6.68 | | |
| | (d, 2H) | | |
| 4 | δδ8.95(d, 1H), 8.50(d, 1H), 8.20(d, | 475.55 | 475.16 |
| | 1H), 8.09(d, 1H), 7.77 (dd, 1H), 7.52(dd, | | |
| | 1H), 7.39-7.25 (m, 8H), 7.14 (d, 1H), | | |
| | 7.01-6.96 (m, 4H), 6.68 (d, 2H) | | |
| 6 | δδ7.98 (d, 1H), 7.82(d, 1H), 7.69(d, | 439.47 | 439.12 |
| | 1H), 7.57-7.54 (dd, 2H), 7.39-7.27(m, | | |
| | 5H), 7.14 (d, 1H), 7.01-6.96 (m, 4H), | | |
| | 6.68 (d, 2H) | | |
| 17 | δδ7.90(d, 1H), 7.78(d, 1H), 7.65(d, | 541.65 | 541.20 |
| | 1H), 7.55(d, 1H), 7.47-7.25 (m, 10H), 7.14 | | |
| | (d, 1H), 7.01-6.96 (m, 4H), 6.68 (d, 2H) | | |
| 31 | δδ8.95(d, 2H), 8.50(d, 2H), 8.20(dd, | 677.80 | 677.24 |
| | 2H), 8.09(dd, 2H), 7.77 (dd, 2H), 7.52(dd, | | |
| | 2H), 7.39-7.25 (m, 16H), 6.79 (d, 1H), 6.68 | | |
| | (d, 2H) | | |
| 37 | δδ7.98 (dd, 2H), 7.82(dd, 2H), 7.69(dd, | 757.85 | 757.23 |
| | 2H), 7.57-7.54 (m, 4H), 7.39-7.27(m, | | |
| | 18H), 6.97 (d, 1H), 6.68 (dd, 2H) | | |
| 56 | δδ8.95(d, 1H), 8.55-8.50 (m, 2H), 8.20- | 640.74 | 640.22 |
| | 8.19 (dd, 2H), 8.09(d, 1H), 7.94-7.91 (m, | | |
| | 5H), 7.77 (m, 1H), 7.58-7.50 (m, 3H), 7.39- | | |
| | 7.16 (m, 10H), 6.79 (d, 1H), 6.68 (d, 2H) | | |

### Example 1

As an anode, a Corning 15 D/cm² (1,200 Å) ITO glass substrate was cut to a size of 50 mm×50 mm×0.7 mm, sonicated with isopropyl alcohol and pure water each for 5 minutes, and then cleaned by exposure to ultraviolet rays and ozone for 30 minutes. Then, the ITO glass substrate was provided to a vacuum deposition apparatus.

2-TNATA was vacuum-deposited on the ITO glass substrate to form a hole injection layer having a thickness of 600 Å, and Compound 1 was vacuum-deposited on the hole injection layer to form a hole transport layer having a thickness of 300 Å.

9,10-di(naphthalene-2-yl)anthracene (DNA) and 4,4′-bis[2-(4-(N,N′-diphenylamino)phenyl)vinyl]biphenyl (DPAVBi) were co-deposited on the hole transport layer at a weight ratio of 98:2 to form an emission layer having a thickness of 300 Å.

Alq₃ was deposited on the emission layer to form an electron transport layer having a thickness of 300 Å, an alkali metal halide LiF was deposited on the electron transport layer to form an electron injection layer having a thickness of 10 Å, and Al was vacuum-deposited on the electron injection layer to form a LiF/Al electrode having a thickness of 3,000 Å, thereby completing the manufacture of an organic light-emitting device.

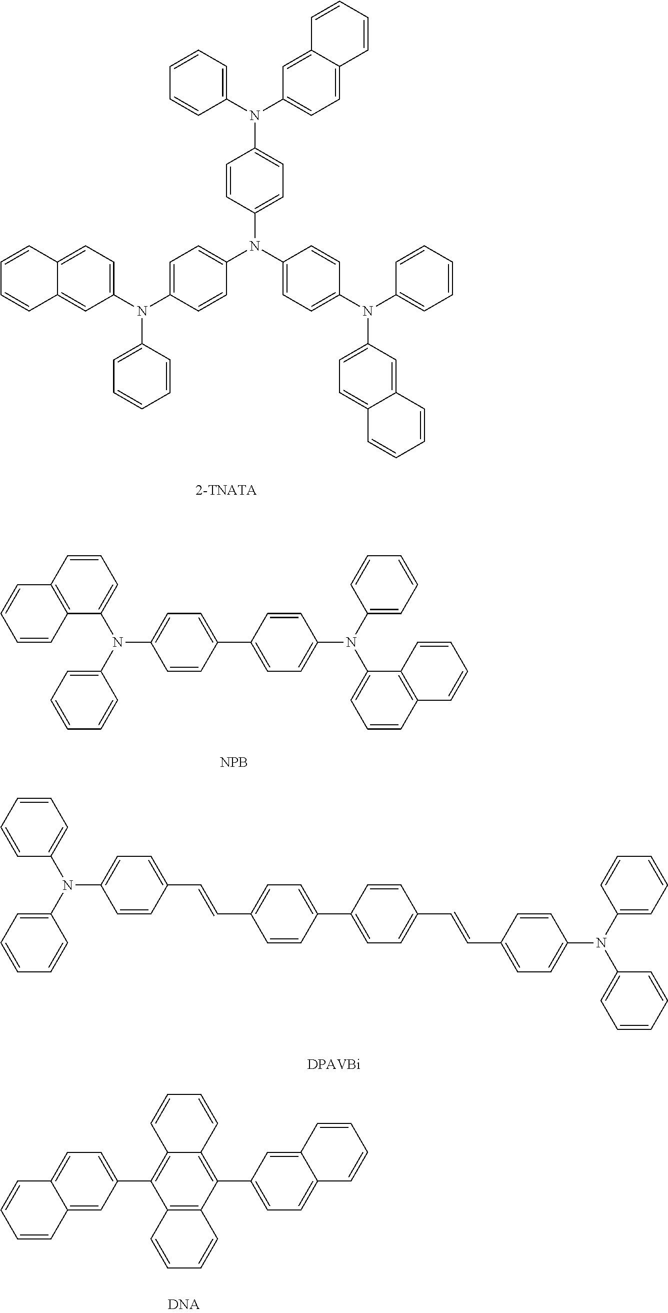

### Examples 2 to 7 and Comparative Examples 1 to 5

Organic light-emitting devices were manufactured in substantially the same manner as in Example 1, except that Compounds shown in Table 2 were each used instead of Compound 1 in forming an emission layer.

### Evaluation Example 1

The device performance (driving voltage, luminance, efficiency, and color coordinates) of the organic light-emitting devices manufactured according to Examples 1 to 7 and Comparative Examples 1 to 5 were measured at a current density of 50 mA/cm² and an amount of time (half lifespan) that lapsed when luminance was 50% of initial luminance (100%) was measured at a current density of 100 mA/cm². Results thereof are shown in Table 2.
Luminance: Power was supplied from a current-voltage meter (Keithley SMU 236), and luminance was measured by using a luminance meter PR650.
Efficiency: Power was supplied from a current-voltage meter (Keithley SMU 236), and efficiency was measured by using a luminance meter PR650.

TABLE 2

| | | | | | | | Half |
|---|---|---|---|---|---|---|---|
| | | | | | | | lifespan |
| | | | | | | | (hr |
| | Hole | Driving | Current | | | | @100 |
| | transporting | voltage | density | Luminance | Efficiency | Emission | mA/ |
| | material | (V) | (mA/cm²) | (cd/m²) | (cd/A) | color | cm²) |
| Comparative | NPB | 7.01 | 50 | 2645 | 5.29 | blue | 258 |
| Example 1 | | | | | | | |
| Comparative | Compound | 4.02 | 50 | 2210 | 4.71 | blue | 214 |
| Example 2 | A | | | | | | |
| Comparative | Compound | 4.22 | 50 | 3100 | 6.05 | blue | 280 |
| Example 3 | B | | | | | | |
| Comparative | Compound | 4.01 | 50 | 2307 | 4.44 | blue | 200 |
| Example 4 | C | | | | | | |
| Comparative | Compound | 6.20 | 50 | 3346 | 7.00 | blue | 299 |
| Example 5 | D | | | | | | |
| Example 1 | Compound | 4.32 | 50 | 3670 | 7.34 | blue | 362 |
| | 1 | | | | | | |
| Example 2 | Compound | 4.21 | 50 | 3715 | 7.43 | blue | 353 |
| | 4 | | | | | | |
| Example 3 | Compound | 4.22 | 50 | 3665 | 7.33 | blue | 372 |
| | 6 | | | | | | |
| Example 4 | Compound | 4.26 | 50 | 3730 | 7.46 | blue | 374 |
| | 17 | | | | | | |
| Example 5 | Compound | 4.26 | 50 | 3730 | 7.46 | blue | 374 |
| | 31 | | | | | | |
| Example 6 | Compound | 4.25 | 50 | 3630 | 7.26 | blue | 384 |
| | 37 | | | | | | |
| Example 7 | Compound | 4.41 | 50 | 3725 | 7.45 | blue | 343 |
| | 56 | | | | | | |

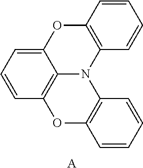

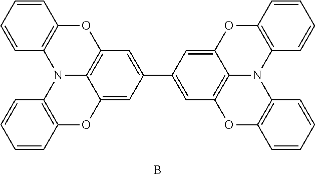

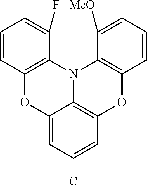

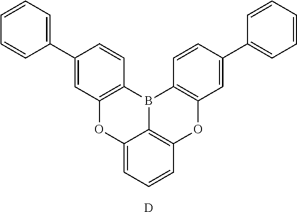

From Table 2, it can be seen that when the compound according to one or more embodiments is used as a hole transporting material, excellent characteristics such as an equal or lower driving voltage, high luminescence efficiency, high luminance, and a long lifespan are provided, as compared with a case in which Compounds of Comparative Examples 1 to 5 are used.

The heterocyclic compound may have excellent charge transporting capability, and the organic light-emitting device including the heterocyclic compound may have excellent characteristics such as a low driving voltage, high efficiency, high luminance, and a long lifespan.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the present disclosure as defined by the following claims, and equivalents thereof.

What is claimed is:

1. An organic light-emitting device comprising:
a first electrode;
a second electrode facing the first electrode; and
an organic layer between the first electrode and the second electrode and comprising an emission layer,
wherein the organic layer comprises at least one of a heterocyclic compound represented by Formula 1: 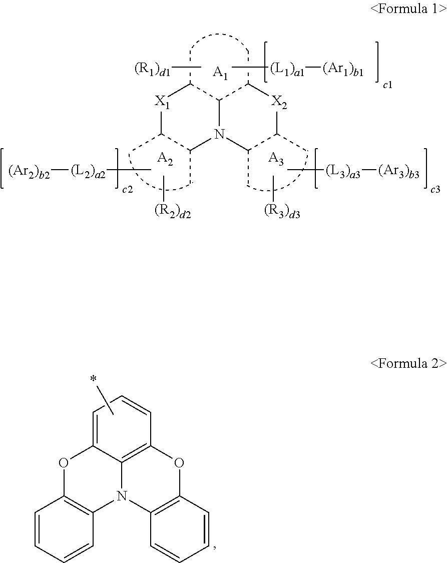
wherein, in Formula 1,
ring A₁ to ring A₃ are each independently a C₅-C₆₀ carbocyclic group or a C₁-C₆₀ heterocyclic group,
X₁ and X₂ are each independently O or S,
L₁ to L₃ are each independently a substituted or unsubstituted C₅-C₆₀ carbocyclic group or a substituted or unsubstituted C₁-C₆₀ heterocyclic group,
a1 to a3 are each independently an integer from 0 to 3,
Ar₁ to Ar₃ are each independently a substituted or unsubstituted C₅-C₆₀ carbocyclic group or a substituted or unsubstituted C₁-C₆₀ heterocyclic group, wherein each of Ar₁ to Ar₃ is not a group represented by Formula 2,
b1 to b3 are each independently an integer from 1 to 3,
c1 to c3 are each independently an integer from 0 to 4,
c1+c2+c3≥1,
R₁ to R₃ are each independently selected from deuterium, —F, —Cl, —Br, —I, a hydroxyl group, a cyano group, a nitro group, an amino group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁ heterocycloalkenyl group, a substituted or unsubstituted C₆-C₆₀ aryl group, a substituted or unsubstituted C₃-C₁₀ aryloxy group, a substituted or unsubstituted C₆-C₆₀ arylthio group, a substituted or unsubstituted C₂-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₁)(Q₂), —B(Q₁)(Q₂), —C(═O)(Q₁), —S(═O)₂(Q₁), and —P(═O)(Q₁)(Q₂),
d1 to d3 are each independently selected from 0 to 4,
at least one substituent of the substituted C₅-C₆₀ carbocyclic group, the substituted C₁-C₆₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₂-C₆₀ heteroaryl group, the substituted monovalent non-aromatic condensed polycyclic group, and the substituted monovalent non-aromatic condensed heteropolycyclic group is selected from:
deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one selected from deuterium, —Cl, —Br, —I, a hydroxyl group, a cyano group, a nitro group, an amidino group, a hydrazino group, a hydrazono group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), and —P(═O)(Q₁₁)(Q₁₂);
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, a biphenyl group, and a terphenyl group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, a biphenyl group, and a terphenyl group, each substituted with at least one selected from deuterium, —C, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, a biphenyl group, a terphenyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), and —P(═O)(Q₂₁)(Q₂₂); and
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂),
Q₁ to Q₃, Q₁₁ to Q₁₃, Q₂₁ to Q₂₃, and Q₃₁ to Q₃₃ are each independently selected from hydrogen, deuterium, —C, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with a C₁-C₆₀ alkyl group, a C₁-C₆₀ heteroaryl group, a monovalent non-aromatic condensed polycyclic group, a monovalent non-aromatic condensed heteropolycyclic group, a biphenyl group, and a terphenyl group,
* indicates a binding site to a neighboring atom.

2. The organic light-emitting device of claim 1, wherein,
the first electrode is an anode,
the second electrode is a cathode,
the organic layer further comprises a hole transport region between the first electrode and the emission layer and an electron transport region between the emission layer and the second electrode,
the hole transport region comprises a hole injection layer, a hole transport layer, an emission auxiliary layer, an electron blocking layer, or any combination thereof, and
the electron transport region comprises a hole blocking layer, an electron transport layer, an electron injection layer, or any combination thereof.

3. The organic light-emitting device of claim 2, wherein,
the hole transport region comprises the at least one of the heterocyclic compound.

4. The organic light-emitting device of claim 2, wherein,
the hole transport region comprises a hole transport layer, and
the hole transport layer comprises the at least one of the heterocyclic compound.

5. The organic light-emitting device of claim 2, wherein,
the hole transport region comprises a p-dopant, and
the p-dopant has a lowest unoccupied molecular orbital (LUMO) energy level of about −3.5 eV or less.

6. The organic light-emitting device of claim 5, wherein,
the p-dopant comprises at least one selected from a quinone derivative, a metal oxide, and a cyano group-containing compound.

7. The organic light-emitting device of claim 2, wherein,
the electron transport region comprises an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or any combination thereof.

8. A heterocyclic compound represented by Formula 1: 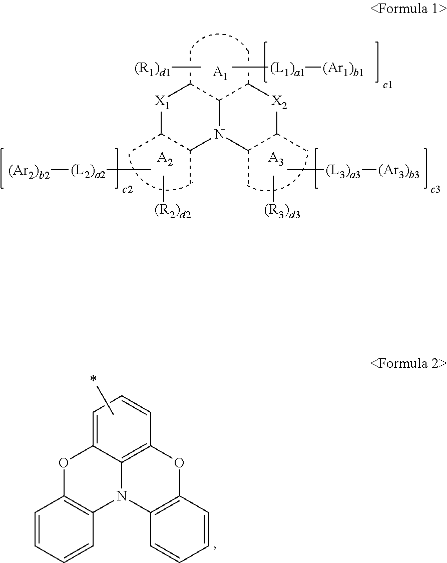
wherein, in Formula 1,
ring A₁ to ring A₃ are each independently a C₅-C₆₀ carbocyclic group or a C₁-C₆₀ heterocyclic group,
X₁ and X₂ are each independently 0 or S,
L₁ to L₃ are each independently a substituted or unsubstituted C₅-C₆₀ carbocyclic group or a substituted or unsubstituted C₁-C₆₀ heterocyclic group,
a1 to a3 are each independently an integer from 0 to 3,
Ar₁ to Ar₃ are each independently a substituted or unsubstituted C₅-C₆₀ carbocyclic group or a substituted or unsubstituted C₁-C₆₀ heterocyclic group, wherein each of Ar₁ to Ar₃ is not a group represented by Formula 2,
b1 to b3 are each independently an integer from 1 to 3,
c1 to c3 are each independently an integer from 0 to 4,
c1+c2+c3≥1,
R₁ to R₃ are each independently selected from deuterium, —F, —Cl, —Br, —I, a hydroxyl group, a cyano group, a nitro group, an amino group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₂-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₁)(Q₂), —B(Q₁)(Q₂), —C(═O)(Q₁), —S(═O)₂(Q₁), and —P(═O)(Q₁)(Q₂),
d1 to d3 are each independently selected from 0 to 4,
at least one substituent of the substituted C₅-C₆₀ carbocyclic group, the substituted C₁-C₆₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₂-C₆₀ heteroaryl group, the substituted monovalent non-aromatic condensed polycyclic group, and the substituted monovalent non-aromatic condensed heteropolycyclic group is selected from:
deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one selected from deuterium, —Cl, —Br, —I, a hydroxyl group, a cyano group, a nitro group, an amidino group, a hydrazino group, a hydrazono group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), and —P(═O)(Q₁₁)(Q₁₂);
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, a biphenyl group, and a terphenyl group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, a biphenyl group, and a terphenyl group, each substituted with at least one selected from deuterium, —C, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, a biphenyl group, a terphenyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), and —P(═O)(Q₂₁)(Q₂₂); and
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂),
Q₁ to Q₃, Q₁₁ to Q₁₃, Q₂₁ to Q₂₃, and Q₃₁ to Q₃₃ are each independently selected from hydrogen, deuterium, —C, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with a C₁-C₆₀ alkyl group, a C₁-C₆₀ heteroaryl group, a monovalent non-aromatic condensed polycyclic group, a monovalent non-aromatic condensed heteropolycyclic group, a biphenyl group, and a terphenyl group, and
* indicates a binding site to a neighboring atom.

9. The heterocyclic compound of claim 8, wherein,
ring A₁ to ring A₃ are each independently selected from a benzene group, a pentalene group, an indene group, a naphthalene group, an anthracene group, a fluorene group, a phenanthrene group, a triphenylene group, a pyrene group, a chrysene group, a naphthacene group, a pyrrole group, an imidazole group, a pyrazole group, a pyridine group, a pyrimidine group, a pyrazine group, a pyridazine group, an indole group, an isoindole group, an indazole group, a quinoline group, an isoquinoline group, a benzoquinoline group, a naphthyridine group, a quinoxaline group, a quinazoline group, a cinnoline group, a phenanthridine group, an acridine group, a phenanthroline group, a phenazine group, a benzimidazole group, a furan group, a benzofuran group, a thiophene group, a benzothiophene group, a thiazole group, an isothiazole group, a benzothiazole group, an oxazole group, an isoxazole group, a benzoxazole group, a triazole group, an oxadiazole group, a triazine group, a carbazole group, a dibenzofuran group, a dibenzothiophene group, a benzocarbazole group, and a dibenzocarbazole group.

10. The heterocyclic compound of claim 8, wherein,
ring A₁ to ring A₃ are identical to each other.

11. The heterocyclic compound of claim 8, wherein,
each of X₁ and X₂ is O.

12. The heterocyclic compound of claim 8, wherein,
L₁ to L₃ are each independently selected from:
a benzene group, a pentalene group, an indene group, a naphthalene group, an azulene group, a heptalene group, an indacene group, an acenaphthalene group, a fluorene group, a spiro-bifluorene group, a benzofluorene group, a dibenzofluorene group, a phenalene group, a phenanthrene group, an anthracene group, a fluoranthene group, a pyrene group, a chrysene group, a naphthacene group, a picene group, a perylene group, a pyrrole group, a thiophene group, a furan group, a silole group, an imidazole group, a pyrazole group, a thiazole group, an isothiazole group, an oxazole group, an isoxazole group, a pyridine group, a pyrazine group, a pyrimidine group, a pyridazine group, a triazine group, a benzofuran group, a benzothiophene group, a dibenzofuran group, a dibenzothiophene group, a carbazole group, a benzosilole group, a dibenzosilole group, a quinoline group, an isoquinoline group, a benzimidazole group, an imidazopyridine group, and an imidazopyrimidine group; and
a benzene group, a pentalene group, an indene group, a naphthalene group, an azulene group, a heptalene group, an indacene group, an acenaphthalene group, a fluorene group, a spiro-bifluorene group, a benzofluorene group, a dibenzofluorene group, a phenalene group, a phenanthrene group, an anthracene group, a fluoranthene group, a pyrene group, a chrysene group, a naphthacene group, a picene group, a perylene group, a pyrrole group, a thiophene group, a furan group, a silole group, an imidazole group, a pyrazole group, a thiazole group, an isothiazole group, an oxazole group, an isoxazole group, a pyridine group, a pyrazine group, a pyrimidine group, a pyridazine group, a triazine group, a benzofuran group, a benzothiophene group, a dibenzofuran group, a dibenzothiophene group, a carbazole group, a benzosilole group, a dibenzosilole group, a quinoline group, an isoquinoline group, a benzimidazole group, an imidazopyridine group, and an imidazopyrimidine group, each substituted with at least one selected from deuterium, —F, —Cl, —Br, —I, a hydroxyl group, a cyano group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a pyrenyl group, a chrysenyl group, a naphthacenyl group, a picenyl group, a perylenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, a benzofuranyl group, a benzothiophenyl group, a dibenzofuranyl group, a dibenzothiophenyl group, a carbazolyl group, a benzosilolyl group, a dibenzosilolyl group, a quinolinyl group, an isoquinolinyl group, a benzimidazolyl group, an imidazopyridinyl group, an imidazopyrimidinyl group, —Si(Q₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂), and
Q₃₁ to Q₃₃ are each independently selected from a C₁-C₂₀ alkyl group, a C₁-C₂₀ alkoxy group, a phenyl group, a biphenyl group, a terphenyl group, a naphthyl group, and a pyridinyl group.

13. The heterocyclic compound of claim 8, wherein,
L₁ to L₃ are each independently a group represented by one of Formulae 3-1 to 3-39: 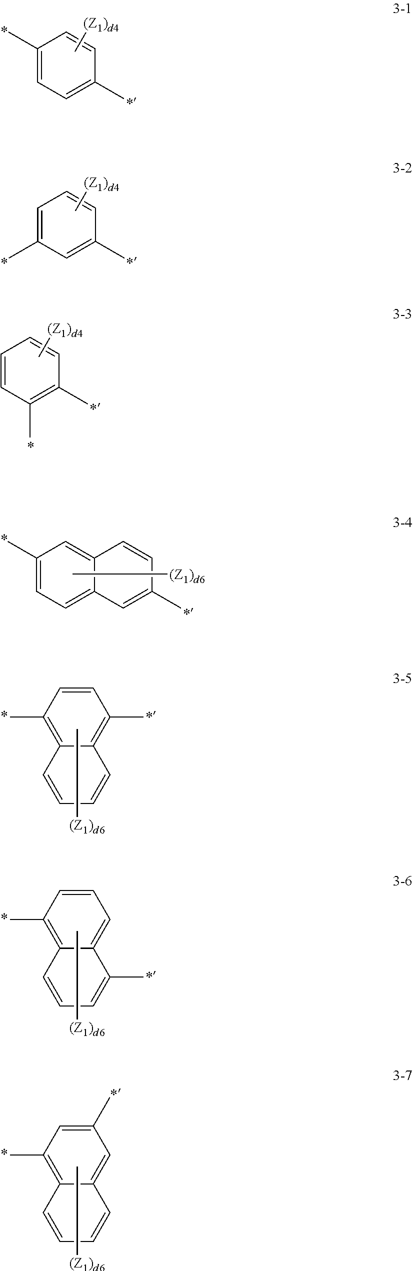 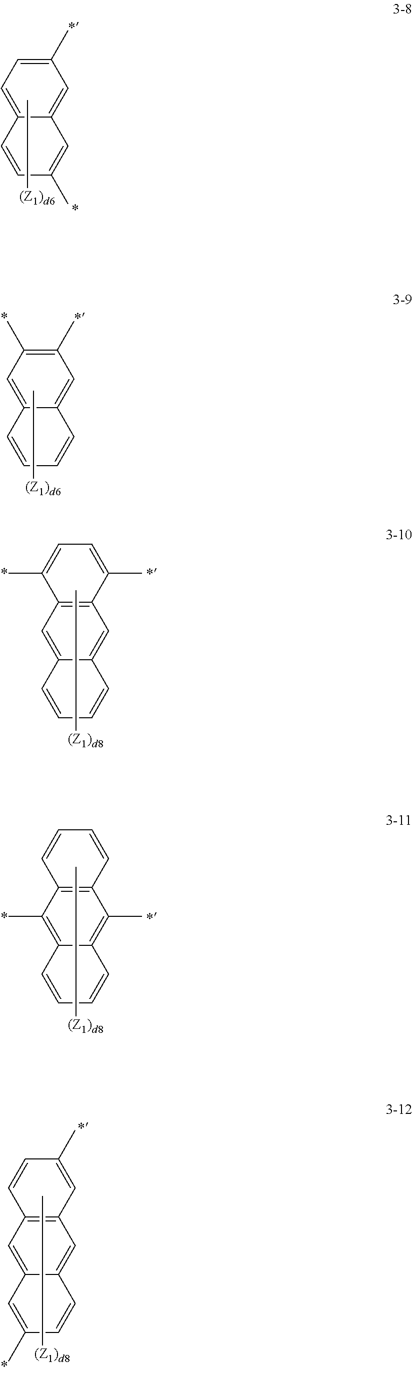 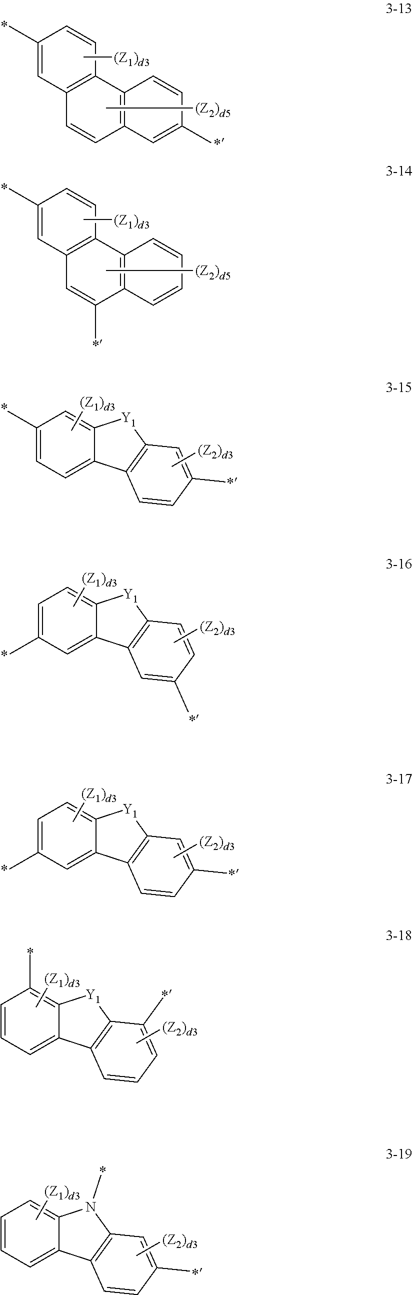 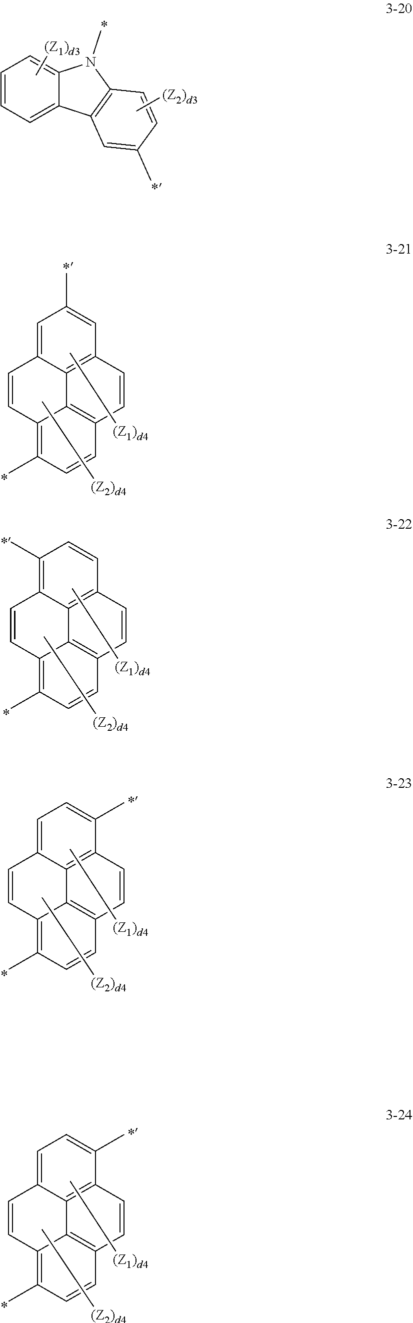 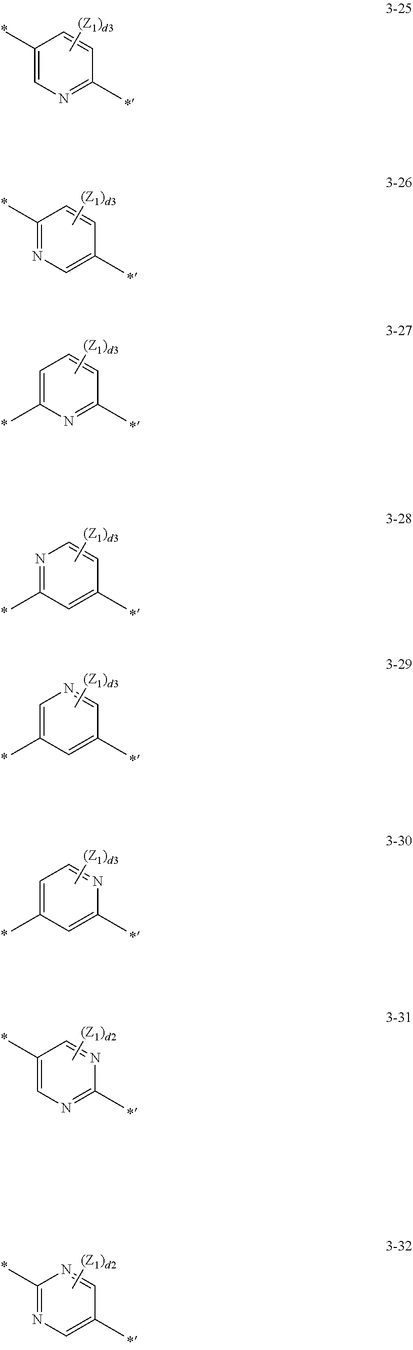 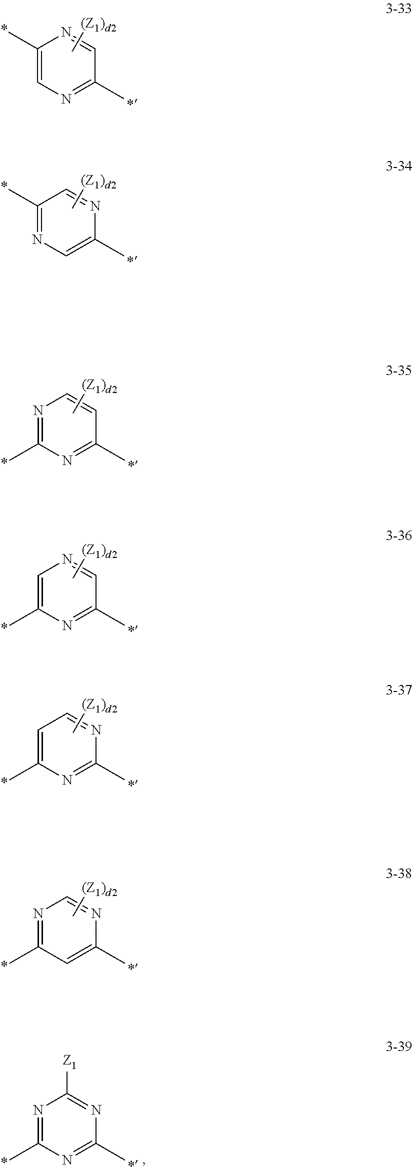
wherein, in Formulae 3-1 to 3-39,
Y₁ is O, S, C(Z₃)(Z₄), N(Z₃), or Si(Z₃)(Z₄),
Z₁ to Z₄ are each independently selected from hydrogen, deuterium, —F, —Cl, —Br, —I, a hydroxyl group, a cyano group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a naphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a pyrenyl group, a chrysenyl group, a naphthacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, a benzofuranyl group, a benzothiophenyl group, a dibenzofuranyl group, a dibenzothiophenyl group, a carbazolyl group, a benzosilolyl group, a dibenzosilolyl group, a quinolinyl group, an isoquinolinyl group, a benzimidazolyl group, an imidazopyridinyl group, an imidazopyrimidinyl group, and —Si(Q₃₁)(Q₃₂)(Q₃₃),
Q₃₁ to Q₃₃ are each independently selected from a C₁-C₂₀ alkyl group, a C₁-C₂₀ alkoxy group, a phenyl group, a biphenyl group, a terphenyl group, a naphthyl group, and a pyridinyl group,
Z₃ and Z₄ are optionally linked to each other to form a substituted or unsubstituted C₅-C₁₀ carbocyclic group or a substituted or unsubstituted C₁-C₁₀ heterocyclic group,
d2 is an integer from 0 to 2,
d3 is an integer from 0 to 3,
d4 is an integer from 0 to 4,
d5 is an integer from 0 to 5,
d6 is an integer from 0 to 6,
d8 is an integer from 0 to 8, and
* and *′ each indicate a binding site to a neighboring atom.

14. The heterocyclic compound of claim 8, wherein,
Ar₁ to Ar₃ are each independently selected from:
a benzene group, a pentalene group, an indene group, a naphthalene group, an azulene group, a heptalene group, an indacene group, an acenaphthalene group, a fluorene group, a spiro-bifluorene group, a benzofluorene group, a dibenzofluorene group, a phenalene group, a phenanthrene group, an anthracene group, a fluoranthene group, a pyrene group, a chrysene group, a naphthacene group, a picene group, a perylene group, a pyrrole group, a thiophene group, a furan group, a silole group, an imidazole group, a pyrazole group, a thiazole group, an isothiazole group, an oxazole group, an isoxazole group, a pyridine group, a pyrazine group, a pyrimidine group, a pyridazine group, a triazine group, a benzofuran group, a benzothiophene group, a dibenzofuran group, a dibenzothiophene group, a carbazole group, a benzosilole group, a dibenzosilole group, a quinoline group, an isoquinoline group, a benzimidazole group, an imidazopyridine group, and an imidazopyrimidine group; and
a benzene group, a pentalene group, an indene group, a naphthalene group, an azulene group, a heptalene group, an indacene group, an acenaphthalene group, a fluorene group, a spiro-bifluorene group, a benzofluorene group, a dibenzofluorene group, a phenalene group, a phenanthrene group, an anthracene group, a fluoranthene group, a pyrene group, a chrysene group, a naphthacene group, a picene group, a perylene group, a pyrrole group, a thiophene group, a furan group, a silole group, an imidazole group, a pyrazole group, a thiazole group, an isothiazole group, an oxazole group, an isoxazole group, a pyridine group, a pyrazine group, a pyrimidine group, a pyridazine group, a triazine group, a benzofuran group, a benzothiophene group, a dibenzofuran group, a dibenzothiophene group, a carbazole group, a benzosilole group, a dibenzosilole group, a quinoline group, an isoquinoline group, a benzimidazole group, an imidazopyridine group, and an imidazopyrimidine group, each substituted with at least one selected from deuterium, —F, —Cl, —Br, —I, a hydroxyl group, a cyano group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a pyrenyl group, a chrysenyl group, a naphthacenyl group, a picenyl group, a perylenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyrdinyl group, a pyrazinyl group, a pyrimidinyl group, a pyridazinyl group, a triazinyl group, a benzofuranyl group, a benzothiophenyl group, a dibenzofuranyl group, a dibenzothiophenyl group, a carbazolyl group, a benzosilolyl group, a dibenzosilolyl group, a quinolinyl group, an isoquinolinyl group, a benzimidazolyl group, an imidazopyridinyl group, an imidazopyrimidinyl group, —Si(Q₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂), and
Q₃₁ to Q₃₃ are each independently selected from a C₁-C₂₀ alkyl group, a C₁-C₂₀ alkoxy group, a phenyl group, a biphenyl group, a terphenyl group, a naphthyl group, and a pyrdinyl group.

15. The heterocyclic compound of claim 8, wherein,
Ar₁ to Ar₃ are each independently a group represented by one of Formulae 4-1 to 4-24: 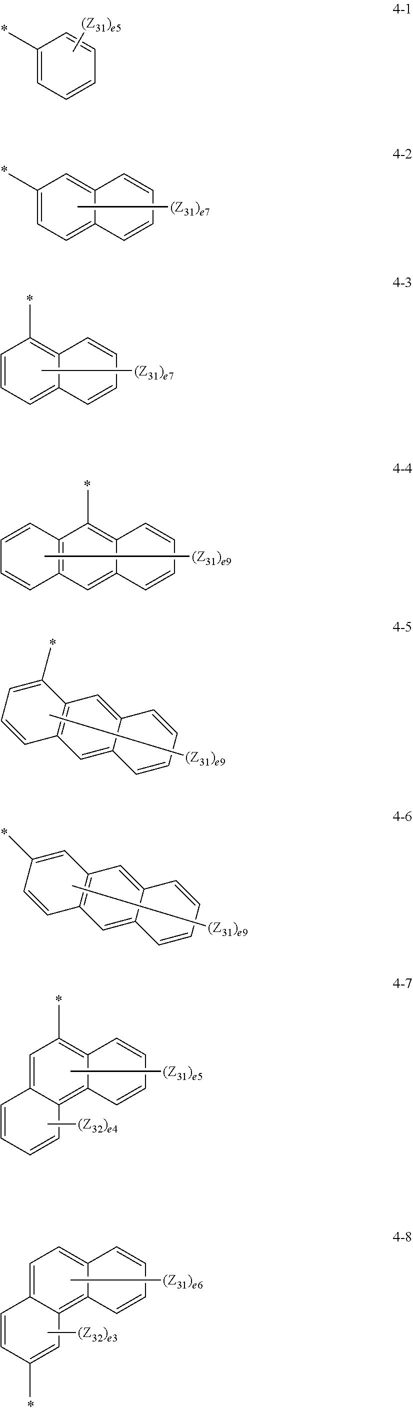 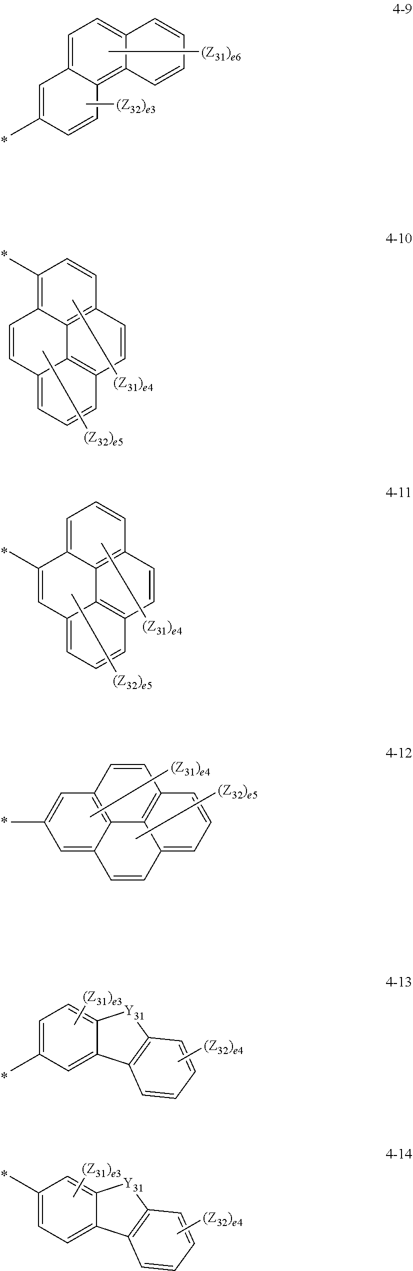 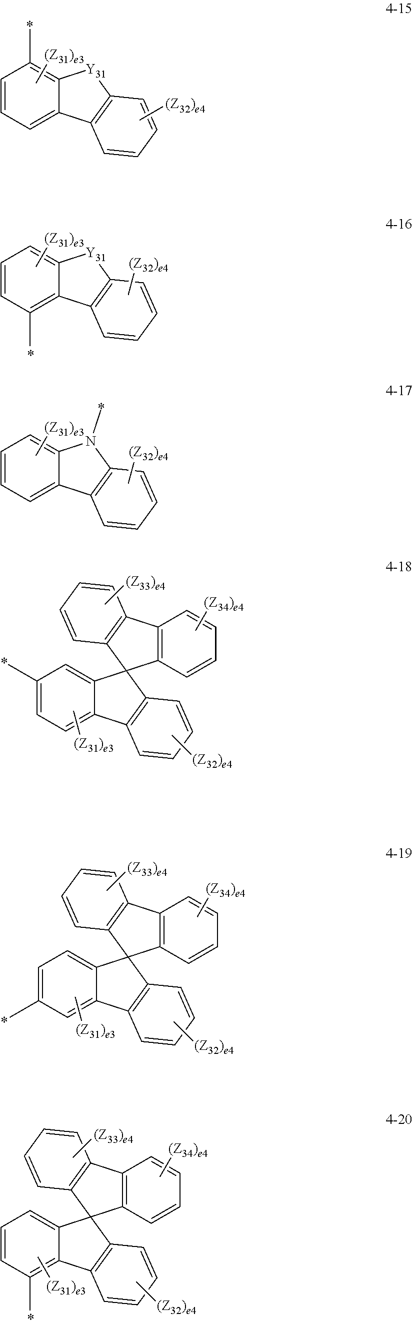 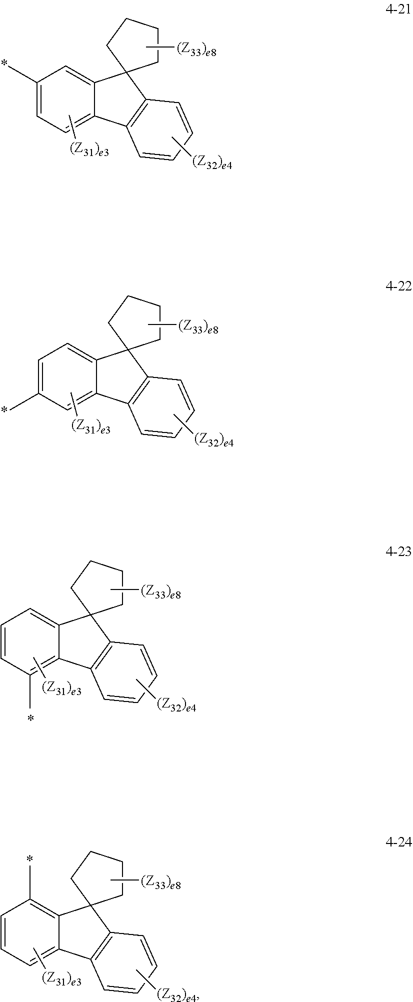
wherein, in Formulae 4-1 to 4-24,
Y₃₁ is O, S, C(Z₃₅)(Z₃₆), N(Z₃₅), or Si(Z₃₅)(Z₃₆),
Z₃₁ to Z₃₆ are each independently selected from hydrogen, deuterium, —F, —Cl, —Br, —I, a hydroxyl group, a cyano group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a naphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a pyrenyl group, a chrysenyl group, a naphthacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, a benzofuranyl group, a benzothiophenyl group, a dibenzofuranyl group, a dibenzothiophenyl group, a carbazolyl group, a benzosilolyl group, a dibenzosilolyl group, a quinolinyl group, an isoquinolinyl group, a benzimidazolyl group, an imidazopyridinyl group, an imidazopyrimidinyl group, and —Si(Q₃₁)(Q₃₂)(Q₃₃),
Q₃₁ to Q₃₃ are each independently selected from a C₁-C₂₀ alkyl group, a C₁-C₂₀ alkoxy group, a phenyl group, a biphenyl group, a terphenyl group, a naphthyl group, and a pyridinyl group,
e3 is an integer from 0 to 3,
e4 is an integer from 0 to 4,
e5 is an integer from 0 to 5,
e6 is an integer from 0 to 6,
e7 is an integer from 0 to 7,
e8 is an integer from 0 to 8,
e9 is an integer from 0 to 9, and
* indicates a binding site to a neighboring atom.

16. The heterocyclic compound of claim 8, wherein,
Ar₁ to Ar₃ are each independently a group represented by one of Formulae 5-1 to 5-41: 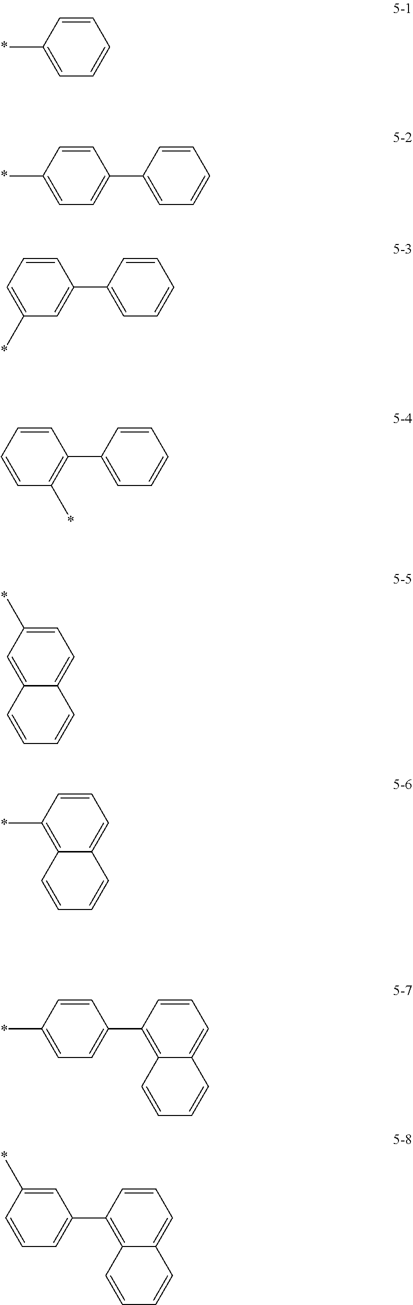 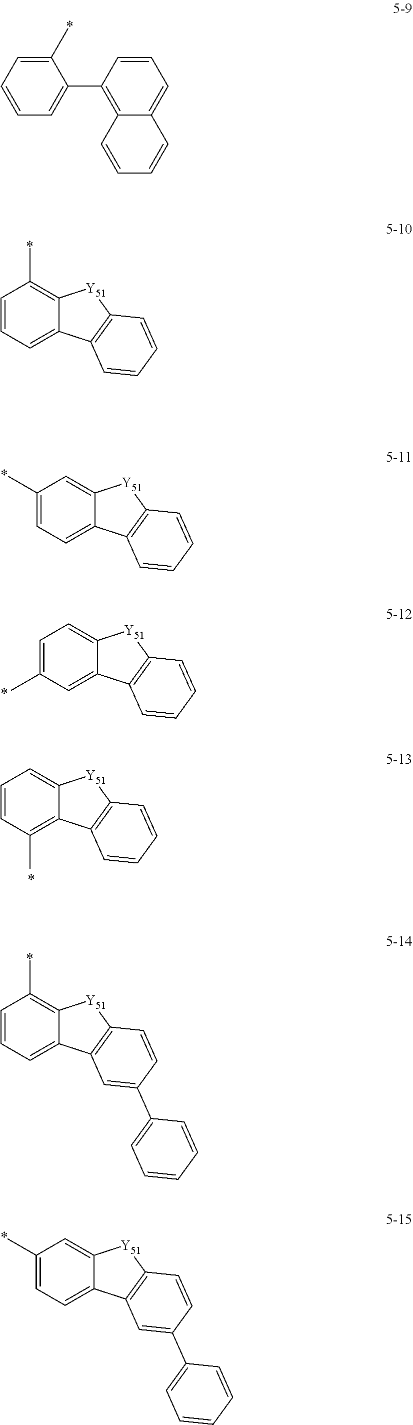 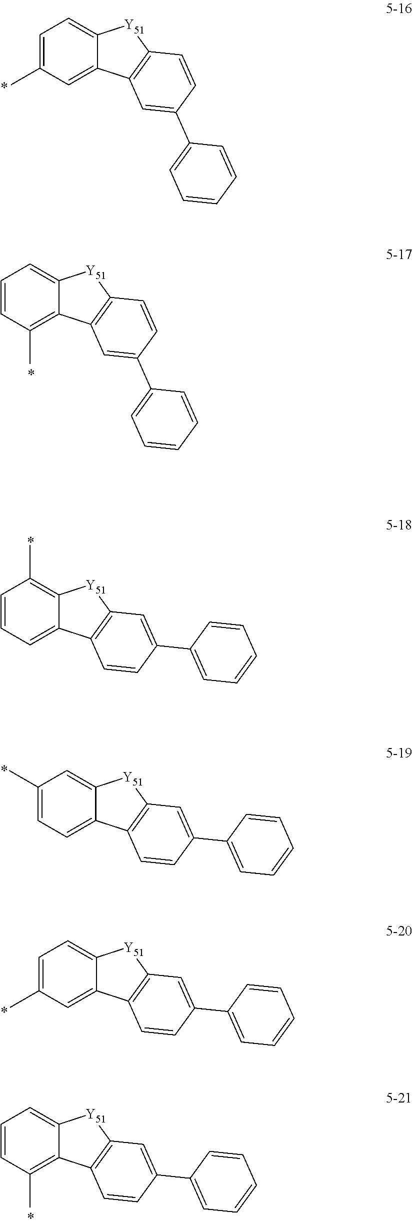 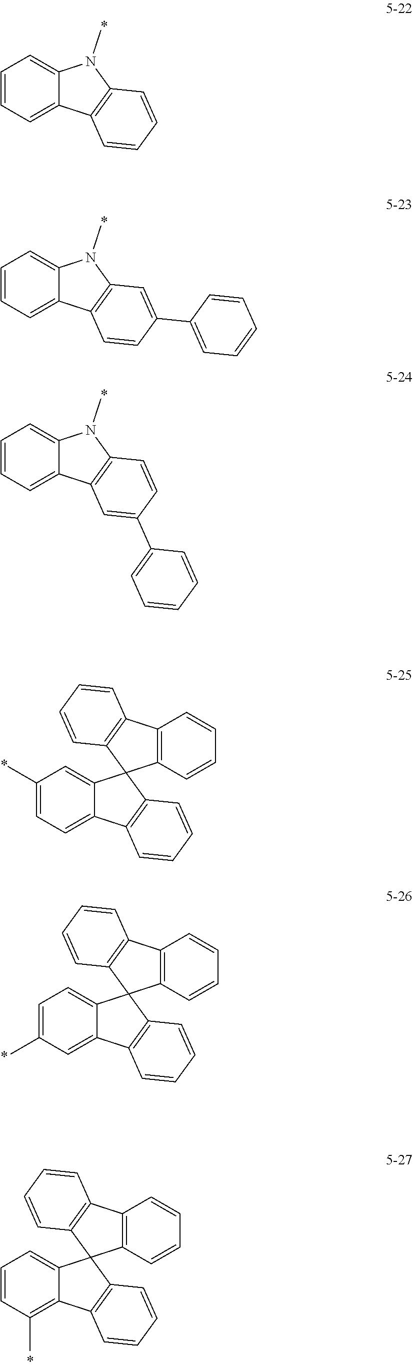 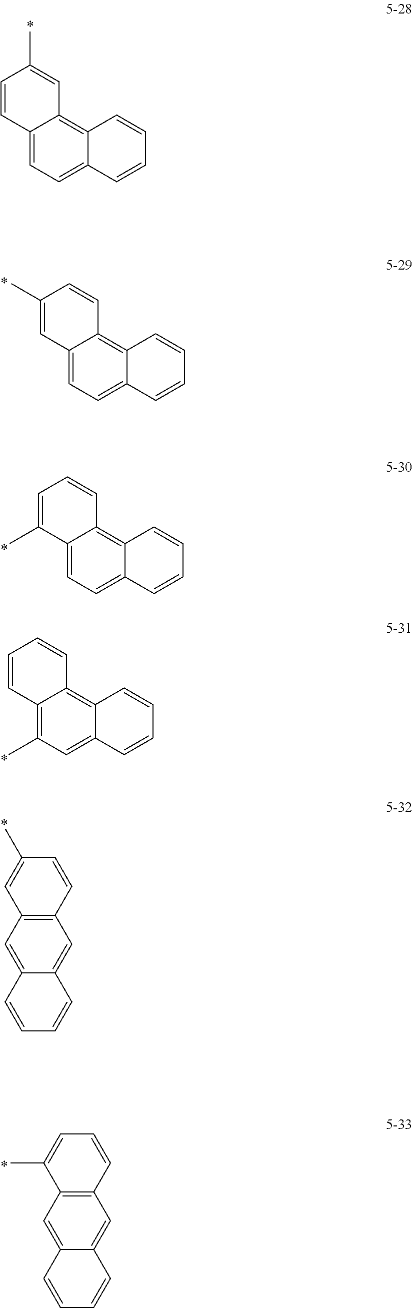 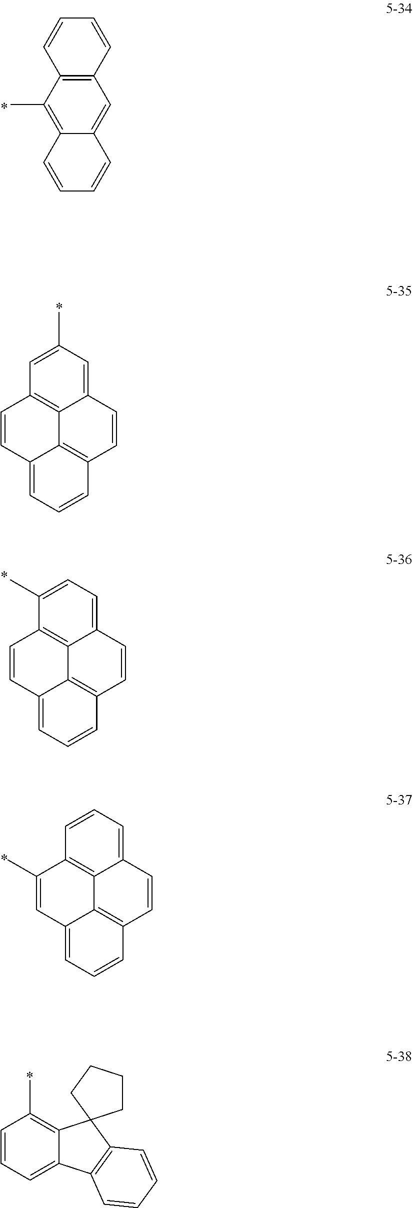 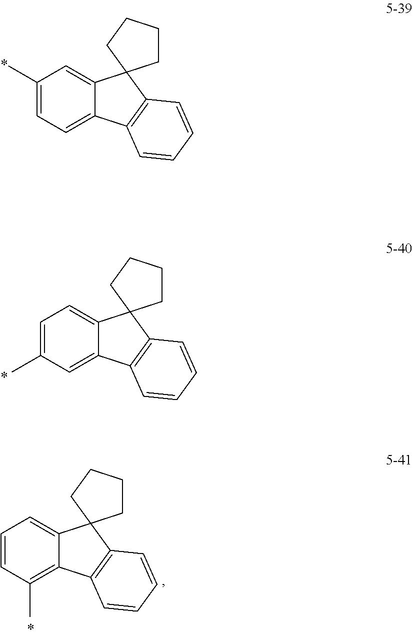
wherein, in Formulae 5-10 to 5-41,
Y₅₁ is O, S, C(Z₅₁)(Z₅₂), N(Z₅₁) or Si(Z₅₁)(Z₅₂),
Z₅₁ to Z₅₂ are each independently selected from hydrogen, deuterium, —F, —Cl, —Br, —I, a hydroxyl group, a cyano group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a naphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a pyrenyl group, a chrysenyl group, a naphthacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, a benzofuranyl group, a benzothiophenyl group, a dibenzofuranyl group, a dibenzothiophenyl group, a carbazolyl group, a benzosilolyl group, a dibenzosilolyl group, a quinolinyl group, an isoquinolinyl group, a benzimidazolyl group, an imidazopyridinyl group, an imidazopyrimidinyl group, and —Si(Q₃₁)(Q₃₂)(Q₃₃),
Q₃₁ to Q₃₃ are each independently selected from a C₁-C₂₀ alkyl group, a C₁-C₂₀ alkoxy group, a phenyl group, a biphenyl group, a terphenyl group, a naphthyl group, and a pyridinyl group, and
* indicates a binding site to a neighboring atom.

17. The heterocyclic compound of claim 8, wherein,
R₁ to R₃ are each independently selected from:
deuterium, a methyl group, an ethyl group, an n-propyl group, an isopropyl group, an n-butyl group, an isobutyl group, a sec-butyl group, a tert-butyl group, an n-pentyl group, a tert-pentyl group, a neo-pentyl group, an isopentyl group, a sec-pentyl group, 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a secoctyl group, a tert-octyl group, an n-nonyl group, an isononyl group, a sec-nonyl group, a tert-nonyl group, an n-decyl group, an isodecyl group, a sec-decyl group, and a tert-decyl group; and
a methyl group, an ethyl group, an n-propyl group, an isopropyl group, an n-butyl group, an isobutyl group, a sec-butyl group, a tert-butyl group, an n-pentyl group, a tert-pentyl group, a neo-pentyl group, an isopentyl group, a sec-pentyl group, 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group, each substituted with at least one selected from deuterium, —F, —Cl, —Br, and —I.

18. The heterocyclic compound of claim 8, wherein,
d1 to d3 are each 0.

19. The heterocyclic compound of claim 8, wherein,
the heterocyclic compound represented by Formula 1 is represented by one of Formulae 1-1 to 1-7: 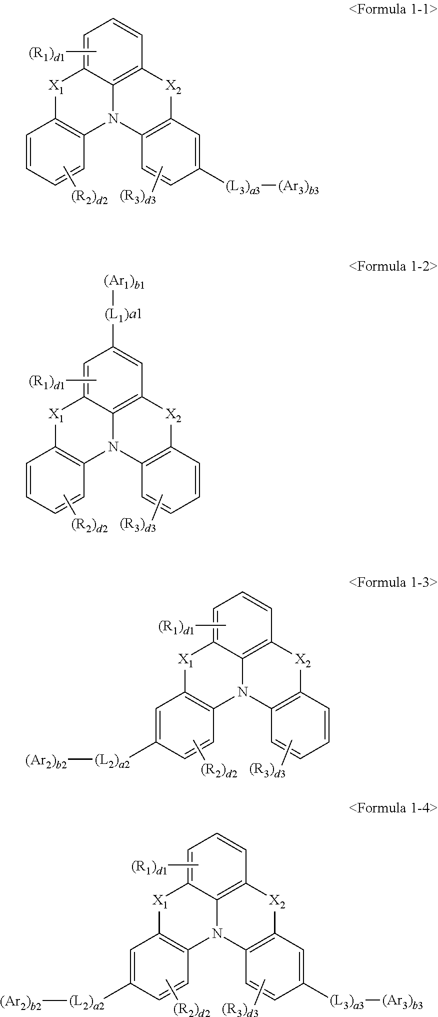 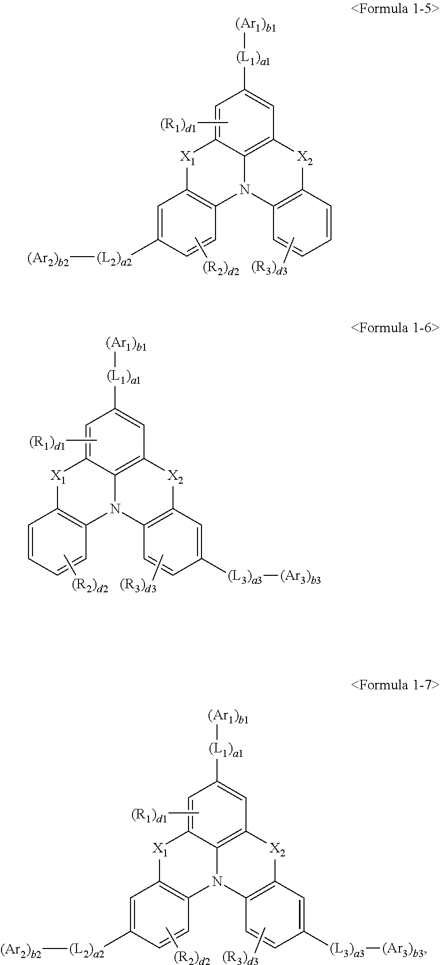
wherein, in Formulae 1-1 to 1-7,
X₁, X₂, L₁ to L₃, a1 to a3, Ar₁ to Ar₃, b1 to b3, R₁ to R₃, and d1 to d3 are each independently the same as described in claim 8.

20. The heterocyclic compound of claim 8, wherein,
the heterocyclic compound is selected from Compounds 1 to 144: 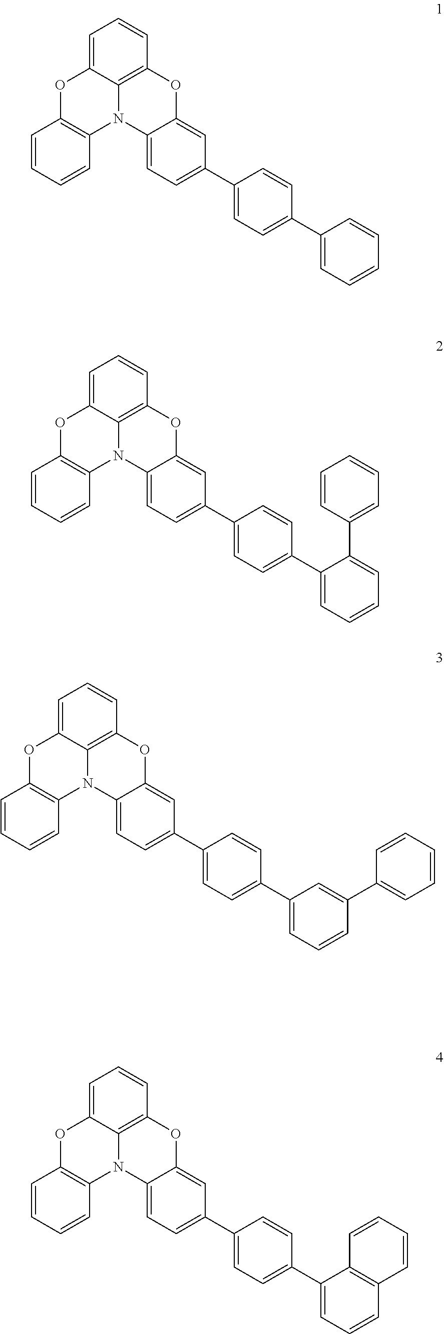 [SMILES: c1ccc(-c2ccc(-c3ccc4c(c3)Oc3cccc5c3N4c3ccccc3O5)cc2)cc1 ; c1ccc(-c2ccccc2-c2ccc(-c3ccc4c(c3)Oc3cccc5c3N4c3ccccc3O5)cc2)cc1 ; c1ccc(-c2cccc(-c3ccc(-c4ccc5c(c4)Oc4cccc6c4N5c4ccccc4O6)cc3)c2)cc1 ; c1ccc2c(c1)Oc1cccc3c1N2c1ccc(-c2ccc(-c4cccc5ccccc45)cc2)cc1O3] 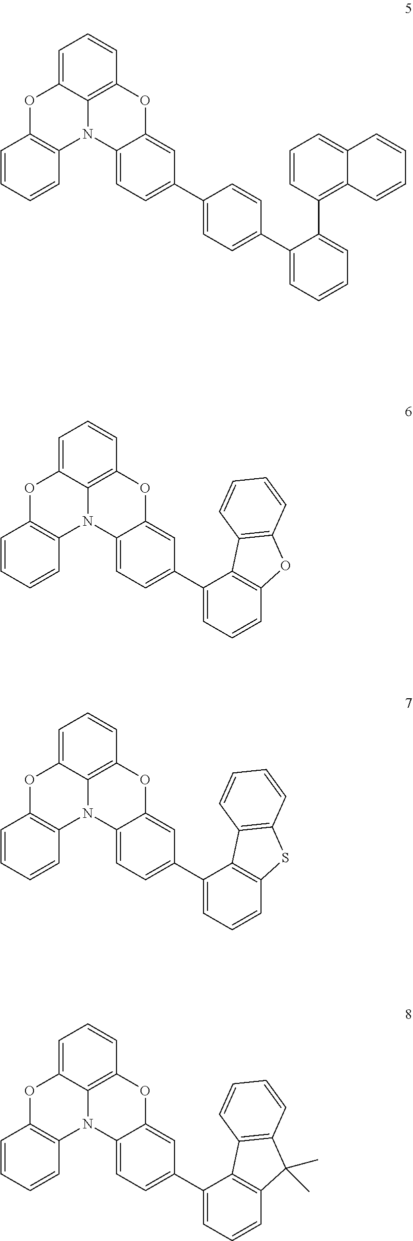 [SMILES: c1ccc2c(c1)Oc1cccc3c1N2c1ccc(-c2ccc(-c4ccccc4-c4cccc5ccccc45)cc2)cc1O3 ; c1ccc2c(c1)Oc1cccc3c1N2c1ccc(-c2cccc4oc5ccccc5c24)cc1O3 ; c1ccc2c(c1)Oc1cccc3c1N2c1ccc(-c2cccc4sc5ccccc5c24)cc1O3 ; CC1(C)c2ccccc2-c2c(-c3ccc4c(c3)Oc3cccc5c3N4c3ccccc3O5)cccc21] 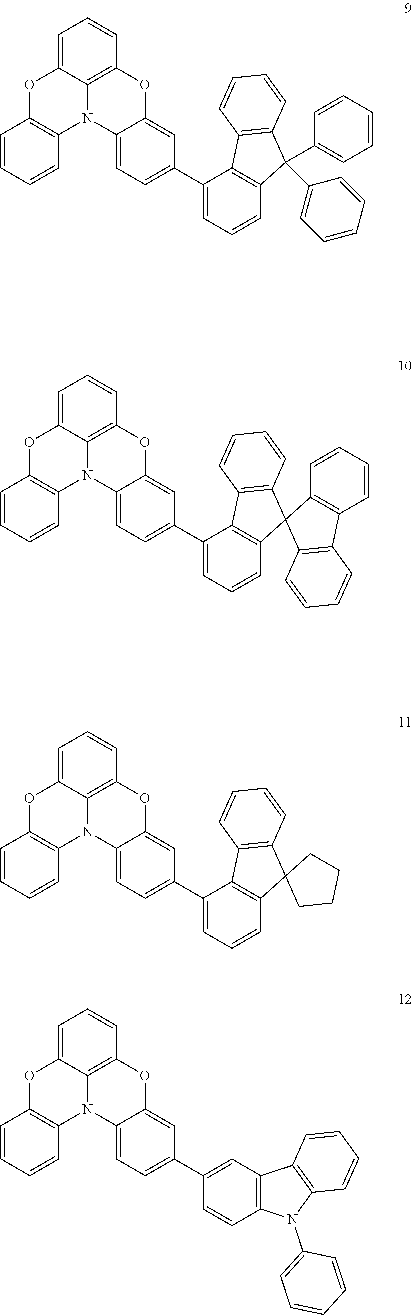 [SMILES: c1ccc(C2(c3ccccc3)c3ccccc3-c3c(-c4ccc5c(c4)Oc4cccc6c4N5c4ccccc4O6)cccc32)cc1 ; c1ccc2c(c1)Oc1cccc3c1N2c1ccc(-c2cccc4c2-c2ccccc2C42c4ccccc4-c4ccccc42)cc1O3 ; c1ccc2c(c1)Oc1cccc3c1N2c1ccc(-c2cccc4c2-c2ccccc2C42CCCC2)cc1O3 ; c1ccc(-n2c3ccccc3c3cc(-c4ccc5c(c4)Oc4cccc6c4N5c4ccccc4O6)ccc32)cc1] 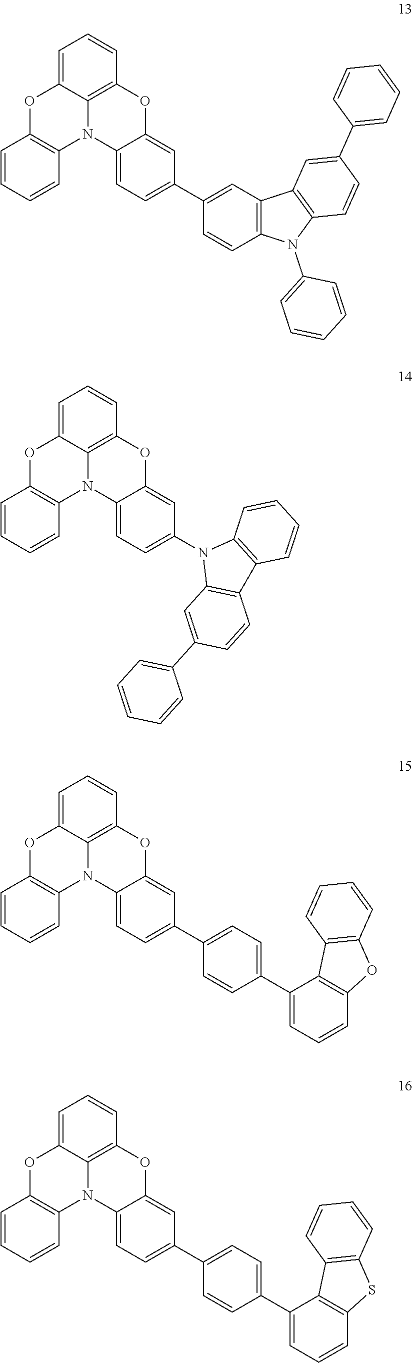 [SMILES: c1ccc(-c2ccc3c(c2)c2cc(-c4ccc5c(c4)Oc4cccc6c4N5c4ccccc4O6)ccc2n3-c2ccccc2)cc1 ; c1ccc(-c2ccc3c4ccccc4n(-c4ccc5c(c4)Oc4cccc6c4N5c4ccccc4O6)c3c2)cc1 ; c1ccc2c(c1)Oc1cccc3c1N2c1ccc(-c2ccc(-c4cccc5oc6ccccc6c45)cc2)cc1O3 ; c1ccc2c(c1)Oc1cccc3c1N2c1ccc(-c2ccc(-c4cccc5sc6ccccc6c45)cc2)cc1O3] 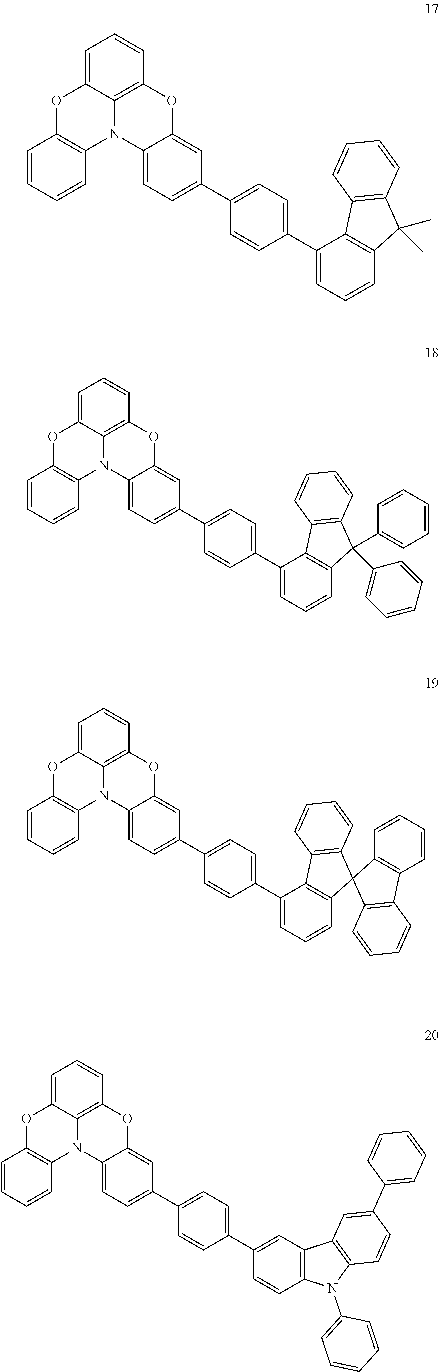 [SMILES: CC1(C)c2ccccc2-c2c(-c3ccc(-c4ccc5c(c4)Oc4cccc6c4N5c4ccccc4O6)cc3)cccc21 ; c1ccc(C2(c3ccccc3)c3ccccc3-c3c(-c4ccc(-c5ccc6c(c5)Oc5cccc7c5N6c5ccccc5O7)cc4)cccc32)cc1 ; c1ccc2c(c1)Oc1cccc3c1N2c1ccc(-c2ccc(-c4cccc5c4-c4ccccc4C54c5ccccc5-c5ccccc54)cc2)cc1O3 ; c1ccc(-c2ccc3c(c2)c2cc(-c4ccc(-c5ccc6c(c5)Oc5cccc7c5N6c5ccccc5O7)cc4)ccc2n3-c2ccccc2)cc1] 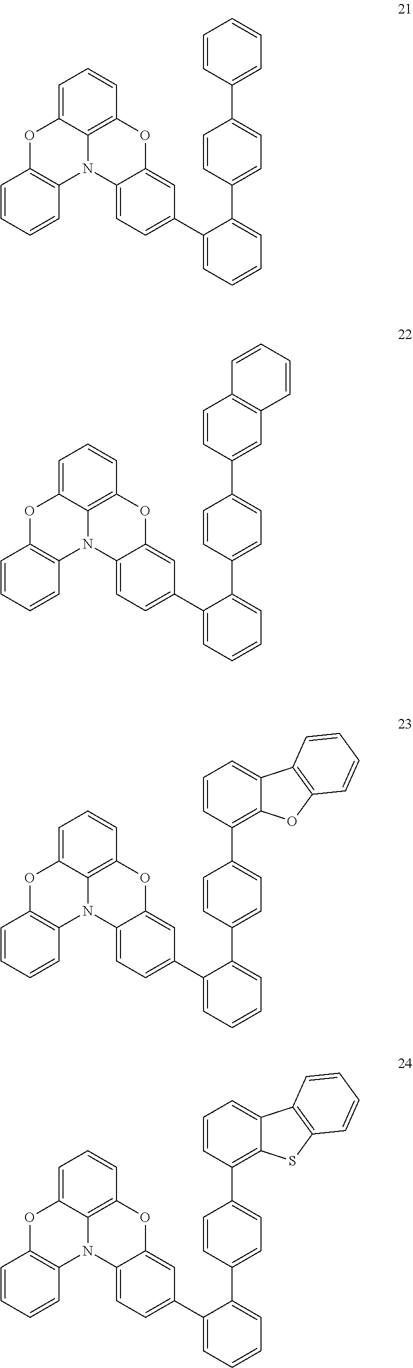 [SMILES: c1ccc(-c2ccc(-c3ccccc3-c3ccc4c(c3)Oc3cccc5c3N4c3ccccc3O5)cc2)cc1 ; c1ccc2c(c1)Oc1cccc3c1N2c1ccc(-c2ccccc2-c2ccc(-c4ccc5ccccc5c4)cc2)cc1O3 ; c1ccc2c(c1)Oc1cccc3c1N2c1ccc(-c2ccccc2-c2ccc(-c4cccc5c4oc4ccccc45)cc2)cc1O3 ; c1ccc2c(c1)Oc1cccc3c1N2c1ccc(-c2ccccc2-c2ccc(-c4cccc5c4sc4ccccc45)cc2)cc1O3] 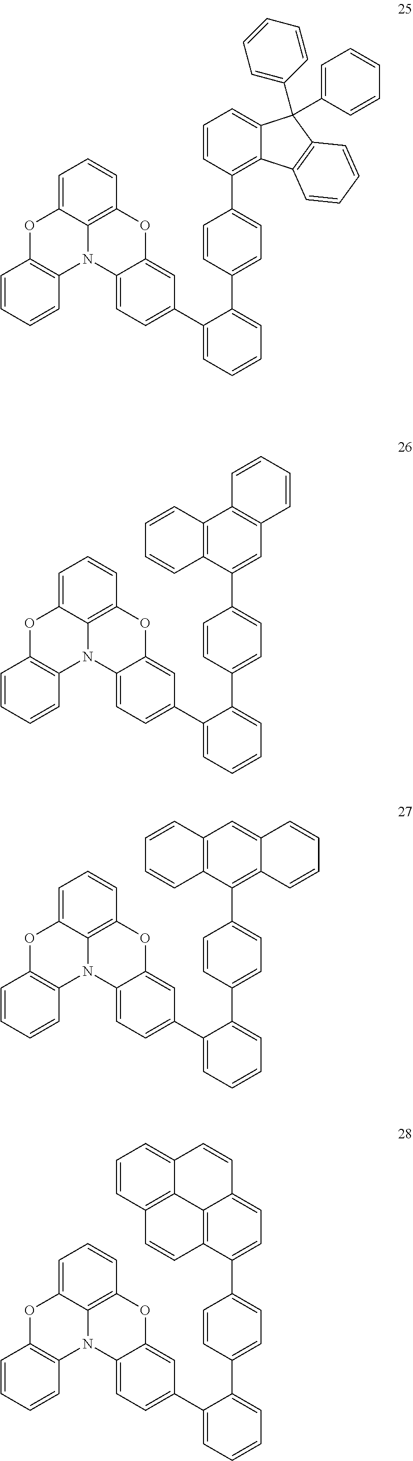 [SMILES: c1ccc(C2(c3ccccc3)c3ccccc3-c3c(-c4ccc(-c5ccccc5-c5ccc6c(c5)Oc5cccc7c5N6c5ccccc5O7)cc4)cccc32)cc1 ; c1ccc2c(c1)Oc1cccc3c1N2c1ccc(-c2ccccc2-c2ccc(-c4cc5ccccc5c5ccccc45)cc2)cc1O3 ; c1ccc2c(c1)Oc1cccc3c1N2c1ccc(-c2ccccc2-c2ccc(-c4c5ccccc5cc5ccccc45)cc2)cc1O3 ; c1ccc2c(c1)Oc1cccc3c1N2c1ccc(-c2ccccc2-c2ccc(-c4ccc5ccc6cccc7ccc4c5c67)cc2)cc1O3] 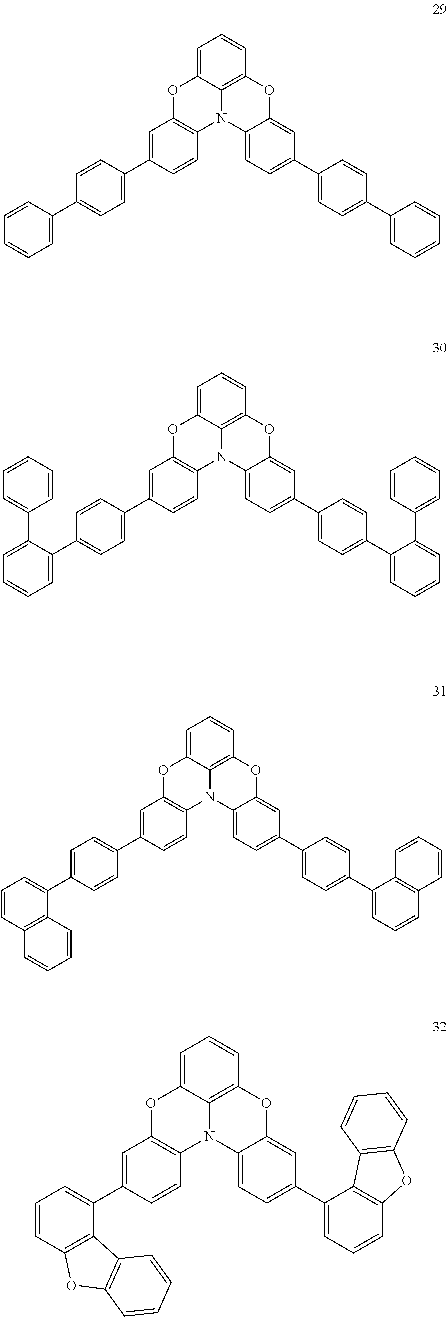 [SMILES: c1ccc(-c2ccc(-c3ccc4c(c3)Oc3cccc5c3N4c3ccc(-c4ccc(-c6ccccc6)cc4)cc3O5)cc2)cc1 ; c1ccc(-c2ccccc2-c2ccc(-c3ccc4c(c3)Oc3cccc5c3N4c3ccc(-c4ccc(-c6ccccc6-c6ccccc6)cc4)cc3O5)cc2)cc1 ; c1cc2c3c(c1)Oc1cc(-c4ccc(-c5cccc6ccccc56)cc4)ccc1N3c1ccc(-c3ccc(-c4cccc5ccccc45)cc3)cc1O2 ; c1cc2c3c(c1)Oc1cc(-c4cccc5oc6ccccc6c45)ccc1N3c1ccc(-c3cccc4oc5ccccc5c34)cc1O2] 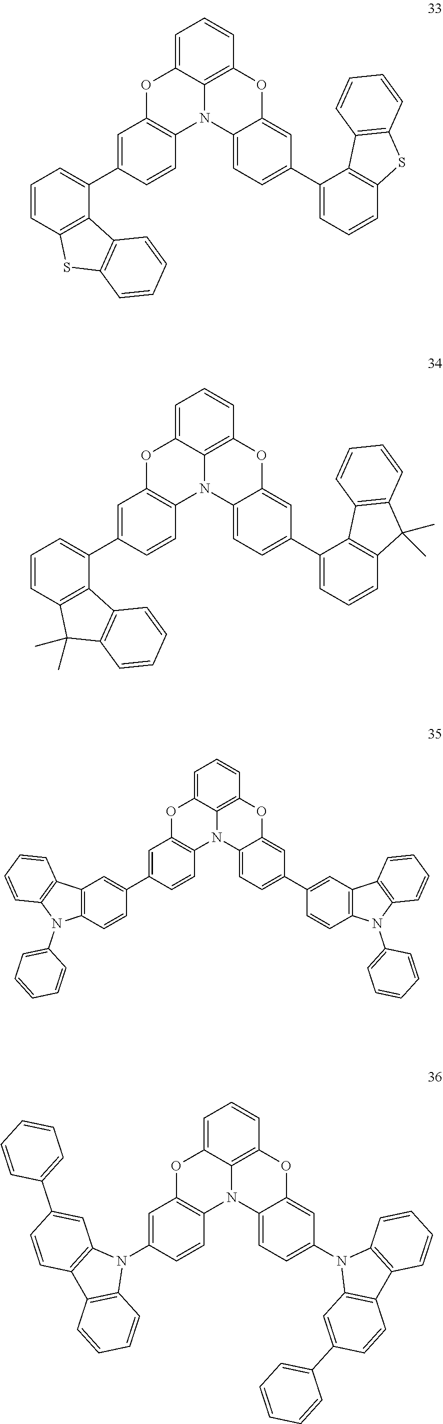 [SMILES: c1cc2c3c(c1)Oc1cc(-c4cccc5sc6ccccc6c45)ccc1N3c1ccc(-c3cccc4sc5ccccc5c34)cc1O2 ; CC1(C)c2ccccc2-c2c(-c3ccc4c(c3)Oc3cccc5c3N4c3ccc(-c4cccc6c4-c4ccccc4C6(C)C)cc3O5)cccc21 ; c1ccc(-n2c3ccccc3c3cc(-c4ccc5c(c4)Oc4cccc6c4N5c4ccc(-c5ccc7c(c5)c5ccccc5n7-c5ccccc5)cc4O6)ccc32)cc1 ; c1ccc(-c2ccc3c4ccccc4n(-c4ccc5c(c4)Oc4cccc6c4N5c4ccc(-n5c7ccccc7c7ccc(-c8ccccc8)cc75)cc4O6)c3c2)cc1] 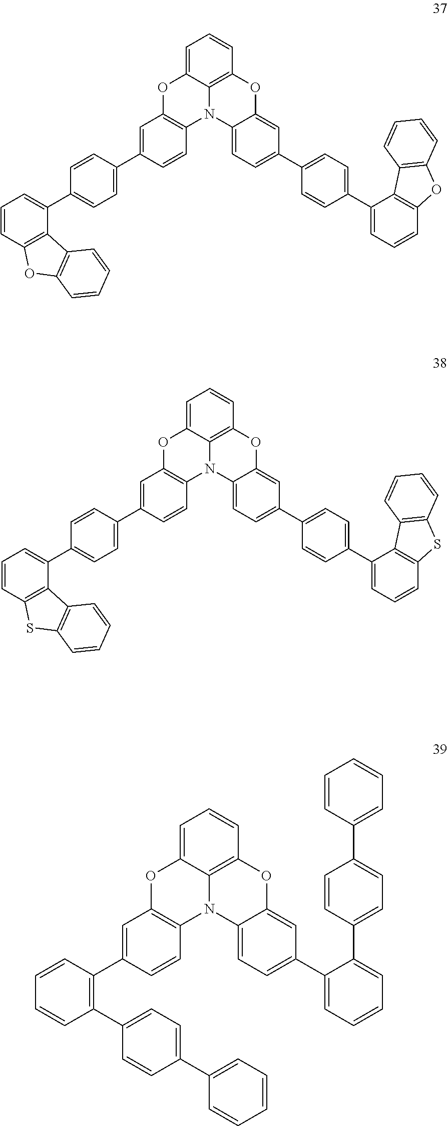 [SMILES: c1cc2c3c(c1)Oc1cc(-c4ccc(-c5cccc6oc7ccccc7c56)cc4)ccc1N3c1ccc(-c3ccc(-c4cccc5oc6ccccc6c45)cc3)cc1O2 ; c1cc2c3c(c1)Oc1cc(-c4ccc(-c5cccc6sc7ccccc7c56)cc4)ccc1N3c1ccc(-c3ccc(-c4cccc5sc6ccccc6c45)cc3)cc1O2 ; c1ccc(-c2ccc(-c3ccccc3-c3ccc4c(c3)Oc3cccc5c3N4c3ccc(-c4ccccc4-c4ccc(-c6ccccc6)cc4)cc3O5)cc2)cc1] 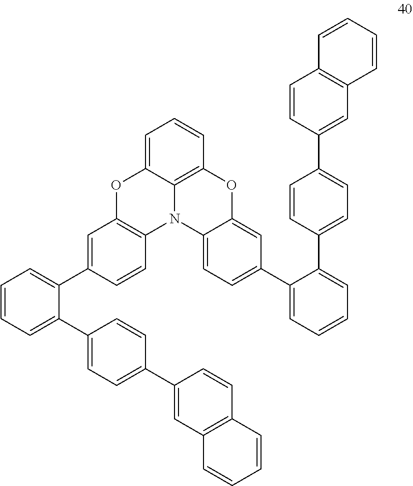 [SMILES: c1cc2c3c(c1)Oc1cc(-c4ccccc4-c4ccc(-c5ccc6ccccc6c5)cc4)ccc1N3c1ccc(-c3ccccc3-c3ccc(-c4ccc5ccccc5c4)cc3)cc1O2] 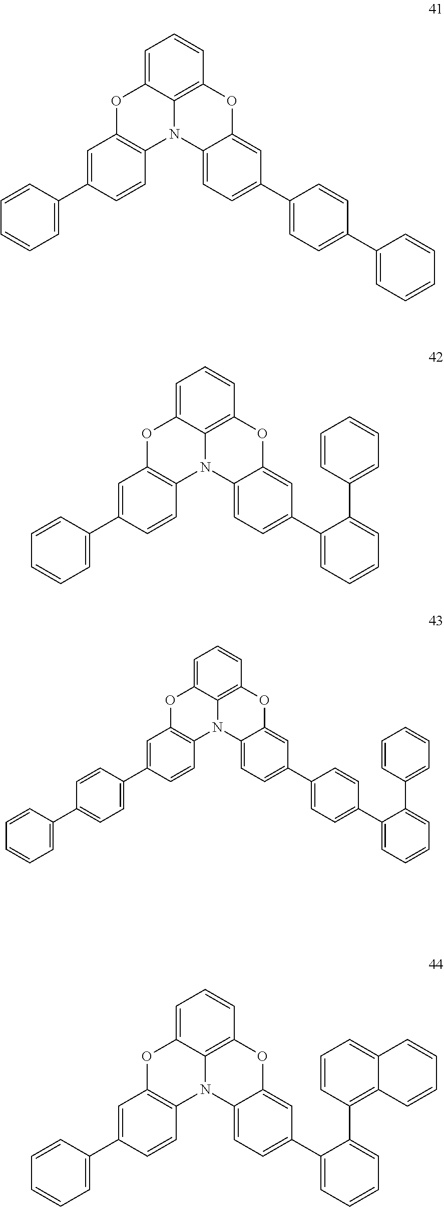 [SMILES: c1ccc(-c2ccc(-c3ccc4c(c3)Oc3cccc5c3N4c3ccc(-c4ccccc4)cc3O5)cc2)cc1 ; c1ccc(-c2ccc3c(c2)Oc2cccc4c2N3c2ccc(-c3ccccc3-c3ccccc3)cc2O4)cc1 ; c1ccc(-c2ccc(-c3ccc4c(c3)Oc3cccc5c3N4c3ccc(-c4ccc(-c6ccccc6-c6ccccc6)cc4)cc3O5)cc2)cc1 ; c1ccc(-c2ccc3c(c2)Oc2cccc4c2N3c2ccc(-c3ccccc3-c3cccc5ccccc35)cc2O4)cc1] 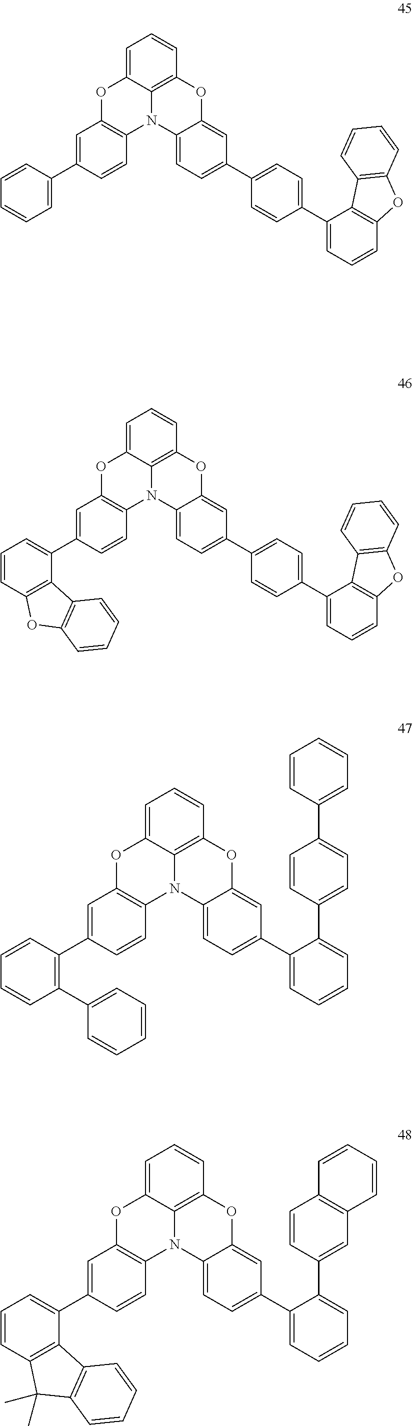 [SMILES: c1ccc(-c2ccc3c(c2)Oc2cccc4c2N3c2ccc(-c3ccc(-c5cccc6oc7ccccc7c56)cc3)cc2O4)cc1 ; c1cc2c3c(c1)Oc1cc(-c4cccc5oc6ccccc6c45)ccc1N3c1ccc(-c3ccc(-c4cccc5oc6ccccc6c45)cc3)cc1O2 ; c1ccc(-c2ccc(-c3ccccc3-c3ccc4c(c3)Oc3cccc5c3N4c3ccc(-c4ccccc4-c4ccccc4)cc3O5)cc2)cc1 ; CC1(C)c2ccccc2-c2c(-c3ccc4c(c3)Oc3cccc5c3N4c3ccc(-c4ccccc4-c4ccc6ccccc6c4)cc3O5)cccc21] 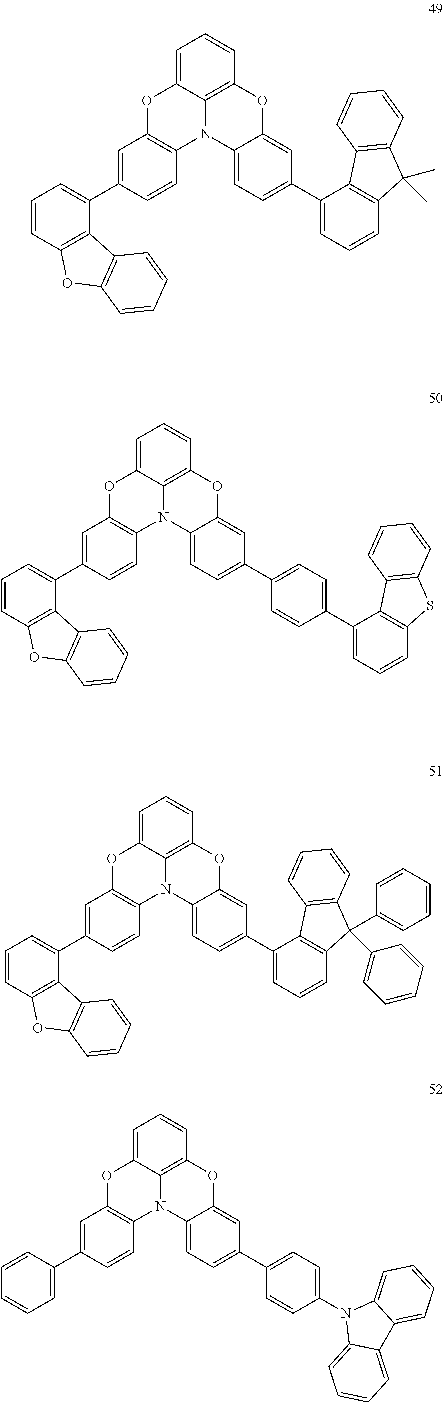 [SMILES: CC1(C)c2ccccc2-c2c(-c3ccc4c(c3)Oc3cccc5c3N4c3ccc(-c4cccc6oc7ccccc7c46)cc3O5)cccc21 ; c1cc2c3c(c1)Oc1cc(-c4cccc5oc6ccccc6c45)ccc1N3c1ccc(-c3ccc(-c4cccc5sc6ccccc6c45)cc3)cc1O2 ; c1ccc(C2(c3ccccc3)c3ccccc3-c3c(-c4ccc5c(c4)Oc4cccc6c4N5c4ccc(-c5cccc7oc8ccccc8c57)cc4O6)cccc32)cc1 ; c1ccc(-c2ccc3c(c2)Oc2cccc4c2N3c2ccc(-c3ccc(-n5c6ccccc6c6ccccc65)cc3)cc2O4)cc1] 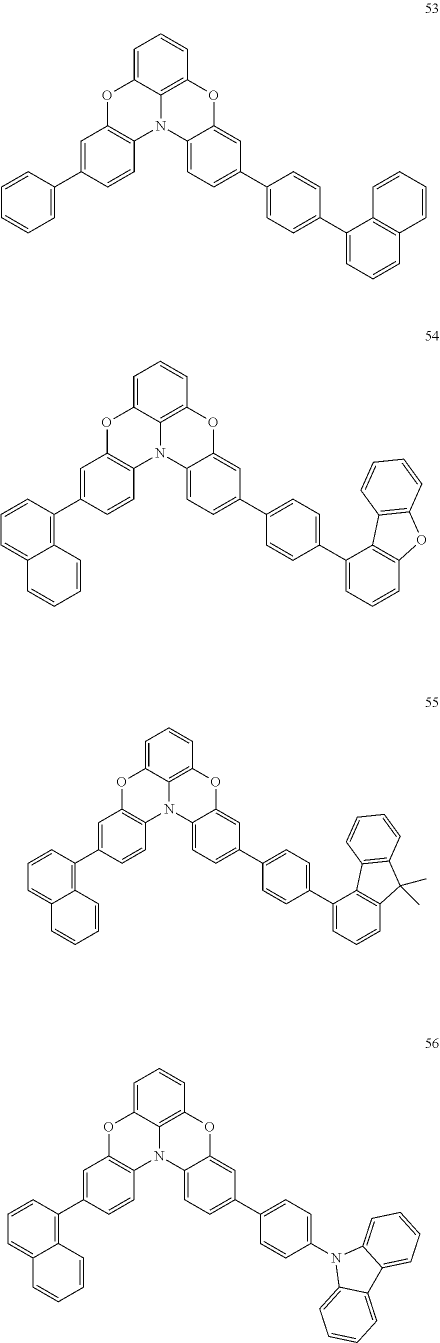 [SMILES: c1ccc(-c2ccc3c(c2)Oc2cccc4c2N3c2ccc(-c3ccc(-c5cccc6ccccc56)cc3)cc2O4)cc1 ; c1cc2c3c(c1)Oc1cc(-c4cccc5ccccc45)ccc1N3c1ccc(-c3ccc(-c4cccc5oc6ccccc6c45)cc3)cc1O2 ; CC1(C)c2ccccc2-c2c(-c3ccc(-c4ccc5c(c4)Oc4cccc6c4N5c4ccc(-c5cccc7ccccc57)cc4O6)cc3)cccc21 ; c1cc2c3c(c1)Oc1cc(-c4cccc5ccccc45)ccc1N3c1ccc(-c3ccc(-n4c5ccccc5c5ccccc54)cc3)cc1O2] 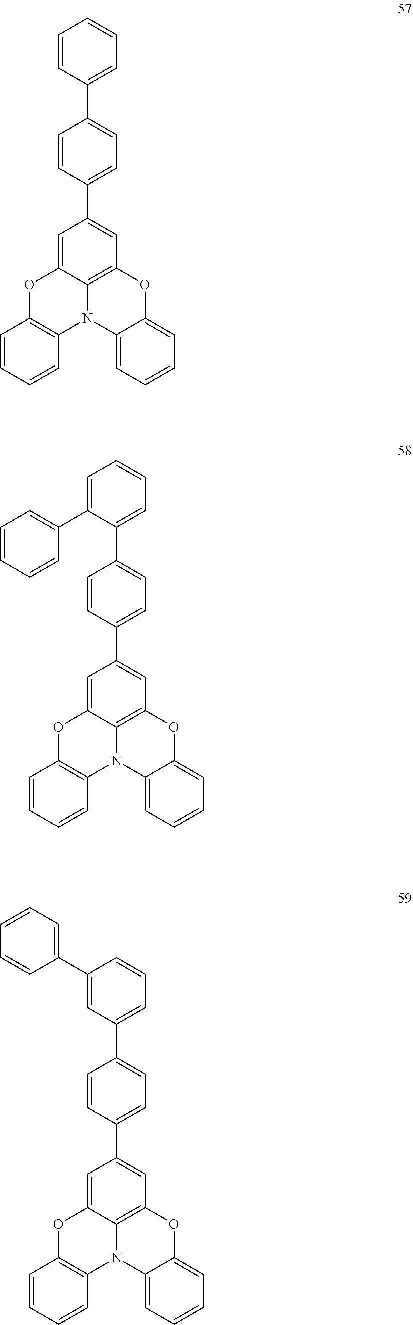 [SMILES: c1ccc(-c2ccc(-c3cc4c5c(c3)Oc3ccccc3N5c3ccccc3O4)cc2)cc1 ; c1ccc(-c2ccccc2-c2ccc(-c3cc4c5c(c3)Oc3ccccc3N5c3ccccc3O4)cc2)cc1 ; c1ccc(-c2cccc(-c3ccc(-c4cc5c6c(c4)Oc4ccccc4N6c4ccccc4O5)cc3)c2)cc1] 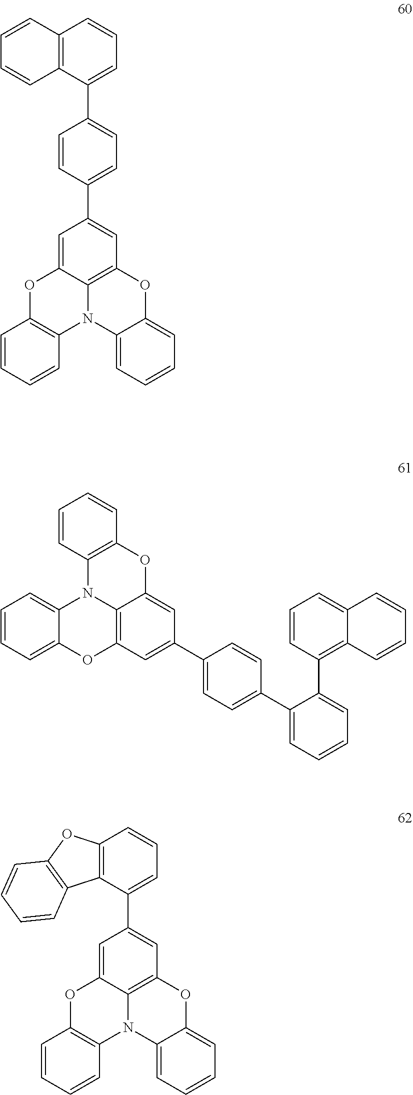 [SMILES: c1ccc2c(c1)Oc1cc(-c3ccc(-c4cccc5ccccc45)cc3)cc3c1N2c1ccccc1O3 ; c1ccc2c(c1)Oc1cc(-c3ccc(-c4ccccc4-c4cccc5ccccc45)cc3)cc3c1N2c1ccccc1O3 ; c1ccc2c(c1)Oc1cc(-c3cccc4oc5ccccc5c34)cc3c1N2c1ccccc1O3] 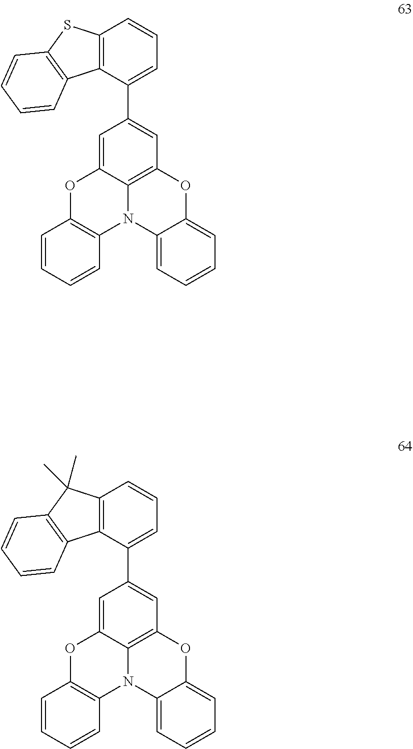 [SMILES: c1ccc2c(c1)Oc1cc(-c3cccc4sc5ccccc5c34)cc3c1N2c1ccccc1O3 ; CC1(C)c2ccccc2-c2c(-c3cc4c5c(c3)Oc3ccccc3N5c3ccccc3O4)cccc21] 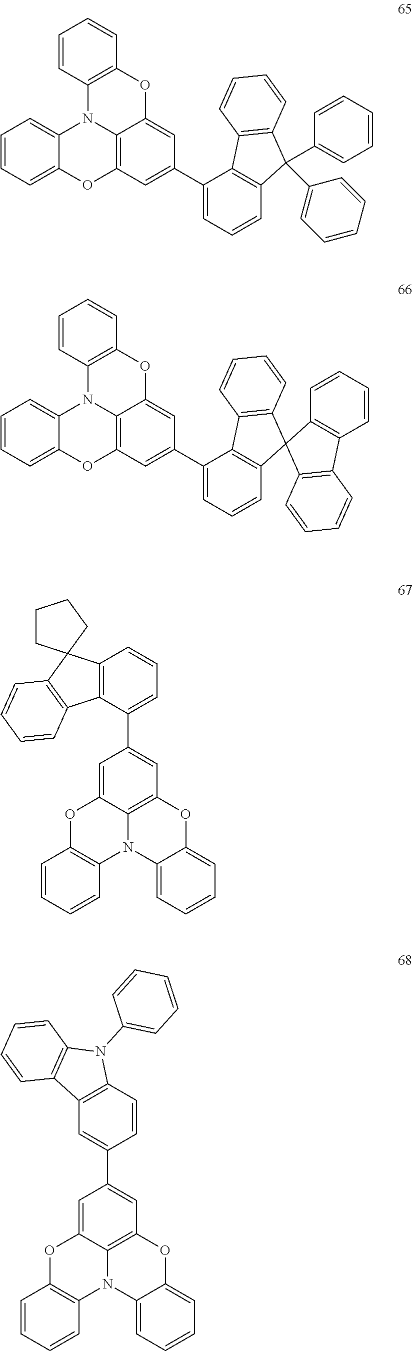 [SMILES: c1ccc(C2(c3ccccc3)c3ccccc3-c3c(-c4cc5c6c(c4)Oc4ccccc4N6c4ccccc4O5)cccc32)cc1 ; c1ccc2c(c1)Oc1cc(-c3cccc4c3-c3ccccc3C43c4ccccc4-c4ccccc43)cc3c1N2c1ccccc1O3 ; c1ccc2c(c1)Oc1cc(-c3cccc4c3-c3ccccc3C43CCCC3)cc3c1N2c1ccccc1O3 ; c1ccc(-n2c3ccccc3c3cc(-c4cc5c6c(c4)Oc4ccccc4N6c4ccccc4O5)ccc32)cc1] 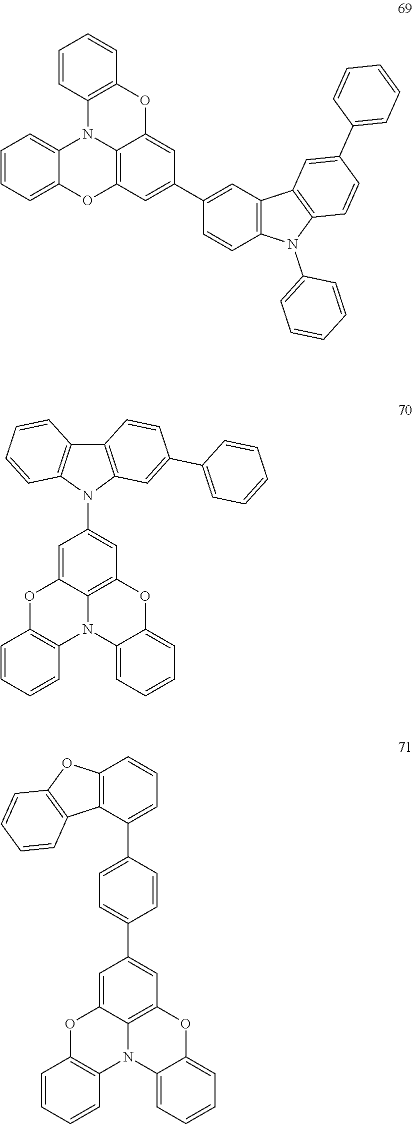 [SMILES: c1ccc(-c2ccc3c(c2)c2cc(-c4cc5c6c(c4)Oc4ccccc4N6c4ccccc4O5)ccc2n3-c2ccccc2)cc1 ; c1ccc(-c2ccc3c4ccccc4n(-c4cc5c6c(c4)Oc4ccccc4N6c4ccccc4O5)c3c2)cc1 ; c1ccc2c(c1)Oc1cc(-c3ccc(-c4cccc5oc6ccccc6c45)cc3)cc3c1N2c1ccccc1O3] 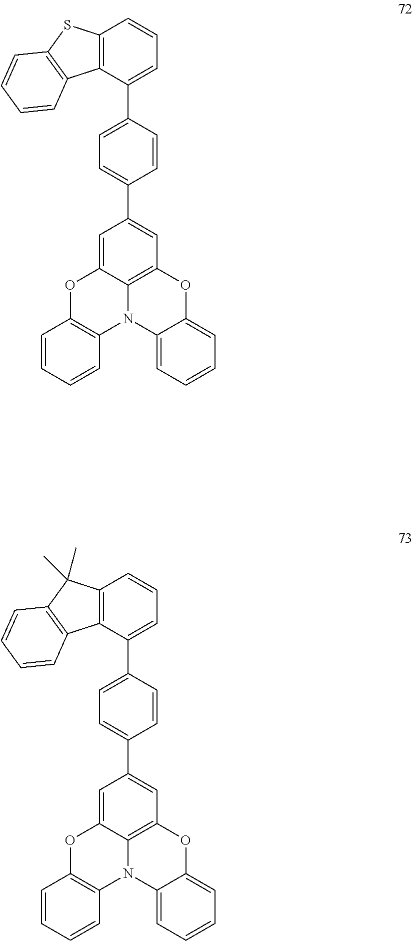 [SMILES: c1ccc2c(c1)Oc1cc(-c3ccc(-c4cccc5sc6ccccc6c45)cc3)cc3c1N2c1ccccc1O3 ; CC1(C)c2ccccc2-c2c(-c3ccc(-c4cc5c6c(c4)Oc4ccccc4N6c4ccccc4O5)cc3)cccc21] 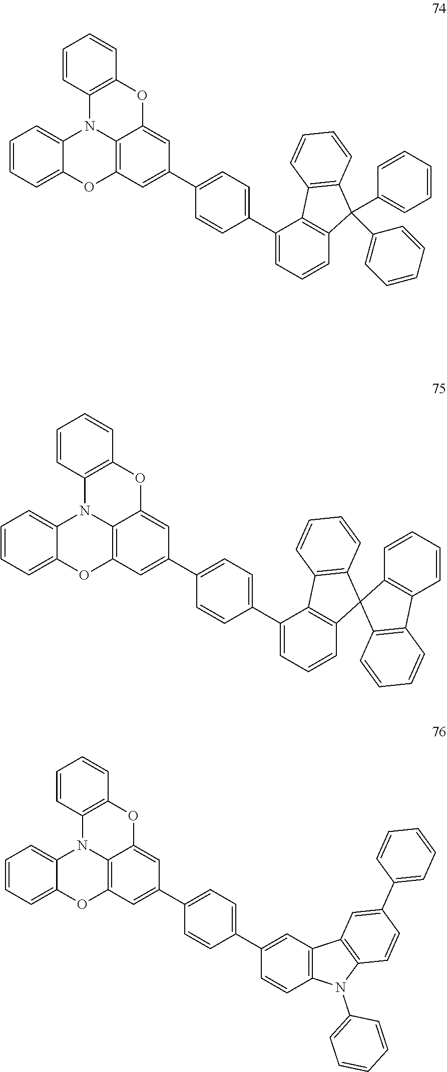 [SMILES: c1ccc(C2(c3ccccc3)c3ccccc3-c3c(-c4ccc(-c5cc6c7c(c5)Oc5ccccc5N7c5ccccc5O6)cc4)cccc32)cc1 ; c1ccc2c(c1)Oc1cc(-c3ccc(-c4cccc5c4-c4ccccc4C54c5ccccc5-c5ccccc54)cc3)cc3c1N2c1ccccc1O3 ; c1ccc(-c2ccc3c(c2)c2cc(-c4ccc(-c5cc6c7c(c5)Oc5ccccc5N7c5ccccc5O6)cc4)ccc2n3-c2ccccc2)cc1] 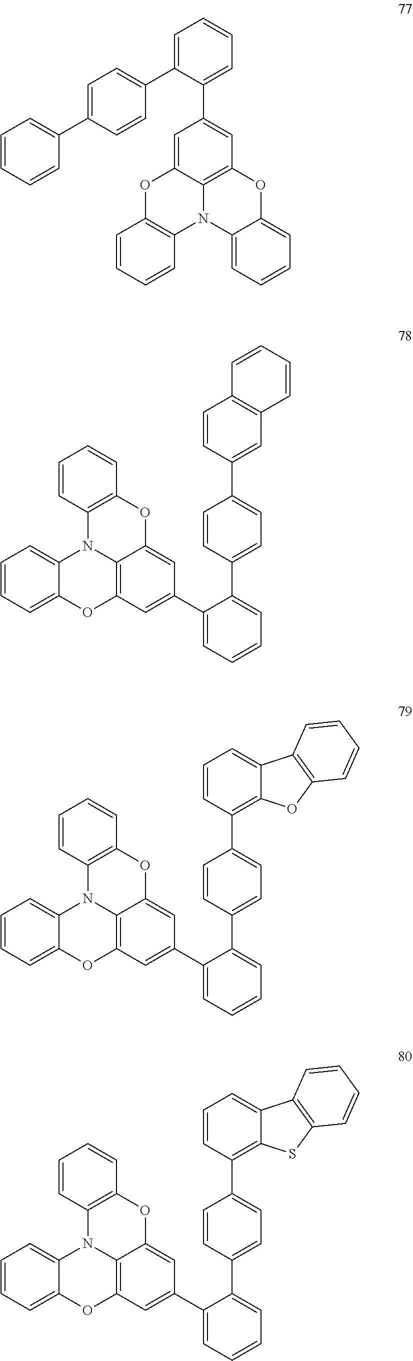 [SMILES: c1ccc(-c2ccc(-c3ccccc3-c3cc4c5c(c3)Oc3ccccc3N5c3ccccc3O4)cc2)cc1 ; c1ccc2c(c1)Oc1cc(-c3ccccc3-c3ccc(-c4ccc5ccccc5c4)cc3)cc3c1N2c1ccccc1O3 ; c1ccc2c(c1)Oc1cc(-c3ccccc3-c3ccc(-c4cccc5c4oc4ccccc45)cc3)cc3c1N2c1ccccc1O3 ; c1ccc2c(c1)Oc1cc(-c3ccccc3-c3ccc(-c4cccc5c4sc4ccccc45)cc3)cc3c1N2c1ccccc1O3] 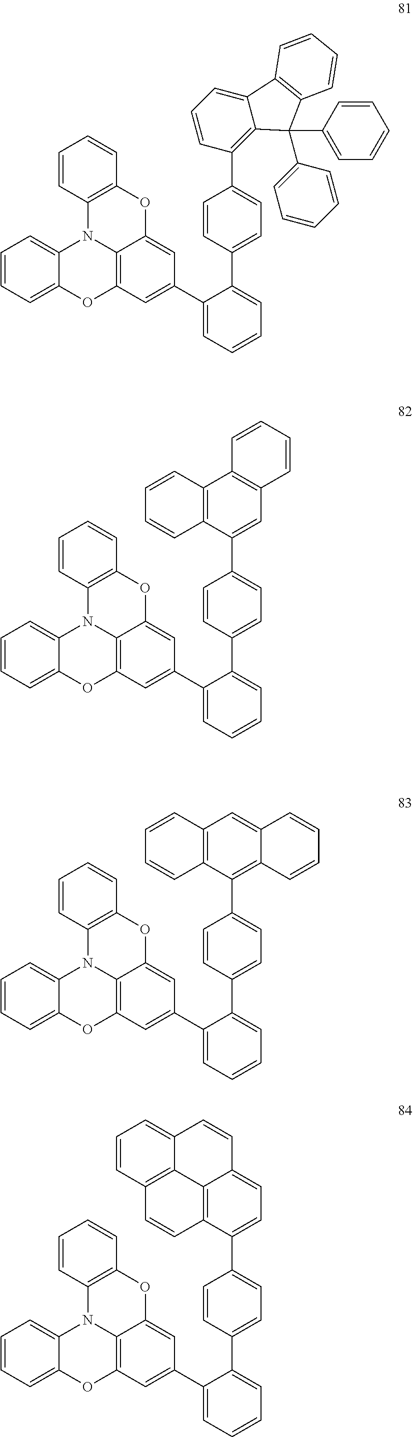 [SMILES: c1ccc(C2(c3ccccc3)c3ccccc3-c3cccc(-c4ccc(-c5ccccc5-c5cc6c7c(c5)Oc5ccccc5N7c5ccccc5O6)cc4)c32)cc1 ; c1ccc2c(c1)Oc1cc(-c3ccccc3-c3ccc(-c4cc5ccccc5c5ccccc45)cc3)cc3c1N2c1ccccc1O3 ; c1ccc2c(c1)Oc1cc(-c3ccccc3-c3ccc(-c4c5ccccc5cc5ccccc45)cc3)cc3c1N2c1ccccc1O3 ; c1ccc2c(c1)Oc1cc(-c3ccccc3-c3ccc(-c4ccc5ccc6cccc7ccc4c5c67)cc3)cc3c1N2c1ccccc1O3] 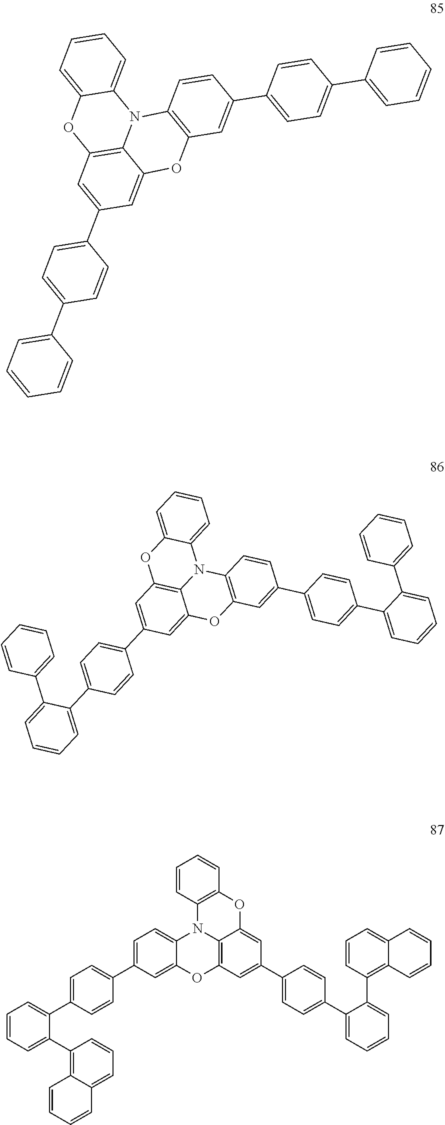 [SMILES: c1ccc(-c2ccc(-c3ccc4c(c3)Oc3cc(-c5ccc(-c6ccccc6)cc5)cc5c3N4c3ccccc3O5)cc2)cc1 ; c1ccc(-c2ccccc2-c2ccc(-c3ccc4c(c3)Oc3cc(-c5ccc(-c6ccccc6-c6ccccc6)cc5)cc5c3N4c3ccccc3O5)cc2)cc1 ; c1ccc2c(c1)Oc1cc(-c3ccc(-c4ccccc4-c4cccc5ccccc45)cc3)cc3c1N2c1ccc(-c2ccc(-c4ccccc4-c4cccc5ccccc45)cc2)cc1O3] 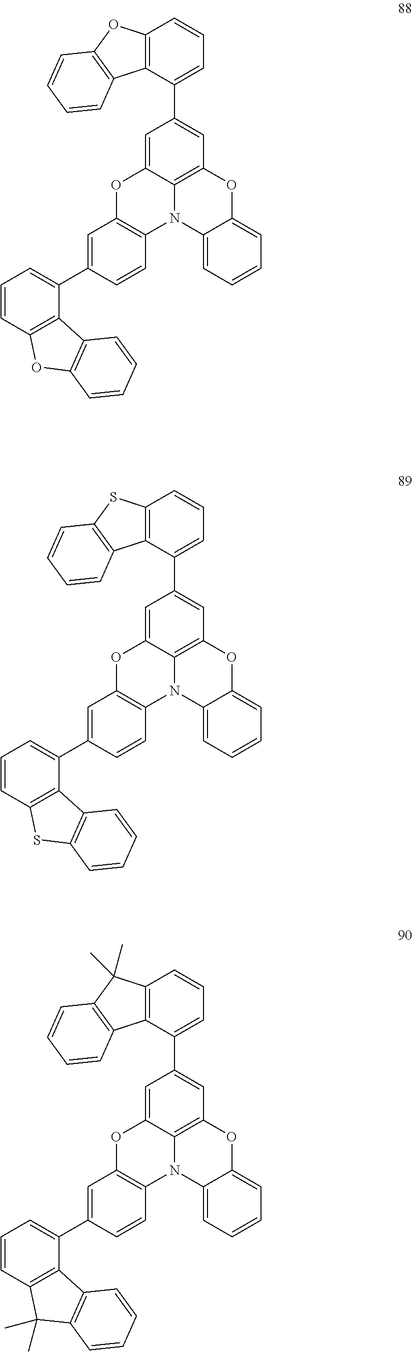 [SMILES: c1ccc2c(c1)Oc1cc(-c3cccc4oc5ccccc5c34)cc3c1N2c1ccc(-c2cccc4oc5ccccc5c24)cc1O3 ; c1ccc2c(c1)Oc1cc(-c3cccc4sc5ccccc5c34)cc3c1N2c1ccc(-c2cccc4sc5ccccc5c24)cc1O3 ; CC1(C)c2ccccc2-c2c(-c3ccc4c(c3)Oc3cc(-c5cccc6c5-c5ccccc5C6(C)C)cc5c3N4c3ccccc3O5)cccc21] 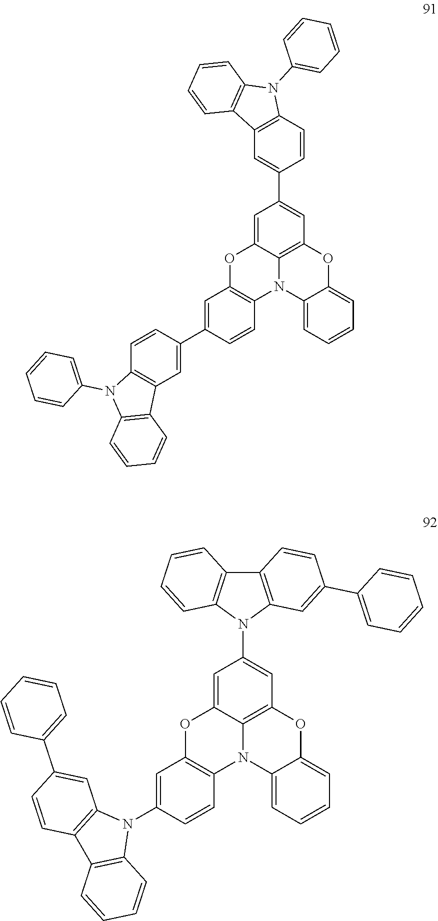 [SMILES: c1ccc(-n2c3ccccc3c3cc(-c4ccc5c(c4)Oc4cc(-c6ccc7c(c6)c6ccccc6n7-c6ccccc6)cc6c4N5c4ccccc4O6)ccc32)cc1 ; c1ccc(-c2ccc3c4ccccc4n(-c4ccc5c(c4)Oc4cc(-n6c7ccccc7c7ccc(-c8ccccc8)cc76)cc6c4N5c4ccccc4O6)c3c2)cc1] 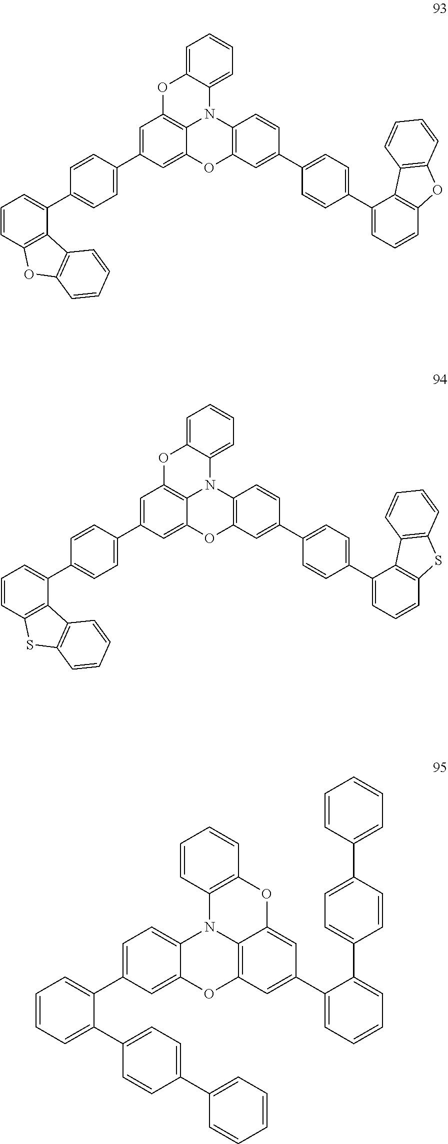 [SMILES: c1ccc2c(c1)Oc1cc(-c3ccc(-c4cccc5oc6ccccc6c45)cc3)cc3c1N2c1ccc(-c2ccc(-c4cccc5oc6ccccc6c45)cc2)cc1O3 ; c1ccc2c(c1)Oc1cc(-c3ccc(-c4cccc5sc6ccccc6c45)cc3)cc3c1N2c1ccc(-c2ccc(-c4cccc5sc6ccccc6c45)cc2)cc1O3 ; c1ccc(-c2ccc(-c3ccccc3-c3ccc4c(c3)Oc3cc(-c5ccccc5-c5ccc(-c6ccccc6)cc5)cc5c3N4c3ccccc3O5)cc2)cc1] 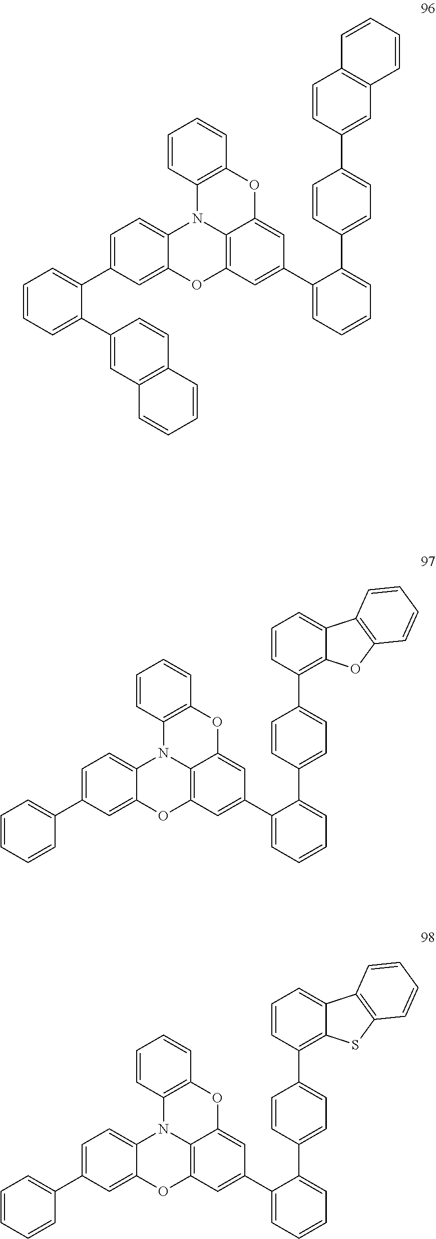 [SMILES: c1ccc2c(c1)Oc1cc(-c3ccccc3-c3ccc(-c4ccc5ccccc5c4)cc3)cc3c1N2c1ccc(-c2ccccc2-c2ccc4ccccc4c2)cc1O3 ; c1ccc(-c2ccc3c(c2)Oc2cc(-c4ccccc4-c4ccc(-c5cccc6c5oc5ccccc56)cc4)cc4c2N3c2ccccc2O4)cc1 ; c1ccc(-c2ccc3c(c2)Oc2cc(-c4ccccc4-c4ccc(-c5cccc6c5sc5ccccc56)cc4)cc4c2N3c2ccccc2O4)cc1] 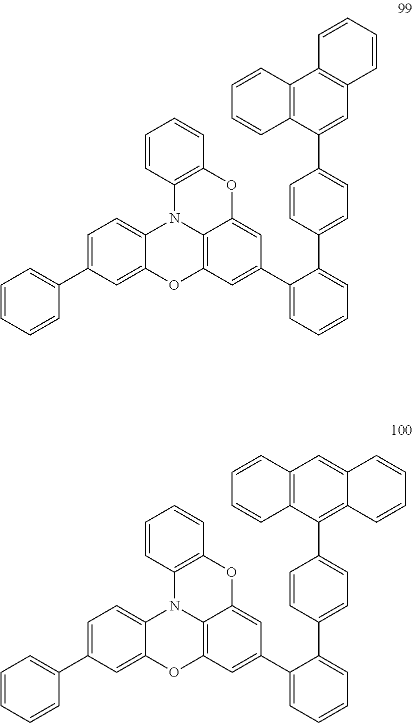 [SMILES: c1ccc(-c2ccc3c(c2)Oc2cc(-c4ccccc4-c4ccc(-c5cc6ccccc6c6ccccc56)cc4)cc4c2N3c2ccccc2O4)cc1 ; c1ccc(-c2ccc3c(c2)Oc2cc(-c4ccccc4-c4ccc(-c5c6ccccc6cc6ccccc56)cc4)cc4c2N3c2ccccc2O4)cc1] 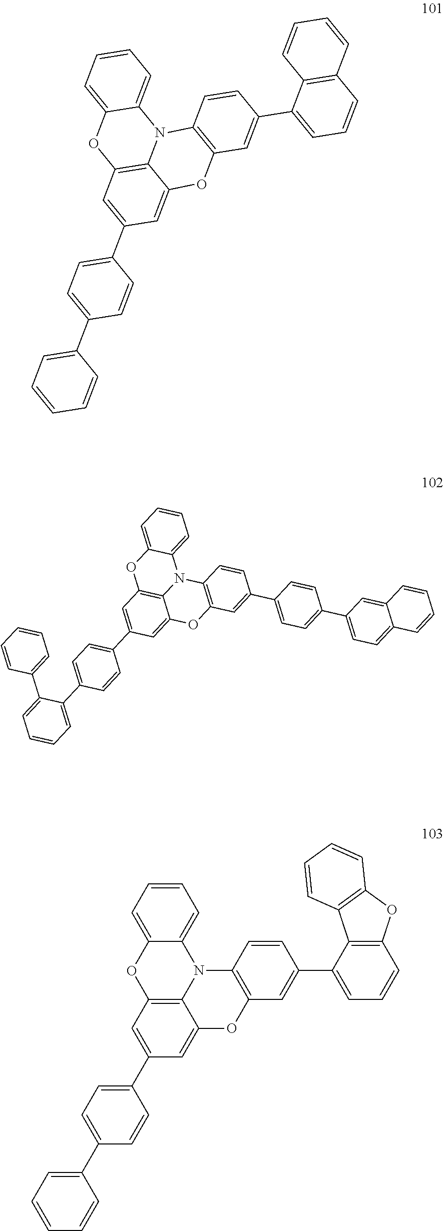 [SMILES: c1ccc(-c2ccc(-c3cc4c5c(c3)Oc3cc(-c6cccc7ccccc67)ccc3N5c3ccccc3O4)cc2)cc1 ; c1ccc(-c2ccccc2-c2ccc(-c3cc4c5c(c3)Oc3cc(-c6ccc(-c7ccc8ccccc8c7)cc6)ccc3N5c3ccccc3O4)cc2)cc1 ; c1ccc(-c2ccc(-c3cc4c5c(c3)Oc3cc(-c6cccc7oc8ccccc8c67)ccc3N5c3ccccc3O4)cc2)cc1] 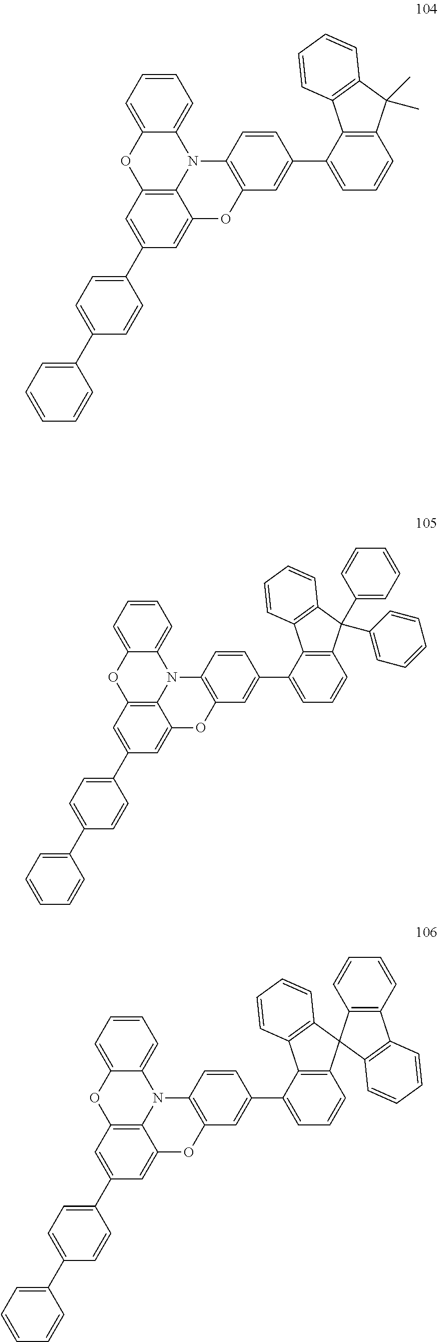 [SMILES: CC1(C)c2ccccc2-c2c(-c3ccc4c(c3)Oc3cc(-c5ccc(-c6ccccc6)cc5)cc5c3N4c3ccccc3O5)cccc21 ; c1ccc(-c2ccc(-c3cc4c5c(c3)Oc3cc(-c6cccc7c6-c6ccccc6C7(c6ccccc6)c6ccccc6)ccc3N5c3ccccc3O4)cc2)cc1 ; c1ccc(-c2ccc(-c3cc4c5c(c3)Oc3cc(-c6cccc7c6-c6ccccc6C76c7ccccc7-c7ccccc76)ccc3N5c3ccccc3O4)cc2)cc1] 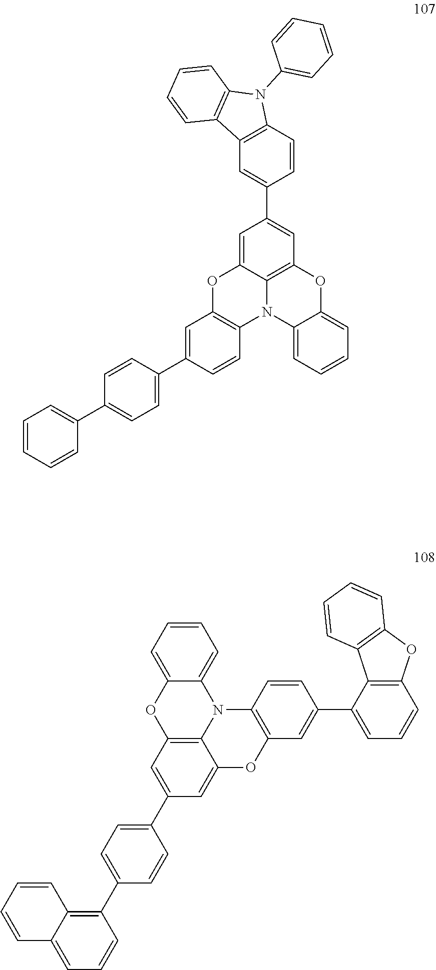 [SMILES: c1ccc(-c2ccc(-c3ccc4c(c3)Oc3cc(-c5ccc6c(c5)c5ccccc5n6-c5ccccc5)cc5c3N4c3ccccc3O5)cc2)cc1 ; c1ccc2c(c1)Oc1cc(-c3ccc(-c4cccc5ccccc45)cc3)cc3c1N2c1ccc(-c2cccc4oc5ccccc5c24)cc1O3] 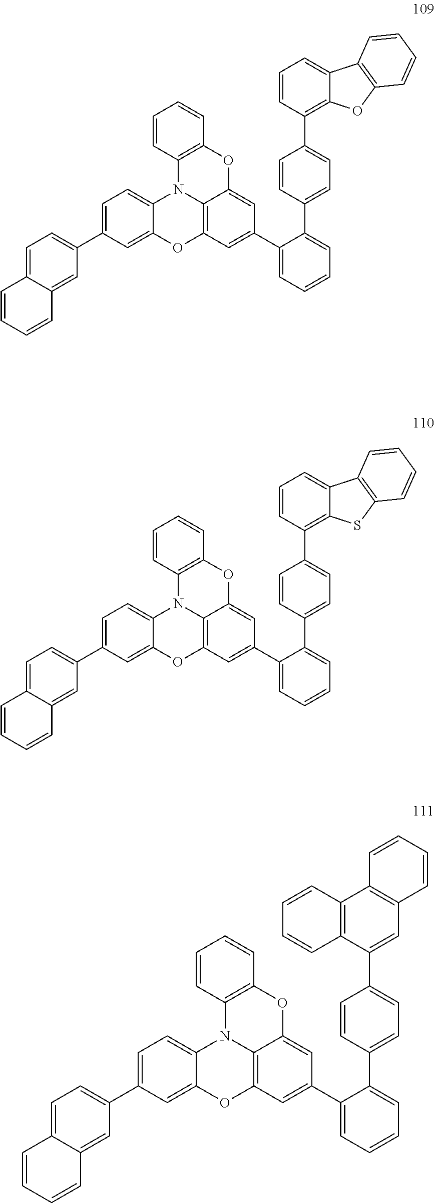 [SMILES: c1ccc2c(c1)Oc1cc(-c3ccccc3-c3ccc(-c4cccc5c4oc4ccccc45)cc3)cc3c1N2c1ccc(-c2ccc4ccccc4c2)cc1O3 ; c1ccc2c(c1)Oc1cc(-c3ccccc3-c3ccc(-c4cccc5c4sc4ccccc45)cc3)cc3c1N2c1ccc(-c2ccc4ccccc4c2)cc1O3 ; c1ccc2c(c1)Oc1cc(-c3ccccc3-c3ccc(-c4cc5ccccc5c5ccccc45)cc3)cc3c1N2c1ccc(-c2ccc4ccccc4c2)cc1O3] 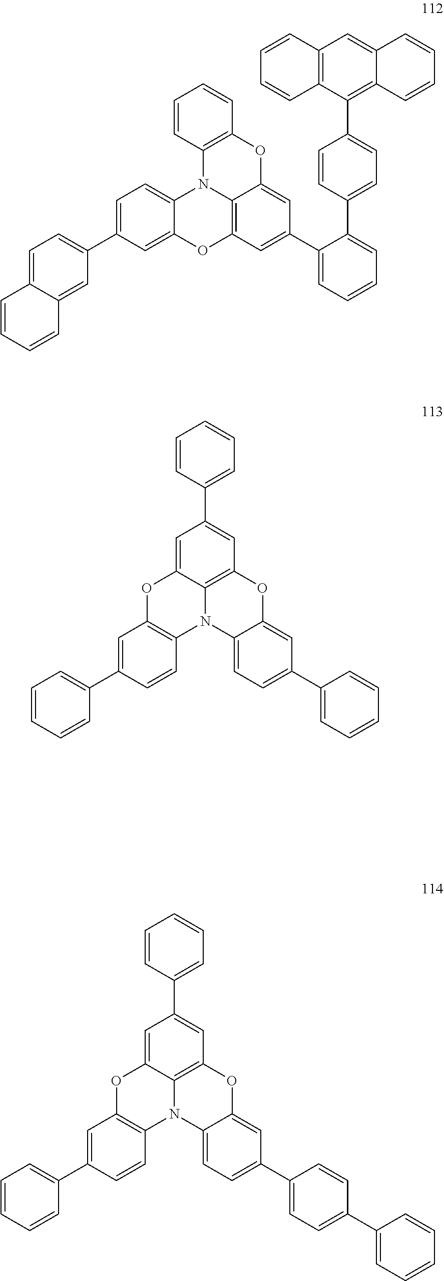 [SMILES: c1ccc2c(c1)Oc1cc(-c3ccccc3-c3ccc(-c4c5ccccc5cc5ccccc45)cc3)cc3c1N2c1ccc(-c2ccc4ccccc4c2)cc1O3 ; c1ccc(-c2ccc3c(c2)Oc2cc(-c4ccccc4)cc4c2N3c2ccc(-c3ccccc3)cc2O4)cc1 ; c1ccc(-c2ccc(-c3ccc4c(c3)Oc3cc(-c5ccccc5)cc5c3N4c3ccc(-c4ccccc4)cc3O5)cc2)cc1] 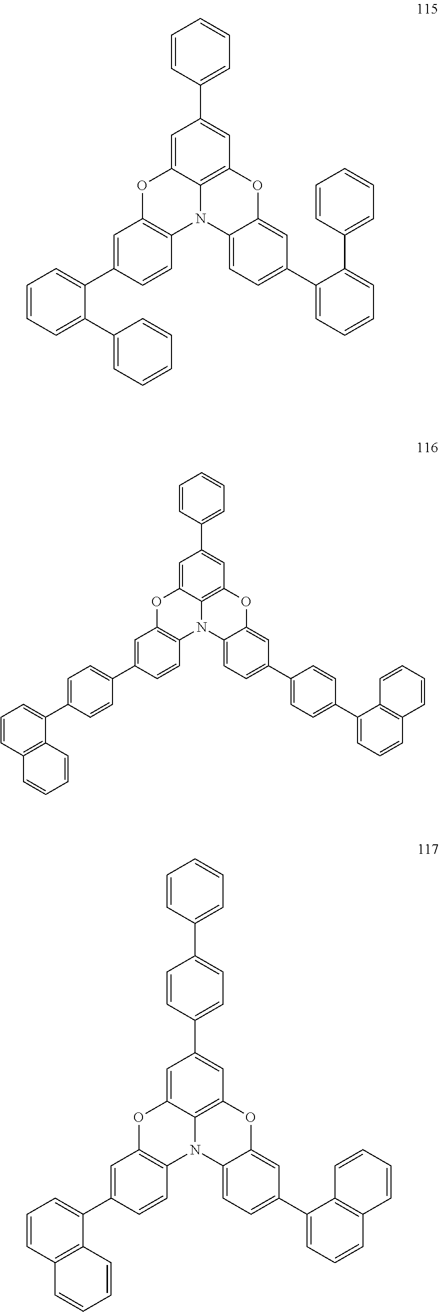 [SMILES: c1ccc(-c2cc3c4c(c2)Oc2cc(-c5ccccc5-c5ccccc5)ccc2N4c2ccc(-c4ccccc4-c4ccccc4)cc2O3)cc1 ; c1ccc(-c2cc3c4c(c2)Oc2cc(-c5ccc(-c6cccc7ccccc67)cc5)ccc2N4c2ccc(-c4ccc(-c5cccc6ccccc56)cc4)cc2O3)cc1 ; c1ccc(-c2ccc(-c3cc4c5c(c3)Oc3cc(-c6cccc7ccccc67)ccc3N5c3ccc(-c5cccc6ccccc56)cc3O4)cc2)cc1] 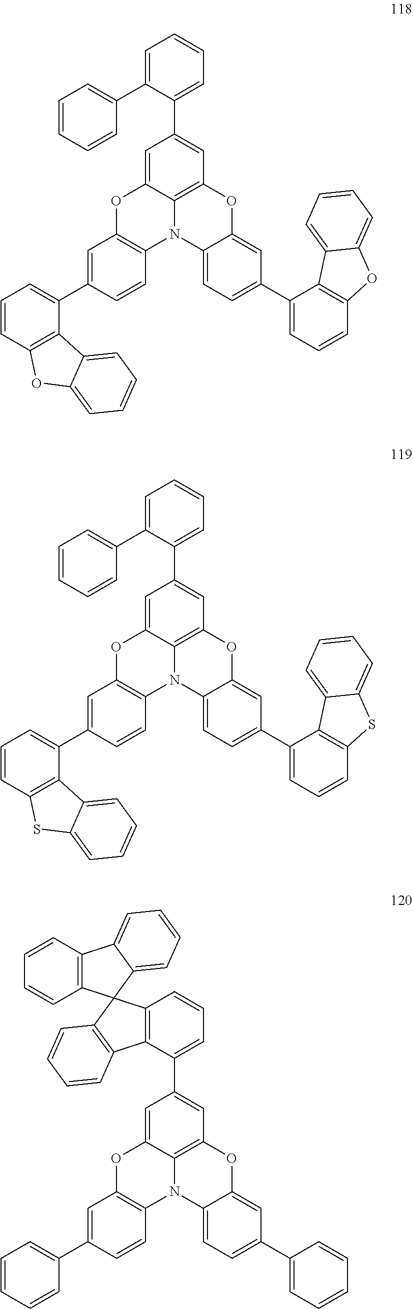 [SMILES: c1ccc(-c2ccccc2-c2cc3c4c(c2)Oc2cc(-c5cccc6oc7ccccc7c56)ccc2N4c2ccc(-c4cccc5oc6ccccc6c45)cc2O3)cc1 ; c1ccc(-c2ccccc2-c2cc3c4c(c2)Oc2cc(-c5cccc6sc7ccccc7c56)ccc2N4c2ccc(-c4cccc5sc6ccccc6c45)cc2O3)cc1 ; c1ccc(-c2ccc3c(c2)Oc2cc(-c4cccc5c4-c4ccccc4C54c5ccccc5-c5ccccc54)cc4c2N3c2ccc(-c3ccccc3)cc2O4)cc1] 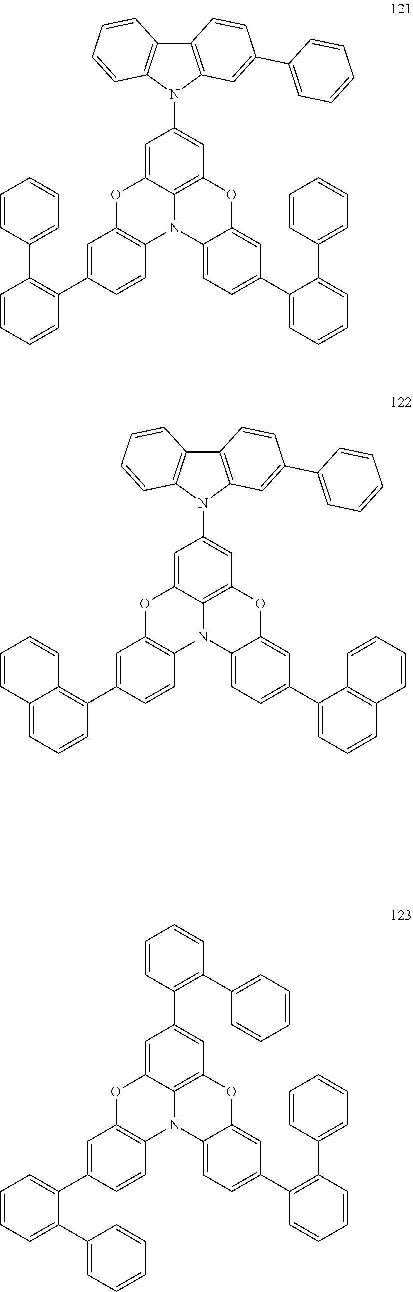 [SMILES: c1ccc(-c2ccc3c4ccccc4n(-c4cc5c6c(c4)Oc4cc(-c7ccccc7-c7ccccc7)ccc4N6c4ccc(-c6ccccc6-c6ccccc6)cc4O5)c3c2)cc1 ; c1ccc(-c2ccc3c4ccccc4n(-c4cc5c6c(c4)Oc4cc(-c7cccc8ccccc78)ccc4N6c4ccc(-c6cccc7ccccc67)cc4O5)c3c2)cc1 ; c1ccc(-c2ccccc2-c2ccc3c(c2)Oc2cc(-c4ccccc4-c4ccccc4)cc4c2N3c2ccc(-c3ccccc3-c3ccccc3)cc2O4)cc1] 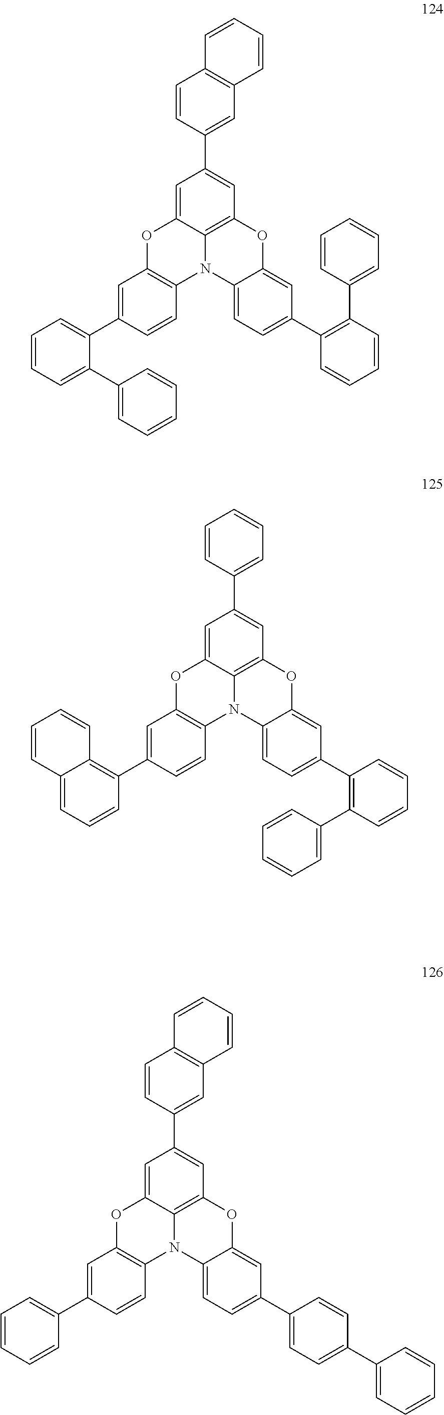 [SMILES: c1ccc(-c2ccccc2-c2ccc3c(c2)Oc2cc(-c4ccc5ccccc5c4)cc4c2N3c2ccc(-c3ccccc3-c3ccccc3)cc2O4)cc1 ; c1ccc(-c2cc3c4c(c2)Oc2cc(-c5cccc6ccccc56)ccc2N4c2ccc(-c4ccccc4-c4ccccc4)cc2O3)cc1 ; c1ccc(-c2ccc(-c3ccc4c(c3)Oc3cc(-c5ccc6ccccc6c5)cc5c3N4c3ccc(-c4ccccc4)cc3O5)cc2)cc1] 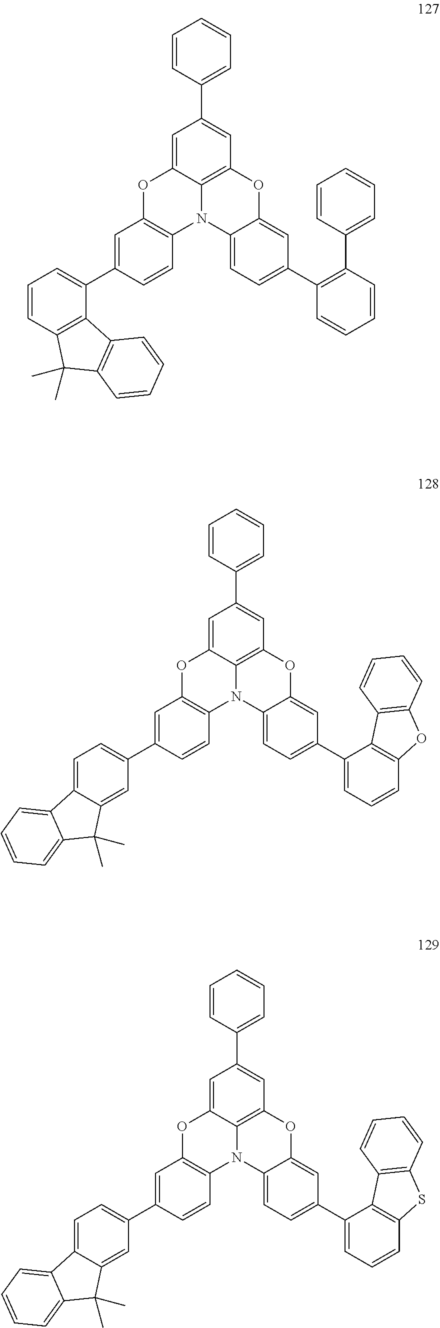 [SMILES: CC1(C)c2ccccc2-c2c(-c3ccc4c(c3)Oc3cc(-c5ccccc5)cc5c3N4c3ccc(-c4ccccc4-c4ccccc4)cc3O5)cccc21 ; CC1(C)c2ccccc2-c2ccc(-c3ccc4c(c3)Oc3cc(-c5ccccc5)cc5c3N4c3ccc(-c4cccc6oc7ccccc7c46)cc3O5)cc21 ; CC1(C)c2ccccc2-c2ccc(-c3ccc4c(c3)Oc3cc(-c5ccccc5)cc5c3N4c3ccc(-c4cccc6sc7ccccc7c46)cc3O5)cc21] 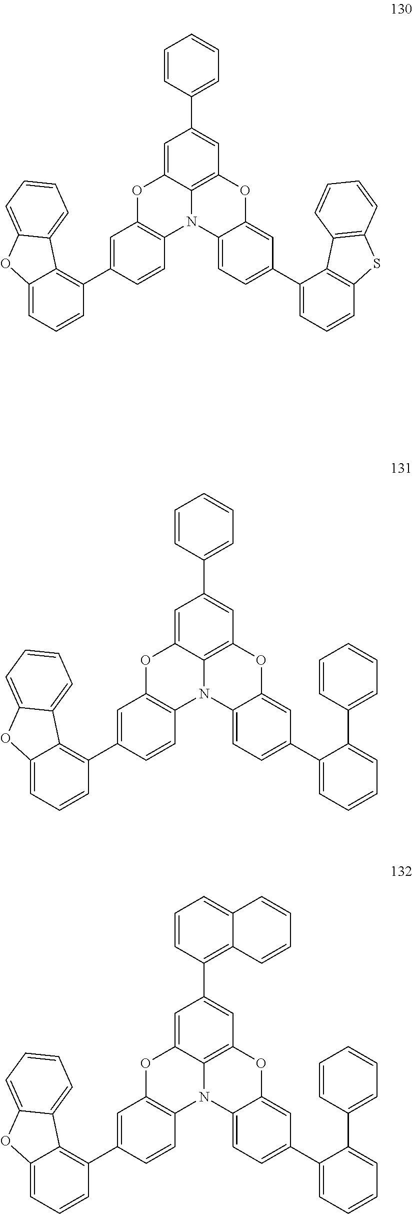 [SMILES: c1ccc(-c2cc3c4c(c2)Oc2cc(-c5cccc6sc7ccccc7c56)ccc2N4c2ccc(-c4cccc5oc6ccccc6c45)cc2O3)cc1 ; c1ccc(-c2cc3c4c(c2)Oc2cc(-c5cccc6oc7ccccc7c56)ccc2N4c2ccc(-c4ccccc4-c4ccccc4)cc2O3)cc1 ; c1ccc(-c2ccccc2-c2ccc3c(c2)Oc2cc(-c4cccc5ccccc45)cc4c2N3c2ccc(-c3cccc5oc6ccccc6c35)cc2O4)cc1] 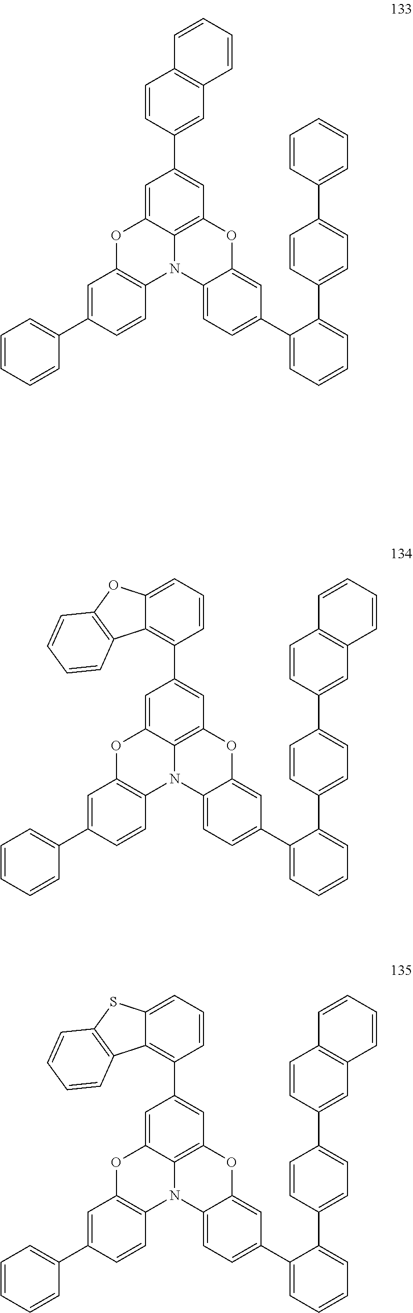 [SMILES: c1ccc(-c2ccc(-c3ccccc3-c3ccc4c(c3)Oc3cc(-c5ccc6ccccc6c5)cc5c3N4c3ccc(-c4ccccc4)cc3O5)cc2)cc1 ; c1ccc(-c2ccc3c(c2)Oc2cc(-c4cccc5oc6ccccc6c45)cc4c2N3c2ccc(-c3ccccc3-c3ccc(-c5ccc6ccccc6c5)cc3)cc2O4)cc1 ; c1ccc(-c2ccc3c(c2)Oc2cc(-c4cccc5sc6ccccc6c45)cc4c2N3c2ccc(-c3ccccc3-c3ccc(-c5ccc6ccccc6c5)cc3)cc2O4)cc1] 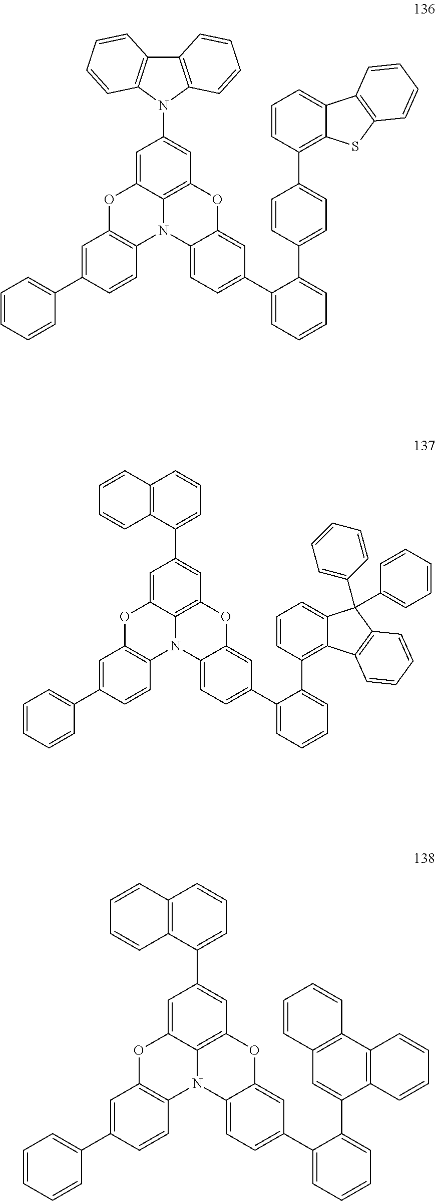 [SMILES: c1ccc(-c2ccc3c(c2)Oc2cc(-n4c5ccccc5c5ccccc54)cc4c2N3c2ccc(-c3ccccc3-c3ccc(-c5cccc6c5sc5ccccc56)cc3)cc2O4)cc1 ; c1ccc(-c2ccc3c(c2)Oc2cc(-c4cccc5ccccc45)cc4c2N3c2ccc(-c3ccccc3-c3cccc5c3-c3ccccc3C5(c3ccccc3)c3ccccc3)cc2O4)cc1 ; c1ccc(-c2ccc3c(c2)Oc2cc(-c4cccc5ccccc45)cc4c2N3c2ccc(-c3ccccc3-c3cc5ccccc5c5ccccc35)cc2O4)cc1] 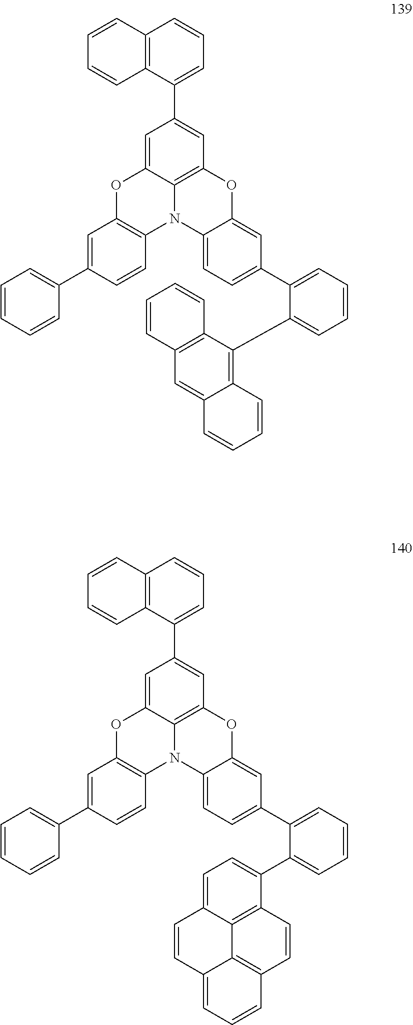 [SMILES: c1ccc(-c2ccc3c(c2)Oc2cc(-c4cccc5ccccc45)cc4c2N3c2ccc(-c3ccccc3-c3c5ccccc5cc5ccccc35)cc2O4)cc1 ; c1ccc(-c2ccc3c(c2)Oc2cc(-c4cccc5ccccc45)cc4c2N3c2ccc(-c3ccccc3-c3ccc5ccc6cccc7ccc3c5c67)cc2O4)cc1] 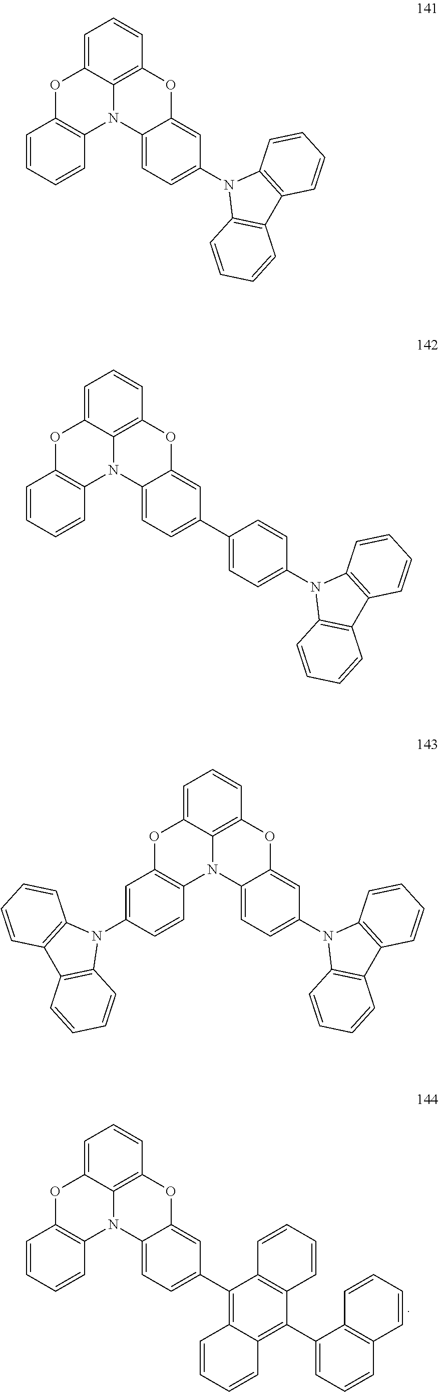 [SMILES: c1ccc2c(c1)Oc1cccc3c1N2c1ccc(-n2c4ccccc4c4ccccc42)cc1O3 ; c1ccc2c(c1)Oc1cccc3c1N2c1ccc(-c2ccc(-n4c5ccccc5c5ccccc54)cc2)cc1O3 ; c1cc2c3c(c1)Oc1cc(-n4c5ccccc5c5ccccc54)ccc1N3c1ccc(-n3c4ccccc4c4ccccc43)cc1O2 ; c1ccc2c(c1)Oc1cccc3c1N2c1ccc(-c2c4ccccc4c(-c4cccc5ccccc45)c4ccccc24)cc1O3]
